SEQUENCE LISTING (1 sequence)

SEQ ID NO: 1 - protein, 13 aa, synthetic construct
  SITE 1: note = X = penicillamine; note = N-terminal acetylation
  SITE 4: note = 7-methyltryptophan
  MOD_RES 5: note = Acetylation
  SITE 6: note = X = penicillamine
  DISULFID 1..6: note = Disulfide bond between sidechainds of penicillamine at position 1 and penicillamine at position 6
  SITE 7: note = X = 4-(2-aminoethoxy)-phenylalanine
  SITE 8: note = X = 2-napthyl-L-alanine
  SITE 9: note = X = 4-amino-tetrahydropyran-4-carboxylic acid
  SITE 13: note = X = MeGly
  SITE 12: note = X = 3-(3-pyridyl)-L-alanine
  MOD_RES 13: note = Amidation
XNTWKXXXXENXX

(19) United States
(12) Patent Application Publication
(10) Pub. No.: US 2026/0232767 A1
(43) Pub. Date: Aug. 13, 2026

(54) INTERLEUKIN-23 RECEPTOR ANTAGONIST PEPTIDES FOR USE IN THE TREATMENT OF PSORIASIS

(71) Applicant: Janssen Pharmaceutica NV, Beerse (BE)

(72) Inventors: Emily Bozenhardt, Garner, NC (US); Yu Kyoung Cho, Columbia, MO (US); Cynthia Marie Carver DeKlotz, Bethesda, MD (US); Beverly Knight, San Diego, CA (US); Teddy Kosoglou, Jamison, PA (US); Fabio P. Nunes, Zionsville, IN (US); David Polidori, Rancho Santa Fe, CA (US); Dawei Song, Chalfont, PA (US); Weirong Wang, Harleysville, PA (US); Yuan Xiong, Basking Ridge, NJ (US)

(21) Appl. No.: 19/160,372

(22) PCT Filed: Mar. 1, 2024
(86) PCT No.: PCT/US2024/018020

§ 371 Date: Aug. 27, 2025

Related U.S. Application Data
(60) Provisional application No. 63/488,419, filed on Mar. 3, 2023.
(60) Provisional application No. 63/511,837, filed on Jul. 3, 2023.
(60) Provisional application No. 63/589,553, filed on Oct. 11, 2023.

(51) Int. Cl. A61K 38/20; A61P 17/06

(52) U.S. Cl. CPC A61K 38/20; A61P 17/06

(57) ABSTRACT

The present invention relates to methods of administering a cyclic peptide inhibitor of the interleukin-23 receptor (IL-23R) or pharmaceutically acceptable salt or solvate forms thereof, corresponding compositions, assays, methods, and/or uses for treatment of psoriasis.

### CROSS-REFERENCE TO RELATED APPLICATIONS

This application claims priority to U.S. Provisional Patent Application Ser. No. 63/488,419, filed Mar. 3, 2023, U.S. Provisional Patent Application Ser. No. 63/511,837, filed Jul. 3, 2023, and U.S. Provisional Patent Application Ser. No. 63/589,553, filed Oct. 11, 2023, each of which is incorporated by reference herein in its entirety.

### REFERENCE TO SEQUENCE LISTING SUBMITTED AS AN XML FILE

This application contains a sequence listing, which is submitted electronically as an XML formatted sequence listing with a file name “PRD4263WOPCT1_Sequencelisting.xml”, creation date of Feb. 27, 2024, and size of 6,202 bytes. The sequence listing is part of the specification and is herein incorporated by reference in its entirety.

The present invention relates to methods of administering a peptide inhibitor of the interleukin-23 receptor (IL-23R), or a pharmaceutically acceptable salt or solvate form thereof, and other corresponding assays, methods and/or uses for treatment of psoriasis.

### BACKGROUND

Psoriasis, a chronic skin disease affecting about 2%-3% of the general population, has been shown to be mediated by the body's T cell inflammatory response mechanisms. IL-23 is one of several interleukins implicated as a key player in the pathogenesis of psoriasis, purportedly by maintaining chronic autoimmune inflammation via the induction of interleukin-17, regulation of T memory cells, and activation of macrophages. Expression of IL-23 and the IL-23 receptor (IL-23R) has been shown to be increased in tissues of patients with psoriasis, and antibodies that neutralize IL-23 showed IL-23-dependent inhibition of psoriasis development in animal models of psoriasis.

IL-23 is a heterodimer composed of a unique p19 subunit and the p40 subunit shared with IL-12, which is a cytokine involved in the development of interferon-γ (IFN-γ)-producing T helper 1 (T_{H}1) cells. Although IL-23 and IL-12 both contain the p40 subunit, they have different phenotypic properties. For example, animals that are deficient in IL-12 are susceptible to inflammatory autoimmune diseases, whereas IL-23 deficient animals are resistant to such diseases, presumably due to a reduced number of CD4⁺ T cells producing IL-6, IL-17, and TNF in the CNS of IL-23-deficient animals. IL-23 binds to IL-23R. Binding of IL-23 to IL-23R activates the Jak-Stat signaling molecules, Jak2, Tyk2, and Stat1, Stat 3, Stat 4, and Stat 5, although Stat 4 activation is substantially weaker. In addition, different DNA-binding Stat complexes form in response to IL-23 as compared with IL-12. IL-23R associates constitutively with Jak2 and in a ligand-dependent manner with Stat 3. In contrast to IL-12, which acts mainly on naive CD4(+) T cells, IL-23 preferentially acts on memory CD4(+) T cells.

According to the World Psoriasis Day consortium, 125 million people worldwide, about 2 to 3 percent of the total population have psoriasis. In the United States, it is estimated that more than 8 million Americans suffer from this disease. There is a need for an effective treatment, specifically oral treatment, for psoriasis patients.

### SUMMARY

The present invention addresses these needs by providing peptide inhibitors or pharmaceutically acceptable salt or solvate forms thereof which are suitable for oral administration that bind IL-23R to inhibit IL-23 binding and signaling. Some embodiments relate to a method for treating psoriasis in a subject in need thereof, comprising administering an IL-23 receptor antagonist peptide, wherein after treating with the IL-23 receptor antagonist peptide, the subject is a responder to treatment by at least one measure of response to treatment selected from the group consisting of (i) a 75% or higher reduction in Psoriasis Area and Severity Index (PASI) compared to a baseline PASI (PASI 75), (ii) an Investigator's Global Assessment (IGA) Score of 2 or lower after treatment, (iii) reduction from baseline in Psoriasis Symptoms and Signs Diary (PSSD) Symptoms Score by 1 or higher, and (iv) a reduction of Dermatological Life Quality Index (DLQI) score.

Some embodiments relate to a method for treating psoriasis in a subject in need thereof, comprising administering a compound of Formula (I):

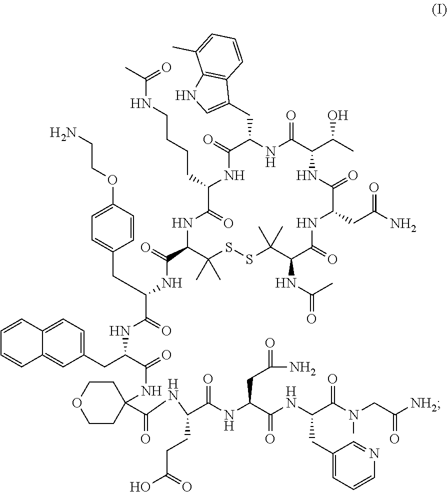 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to a patient in need thereof; wherein after treating with the IL-23 receptor antagonist peptide, the subject is a responder to treatment by at least one measure of response to treatment selected from the group consisting of (i) a 75% or higher reduction in Psoriasis Area and Severity Index (PASI) compared to a baseline PASI, (ii) an Investigator's Global Assessment (IGA) Score of 2 or lower after treatment, (iii) reduction from baseline in Psoriasis Symptoms and Signs Diary (PSSD) Symptoms Score by 1 or higher, and (iv) a reduction of Dermatological Life Quality Index (DLQI) score.

Some embodiments relate to a method for treating psoriasis in a subject in need thereof, comprising administering a compound of Formula (I) or a pharmaceutically acceptable salt or solvate thereof, to a patient in need thereof; wherein after treating with the IL-23 receptor antagonist peptide, the subject is a responder to treatment by achieving a 90% or higher reduction in Psoriasis Area and Severity Index (PASI) compared to a baseline PASI (PASI 90).

Some embodiments relate to a method for treating psoriasis in a subject in need thereof, comprising orally administering a compound of Formula (I) or a pharmaceutically acceptable salt or solvate thereof, to a patient in need thereof; wherein after treating with the IL-23 receptor antagonist peptide, the subject is a responder to treatment by achieving a 90% or higher reduction in Psoriasis Area and Severity Index (PASI) compared to a baseline PASI (PASI 90).

Some embodiments relate to a method for treating psoriasis in a subject in need thereof, comprising orally administering to the subject about 200 mg of a compound of Formula (I) or a pharmaceutically acceptable salt or solvate thereof. In some embodiments, the subject is a human diagnosed with moderate to severe plaque psoriasis.

Some embodiments relate to a method for treating psoriasis in a subject in need thereof, comprising orally administering a compound of Formula (I)

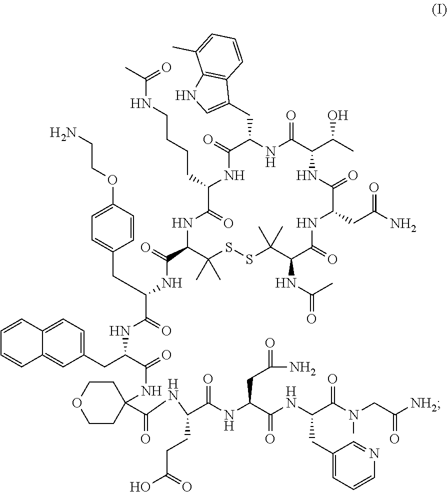 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to a patient in need thereof; wherein after treating with the IL-23 receptor antagonist peptide, the subject is a responder to treatment by at least one measure of response to treatment selected from the group consisting of (i) a 90% or higher reduction in Psoriasis Area and Severity Index (PASI) compared to a baseline PASI (PASI 90), and (ii) an Investigator's Global Assessment (IGA) Score of 1 or lower after treatment and >2-grade improvement from baseline IGA.

Some embodiments relate to a method for treating psoriasis in a subject in need thereof, comprising administering a compound of Formula (I):

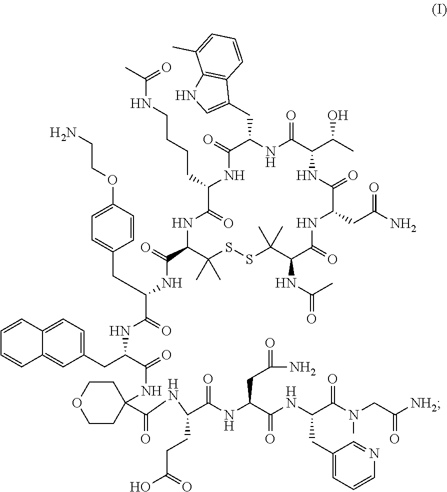 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to a patient in need thereof; wherein after treating with the IL-23 receptor antagonist peptide, the subject is a responder to treatment by at least one measure of response to treatment selected from the group consisting of (i) a 90% or higher reduction in Psoriasis Area and Severity Index (PASI) compared to a baseline PASI (PASI 90), and (ii) an Investigator's Global Assessment (IGA) Score of 1 or lower after treatment and ≥2-grade improvement from baseline IGA.

Some embodiments relate to a method of treating psoriasis in a subject in need thereof, comprising administering a compound of Formula (I) or a pharmaceutically acceptable salt or solvate thereof, wherein the subject achieves a 75% or higher reduction in Psoriasis Area and Severity Index (PASI) score compared to a baseline PASI score (PASI 75).

In some embodiments, the subject archives a scalp specific IGA (ss-IGA) score of 0 or 1. In some embodiments, the subject archives a scalp specific IGA (ss-IGA) score of 0. In some embodiments, the subject archives≥2-grade improvement from baseline ss-IGA. In some embodiments, the subject achieves at least about a 90% reduction in PASI score compared to the baseline PASI score. In some embodiments, the subject achieves about 100% reduction in PASI score compared to the baseline PASI score. In some embodiments, the subject achieves an Investigator's Global Assessment (IGA) Score of 2 or lower. In some embodiments, the subject achieves an IGA score of 1 or lower. In some embodiments, the subject achieves an IGA score of 0. In some embodiments, the patient achieves an Investigator's Global Assessment (IGA) Score of 2 or lower after treatment. In some embodiments, the patient achieves an IGA score of 1 or lower after treatment. In some embodiments, the patient achieves an IGA score of 0 after treatment. In some embodiments, the reduction in PASI score is measured at least 2 weeks after the beginning of treatment. In some embodiments, the psoriasis clinical endpoint is measured at least 4 weeks after the beginning of treatment. In some embodiments, the psoriasis clinical endpoint is measured at least 8 weeks after the beginning of treatment. In some embodiments, the psoriasis clinical endpoint is measured at least 16 weeks after the beginning of treatment. In some embodiments, the psoriasis clinical endpoint is measured at least 24 weeks after the beginning of treatment.

In some embodiments, the measure of response to treatment includes one or more selected from the group consisting of scalp specific IGA (ss-IGA), s-PGA (Static Physician's Global Assessment of Genitalia), Physician's Global Assessment of Hand and/or Feet (hf-PGA), Fingernail Physician's Global Assessment (f-PGA), and Nail Psoriasis Area and Severity Index (NAPSI). In some embodiments, the measure of response to treatment includes scalp specific IGA.

In some embodiments, the psoriasis is plaque psoriasis. In some embodiments, the psoriasis is moderate to severe plaque psoriasis. In some embodiments, the subject is a candidate for phototherapy or systematic therapy.

In some embodiments, the compound of Formula (I) or pharmaceutically acceptable salt or solvate thereof is a hydrochloride salt of the compound of Formula (I) or solvate thereof. In some embodiments, the treatment comprises orally administering the compound of Formula (I) or pharmaceutically acceptable salt or solvate thereof. In some embodiments, the treatment comprises administering the compound of Formula (I) or pharmaceutically acceptable salt or solvate thereof daily.

In some embodiments, the treatment comprises administering the compound of Formula (I) or pharmaceutically acceptable salt or solvate thereof in an amount of about 5 mg to about 800 mg daily. In some embodiments, the treatment comprises administering the compound of Formula (I) or pharmaceutically acceptable salt or solvate thereof in an amount of about 25 mg to about 500 mg daily. In some embodiments, the treatment comprises administering the compound of Formula (I) or pharmaceutically acceptable salt or solvate thereof in an amount of about 50 mg to about 500 mg daily. In some embodiments, the treatment comprises administering the compound of Formula (I) or pharmaceutically acceptable salt or solvate thereof in an amount of about 25 mg daily. In some embodiments, the treatment comprises administering the compound of Formula (I) or pharmaceutically acceptable salt or solvate thereof in an amount of about 50 mg daily. In some embodiments, the treatment comprises administering the compound of Formula (I) or pharmaceutically acceptable salt or solvate thereof in an amount of about 100 mg daily. In some embodiments, the treatment comprises administering the compound of Formula (I) or pharmaceutically acceptable salt or solvate thereof in an amount of about 200 mg daily.

In some embodiments, the patient has a Body Surface Area (BSA) of at least 10% prior to treatment. In some embodiments, the patient has a PASI score of about 12 to 72 prior to treatment. In some embodiments, the subject has an Investigator's Global Assessment (IGA) of at least 3 prior to treatment. In some embodiments, the subject has a Psoriasis Symptom and Sign Diary (PSSD) symptoms score of at least 1 prior to treatment. In some embodiments, the subject has a Dermatological Life Quality Index (DLQI) score of greater than 1 prior to treatment. In some embodiments, before the start of treatment with the compound of Formula (I) or pharmaceutically acceptable salt or solvate thereof, the subject has not been treated with a biologic agent for psoriasis. In some embodiments, before the start of treatment with the compound of Formula (I) or pharmaceutically acceptable salt or solvate thereof, the subject has not been treated with any IL-23 receptor antagonist peptide for psoriasis.

Some embodiments relate to a method of treating a patient population diagnosed with psoriasis, comprising administering a compound of Formula (I):

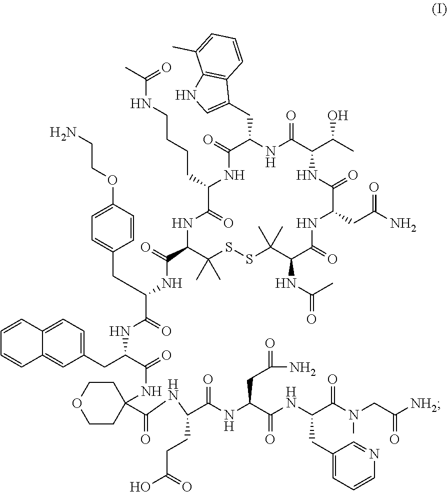 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to said patient population, wherein a proportion of the patient population responds to treatment by at least one measure of response to treatment selected from the group consisting of (i) a 75% or higher reduction in Psoriasis Area and Severity Index (PASI) compared to a baseline PASI (PASI75), (ii) an Investigator's Global Assessment (IGA) Score of 2 or lower after treatment, (iii) reduction from baseline in Psoriasis Symptoms and Signs Diary (PSSD) Symptoms Score by 1 or higher, and (iv) a reduction of Dermatological Life Quality Index (DLQI) score.

Some embodiments relate to a method of treating a patient population diagnosed with psoriasis, comprising administering a compound of Formula (I):

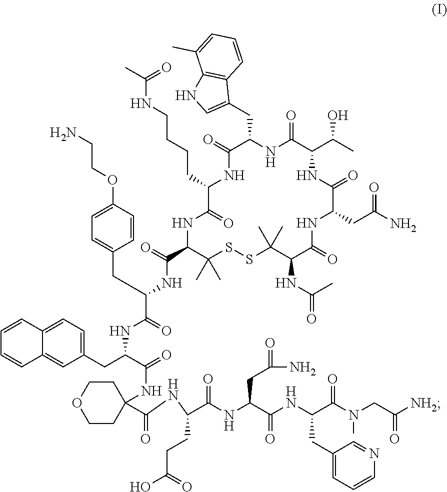 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to said patient population, wherein about 50% or higher of the patient population responds to treatment as measured by a 75% or higher reduction in Psoriasis Area and Severity Index (PASI) compared to a baseline PASI (PASI 75).

In some embodiments, the administration of the compound of Formula (I) or a pharmaceutically acceptable salt or solvate thereof, provides a reduction in Psoriasis Area and Severity Index score compared to the baseline relative to a patient population that has been administered placebo. In some embodiments, the patient population has an average response rate of about 25% or greater to treatment as measured by a 90% reduction in PASI score compared to the baseline PASI score (PASI 90). In some embodiments, the patient population has an average response rate of about 10% or greater to treatment as measured by a 100% reduction in PASI score compared to the baseline PASI score (PASI 100). In some embodiments, the patient population has an average response rate of about 39% or greater to treatment as measured by achieving an Investigator's Global Assessment (IGA) Score of 1 or lower after treatment. In some embodiments, the patient population has an average response rate of about 15% or greater to treatment as measured by achieving an Investigator's Global Assessment (IGA) Score of 0 after treatment. In some embodiments, the psoriasis clinical endpoint is measured at least 16 weeks after the beginning of treatment.

In some embodiments, about 25% or greater of the patient population responds to treatment as measured by a 90% reduction in PASI score compared to the baseline PASI score. In some embodiments, about 10% or greater of the patient population responds to treatment as measured by a 100% reduction in PASI score compared to the baseline PASI score. In some embodiments, about 39% or greater of the patient population responds to treatment as measured by achieving an Investigator's Global Assessment (IGA) Score of 1 or lower after treatment. In some embodiments, about 15% or greater of the patient population responds to treatment as measured by achieving an Investigator's Global Assessment (IGA) Score of 0 after treatment.

Some embodiments relate to a pharmaceutical product for treating a subject diagnosed with psoriasis, comprising a compound of Formula (I):

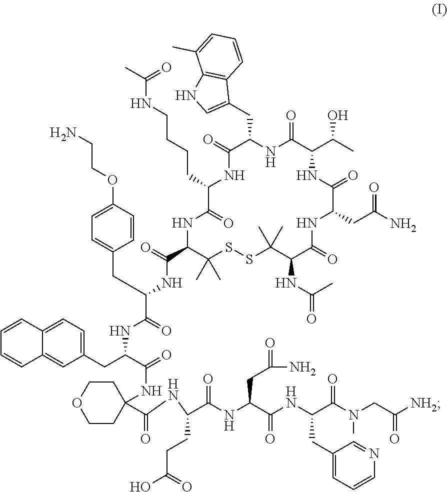 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, that when administered to said subject, in an amount of at least 50 mg, induces a mean reduction in the Psoriasis Area and Severity Index (PASI) by about 75% or greater, when measured from a baseline PASI score.

In some embodiments, the subject achieves at least about a 90% reduction in Psoriasis Area and Severity Index score compared to the baseline. In some embodiments, the subject achieves about 100% reduction in Psoriasis Area and Severity Index score compared to the baseline. In some embodiments, the patient achieves an Investigator's Global Assessment (IGA) Score of 2 or lower after treatment. In some embodiments, the compound of Formula (I) or pharmaceutically acceptable salt or solvate thereof is in an amount of about 5 mg to about 800 mg. In some embodiments, the compound of Formula (I) or pharmaceutically acceptable salt or solvate thereof is in an amount of about 25 mg to about 500 mg. In some embodiments, the compound of Formula (I) or pharmaceutically acceptable salt or solvate thereof is in an amount of about 50 mg to about 500 mg. In some embodiments, the compound of Formula (I) or pharmaceutically acceptable salt or solvate thereof is in an amount of about 25 mg. In some embodiments, the compound of Formula (I) or pharmaceutically acceptable salt or solvate thereof is in an amount of about 50 mg. In some embodiments, the compound of Formula (I) or pharmaceutically acceptable salt or solvate thereof is in an amount of about 100 mg.

In some embodiments, the compound of Formula (I) or pharmaceutically acceptable salt or solvate thereof is in an amount of about 5 mg to about 800 mg daily. In some embodiments, the compound of Formula (I) or pharmaceutically acceptable salt or solvate thereof is in an amount of about 25 mg to about 500 mg daily. In some embodiments, the compound of Formula (I) or pharmaceutically acceptable salt or solvate thereof is in an amount of about 50 mg to about 500 mg daily. In some embodiments, the compound of Formula (I) or pharmaceutically acceptable salt or solvate thereof is in an amount of about 25 mg daily. In some embodiments, the compound of Formula (I) or pharmaceutically acceptable salt or solvate thereof is in an amount of about 50 mg daily. In some embodiments, the compound of Formula (I) or pharmaceutically acceptable salt or solvate thereof is in an amount of about 100 mg daily. In some embodiments, the compound of Formula (I) or pharmaceutically acceptable salt or solvate thereof is administered once daily. In some embodiments, the compound of Formula (I) or pharmaceutically acceptable salt or solvate thereof is administered twice daily. In some embodiments, the compound of Formula (I) or pharmaceutically acceptable salt or solvate thereof is administered in a fasted state.

In some embodiments, the present invention relates to a method and/or use for treating psoriasis, which comprises administering a composition that includes:
(a) a compound of Formula (I):

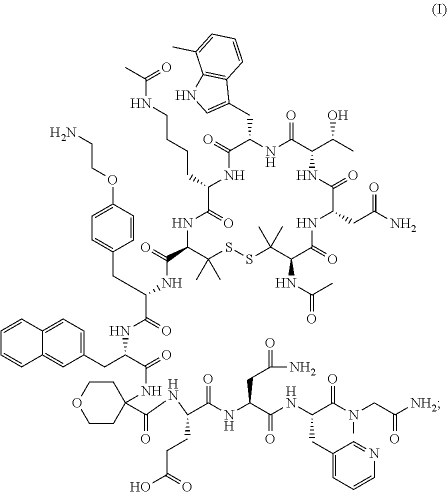 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof; and
(b) one or more pharmaceutically acceptable excipients;
to a subject in need thereof;
where the composition is administered to the subject for at least 16 weeks.

In some embodiments, the present invention relates to a method and/or use for treating psoriasis, which comprises administering a composition that includes:
(a) a compound of Formula (I):

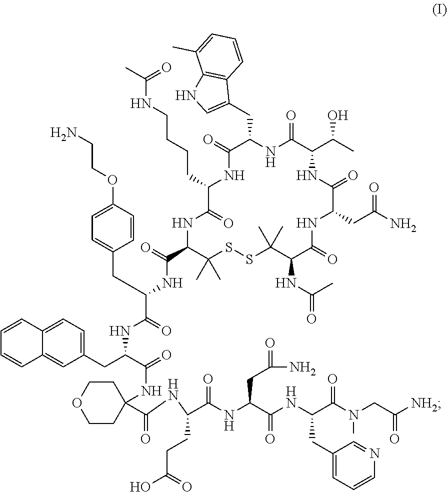 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof; and
(b) one or more pharmaceutically acceptable excipients;
to a subject in need thereof;
where the composition is administered to the subject for at least 52 weeks.

In some embodiments, the present invention relates to a method and/or use for treating moderate to severe plaque psoriasis, which comprises orally administering daily a composition that includes: (a) from about 25 mg to about 500 mg of a hydrochloride salt of a compound of Formula (I); or a solvate thereof; and (b) one or more pharmaceutically acceptable excipients; to a subject in need thereof; where the subject is a candidate for phototherapy or systemic therapy; and the hydrochloride salt of a compound of Formula (I) or solvate thereof is administered to the subject daily for at least 16 weeks.

In some embodiments, the present invention relates to a method and/or use for treating moderate to severe plaque psoriasis, which comprises orally administering daily a composition that includes: (a) from about 25 mg to about 500 mg of a hydrochloride salt of a compound of Formula (I); or a solvate thereof; and (b) one or more pharmaceutically acceptable excipients; to a subject in need thereof; where the subject is a candidate for phototherapy or systemic therapy; and the hydrochloride salt of a compound of Formula (I) or solvate thereof is administered to the subject daily for at least 52 weeks.

### BRIEF DESCRIPTION OF THE DRAWINGS

FIG. 1 shows a schematic overview of the study protocol of Example 1. R=randomization; BID=twice daily; QD=once daily; LTE^{a}=Long Term Extension; ^{b}All ineligible subjects for the LTE and eligible subjects for the LTW who choose not to enroll in the LTE complete 4 weeks of safety follow-up to complete the study; ° Final safety follow-up for subjects who do not enter the LTE.

FIG. 2 shows the results of patients achieving PASI 75 response at Week 16.

FIG. 3A shows the results of patients achieving PASI 90 response at Week 16.

FIG. 3B shows the results of patients achieving PASI 100 response at Week 16.

FIG. 3C shows the results of patients achieving IGA0/1 response at Week 16.

FIG. 3D shows the results of patients achieving IGA 0 response at Week 16.

FIG. 4 shows PAST responses over time through 16 weeks.

FIG. 5 shows the IL-23 receptor antagonist peptide median trough plasma concentration (ng/mL) through Week 16.

FIG. 6A depicts the median trough plasma concentration (ng/mL) of subjects receiving different doses of IL-23 receptor antagonist peptide through Week 16 by Baseline Body Weight with baseline body weight≤median.

FIG. 6B depicts the median trough plasma concentration (ng/mL) of subjects receiving different doses of IL-23 receptor antagonist peptide through Week 16 by Baseline Body Weight with baseline body weight≥median.

FIG. 7 shows the percent of subjects achieving PASI 75, 90, and 100 responses at Week 16 varied by IL-23 receptor antagonist peptide trough plasma concentration (ng/mL).

FIG. 8 shows the percent of subjects achieving IGA score 0 or 1 and 0 at Week 16 varied by IL-23 receptor antagonist peptide trough plasma concentration (ng/mL).

FIG. 9 shows the percent of subjects achieving PASI 75, 90, and 100 responses at Week 16 varied by IL-23 receptor antagonist peptide intermediate plasma concentration (ng/mL).

FIG. 10 shows the percent of subjects achieving IGA score 0 or 1 and IGA 0 at Week 16 varied by IL-23 receptor antagonist peptide intermediate plasma concentration (ng/mL).

FIG. 11 shows the percent of subjects achieving PASI 75, 90, and 100 responses at Week 16 varied by IL-23 receptor antagonist peptide peak plasma concentration (ng/mL).

FIG. 12 shows the percent of subjects achieving IGA score 0 or 1 and 0 at Week 16 varied by IL-23 receptor antagonist peptide peak plasma concentration (ng/mL).

FIG. 13 shows the decrease in BD-2 serum levels over time for varying doses of the IL-23 receptor antagonist peptide. *: p<0.05 for all doses vs placebo at all time points; † p<0.05 100 mg BID vs other active dose arms at that time point.

FIG. 14 shows the decrease in IL-22 serum levels over time for varying doses of the IL-23 receptor antagonist peptide. *: p<0.05 for all doses vs placebo at all time points; †, p<0.05 100 mg BID vs other active dose arms at that time point.

FIG. 15 shows the decrease in IL-17A serum levels over time for varying doses of the IL-23 receptor antagonist peptide. *: p<0.05 for all doses vs placebo at all time points.

FIG. 16 shows the decrease in IL-17F serum levels over time for varying doses of the IL-23 receptor antagonist peptide. *: p<0.05 for all doses vs placebo at all time points.

FIG. 17 shows the response rates for scalp psoriasis for all doses were higher than placebo at Week 16. Scalp psoriasis response was determined as at least a 2-grade improvement from baseline and an ss-IGA score of 0 or 1 (left) or at least a 2-grade improvement from baseline and an ss-IGA score of 0 (right).

FIG. 18 shows the BD-2 serum level response in various dose groups. *: p<0.05 for all doses vs placebo at all time points; t: p<0.05 100 mg BID vs other active dose arms at that time point.

FIG. 19 shows the IL22 serum level response in various dose groups. *: p<0.05 for all doses vs placebo at all time points; †: p<0.05 100 mg BID vs other active dose arms at that time point.

FIG. 20A shows the IL-17A serum level responses in various dose groups. *: p<0.05 for all doses vs placebo at all time points.

FIG. 20B shows the IL-17F serum level responses in various dose groups. *: p<0.05 for all doses vs placebo at all time points

FIG. 21 shows a schematic overview of the study protocol of Example 2, with subjects that completed Example 1 (Week 0-16) continuing for an additional 36 weeks of treatment followed by 4 weeks of safety follow-up. Subjects that were randomized to receive the placebo in Example 1 were transitioned to 100 mg QD at Week 16. BID=twice daily; QD=once daily.

FIG. 22A depicts representative percentages of subjects achieving PASI 75 responses over time through Week 52.

FIG. 22B depicts representative percentages of subjects achieving PASI 90 responses over time through Week 52 of treatment.

FIG. 22C depicts representative percentages of subjects achieving PASI 100 responses over time through Week 52 of treatment.

FIG. 22D depicts representative percentages of subjects achieving an IGA score of 1 or 0 over time through Week 52 of treatment.

FIG. 22E depicts representative percentages of subjects achieving an IGA score 0 over time through Week 52 of treatment.

FIG. 23A depicts representative percentages of subjects achieving PASI 90 after Week 16 of treatment (left) and Week 52 of treatment (right).

FIG. 23B depicts representative percentages of subjects achieving PASI 100 after Week 16 of treatment (left) and Week 52 of treatment (right).

FIG. 23C depicts representative percentages of subjects achieving an IGA score of 1 or 0 after Week 16 of treatment (left) and Week 52 of treatment (right).

FIG. 23D depicts representative percentages of subjects achieving an IGA score of 0 after Week 16 of treatment (left) and Week 52 of treatment (right).

FIG. 24 depicts representative change from baseline in PASI score over 52 weeks of treatment.

FIG. 25A depicts representative percentages of subjects achieving a PSSD symptoms score of 0 over 52 weeks of treatment.

FIG. 25B depicts representative percentages of subjects achieving a PSSD signs score of 0 over 52 weeks of treatment.

FIG. 26A depicts representative decreases from baseline in PSSD symptoms score over 52 weeks of treatment.

FIG. 26B depicts representative decreases from baseline in PSSD signs score over 52 weeks of treatment.

FIG. 27A depicts representative decreases from baseline in PSSD symptoms score after Week 16 of treatment (left) and Week 52 of treatment (right).

FIG. 27B depicts representative decreases from baseline in PSSD signs score after Week 16 of treatment (left) and Week 52 of treatment (right).

FIG. 28 shows a schematic overview of the study protocol of Example 3. R=randomization; PE=primary endpoint; Q12w=every 12 weeks; SE=secondary endpoint; SFU=safety follow-up visit.

FIG. 29 shows a representative exposure-response model for PASI 75 response rate versus C_{trough} at Week 16. Cₜᵣ is serum trough concentration at steady state. The solid line represents model-predicted response rate, the dashed line represents predicted median exposure metrics (Cₜᵣ=0.469 ng/mL) of 200 mg QD based on a model from 1,000 simulated subjects similar to those from Example 1. Horizontal bars represent 25^{th}-75^{th} percentile of pharmacokinetic (PK) post hoc exposure metrics of each dosing regimen. The boxes and whiskers represent the observed, placebo-corrected, response rates and corresponding 95% confidence intervals.

FIG. 30 shows a representative exposure-response model for PASI 75 response rate versus C_{avg} at Week 16. C_{avg} is serum trough concentration at steady state. The solid line represents model-predicted response rate, the dashed line represents predicted median exposure metrics (Cₜᵣ1.36 ng/mL) of 200 mg QD based on a model from 1,000 simulated subjects similar to those from Example 1. Horizontal bars represent 25^{th}-75^{th} percentile of pharmacokinetic (PK) post hoc exposure metrics of each dosing regimen. The boxes and whiskers represent the observed, placebo-corrected, response rates and corresponding 95% confidence intervals.

FIG. 31 shows a representative exposure-response model for IGA 0/1 response rate versus C_{trough} at Week 16. The solid line represents model-predicted response rate, the dashed line represents predicted median exposure metrics (Cₜᵣ=0.469 ng/mL) of 200 mg QD based on a model from 1,000 simulated subjects similar to those from Example 1. Horizontal bars represent 25^{th}-75^{th} percentile of pharmacokinetic (PK) post hoc exposure metrics of each dosing regimen. The boxes and whiskers represent the observed, placebo-corrected, response rates and corresponding 95% confidence intervals.

FIG. 32 shows a representative exposure-response model for IGA 0/1 response rate versus C_{avg} at Week 16. The solid line represents model-predicted response rate, the dashed line represents predicted median exposure metrics (Cₜᵣ1.36 ng/mL) of 200 mg QD based on a model from 1,000 simulated subjects similar to those from Example 1. Horizontal bars represent 25^{th}-75^{th} percentile of pharmacokinetic (PK) post hoc exposure metrics of each dosing regimen. The boxes and whiskers represent the observed, placebo-corrected, response rates and corresponding 95% confidence intervals.

FIG. 33 shows an XRPD pattern of a crystalline form of a hydrochloride salt of a compound of Formula (I).

### DETAILED DESCRIPTION

The invention herein, provides an effective treatment of psoriasis using an oral IL-23 receptor antagonist. The IL-23 receptor antagonist, as described below and supported by clinical trial studies, provides a new and effective therapy for psoriasis patients, with both superior efficacy, ease of administration, improved patient adherence, low anti-drug antibody (ADA) risk, and a better safety profile.

“A,” “an,” or “a(n)”, is an indefinite article when used in reference to a group of substituents or “substituent group” herein, mean at least one.

“About” when referring to a value includes the stated value+/−10% of the stated value. For example, about 50% includes a range of from 45% to 55%, while about 20 molar equivalents includes a range of from 18 to 22 molar equivalents. Accordingly, when referring to a range, “about” refers to each of the stated values+/−10% of the stated value of each end of the range. For instance, a ratio of from about 1 to about 3 (weight/weight) includes a range of from 0.9 to 3.3.

“Administering” refers to administration of the composition of the present invention to a subject.

“Composition” as used herein is intended to encompass a product that includes the specified active product ingredient (API) (i.e., as defined in the instant specification as a compound of Formula (I) or a pharmaceutically acceptable salt or solvate thereof) and pharmaceutically acceptable excipients, carriers or diluents as described herein, which results from combination of specific components.

An “empty stomach” or “fasted” state refers to abstaining from food intake for at least 2 hours before administration of the composition of the present invention and abstaining from food and liquid intake for at least 30 minutes after administration of the composition of the present invention.

A “PASI score” refers to a numerical value resulting from evaluation using the Psoriasis Area and Severity Index (PASI). PASI is a system used for assessing and grading the severity of psoriatic lesions and their response to therapy.

As used herein, a “PASI xx response” indicates a greater than or equal to xx % reduction in Psoriasis Area and Severity Index (PASI) score after treatment as compared to before treatment. In an illustrative example, a PASI 50 response in a subject treated with the composition of the present invention is a subject that has a greater than or equal to 50% reduction in PASI score after treatment. Hence, a subject having a PASI 72 score before treatment and having a PASI 36 score or lower after treatment is said to achieve a PASI 50 response.

“Patient” or “subject” refers to a living organism, which includes, but is not limited to a human subject suffering from or prone to a disease or condition that can be treated by administration of a pharmaceutical composition as provided herein. Further non-limiting examples may include, but are not limited to, humans or other mammals. In some embodiments, the subject is a human.

“Pharmaceutically acceptable excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals or as conventionally understood in or by skilled artisans who work in the formulary arts.

Pharmaceutically acceptable salts of the compounds of the present invention are salts formed with acids, such as of mineral acids, organic carboxylic and organic sulfonic acids, hydrochloric acid, methanesulfonic acid, maleic acid, are also possible provided a basic group, such as an amine, constitutes part of the structure.

“Salt” as used herein refers to acid or base salts of the compounds used in the methods of the present invention. Illustrative examples of pharmaceutically acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. It is understood that the pharmaceutically acceptable salts are non-toxic.

The compounds of the invention may form solvates, for example with water (i.e. hydrates) or common organic solvents. As used herein, the term “solvate” means a physical association of the compounds of the present invention with one or more solvent molecules. This physical association involves varying degrees of ionic and covalent bonding, including hydrogen bonding. In certain instances, the solvate will be capable of isolation, for example when one or more solvent molecules are incorporated in the crystal lattice of the crystalline solid. The term “solvate” is intended to encompass both solution-phase and isolatable solvates. Non-limiting examples of suitable solvates include compounds of the invention in combination with water, isopropanol, ethanol, methanol, dimethyl sulfoxide, ethyl acetate, acetic acid or ethanolamine and the like. The compounds of the invention may exert their biological effects when they are in solution. Solvates are well known in the pharmaceutical industry. They can be important to the process for the preparation of a substance (e.g. in relation to their purification, the storage of the substance (e.g. its stability) and the ease of handling the substance and are often formed as part of the isolation or purification stages of a chemical synthesis. A person skilled in the art can determine whether a hydrate or other solvate has formed by the isolation conditions or purification conditions used to prepare a given compound using techniques such as thermogravimetric analysis (TGA), differential scanning calorimetry (DSC), X-ray crystallography (e.g. single X-ray crystallography or X-ray powder diffraction) and Solid-State NMR (SS-NMR also known as Magic Angle Spinning NMR or MAS-NMR).

“Therapeutically effective amount” refers to an amount of a compound or of a pharmaceutical composition useful for treating or ameliorating an identified disease or condition, or for exhibiting a detectable therapeutic or inhibitory effect. “Therapeutically effective amount” further includes within its meaning a non-toxic but sufficient amount of the particular drug to which it is referring to provide the desired therapeutic effect. The exact amount required will vary from subject to subject depending on factors such as the patient's general health, the patient's age, etc. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Safe and effective amount” as used herein in the context of a dose, dosage regimen, treatment or method refer to the effectiveness of a particular dose, dosage, or treatment regimen. Efficacy can be measured based on change in the course of the disease in response to an agent of the present invention. For example, the IL-23 receptor antagonist of the present invention (e.g., the compound of formula (I) or a pharmaceutically acceptable salt or solvate thereof) is administered to a subject in an amount and for a time sufficient to induce an improvement, preferably a sustained improvement, in at least one indicator that reflects the severity of the disorder that is being treated. Various indicators that reflect the extent of the subject's illness, disease or condition can be assessed for determining whether the amount and time of the treatment is sufficient. Such indicators include, for example, clinically recognized indicators of disease severity, symptoms, or manifestations of the disorder in question. The degree of improvement generally is determined by a physician, who can make this determination based on signs, symptoms, biopsies, or other test results, and who can also employ questionnaires that are administered to the subject, such as quality-of-life questionnaires developed for a given disease. For example, an IL-23 receptor antagonist of the present invention can be administered to achieve an improvement in a subject's condition related to psoriasis.

Improvement can be indicated by an improvement in an index of disease activity, by amelioration of clinical symptoms or by any other measure of disease activity. Once such index of disease is the Area and Severity Index (PASI), which is a system used for assessing and grading the severity of psoriatic lesions and their response to therapy. Other index can include an Investigator's Global Assessment (IGA) Score, Psoriasis Symptoms and Signs Diary (PSSD) Symptoms Score, and Dermatological Life Quality Index (DLQI) score.

The term “clinically safe,” as it relates to a dose, dosage regimen, treatment or method with IL-23 receptor antagonist of the present invention (e.g., the compound of formula I or a pharmaceutically acceptable salt or solvate thereof), refers to a favorable risk:benefit ratio with an acceptable frequency and/or acceptable severity of treatment-emergent adverse events (referred to as AEs or TEAEs) compared to the standard of care or to another comparator. As used herein, “adverse event,” “treatment-emergent adverse event,” and “adverse reaction” mean any harm, unfavorable, unintended or undesired sign or outcome associated with or caused by administration of a pharmaceutical composition or therapeutic. It is an untoward medical occurrence in a subject administered a medicinal product. However, abnormal values or observations are not reported as adverse events unless considered clinically significant by the investigator. As used herein, when referring to an adverse event, “clinically apparent” means clinically significant as determined by a medical doctor or an investigator using standard acceptable to those of ordinary skill in the art. When the harm or undesired outcome of adverse events reaches such a level of severity, a regulatory agency can deem the pharmaceutical composition or therapeutic unacceptable for the proposed use. In particular, “safe” as it relates to a dose, dosage regimen or treatment with IL-23 receptor antagonist of the present invention refers to with an acceptable frequency and/or acceptable severity of adverse events associated with treatment if attribution is considered to be possible, probable, or very likely due to the use of the IL-23 receptor antagonist.

“Treat”, “treating” and “treatment” refer to any indicia of success in the treatment or amelioration of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The treatment or amelioration of symptoms can be based on objective or subjective parameters; including the results of a physical examination, neuropsychiatric exams, and/or a psychiatric evaluation.

The term, “pharmaceutical product” is product that contains an active pharmaceutical ingredient that has shown to be a safe and effective treatment of one or more indications as supported by findings from clinical trials regulated by a governmental authority, e.g., the Food and Drug Administration or the similar authority in other countries.

The term, “subject” or “subjects” refers to a human patient. Examples of human patient include but are not limited to an adult (≥18 years old) human patient and adolescent (≥12 years old and <18 years old) human patient.

In describing the present invention, abbreviations and symbols utilized herein are in accordance with the common usage of such abbreviations and symbols by those skilled in the chemical and biological arts. Specifically, the following abbreviations may be used in the examples and throughout the specification:

### List of Standard Chemical Definitions

| Acronym or | |
|---|---|
| Abbreviation | Definition |
| Ac | acetate |
| ACN | acetonitrile |
| AEF | 4-(2-aminocthoxy)-L-phenylalanine |
| Amt. or amt. | amount |
| amu | atomic mass unit(s) |
| Asn, N | L-asparagine |
| BOC or Boc | t-butoxy- |
| DIC | diisopropylcarbodiimide |
| DMF | dimethyl formamide |
| Equiv., equiv., | equivalents |
| or eq. | |
| FMOC or Fmoc | fluorenylmethyloxycarbonyl |
| Glu, E | L-glutamic acid |
| HPLC | high performance liquid chromatography |
| IPA | isopropanol |
| Lys | L-lysine |
| MBHA | 4-methylbenzhydrylamine |
| 2-Nal | 2-naphthyl-L-alanine |
| 3-Pal | 3-(3-pyridyl)-L-alanine |
| Pen | penicillamine |
| Phe | L-phenylalanine |
| Sarc | sarcosine |
| SPPS | solid phase peptide synthesis |
| tBu | t-butyl |
| TFA | trifluoroacetic acid |
| THP | 4-amino-tetrahydropyran-4-carboxylic acid |
| Thr, T | L-threonine |
| TIS | triisopropyl silane |
| Trp, W | L-tryptophan |
| Trt | trityl |
| UHPLC | ultra high performance liquid chromatography |
| UV | ultraviolet |

### Compound

The present invention relates to a IL-23 receptor antagonist useful for treating psoriasis. In some embodiments, the IL-23 receptor antagonist is a peptide. In some embodiments, the IL-23 receptor antagonist is a cyclic peptide.

The present invention relates to a compound of Formula (I), Ac[Pen]*-N-T-[W(7-Me][Lys(Ac)]-[Pen]*-Phe[4-(2-aminoethoxy)]-[2-Nal]-[THP]-E-N-[3-Pal]-Sarc-NH₂ (*[Pen]-[Pen]* form disulfide bond) (SEQ ID No: 1):

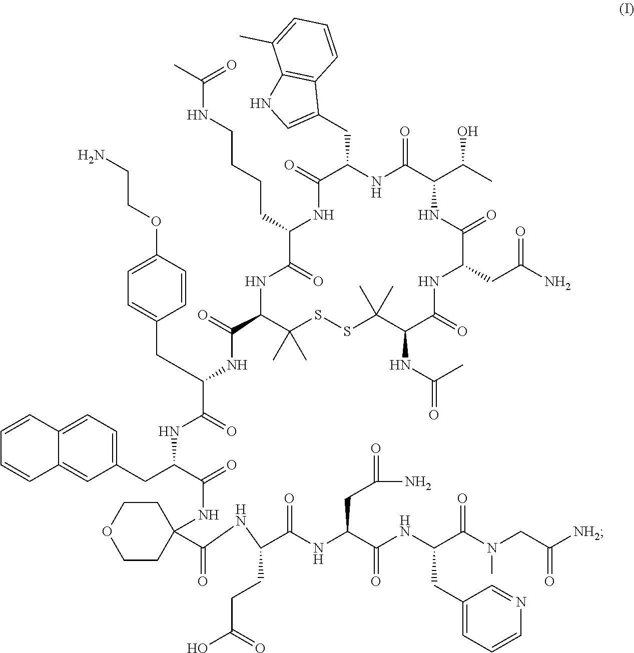 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof. The compound of formula (I) has an amino acid string of Ac-[Pen]*-N-T-[W(7-Me)]-[Lys(Ac)]-[Pen]*-Phe[4-(2-aminoethoxy)]-[2-Nal]-[THP]-E-N-[3-Pal]-Sarc-NH₂ (*[Pen]-[Pen]* form disulfide bond)(SEQ ID No:1), wherein all amino acid residues are all in L forms.

In some embodiments, the compound of Formula (I) or a pharmaceutically acceptable salt or solvate thereof may be present in any form, such as a pharmaceutically acceptable salt, hydrate, or other solvate. In some embodiments, the compound of Formula (I) or a pharmaceutically acceptable salt or solvate thereof may be provided in crystalline form, in an amorphous form, or a semi-crystalline form.

In some embodiments, the compound of Formula (I) or a pharmaceutically acceptable salt or solvate thereof is a salt. In some embodiments, the compound of Formula (I) or a pharmaceutically acceptable salt or solvate thereof is an acetate salt. In some embodiments, the compound of Formula (I) or a pharmaceutically acceptable salt or solvate thereof is a bis-acetate salt. In some embodiments, the compound of Formula (I) or a pharmaceutically acceptable salt or solvate thereof is an acetate. In some embodiments, the acetate of the compound of Formula (I) or a pharmaceutically acceptable salt or solvate thereof is an amorphous form. In some embodiments, the acetate of the compound of Formula (I) or a pharmaceutically acceptable salt or solvate thereof is the acetate salt. In some embodiments, the acetate of the compound of Formula (I) or a pharmaceutically acceptable salt or solvate thereof is the bis-acetate salt. In some embodiments, the acetate salt of the composition of the present invention is an amorphous form. In some embodiments, the compound of Formula (I) or a pharmaceutically acceptable salt or solvate thereof is a solvate. In some embodiments, the acetate form of the compound of Formula (I) is the acetate solvate.

The present invention also provides a crystalline form of a peptide of SEQ ID NO: 1, or a pharmaceutically acceptable salt thereof, or a solvate of the foregoing. The pharmaceutically acceptable salts of a peptide of SEQ ID NO: 1 provided herein include hydrochloride salts, bis-hydrochloride salts, acetate salts, fumarate salts, glutarate salts, glycolate salts, mesylate salts, sulfate salts, and citrate salts.

The present invention also provides a crystalline form of a compound of Formula (I), or a pharmaceutically acceptable salt thereof, or a solvate of the foregoing. The pharmaceutically acceptable salts of the compound of Formula (I) provided herein include hydrochloride salt, bis-hydrochloride salt, acetate salt, fumarate salt, glutarate salt, glycolate salt, mesylate salt, sulfate salt, and citrate salt.

In particular, the present invention provides a crystalline hydrochloride salt form of a compound of Formula (I) that has the structure:

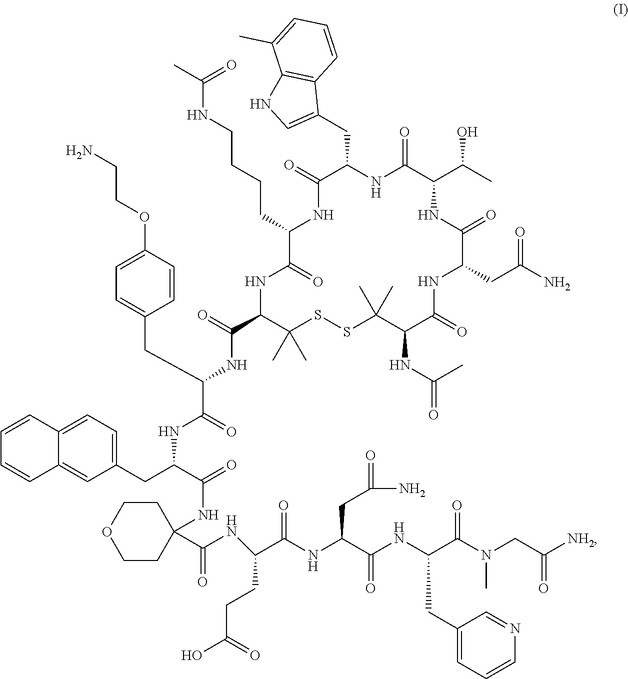 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a solvate thereof.

In some embodiments, the compound of Formula (I) or a pharmaceutically acceptable salt or solvate form thereof is a hydrochloride salt of the compound of Formula (I) or solvate thereof.

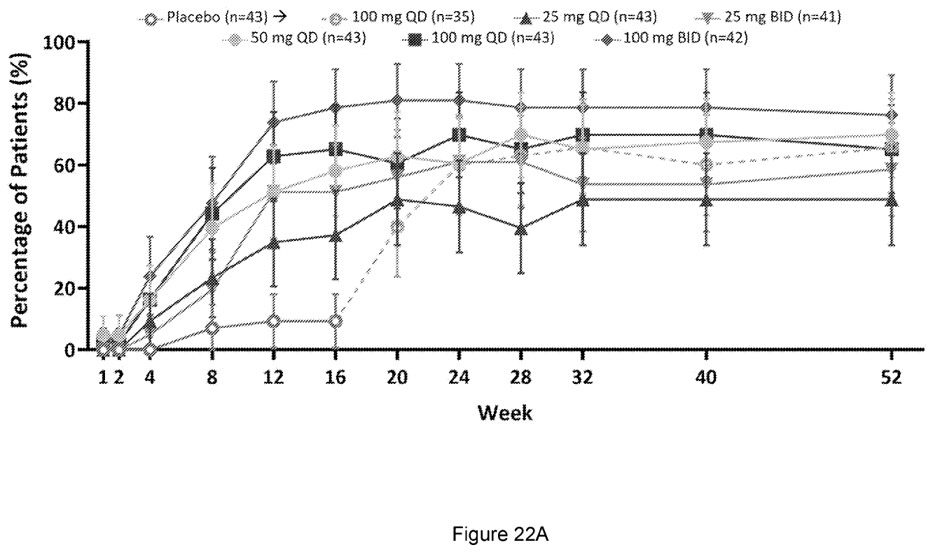

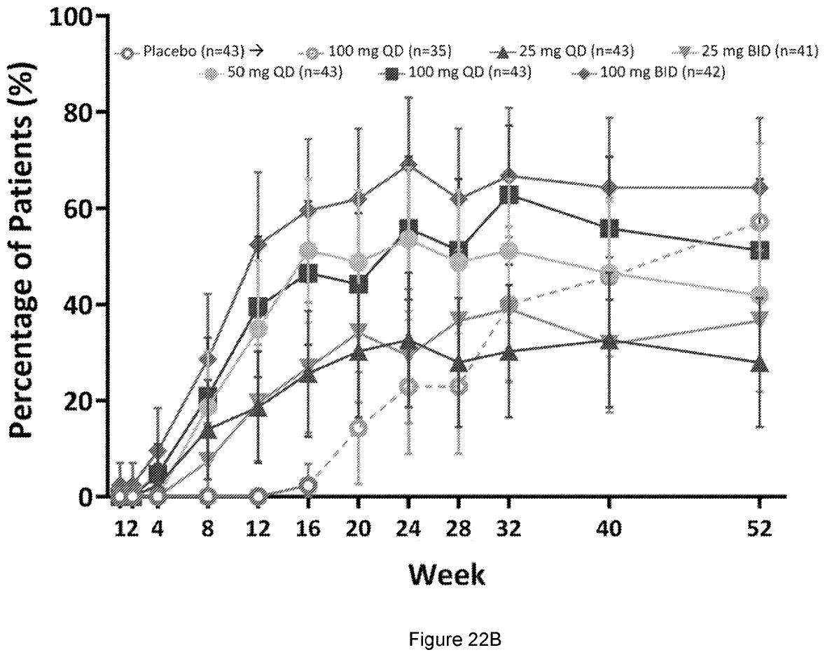

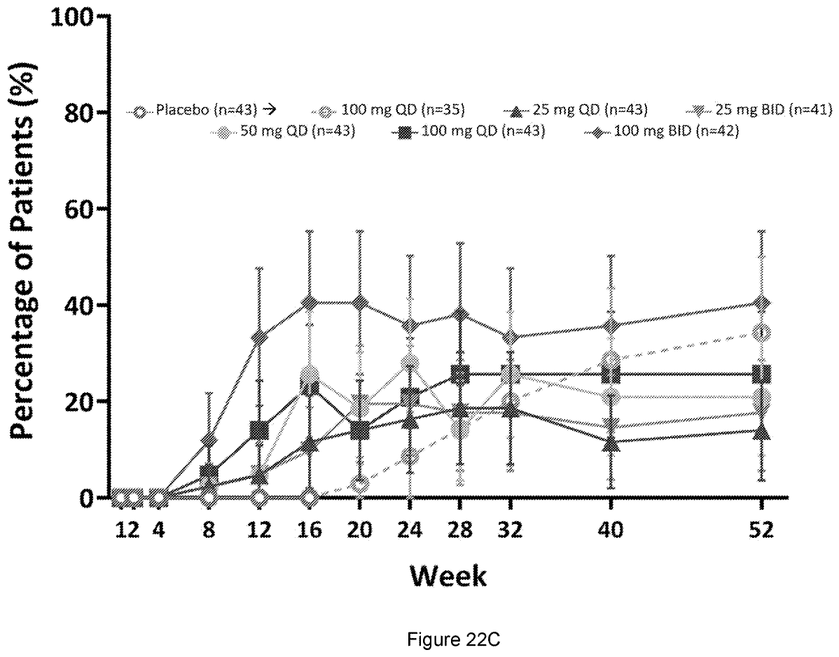

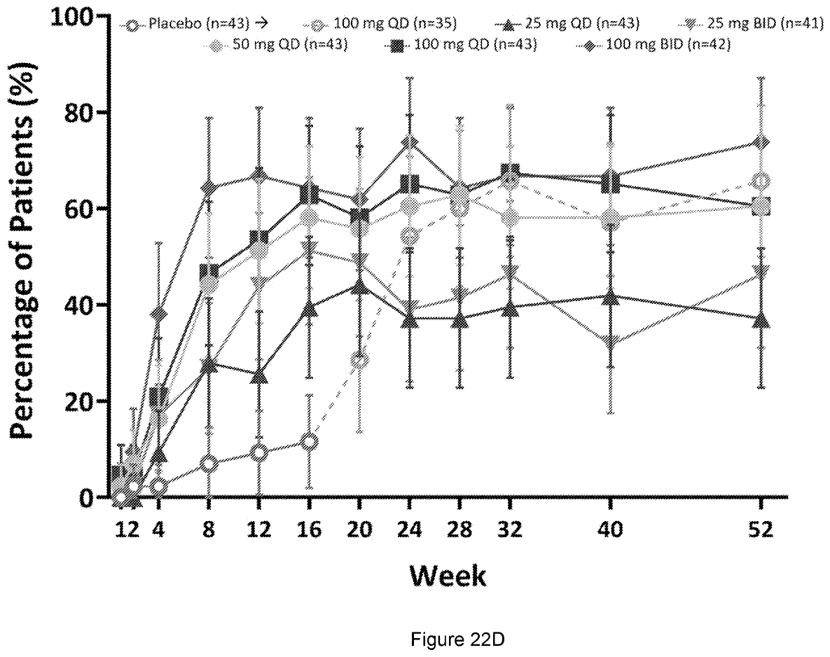

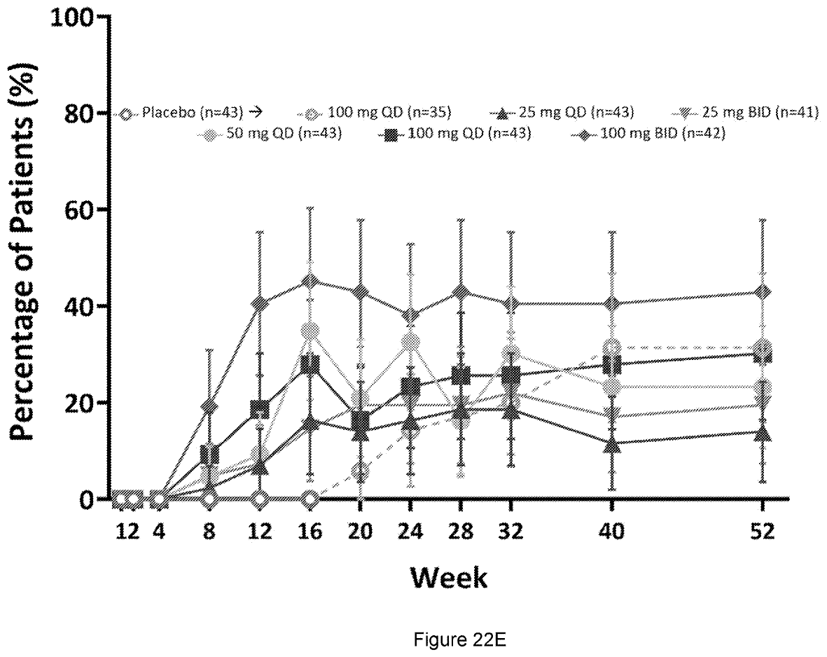

In some embodiments, the compound of formula (I) or pharmaceutically acceptable salt thereof is a hydrochloride salt form of the peptide of SEQ ID NO: 1. In some embodiments, the compound of formula (I) or pharmaceutically acceptable salt thereof is a hydrochloride salt form of peptide of SEQ ID NO: 1 characterized by an XRPD pattern substantially as set forth in FIG. 22.

In some embodiments, the compound of Formula (I) or pharmaceutically acceptable salt or solvate thereof is crystalline and in the form of a solvate. In some embodiments, the compound of Formula (I) or pharmaceutically acceptable salt or solvate thereof is crystalline and in the form of a salt.

In some embodiments, the crystalline hydrochloride salt of the compound of Formula (I) or solvate thereof is characterized as having an XRPD pattern having two or more diffraction peaks at two theta angles selected from 4.2, 6.9, 7.6, and 9.2+/−0.2 degrees two theta. In some embodiments, the crystalline hydrochloride salt of the compound of Formula (I) or solvate thereof is characterized as having an XRPD pattern having two or more diffraction peaks at two theta angles selected from 4.2, 6.9, 7.6, and 9.2+/−0.3 degrees two theta. In some embodiments, the crystalline hydrochloride salt of the compound of Formula (I) or solvate thereof is characterized as having an XRPD pattern having two or more diffraction peaks at two theta angles selected from 4.2, 6.9, 7.6, and 9.2+/−0.4 degrees two theta.

In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.3, 10.0, 10.7, 12.2, 13.9, 15.7, or 17.1+/−0.2 degrees two theta. In some embodiments, the crystalline hydrochloride salt of the compound of SEQ ID NO: 1 or solvate thereof is characterized as having an XRPD pattern having two or more diffraction peaks at two theta angles selected from 3.8, 4.2, 6.9, 7.6, 8.5, 9.3, 10.0, 10.7, 12.2, 13.9, 15.8, or 17.1+/−0.3 degrees two theta. In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.3, 10.0, 10.7, 12.2, 10.0, 10.7, 12.1, 13.9, 15.8, or 17.1+/−0.3 degrees two theta. In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.2, 10.0, 10.7, 12.1, 13.9, 15.8, or 17.1+/−0.4 degrees two theta.

In other embodiments, the crystalline hydrochloride salt of the compound of Formula (I) or solvate thereof is characterized as having an XRPD pattern having two or more diffraction peaks at two theta angles selected from 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.2 degrees two theta. In other embodiments, the crystalline hydrochloride salt of the compound of Formula (I) or solvate thereof is characterized as having an XRPD pattern having two or more diffraction peaks at two theta angles selected from 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.3 degrees two theta. In other embodiments, the crystalline hydrochloride salt of the compound of Formula (I) or solvate thereof is characterized as having an XRPD pattern having two or more diffraction peaks at two theta angles selected from 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.4 degrees two theta.

In some embodiments, the crystalline hydrochloride salt of the compound of Formula (I) or solvate thereof is characterized as having an XRPD pattern having diffraction peaks at two theta angles of at least 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.2 degrees two theta. In some embodiments, the crystalline hydrochloride salt of the compound of Formula (I) or solvate thereof is characterized as having an XRPD pattern having diffraction peaks at two theta angles of at least 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.3 degrees two theta. In some embodiments, the crystalline hydrochloride salt of the compound of Formula (I) or solvate thereof is characterized as having an XRPD pattern having diffraction peaks at two theta angles of at least 4.3, 6.9, 7.7, 8.6, 9.3, 10.0, 10.7, 11.5, 12.0, 13.1, 13.3, 14.0, 14.8, 15.8, 17.1, 17.6, 18.1, 18.6, 19.3, 20.5, 20.7, or 21.8+/−0.4 degrees two theta.

In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.3, 9.4, 10.0, 10.8, 11.5, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.2 degrees two theta. In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.3, 9.4, 10.0, 10.8, 11.6, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.3 degrees two theta. In some embodiments, the crystalline hydrochloride salt of the compound of Formula (I) or solvate thereof is characterized as having an XRPD pattern having two or more diffraction peaks at two theta angles selected from 3.8, 4.2, 6.9, 7.6, 8.5, 9.3, 9.4, 10.0, 10.8, 11.5, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.4 degrees two theta.

In some embodiments, the crystalline hydrochloride salt of the compound of Formula (I) or solvate thereof is characterized as having an XRPD pattern having diffraction peaks at two theta angles of at least 3.8, 4.2, 6.9, 7.6, 8.5, 9.3, 9.4, 10.0, 10.8, 11.5, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.2 degrees two theta. In some embodiments, the crystalline hydrochloride salt of the compound of Formula (I) or solvate thereof is characterized as having an XRPD pattern having diffraction peaks at two theta angles of at least 3.8, 4.2, 6.9, 7.6, 8.5, 9.3, 9.4, 10.0, 10.8, 11.5, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.3 degrees two theta. In some embodiments, the crystalline hydrochloride salt of the compound of Formula (I) or solvate thereof is characterized as having an XRPD pattern having diffraction peaks at two theta angles of at least 3.8, 4.2, 6.9, 7.6, 8.5, 9.3, 9.4, 10.0, 10.8, 11.5, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8+/−0.4 degrees two theta.

In some embodiments, the crystalline hydrochloride salt of the compound of Formula (I) or solvate thereof is characterized as having endotherm peaks at about 81.4° C., as determined by differential scanning calorimetry (DSC). In some embodiments, the crystalline hydrochloride salt of the compound of Formula (I) or solvate thereof is characterized as having a weight loss of about 5.6% from about 26.5° C. to about 160.0° C., as determined by thermogravimetric analysis (TGA).

In one aspect, the hydrochloride salt of a compound of Formula (I) or solvate thereof is a hemi hydrochloride salt. In some embodiments, the hemi hydrochloride salt has from about 0.1 to about 0.9, such as from about 0.2 to about 0.8 or from about 0.3 to about 0.7, molar equivalents of hydrogen chloride compared to the compound of Formula (I). In some embodiments, the hemi hydrochloride salt has about 0.1, 0.2, 0.3, 0.4, 0.5, 0.6, 0.7, 0.8, or about 0.9 molar equivalents of hydrogen chloride compared to the compound of Formula (I). In some embodiments, the hemi hydrochloride salt has about 0.5 molar equivalents of hydrogen chloride compared to the compound of Formula (I).

In some embodiments, the molar equivalents of a chloride anion of a crystalline hydrochloride salt of a compound of Formula (I) relative to one mole of the compound of Formula (I) is from about 0.2 to about 2.0. In some embodiments, the molar equivalents of a chloride anion of a crystalline hydrochloride salt of a compound of Formula (I) relative to one mole of the compound of Formula (I) is from about 0.4 to about 1.5. In other embodiments, the molar equivalents of a chloride anion of a crystalline hydrochloride salt of a compound of Formula (I) relative to one mole of the compound of Formula (I) is from about 0.5 to about 1.0. In certain embodiments, the molar equivalents of a chloride anion of a crystalline hydrochloride salt of the compound of Formula (I) relative to one mole of the compound of Formula (I) is from about 0.6 to about 0.7.

In some embodiments, the hydrochloride salt of a compound of Formula (I) or solvate thereof may be a hydrate. In some embodiments, the hydrate of the hydrochloride salt of a compound of Formula (I) has from about 0.2 to about 10 molar equivalents of water compared to the compound of Formula (I).

Alternatively, the skilled person can deliberately form a solvate using crystallization conditions that include an amount of the solvent required for the particular solvate. Thereafter the methods described above, can be used to establish whether solvates had formed.

### Compositions

The present invention also relates to compositions of a compound of Formula (I). Suitable compositions of the present invention may be in different forms, including, but are not limited to a liquid composition, such as a solution composition, or a tablet composition. When the composition is a tablet, the tablet can include two or more different phases, including an internal phase and an external phase that can contain a core. The tablet composition can also include one or more coatings.

Compositions intended for oral use may contain one or more excipients including sweetening agents, flavoring agents, coloring agents and preserving agents, in order to provide a palatable preparation. Tablets containing the active ingredient in admixture with non-toxic pharmaceutically acceptable excipients which are suitable for manufacture of tablets are acceptable. These excipients may be, for example, inert diluents, such as calcium or sodium carbonate, lactose, lactose monohydrate, croscarmellose sodium, povidone, calcium or sodium phosphate; granulating and disintegrating agents, such as maize starch, or alginic acid; binding agents, such as cellulose, microcrystalline cellulose, starch, gelatin or acacia; and lubricating agents, such as magnesium stearate, stearic acid or talc.

In some embodiments, the composition comprises the compound of Formula (I) or a pharmaceutically acceptable salt or solvate form thereof in an amount of about 5 mg to about 600 mg; and one or more pharmaceutically acceptable excipients. In some embodiments, the composition comprises the compound of Formula (I) or a pharmaceutically acceptable salt or solvate form thereof in an amount of about 5 mg to about 600 mg; and one or more pharmaceutically acceptable excipients. In some embodiments, the composition comprises the compound of Formula (I) or a pharmaceutically acceptable salt or solvate form thereof in an amount of about 5 mg to about 500 mg; and one or more pharmaceutically acceptable excipients. In some embodiments, the composition comprises the compound of Formula (I) or a pharmaceutically acceptable salt or solvate form thereof in an amount of about 10 mg to about 500 mg; and one or more pharmaceutically acceptable excipients. In some embodiments, the composition comprises the compound of Formula (I) or a pharmaceutically acceptable salt or solvate form thereof in an amount of about 10 mg to about 300 mg; and one or more pharmaceutically acceptable excipients. In some embodiments, the composition comprises the compound of Formula (I) or a pharmaceutically acceptable salt or solvate form thereof in an amount of about 10 mg to about 250 mg; and one or more pharmaceutically acceptable excipients. In some embodiments, the composition comprises the compound of Formula (I) or a pharmaceutically acceptable salt or solvate form thereof in an amount of about 50 mg to about 250 mg; and one or more pharmaceutically acceptable excipients. In some embodiments, the composition comprises the compound of Formula (I) or a pharmaceutically acceptable salt or solvate form thereof in an amount of about 100 mg to about 250 mg; and one or more pharmaceutically acceptable excipients. In some embodiments, the composition comprises the compound of Formula (I) or a pharmaceutically acceptable salt or solvate form thereof in an amount of about 150 mg to about 250 mg; and one or more pharmaceutically acceptable excipients.

In some embodiments, the composition includes 25 mg of a compound of Formula (I), or a pharmaceutically acceptable salt or solvate form thereof; and one or more pharmaceutically acceptable excipients. In some embodiments, the composition is a tablet. In some embodiments, the tablet is film coated. In some embodiments, the tablet is an immediate-release tablet. In some embodiments, the tablet is an immediate-release film-coated tablet. In some embodiments, the composition is a solution.

In some embodiments, the composition is an oral immediate-release film-coated tablet containing 25 mg of a compound of Formula (I) or a pharmaceutically acceptable salt or solvate form thereof, and one or more pharmaceutically acceptable excipients.

In some embodiments, the composition includes 50 mg of a compound of Formula (I), or a pharmaceutically acceptable salt or solvate form thereof; and one or more pharmaceutically acceptable excipients. In some embodiments, the composition is a tablet. In some embodiments, the tablet is film coated. In some embodiments, the tablet is an immediate-release tablet. In some embodiments, the tablet is an immediate-release film-coated tablet. In some embodiments, the composition is a solution.

In some embodiments, the composition is an oral immediate-release film-coated tablet containing 50 mg of a compound of Formula (I) or a pharmaceutically acceptable salt or solvate form thereof, and one or more pharmaceutically acceptable excipients.

In some embodiments, the composition includes 100 mg of a compound of Formula (I), or a pharmaceutically acceptable salt or solvate form thereof, and one or more pharmaceutically acceptable excipients. In some embodiments, the composition is a tablet. In some embodiments, the tablet is film coated. In some embodiments, the tablet is an immediate-release tablet. In some embodiments, the tablet is an immediate-release film-coated tablet. In some embodiments, the composition is a solution.

In some embodiments, the composition is an oral immediate-release film-coated tablet containing 100 mg of a compound of Formula (I) or a pharmaceutically acceptable salt or solvate form thereof, and one or more pharmaceutically acceptable excipients.

In some embodiments, the composition includes 200 mg of a compound of Formula (I), or a pharmaceutically acceptable salt or solvate form thereof, and one or more pharmaceutically acceptable excipients. In some embodiments, the composition is a tablet. In some embodiments, the tablet is film coated. In some embodiments, the tablet is an immediate-release tablet. In some embodiments, the tablet is an immediate-release film-coated tablet. In some embodiments, the composition is a solution.

In some embodiments, the composition is an oral immediate-release film-coated tablet containing 200 mg of a compound of Formula (I) or a pharmaceutically acceptable salt or solvate form thereof, and one or more pharmaceutically acceptable excipients.

Methods of Administration, Treatment, Assays, and/or Uses

In some embodiments, the present invention relates to a method and/or use for administering to the subject a composition disclosed herein.

In some embodiments, the present invention relates to a method for administering a composition, which comprises:
(a) a compound of Formula (I):

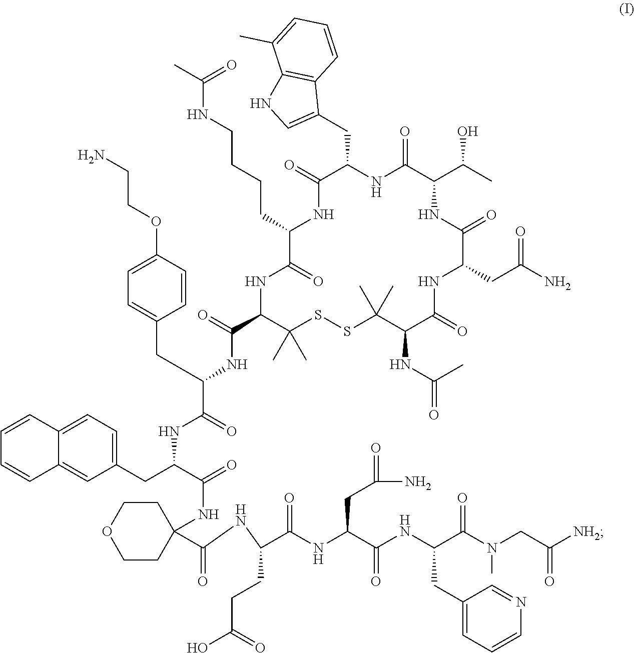 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to a method and/or use for treating psoriasis by administering a composition, which comprises:
(a) a compound of Formula (I):

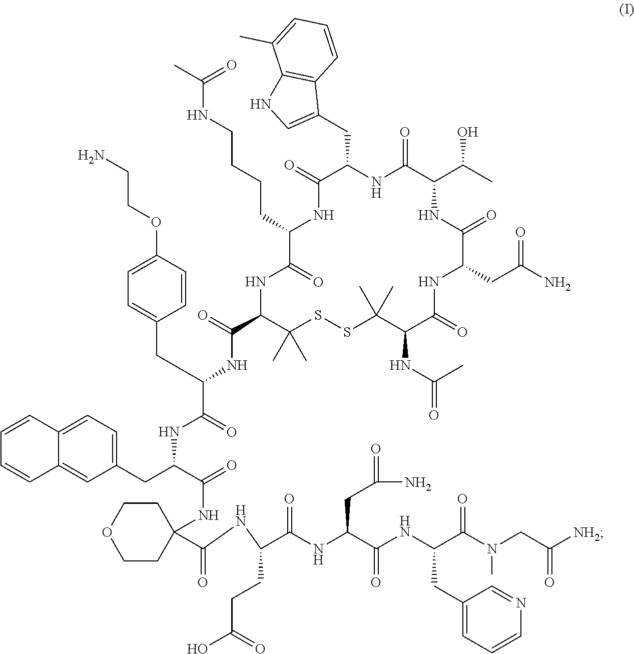 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to the method and/or use, which comprises administering multiple doses of the composition of the present invention.

In some embodiments, the present invention relates to a method and/or use for treating psoriasis by administering a composition, which comprises:
(a) a compound of Formula (I):

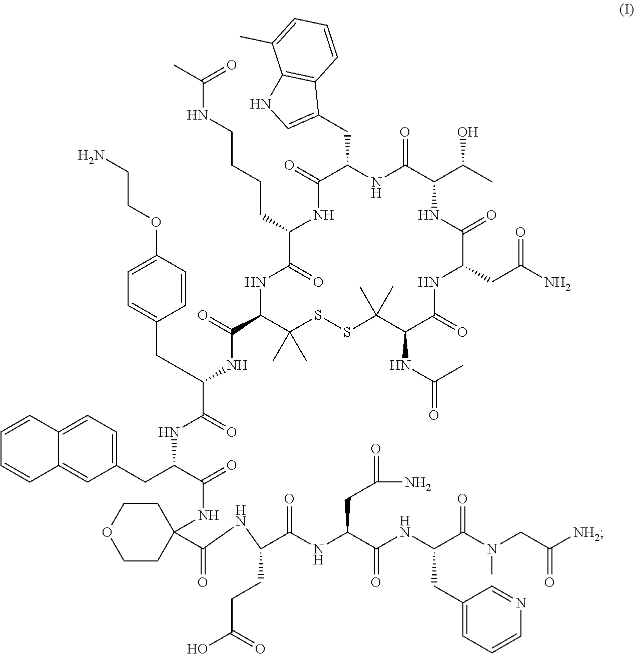 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof;
where the composition is administered to the subject for at least 16 weeks.

In some embodiments, the present invention relates to a method and/or use, which comprises the compound of Formula (I) or pharmaceutically acceptable salt or solvate thereof which is a hydrochloride salt of the compound of Formula (I) or solvate thereof.

In some embodiments, the present invention relates to a method and/or use for the treatment of plaque psoriasis, nail psoriasis, and inverse psoriasis.

Some embodiments relate to a method and/or use for the treatment of plaque psoriasis comprising administering an IL-23 receptor antagonist peptide, wherein after treating with the IL-23 receptor antagonist peptide, the subject is a responder to treatment by at least one measure of response to treatment selected from the group consisting of (i) a decrease in serum levels of psoriasis-associated biomarker BD-2, (ii) a decrease in serum levels of psoriasis-associated biomarker IL-22, (iii) a decrease in serum levels of psoriasis-associated biomarker IL-17A, and (iv) a decrease in serum levels of psoriasis-associated biomarker IL-17F.

In some embodiments, the present invention relates to a method and/or use for the treatment of plaque psoriasis. In some embodiments, the psoriasis is moderate to severe plaque psoriasis.

The compositions of the present invention can be administered to a subject or patient by any means in accordance with therapeutic administration, which accomplishes the intended purpose or pharmaceutical efficacy. Examples include administration by oral, parenteral, subcutaneous, intravenous, intramuscular, intraperitoneal, transdermal, topical, buccal or ocular routes. In some embodiments, the administration of the composition of the present invention is adapted for oral administration.

In some embodiments of the present invention, the method and/or use comprises orally administering the composition.

In some embodiments of the present invention, the method and/or use comprises administering the composition daily.

In some embodiments of the method and/or use of the invention, the composition is a tablet or the composition is formulated in a tablet comprised of a compound of Formula (I) or a pharmaceutically acceptable salt or solvate thereof and at least one or more pharmaceutically acceptable excipients. In some embodiments of the method and/or use of the invention, the composition is a tablet.

In some embodiments, the IL-23 receptor antagonist (e.g., the compound of formula (I) or pharmaceutically acceptable salt or solvate) has systemic activity. In some embodiments, the IL-23 receptor antagonist (the compound of formula (I) or pharmaceutically acceptable salt or solvate) is orally administered once daily. In some embodiments, the IL-23 receptor antagonist (the compound of formula (I) or pharmaceutically acceptable salt or solvate) is orally administered twice daily.

In some embodiments, the present invention relates to the method and/or use, which comprises orally administering the compound of formula (I) or pharmaceutically acceptable salt or solvate thereof. In some embodiments, the present invention relates to the method and/or use, which comprises orally administering the composition or the compound of formula (I) or pharmaceutically acceptable salt or solvate thereof in a fasted state.

In some embodiments, the present invention relates to the method and/or use, which comprises orally administering the composition or compound in a fed state.

In some embodiments, the subject is administered the composition in concurrent or sequential treatment periods. In some embodiments, the subject is administered the composition in concurrent treatment periods. In some embodiments, the subject is administered the composition in sequential treatment periods.

In some embodiments, the present invention relates to a method and/or use, the compound of Formula (I) or pharmaceutically acceptable salt or solvate thereof is administered to the subject for from about 12 weeks to about 52 weeks, such as from about 12 weeks to about 48 weeks, from about 12 weeks to about 44 weeks, from about 12 weeks to about 40 weeks, from about 12 weeks to about 36 weeks, from about 12 weeks to about 32 weeks, from about 12 weeks to about 28 weeks, from about 12 weeks to about 24 weeks, from about 12 weeks to about 20 weeks, or from about 14 weeks to about 18 weeks. In some embodiments, the present invention relates to a method and/or use, the compound of Formula (I) or pharmaceutically acceptable salt or solvate thereof is administered to the subject for about 12 weeks, about 13 weeks, about 14 weeks, about 15 weeks, about 16 weeks, about 17 weeks, about 18 weeks, about 19 weeks, or about 20 weeks. In some embodiments, the present invention relates to a method and/or use, the compound of Formula (I) or pharmaceutically acceptable salt or solvate thereof is administered to the subject for about 14 weeks. In some embodiments, the present invention relates to a method and/or use, the compound of Formula (I) or pharmaceutically acceptable salt or solvate thereof is administered to the subject for about 15 weeks. In some embodiments, the present invention relates to a method and/or use, the compound of Formula (I) or pharmaceutically acceptable salt or solvate thereof is administered to the subject for about 16 weeks. In some embodiments, the present invention relates to a method and/or use, the compound of Formula (I) or pharmaceutically acceptable salt or solvate thereof is administered to the subject for about 17 weeks. In some embodiments, the present invention relates to a method and/or use, the compound of Formula (I) or pharmaceutically acceptable salt or solvate thereof is administered to the subject for about 18 weeks.

In some embodiments, the present invention relates to a method and/or use, the compound of Formula (I) or pharmaceutically acceptable salt or solvate thereof is administered to the subject daily for 16 weeks.

In some embodiments, the present invention relates to the method and/or use for treating psoriasis, which includes the composition that has: (a) 25 mg of the compound of Formula (I) or a pharmaceutically acceptable salt or solvate form thereof; and (b) one or more pharmaceutically acceptable excipients.

In some embodiments, the method and/or use as described throughout the specification includes administering a composition, which comprises:
(a) 25 mg of the compound of Formula (I):

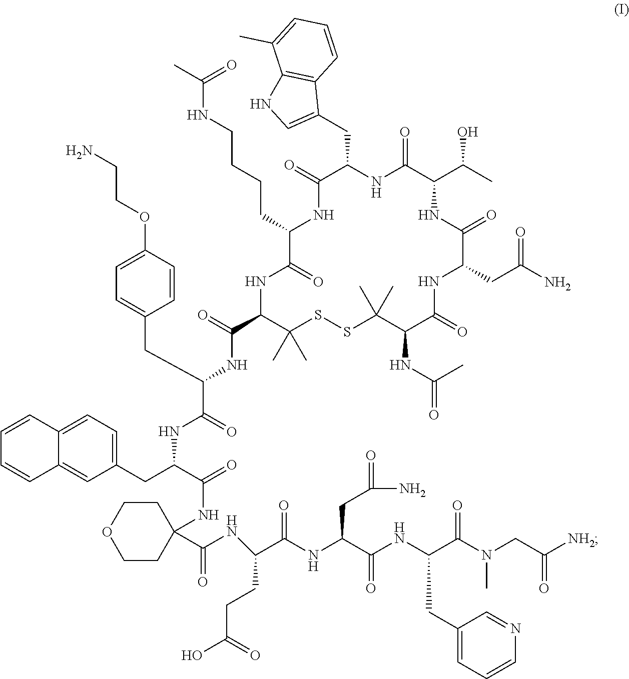 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to the method and/or use for treating psoriasis, which includes the composition that has: (a) 50 mg of the compound of Formula (I) or a pharmaceutically acceptable salt or solvate form thereof; and (b) one or more pharmaceutically acceptable excipients.

In some embodiments, the method and/or use as described throughout the specification includes administering a composition, which comprises:
(a) 50 mg of the compound of Formula (I):

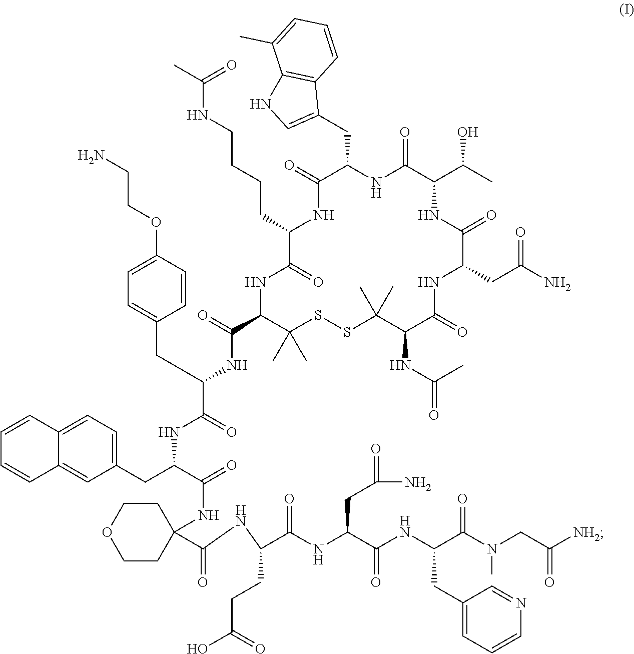 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to the method and/or use for treating psoriasis, which includes the composition that has: (a) 100 mg of the compound of Formula (I) or a pharmaceutically acceptable salt or solvate form thereof; and (b) one or more pharmaceutically acceptable excipients.

In some embodiments, the method and/or use as described throughout the specification includes administering a composition, which comprises:
(a) 100 mg of the compound of Formula (I):

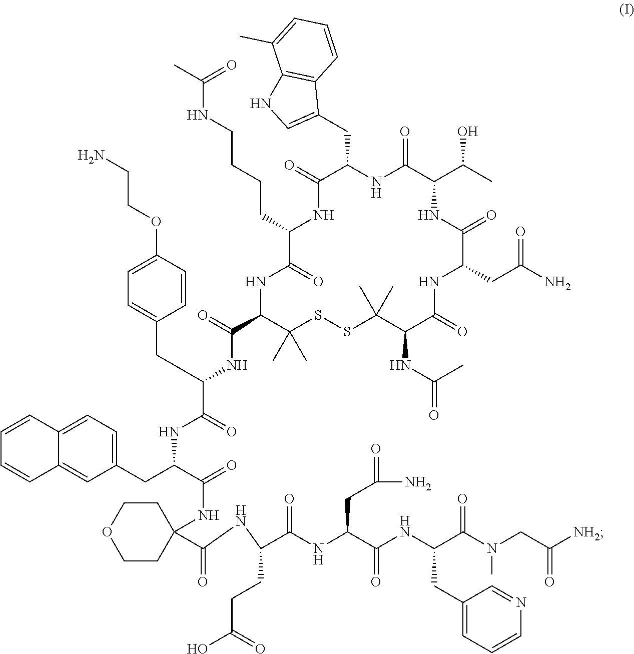 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to the method and/or use for treating psoriasis by administering a composition that comprises: (a) 200 mg of the compound of Formula (I) or a pharmaceutically acceptable salt or solvate form thereof, and (b) one or more pharmaceutically acceptable excipients.

In some embodiments, the method and/or use as described throughout the specification includes administering a composition, which comprises:
(a) 200 mg of the compound of Formula (I):

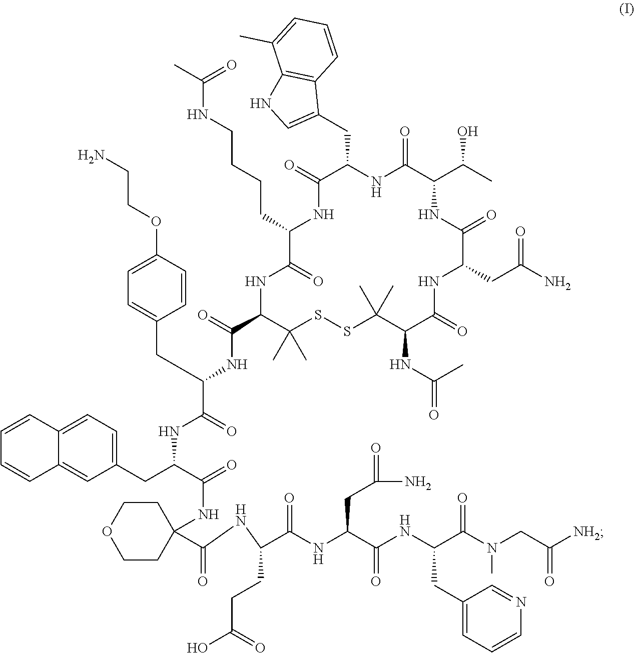 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate form thereof; and
(b) one or more pharmaceutically acceptable excipients;
to a subject in need thereof.

In some embodiments, the present invention relates to a method and/or use that includes administering once daily the composition including about 25 mg of the compound of Formula (I) or pharmaceutically acceptable salt or solvate thereof.

In some embodiments, the present invention relates to a method and/or use that includes administering once daily the composition including about 50 mg of the compound of Formula (I) or pharmaceutically acceptable salt or solvate thereof.

In some embodiments, the present invention relates to a method and/or use that includes administering once daily the composition including about 100 mg of the compound of Formula (I) or pharmaceutically acceptable salt or solvate thereof.

In some embodiments, the present invention relates to a method and/or use that includes administering once daily the composition including about 200 mg of the compound of Formula (I) or pharmaceutically acceptable salt or solvate thereof.

In some embodiments, the present invention relates to a method and/or use that includes administering twice daily the composition including about 25 mg of the compound of Formula (I) or pharmaceutically acceptable salt or solvate thereof.

In some embodiments, the present invention relates to a method and/or use that includes administering twice daily the composition including about 100 mg of the compound of Formula (I) or pharmaceutically acceptable salt or solvate thereof.

In some embodiments, the present invention relates to the method and/or use, where the subject is a candidate for phototherapy or systemic therapy.

Subjects suitable for the method of the present invention satisfy one or more criteria for inclusion, as described below. In some embodiments, the present invention relates to the method and/or use, where the subject has a psoriasis effected Body Surface Area (BSA) of at least 10% before the start of treatment. Herein the term BSA will mean the measure of the percentage of total area of your body affected by psoriasis.

The Psoriasis Area and Severity Index (PASI) is a system used for assessing and grading the severity of psoriatic lesions and their response to therapy. In the PASI system, the body is divided into four regions: the head, trunk, upper extremities, and lower extremities. Each of these areas is assessed and scored separately for erythema, induration, and scaling, which are each rated on a scale of 0 to 4 and extent of involvement on a scale of 0 to 6. The PASI produces a numeric score (a “PASI score”) that can range from 0 to 72. A higher score indicates more severe disease.

In some embodiments, the present invention relates to the method and/or use, where the subject has a psoriasis area and severity index (PASI) score of from 12 to 72 before the start of treatment.

The Investigator's Global Assessment (IGA) documents the investigator's assessment of the participant's psoriasis at a given time point. Overall lesions are graded for induration, erythema, and scaling. The participant's psoriasis is assessed as cleared (0), minimal (1), mild (2), moderate (3), or severe (4).

In some embodiments, the present invention relates to the method and/or use, where the subject has an Investigator's Global Assessment (IGA) of at least 3 before the start of treatment.

The Psoriasis Symptom and Sign Diary (PSSD) includes patient-reported outcome (PRO) questionnaires designed to measure the severity of psoriasis symptoms and signs for the assessment of treatment benefit. This study uses a 7-day recall version of the PSSD that asks the participant to answer the questions thinking about the last 7 days. The PSSD is a self-administered PRO instrument and includes 11 items covering symptoms (itch, pain, stinging, burning, and skin tightness) and patient-observable signs (skin dryness, cracking, scaling, shedding or flaking, redness, and bleeding) using 0 to 10 numerical rating scales for severity. Two subscores will be derived each ranging from 0 to 100: the psoriasis symptom score and the psoriasis sign score. A higher score indicates more severe disease.

In some embodiments, the present invention relates to the method and/or use, where the subject has a Psoriasis Symptom and Sign Diary (PSSD) symptoms score of at least 1 before the start of treatment.

In some embodiments, the present invention relates to the method and/or use, where the subject has a Psoriasis Symptom and Sign Diary (PSSD) signs score of at least 1 before the start of treatment.

The Dermatological Life Quality Index (DLQI) is a dermatology-specific health-related quality of life (HRQoL) instrument designed to assess the impact of the disease on a participant's HRQoL. It is a 10-item questionnaire that assesses HRQoL over the past week and in addition to evaluating overall HRQoL, can be used to assess 6 different aspects that may affect quality of life: symptoms and feelings, daily activities, leisure, work or school performance, personal relationships, and treatment. The total score ranges from 0 to 30 with a higher score indicating greater impact on quality of life (QoL).

In some embodiments, the present invention relates to the method and/or use, where the subject has a Dermatological Life Quality Index (DLQI) score of greater than 1 before the start of treatment.

In some embodiments, anchor-based methods can link scores on the patient reported outcomes to an external criterion that identifies participants who have experienced an important change in their condition. The Patient Global Impression-Severity (PGI-S) and Change (PGI-C) can be used as anchors, external criteria, to determine meaningful change in scores for other PROs in this population. The PGI-S contains 1 question on how the participant would currently rate severity of disease in the past 7 days, with responses ranging from 1=“None” to 5=“Very severe.” The PGI-C contains 1 question on how the participant would rate the change from their first treatment in this study. The response options are presented on a 7-point scale from 1=“A lot better now” to 7=“A lot worse now.”

In some embodiments, the present invention relates to the method and/or use, where before the start of treatment with the compound of Formula (I) or pharmaceutically acceptable salt or solvate thereof, the subject has not been treated with a biologic agent for psoriasis. In some embodiments, the present invention relates to the method and/or use, where the biologic agent for psoriasis is an anti-IL-23 antibody, an anti-IL-17 or anti-IL-12/23 antibody, an anti-TNFα antibody, an agent that modulates B cells, or an agent that modulates T cells.

In some embodiments, the present invention relates to the method and/or use, where before the start of treatment with the compound of Formula (I) or pharmaceutically acceptable salt or solvate thereof, the subject has not been treated with a JAK inhibitor or a PDE4 inhibitor.

In some embodiments, the present invention relates to the method and/or use, where before the start of treatment with the compound of Formula (I) or pharmaceutically acceptable salt or solvate thereof, the subject has not been treated with an immunosuppressant. In some embodiments, the present invention relates to the method and/or use, where the immunosuppressant is methotrexate, azathioprine, cyclosporine, 6-thioguanine, mercaptopurine, mycophenolate mofetil, or tacrolimus.

Efficacy of the method and/or use of the present invention can be assessed by a number of methods or criteria, as described above and below. In some embodiments, a subject's response to the method and/or use of the present disclosure can be measured by a PASI response.

In some embodiments, the present invention relates to the method and/or use, where the subject achieves a PASI 75 response. In some embodiments, the present invention relates to the method and/or use, where the subject achieves a PASI 75 response at Week 16 of treatment. In some embodiments, the present invention relates to the method and/or use, where the subject achieves a PASI 75 response at Week 52 of treatment. In some embodiments, the present invention relates to the method and/or use, where the subject achieves a PASI 75 response at Week 16 of treatment and maintains the PASI 75 response through Week 52 of treatment.

In some embodiments, the present invention relates to the method and/or use, where the subject achieves a PASI 90 response. In some embodiments, the present invention relates to the method and/or use, where the subject achieves a PASI 90 response at Week 16 of treatment. In some embodiments, the present invention relates to the method and/or use, where the subject achieves a PASI 90 response at Week 52 of treatment. In some embodiments, the present invention relates to the method and/or use, where the subject achieves a PASI 90 response at Week 16 of treatment and maintains the PASI 90 response through Week 52 of treatment.

In some embodiments, the present invention relates to the method and/or use, where the subject achieves a PASI 100 response. In some embodiments, the present invention relates to the method and/or use, where the subject achieves a PASI 100 response at Week 16 of treatment. In some embodiments, the present invention relates to the method and/or use, where the subject achieves a PASI 100 response at Week 52 of treatment. In some embodiments, the present invention relates to the method and/or use, where the subject achieves a PASI 100 response at Week 16 of treatment and maintains the PASI 100 response through Week 52 of treatment.

In some embodiments, the present invention relates to the method and/or use, where the subject achieves a decrease in PASI score from baseline. In some embodiments, the subject achieves at least a 14-point decrease in PASI score. In some embodiments, the subject achieves at least a 15-point decrease in PASI score. In some embodiments, the subject achieves at least a 16-point decrease in PASI score. In some embodiments, the subject achieves at least a 17-point decrease in PASI score. In some embodiments, the subject achieves at least an 18-point decrease in PASI score. In some embodiments, the present invention relates to the method and/or use, where the subject achieves the decrease in PASI score at Week 16 of treatment. In some embodiments, the present invention relates to the method and/or use, where the subject achieves the decrease in PASI score at Week 52 of treatment. In some embodiments, the present invention relates to the method and/or use, where the subject achieves the decrease in PASI score at Week 16 of treatment and maintains the decrease in PASI score through Week 52 of treatment.

In some embodiments, the present invention relates to the method and/or use, where the subject achieves an Investigator's Global Assessment (IGA) score of 0 or 1. In some embodiments, the present invention relates to the method and/or use, where the subject achieves an Investigator's Global Assessment (IGA) score of 0 or 1 at Week 16 of treatment. In some embodiments, the present invention relates to the method and/or use, where the subject achieves an Investigator's Global Assessment (IGA) score of 0 or 1 at Week 52 of treatment. In some embodiments, the present invention relates to the method and/or use, where the subject achieves an Investigator's Global Assessment (IGA) score of 0 or 1 at Week 16 of treatment and maintains the IGA score of 0 or 1 through Week 52 of treatment.

In some embodiments, the present invention relates to the method and/or use, where the subject achieves an Investigator's Global Assessment (IGA) score of 0. In some embodiments, the present invention relates to the method and/or use, where the subject achieves an Investigator's Global Assessment (IGA) score of 0 at Week 16 of treatment. In some embodiments, the present invention relates to the method and/or use, where the subject achieves an Investigator's Global Assessment (IGA) score of 0 at Week 52 of treatment. In some embodiments, the present invention relates to the method and/or use, where the subject achieves an Investigator's Global Assessment (IGA) score of 0 at Week 16 of treatment and maintains the IGA score of 0 through Week 52 of treatment.

In some embodiments, the present invention relates to the method and/or use, where the subject achieves a Psoriasis Symptom and Sign Diary (PSSD) symptoms score of 0. In some embodiments, the present invention relates to the method and/or use, where the subject achieves a Psoriasis Symptom and Sign Diary (PSSD) symptoms score of 0 at Week 16 of treatment. In some embodiments, the present invention relates to the method and/or use, where the subject achieves a Psoriasis Symptom and Sign Diary (PSSD) symptoms score of 0 at Week 52 of treatment. In some embodiments, the present invention relates to the method and/or use, where the subject achieves a Psoriasis Symptom and Sign Diary (PSSD) symptoms score of 0 at Week 16 of treatment and maintains the PSSD symptoms score of 0 through Week 52 of treatment.

In some embodiments, the present invention relates to the method and/or use, where the subject achieves a decrease in PSSD symptoms score from baseline. In some embodiments, the subject achieves at least a 30-point decrease in PSSD symptoms score. In some embodiments, the subject achieves at least a 35-point decrease in PSSD symptoms score. In some embodiments, the subject achieves at least a 40-point decrease in PSSD symptoms score. In some embodiments, the subject achieves at least a 45-point decrease in PSSD symptoms score. In some embodiments, the subject achieves at least a 46-point decrease in PSSD symptoms score. In some embodiments, the subject achieves at least a 47-point decrease in PSSD symptoms score. In some embodiments, the subject achieves at least a 48-point decrease in PSSD symptoms score. In some embodiments, the subject achieves at least a 49-point decrease in PSSD symptoms score. In some embodiments, the subject achieves at least a 50-point decrease in PSSD symptoms score. In some embodiments, the subject achieves at least a 51-point decrease in PSSD symptoms score. In some embodiments, the subject achieves at least a 52-point decrease in PSSD symptoms score. In some embodiments, the subject achieves at least a 53-point decrease in PSSD symptoms score. In some embodiments, the present invention relates to the method and/or use, where the subject achieves the decrease in PSSD symptoms score at Week 16 of treatment. In some embodiments, the present invention relates to the method and/or use, where the subject achieves the decrease in PSSD symptoms score at Week 52 of treatment. In some embodiments, the present invention relates to the method and/or use, where the subject achieves the decrease in PSSD symptoms score at Week 16 of treatment and maintains the decrease in PSSD symptoms score through Week 52 of treatment.

In some embodiments, the present invention relates to the method and/or use, where the subject achieves a Psoriasis Symptom and Sign Diary (PSSD) signs score of 0. In some embodiments, the present invention relates to the method and/or use, where the subject achieves a Psoriasis Symptom and Sign Diary (PSSD) signs score of 0 at Week 16 of treatment. In some embodiments, the present invention relates to the method and/or use, where the subject achieves a Psoriasis Symptom and Sign Diary (PSSD) signs score of 0 at Week 52. In some embodiments, the present invention relates to the method and/or use, where the subject achieves a Psoriasis Symptom and Sign Diary (PSSD) signs score of 0 at Week 16 of treatment and maintains the PSSD signs score of 0 through Week 52.

In some embodiments, the present invention relates to the method and/or use, where the subject achieves a decrease in PSSD signs score from baseline. In some embodiments, the subject achieves at least a 40-point decrease in PSSD signs score. In some embodiments, the subject achieves at least a 41-point decrease in PSSD signs score. In some embodiments, the subject achieves at least a 42-point decrease in PSSD signs score. In some embodiments, the subject achieves at least a 43-point decrease in PSSD signs score. In some embodiments, the subject achieves at least a 44-point decrease in PSSD signs score. In some embodiments, the subject achieves at least a 45-point decrease in PSSD signs score. In some embodiments, the subject achieves at least a 46-point decrease in PSSD signs score. In some embodiments, the subject achieves at least a 47-point decrease in PSSD signs score. In some embodiments, the subject achieves at least a 48-point decrease in PSSD signs score. In some embodiments, the subject achieves at least a 49-point decrease in PSSD signs score. In some embodiments, the subject achieves at least a 50-point decrease in PSSD signs score. In some embodiments, the subject achieves at least a 51-point decrease in PSSD signs score. In some embodiments, the subject achieves at least a 52-point decrease in PSSD signs score. In some embodiments, the subject achieves at least a 53-point decrease in PSSD signs score. In some embodiments, the subject achieves at least a 54-point decrease in PSSD signs score. In some embodiments, the subject achieves at least a 55-point decrease in PSSD signs score. In some embodiments, the present invention relates to the method and/or use, where the subject achieves the decrease in PSSD signs score at Week 16 of treatment. In some embodiments, the present invention relates to the method and/or use, where the subject achieves the decrease in PSSD signs score at Week 52 of treatment. In some embodiments, the present invention relates to the method and/or use, where the subject achieves the decrease in PSSD signs score at Week 16 of treatment and maintains the decrease in PSSD signs score through Week 52 of treatment.

In some embodiments, the present invention relates to the method and/or use, where the subject achieves a Dermatological Life Quality Index (DLQI) score of 0 or 1. In some embodiments, the present invention relates to the method and/or use, where the subject achieves a Dermatological Life Quality Index (DLQI) score of 0 or 1 at Week 16 of treatment. In some embodiments, the present invention relates to the method and/or use, where the subject achieves a Dermatological Life Quality Index (DLQI) score of 0 or 1 at Week 52 of treatment. In some embodiments, the present invention relates to the method and/or use, where the subject achieves a Dermatological Life Quality Index (DLQI) score of 0 or 1 at Week 16 of treatment and maintains the DLQI score of 0 or 1 through Week 52 of treatment.

The Patient-Reported Outcomes Measurement Information System-29 (PROMIS-29) is a 29-item generic health-related quality of life (HRQoL) survey, assessing each of the 7 PROMIS domains (depression; anxiety; physical function; pain interference; fatigue; sleep disturbance; and ability to participate in social roles and activities) with 4 questions. The questions are ranked on a 5-point Likert Scale. There is also one 11-point rating scale for pain intensity.

In some embodiments, the present invention relates to the method and/or use, where the subject achieves a reduction of at least 5 points in a Patient-Reported Outcomes Measurement Information System-29 (PROMIS-29) domain. In some embodiments, the present invention relates to the method and/or use, where the subject achieves a reduction of at least 5 points in a Patient-Reported Outcomes Measurement Information System-29 (PROMIS-29) domain at Week 16 of treatment.

In some embodiments, the present invention relates to the method and/or use, where the pharmacodynamics and pharmacokinetic relationship of the compound of Formula (I) is determined for biomarkers, efficacy, and/or safety in a subject. In some embodiments, one or more of IL-22, IL-17A, IL-17F and/or beta-defensin-2 (BD-2) is evaluated to assess the impact of the composition of the present invention on inflammatory proteins in the serum.

In some embodiments, the present invention relates to the method and/or use, where the subject achieves a decrease in BD-2 serum levels from baseline. In some embodiments, the decrease in BD-2 serum levels is at least a 50% decrease from baseline. In some embodiments, the decrease in BD-2 serum levels is at least a 75% decrease from baseline. In some embodiments, the decrease in BD-2 serum levels is at least an 87.5% decrease from baseline. In some embodiments, the present invention relates to the method and/or use, where the subject achieves the decrease in BD-2 serum levels from baseline at Week 16 of treatment. In some embodiments, the present invention relates to the method and/or use, where the subject the decrease in BD-2 serum levels from baseline at Week 52 of treatment. In some embodiments, the present invention relates to the method and/or use, where the subject achieves the decrease in BD-2 serum levels from baseline at Week 16 of treatment and maintains the decrease in BD-2 serum levels from baseline through Week 52 of treatment.

In some embodiments, the present invention relates to the method and/or use, where the subject achieves a decrease in IL-22 serum levels from baseline. In some embodiments, the decrease in IL-22 serum levels is at least a 30% decrease from baseline. In some embodiments, the decrease in IL-22 serum levels is at least a 50% decrease from baseline. In some embodiments, the present invention relates to the method and/or use, where the subject achieves the decrease in IL-22 serum levels from baseline at Week 16 of treatment. In some embodiments, the present invention relates to the method and/or use, where the subject the decrease in IL-22 serum levels from baseline at Week 52 of treatment. In some embodiments, the present invention relates to the method and/or use, where the subject achieves the decrease in IL-22 serum levels from baseline at Week 16 of treatment and maintains the decrease in IL-22 serum levels from baseline through Week 52 of treatment.

In some embodiments, the present invention relates to the method and/or use, where the subject achieves a decrease in IL-17A serum levels from baseline. In some embodiments, the decrease in IL-17A serum levels is at least a 30% decrease from baseline. In some embodiments, the decrease in IL-17A serum levels is at least a 50% decrease from baseline. In some embodiments, the present invention relates to the method and/or use, where the subject achieves the decrease in IL-17A serum levels from baseline at Week 16 of treatment. In some embodiments, the present invention relates to the method and/or use, where the subject the decrease in IL-17A serum levels from baseline at Week 52 of treatment. In some embodiments, the present invention relates to the method and/or use, where the subject achieves the decrease in IL-17A serum levels from baseline at Week 16 of treatment and maintains the decrease in IL-17A serum levels from baseline through Week 52 of treatment.

In some embodiments, the present invention relates to the method and/or use, where the subject achieves a decrease in IL-17F serum levels from baseline. In some embodiments, the decrease in IL-17F serum levels is at least a 30% decrease from baseline. In some embodiments, the decrease in IL-17F serum levels is at least a 50% decrease from baseline. In some embodiments, the decrease in IL-17F serum levels is at least an 60%. decrease from baseline. In some embodiments, the present invention relates to the method and/or use, where the subject achieves the decrease in IL-17F serum levels from baseline at Week 16 of treatment. In some embodiments, the present invention relates to the method and/or use, where the subject the decrease in IL-17F serum levels from baseline at Week 52 of treatment. In some embodiments, the present invention relates to the method and/or use, where the subject achieves the decrease in IL-17F serum levels from baseline at Week 16 of treatment and maintains the decrease in IL-17F serum levels from baseline through Week 52 of treatment.

In some embodiments, the present invention relates to the method and/or use, where the plasma concentration of the compound of Formula (I) is determined just prior to the beginning or at the end of the dosing interval. In some embodiments, the present invention relates to the method and/or use, where the plasma concentration of the compound of Formula (I) is determined by measuring C_{trough}, Cₘₐₓ, C_{avg}, and/or AUC. In some embodiments, the present invention relates to the method and/or use, where the relationship between pharmacokinetic parameters and pharmacodynamics is determined. In some embodiments, the present invention relates to the method and/or use, where the relationship between pharmacokinetic parameters and pharmacodynamics comprises determining skin, blood cellular and molecular biomarker activity, clinical endpoints, and/or safety parameters. In some embodiments, the present invention relates to the method and/or use, where the incidence of anti-drug antibodies to the compound of Formula (I) is determined.

In some embodiments, the present invention relates to the method and/or use, which comprises treating regional psoriasis.

The Scalp Specific Investigator Global Assessment (ss-IGA) instrument is used to evaluate the disease severity of scalp psoriasis. The lesions are assessed in terms of the clinical signs of redness, thickness, and scaliness which are scored as: absence of disease (0), very mild disease (1), mild disease (2), moderate disease (3), and severe disease (4).

In some embodiments, the present invention relates to the method and/or use, where the subject achieves an ss-IGA score of 0 or 1. In some embodiments, the present invention relates to the method and/or use, where the subject achieves an improvement of at least 2 in ss-IGA score. In some embodiments, the present invention relates to the method and/or use, where the subject has scalp psoriasis and achieves an ss-IGA score of 0 or 1 and an improvement of at least 2 in ss-IGA score. In some embodiments, the present invention relates to the method and/or use, where the subject has scalp psoriasis and achieves an ss-IGA score of 0 or 1 and an improvement of at least 2 in ss-IGA score at Week 16 of treatment. In some embodiments, the present invention relates to the method and/or use, where the subject has scalp psoriasis and achieves an ss-IGA score of 0 or 1 and an improvement of at least 2 in ss-IGA score at Week 52 of treatment. In some embodiments, the present invention relates to the method and/or use, where the subject has scalp psoriasis and achieves an ss-IGA score of 0 or 1 and an improvement of at least 2 in ss-IGA score at Week 16 of treatment and maintains the ss-IGA score of 0 or 1 through Week 52 of treatment.

Nail Psoriasis Area and Severity Index (NAPSI) is an index used for assessing and grading the severity of nail psoriasis. Each of the participant's 20 nails (fingernails and toenails) is divided into quadrants and is assessed for any presence of nail psoriasis in the nail matrix (pitting, leukonychia, red spots in the lunula, and nail plate crumbling) and any presence of nail psoriasis in the nail bed (onycholysis, splinter hemorrhages, oil drop discoloration, and nail bed hyperkeratosis). Both the nail matrix (score 0-4) and nail bed (score 0-4) scores equal the number of quadrants affected by nail/nailbed psoriasis. The total individual nail score is the sum of the nail matrix and nail bed score and ranges from 0 to 8. The sum of all 20 individual nail scores is the total NAPSI score (0 to 160).

In some embodiments, the present invention relates to the method and/or use, where the subject achieves an improvement in NAPSI. In some embodiments, the present invention relates to the method and/or use, where the subject achieves an improvement in NAPSI at Week 16 of treatment.

The Fingernail Physician's Global Assessment (f-PGA) is used to evaluate the current status of a participant's fingernail psoriasis on a scale of 0 to 4 similar to the IGA (clear [0], minimal [1], mild [2], moderate [3], or severe [4]).

In some embodiments, the present invention relates to the method and/or use, where the subject achieves an f-PGA score of 0 or 1. In some embodiments, the present invention relates to the method and/or use, where the subject having an f-PGA score≥2 at Week 0 achieves an f-PGA score of 0 or 1 at Week 16 of treatment.

The severity of hand and foot psoriasis has been assessed in various clinical studies using a Physician's Global Assessment of Hands and/or Feet (hf-PGA) instrument. The plaques on the hands and feet are scored on a 5-point scale as: clear [0], almost clear [1], mild [2], moderate [3], and severe [4].

In some embodiments, the present invention relates to the method and/or use, where the subject achieves an hf-PGA score of 0 or 1. In some embodiments, the present invention relates to the method and/or use, where the subject having an hf-PGA score≥2 at Week 0 achieves an hf-PGA score of 0 or 1 and a reduction in hf-PGA score of at least 2 at Week 16 of treatment.

The Static Physician's Global Assessment of Genitalia (s-PGA-G) is a 6-point numerical rating scale to assess the severity of genital psoriasis at a given time point. The s-PGA-G evaluates erythema, plaque elevation and scale of genital psoriatic lesions. The severity of genital psoriasis is assessed as clear [0], minimal [1], mild [2], moderate [3], severe [4], and very severe [5].

In some embodiments, the present invention relates to the method and/or use, where the subject achieves an s-PGA-G score of 0 or 1. In some embodiments, the present invention relates to the method and/or use, where the subject having an s-PGA-G score≥3 at Week 0 achieves an s-PGA-G score of 0 or 1 at Week 16 of treatment.

The Genital Psoriasis Sexual Frequency Questionnaire (GenPs-SFQ) is a 2-item patient reported survey used to assess the impact of genital psoriasis on the frequency of sexual activity in the last 7-days). Item 1 assesses overall frequency of sexual activity in the last 7-days (none/zero, once, or two or more times) and item 2 assesses how frequently genital psoriasis symptoms have limited the frequency of sexual activity in the last 7-days (never [0], rarely [1], sometimes [2], often [3], or always [4]).

In some embodiments, the present invention relates to the method and/or use, where the subject achieves a GenPs-SFQ score of 0 or 1. In some embodiments, the present invention relates to the method and/or use, where the subject having a GenPs-SFQ score≥2 at Week 0 achieves a GenPs-SFQ score of 0 or 1 at Week 16 of treatment.

In some embodiments, the present invention relates to the method and/or use, which includes determining frequency and type of related adverse events. In some embodiments, the present invention relates to the method and/or use, which includes determining frequency and type of adverse events leading to discontinuation of administering the composition of the present invention.

In some embodiments, the present invention relates to the method and/or use, which includes determining laboratory parameters and change in laboratory parameters in a subject over time. In some embodiments, the present invention relates to the method and/or use, which includes determining systolic and diastolic blood pressure in a subject overtime.

In some embodiments, the present invention relates to the method and/or use, which includes determining change in levels of skin and blood biomarkers in a subject over time.

In some embodiments, the present invention relates to the method and/or use, which includes assessing treatment satisfaction domains using the Treatment Satisfaction Questionnaire for Medications-9 items (TSQM-9). In some embodiments, the present invention relates to the method and/or use, which includes assessing treatment satisfaction domains using the Treatment Satisfaction Questionnaire for Medications-9 items (TSQM-9) at Week 16 of treatment.

In some embodiments, the present invention relates to the method and/or use for treating moderate to severe plaque psoriasis, which comprises orally administering daily a composition that includes:
(a) from about 25 mg to about 100 mg of a hydrochloride salt of a compound of Formula (I):
   or

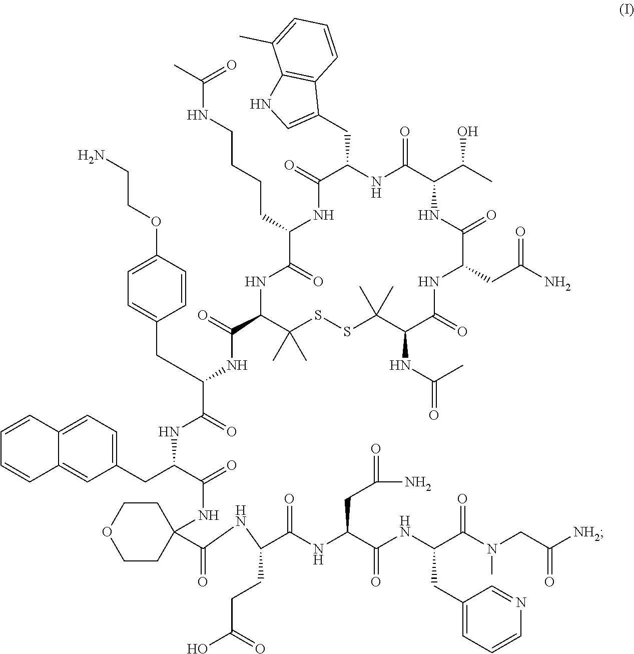 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

a solvate thereof; and
(b) one or more pharmaceutically acceptable excipients;
to an adult human subject in need thereof;
where the adult human subject is a candidate for phototherapy or systemic therapy; and the hydrochloride salt of a compound of Formula (I) or solvate thereof is administered to the adult human subject daily for at least 16 weeks.

In some embodiments, the present invention relates to the method and/or use for treating moderate to severe plaque psoriasis, which comprises orally administering daily a composition that includes:
(a) from about 25 mg to about 100 mg of a hydrochloride salt of a compound of Formula (I):

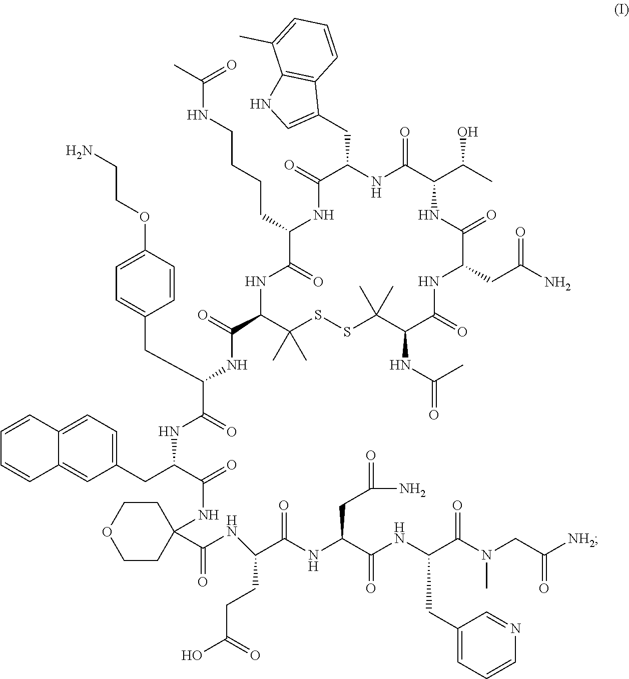 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a solvate thereof; and
(b) one or more pharmaceutically acceptable excipients;
to an adult human subject in need thereof,
where the adult human subject is a candidate for phototherapy or systemic therapy; and the hydrochloride salt of a compound of Formula (I) or solvate thereof is administered to the adult human subject daily for at least 52 weeks.

In some embodiments, the present invention relates to the method and/or use for treating moderate to severe plaque psoriasis, which comprises orally administering daily a composition that includes:
(a) from about 25 mg to about 200 mg of a hydrochloride salt of a compound of Formula (I):

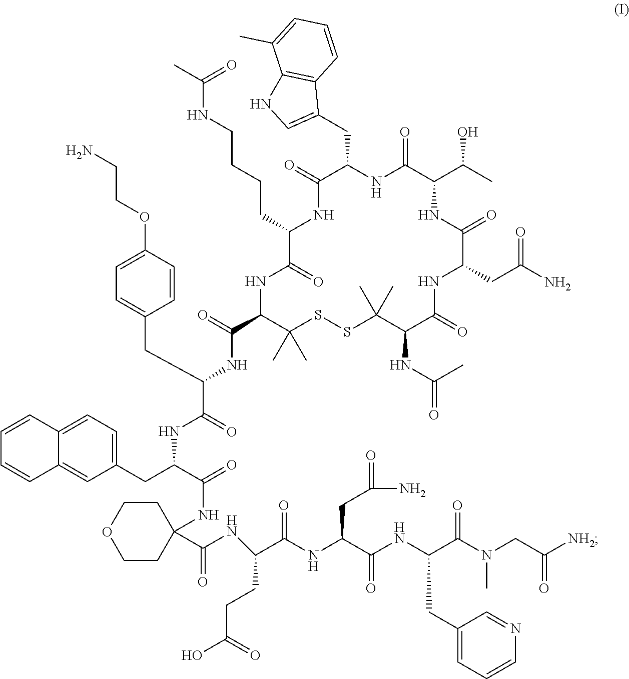 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a solvate thereof; and
(b) one or more pharmaceutically acceptable excipients;
to an adult human subject in need thereof;
where the adult human subject is a candidate for phototherapy or systemic therapy; and the hydrochloride salt of a compound of Formula (I) or solvate thereof is administered to the adult human subject daily for at least 16 weeks.

In some embodiments, the present invention relates to the method and/or use for treating moderate to severe plaque psoriasis, which comprises orally administering daily a composition that includes:
(a) from about 25 mg to about 200 mg of a hydrochloride salt of a compound of Formula (I):

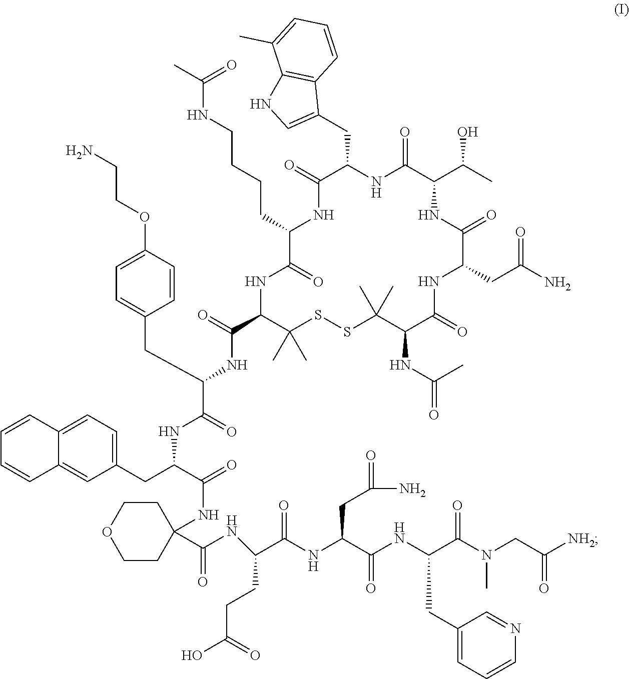 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a solvate thereof; and
(b) one or more pharmaceutically acceptable excipients;
to an adult human subject in need thereof;
where the adult human subject is a candidate for phototherapy or systemic therapy; and the hydrochloride salt of a compound of Formula (I) or solvate thereof is administered to the adult human subject daily for at least 52 weeks.

In some embodiments, the present invention relates to the method and/or use for treating psoriasis, which comprises:
(a) orally administering daily a composition that includes:
   (i) from about 25 mg to about 200 mg of a hydrochloride salt of a compound of Formula (I):

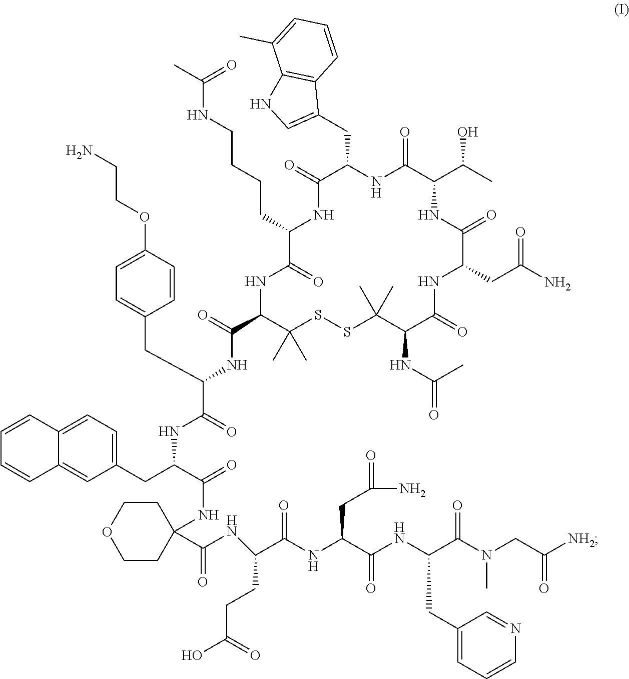 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a solvate thereof; and
   (ii) one or more pharmaceutically acceptable excipients;
to an adult human subject in need thereof; and
(b) achieving at least one psoriasis clinical endpoint selected from:
   (i) a 75% or higher reduction in Psoriasis Area and Severity Index (PASI) compared to a baseline PASI;
   (ii) an Investigator's Global Assessment (IGA) Score of 2 or lower after treatment;
   (iii) reduction from baseline in Psoriasis Symptoms and Signs Diary (PSSD) Symptoms Score by 1 or higher; and
   (iv) a reduction of Dermatological Life Quality Index (DLQI) score.

In some embodiments, step (b) comprises achieving the at least one psoriasis clinical endpoint after daily oral administration of the compound of Formula (I) or solvate thereof for at least 16 weeks. In some embodiments, step (b) comprises achieving the at least one psoriasis clinical endpoint after daily oral administration of the compound of Formula (I) or solvate thereof for at least 52 weeks. In some embodiments, step (b) comprises achieving the at least one psoriasis clinical endpoint after daily oral administration of the compound of Formula (I) or solvate thereof for at least 16 weeks and maintaining the at least one psoriasis clinical endpoint for at least 52 weeks.

In another aspect, the present invention relates to an assay method for evaluating a dose response of a compound of Formula (I) versus placebo in treatment of moderate to severe plaque psoriasis, which comprises steps of
i) orally administering a pharmaceutical composition to a patient or subject in need thereof;
where:
the pharmaceutical composition comprises:
a compound of Formula (I):

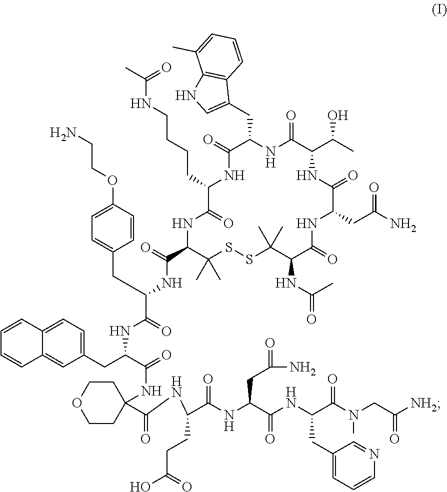 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof in an amount from about 25 mg to about 100 mg; and one or more pharmaceutically acceptable excipients.

In another aspect, the present invention relates to an assay method for evaluating a dose response of a compound of Formula (I) versus placebo in treatment of moderate to severe plaque psoriasis, which comprises steps of
[A] orally administering a pharmaceutical composition to a patient or subject in need thereof;
where:
the pharmaceutical composition comprises:
(1) a compound of Formula (I):

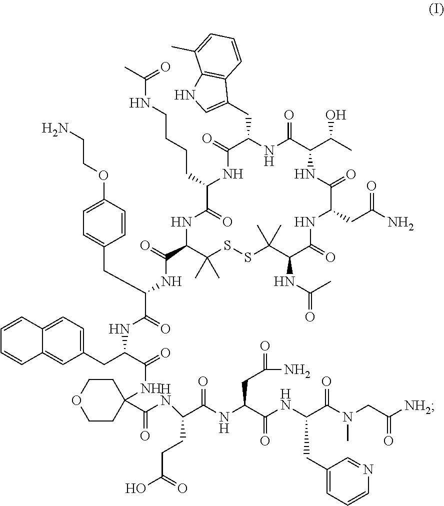 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof in an amount from about 25 mg to about 200 mg; and
(2) one or more pharmaceutically acceptable excipients;
[B] orally administering a placebo to a patient or subject in need thereof, where the patient or subject is different from the patient or subject of step [A];
[C] collecting biological samples and vital signs from the patient or subject in need thereof from steps [A] and [B] to measure laboratory parameters over a period of at least 16 weeks;
where:
the laboratory parameters are selected from:
plasma concentration parameters selected from just prior to beginning or at end of dosing intervals [C_{trough}, Cₘₐₓ, C_{avg}, and/or AUC] of the compound of Formula (I);
skin, blood cellular, DNA or molecular biomarker activities and levels;
corresponding clinical endpoints and safety parameters;
incidence of anti-drug antibodies to the compound of Formula (I) or pharmaceutically acceptable salts thereof;
vital signs selected from heart rate and blood pressure including physical diagnostic systolic and diastolic blood pressures; and/or
optional substudy collections of pharmacogenomic blood samples, ex vivo cytokine release blood samples, skin biopsy samples, and photograph collection samples selected from lesional or lesional and full body;
[D] evaluating pharmacokinetics (PK) and pharmacodynamic (PD) relationships of the compound of Formula (I) or a pharmaceutically acceptable salt there of versus placebo for efficacy, safety, immunogenicity, and biomarkers using the laboratory parameters form Step [C];
[E] characterizing additional efficacy of the compound of Formula (I) or a pharmaceutically acceptable salt there of versus placebo; and
[F] such that each of Steps [A] to [E] result in endpoints that are used to analyze and determine a dose response of a compound of Formula (I) or a pharmaceutically acceptable salt thereof versus placebo to be used for treatment of regional, moderate to severe psoriasis via measuring endpoint determination after administration of the pharmaceutical composition.

In other aspects, the present invention relates to an assay method for evaluating a dose response of a compound of Formula (I) versus placebo in treatment of moderate to severe plaque psoriasis, where the compound of Formula (I) or pharmaceutically acceptable salt is a hydrochloride salt of a compound of Formula (I).

### EMBODIMENTS

1. A method for treating psoriasis in a subject in need thereof, comprising administering a compound of Formula (I):

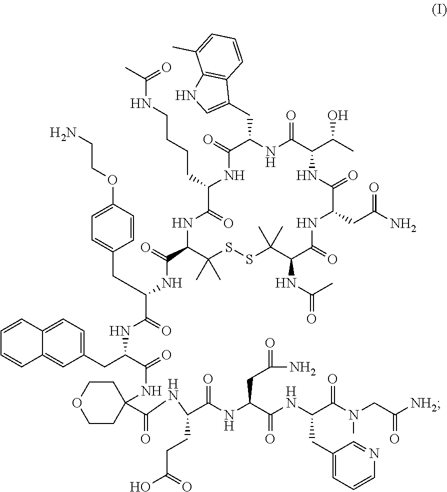 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to a patient in need thereof;
   wherein after treating with the compound of Formula (I) or a pharmaceutically acceptable salt or solvate thereof, the subject is a responder to treatment by at least one psoriasis measure of response to treatment selected from the group consisting of:
      (i) a 75% or higher reduction in Psoriasis Area and Severity Index (PASI) compared to a baseline PASI;
      (ii) an Investigator's Global Assessment (IGA) Score of 2 or lower after treatment;
      (iii) reduction from baseline in Psoriasis Symptoms and Signs Diary (PSSD) Symptoms Score by 1 or higher; and
      (iv) a reduction of Dermatological Life Quality Index (DLQI) score.
2. A method of treating psoriasis in a subject in need thereof, comprising administering a compound of Formula (I):

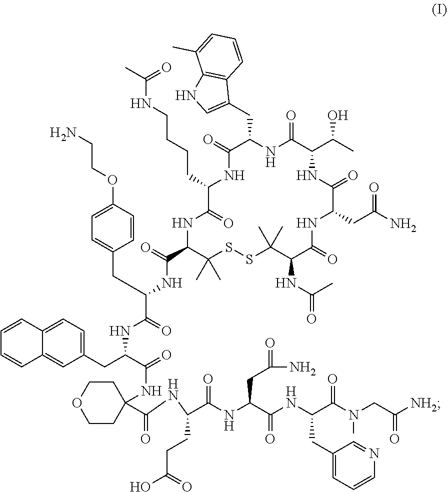 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof,
wherein the subject achieves a psoriasis measure of response of a 75% or higher reduction in Psoriasis Area and Severity Index (PASI) score compared to a baseline PASI score.
3. The method of any one of embodiments 1 to 2, wherein the subject achieves a psoriasis measure of response of at least about a 90% reduction in PASI score compared to the baseline PASI score.
4. The method of any one of embodiments 1 to 3, wherein the subject achieves a psoriasis measure of response of about 100% reduction in PASI score compared to the baseline PASI score.
5. The method of any one of embodiments 1 to 4, wherein the subject achieves a psoriasis measure of response of an Investigator's Global Assessment (IGA) Score of 2 or lower.
6. The method of any one of embodiments 1 to 5, wherein the subject achieves a psoriasis measure of response of an at least 2-point decrease in IGA score.
7. The method of any one of embodiments 1 to 6, wherein the subject achieves a psoriasis measure of response of an IGA score of 1 or lower.
8. The method of any one of embodiments 1 to 7, wherein the subject achieves a psoriasis measure of response of an IGA score of 0.
9. The method of any one of embodiments 1 to 8, wherein the patient achieves a psoriasis measure of response of an Investigator's Global Assessment (IGA) Score of 2 or lower after treatment.
10. The method of any one of embodiments 1 to 9, wherein the patient achieves a psoriasis measure of response of an IGA score of 1 or lower after treatment.
11. The method of any one of embodiments 1 to 10, wherein the patient achieves a psoriasis measure of response of an IGA score of 0 after treatment.
12. The method of any one of embodiments 1 to 11, wherein the psoriasis measure of response is measured at least 2 weeks after the beginning of treatment.
13. The method of any one of embodiments 1 to 12, wherein the psoriasis measure of response is measured at least 4 weeks after the beginning of treatment.
14. The method of any one of embodiments 1 to 13, wherein the psoriasis measure of response is measured at least 8 weeks after beginning of treatment.
15. The method of any one of embodiments 1 to 14, wherein the psoriasis measure of response is measured at least 16 weeks after beginning of treatment.
16. The method of any one of embodiments 1 to 15, wherein the psoriasis measure of response is measured at least 20 weeks after beginning of treatment.
17. The method of any one of embodiments 1 to 16, wherein the psoriasis measure of response is measured at least 24 weeks after beginning of treatment.
18. The method of any one of embodiments 1 to 17, wherein the psoriasis measure of response is measured at least 28 weeks after beginning of treatment.
19. The method of any one of embodiments 1 to 18, wherein the psoriasis measure of response is measured at least 32 weeks after beginning of treatment.
20. The method of any one of embodiments 1 to 19, wherein the psoriasis measure of response is measured at least 40 weeks after beginning of treatment.
21. The method of any one of embodiments 1 to 20, wherein the psoriasis measure of response is measured at least 52 weeks after beginning of treatment.
22. The method of any one of embodiments 1 to 21, wherein the psoriasis is plaque psoriasis.
23. The method of embodiment 22, wherein the psoriasis is moderate to severe plaque psoriasis.
24. The method of any one of embodiments 1 to 23, wherein the subject is a candidate for phototherapy or systematic therapy.
25. The method of any one of embodiments 1 to 24, wherein the compound of Formula (I) or pharmaceutically acceptable salt or solvate thereof is a hydrochloride salt of the compound of Formula (I) or solvate thereof.
26. The method of any one of embodiments 1 to 25, comprising orally administering the compound of Formula (I) or pharmaceutically acceptable salt or solvate thereof.
27. The method of any one of embodiments 1 to 26, comprising administering the compound of Formula (I) or pharmaceutically acceptable salt or solvate thereof daily.
28. The method of any one of embodiments 1 to 27, comprising administering the compound of Formula (I) or pharmaceutically acceptable salt or solvate thereof in an amount of about 5 mg to about 800 mg.
29. The method of any one of embodiments 1 to 28, comprising administering the compound of Formula (I) or pharmaceutically acceptable salt or solvate thereof in an amount of about 25 mg to about 500 mg.
30. The method of any one of embodiments 1 to 29, comprising administering the compound of Formula (I) or pharmaceutically acceptable salt or solvate thereof in an amount of about 50 mg to about 500 mg.
31. The method of any one of embodiments 1 to 29, comprising administering the compound of Formula (I) or pharmaceutically acceptable salt or solvate thereof in an amount of about 25 mg.
32. The method of any one of embodiments 1 to 30, comprising administering the compound of Formula (I) or pharmaceutically acceptable salt or solvate thereof in an amount of about 50 mg.
33. The method of any one of embodiments 1 to 30, comprising administering the compound of Formula (I) or pharmaceutically acceptable salt or solvate thereof in an amount of about 100 mg.
34. The method of any one of embodiments 1 to 30, comprising administering the compound of Formula (I) or pharmaceutically acceptable salt or solvate thereof in an amount of about 200 mg.
35. The method of any one of embodiments 1 to 34, wherein the patient has a Body Surface Area (BSA) of at least 10% prior to treatment.
36. The method of any one of embodiments 1 to 35, wherein the patient has a PASI score of about 12 to 72 prior to treatment.
37. The method of any one of embodiments 1 to 36, wherein the subject has an Investigator's Global Assessment (IGA) of at least 3 prior to treatment.
38. The method of any one of embodiments 1 to 37, wherein the subject has a Psoriasis Symptom and Sign Diary (PSSD) symptoms score of at least 1 prior to treatment.
39. The method of any one of embodiments 1 to 38, wherein the subject has a Dermatological Life Quality Index (DLQI) score of greater than 1 prior to treatment.
40. The method of any one of embodiments 1 to 39, wherein, before the start of treatment with the compound of Formula (I) or pharmaceutically acceptable salt or solvate thereof, the subject has not been treated with a biologic agent for psoriasis.
41. The method of any one of embodiments 1 to 40, wherein, before the start of treatment with the compound of Formula (I) or pharmaceutically acceptable salt or solvate thereof, the subject has not been treated with any IL-23 receptor antagonist for psoriasis.
42. A pharmaceutical product for treating a subject diagnosed with psoriasis, comprising a compound of Formula (I):

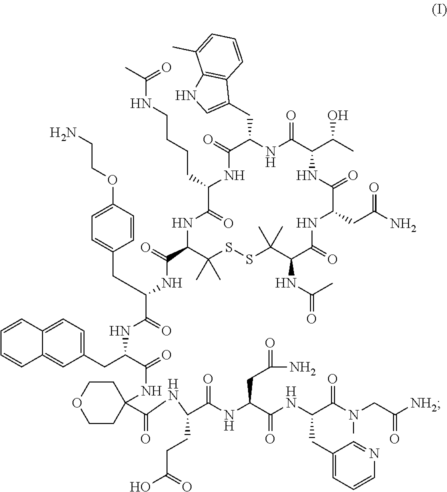 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, that when administered to said subject, in an amount of at least 25 mg, induces a mean reduction in the Psoriasis Area and Severity Index (PASI) by about 75% or greater, or induces a mean reduction in PASI by 90% or greater when measured from a baseline PASI score.
43. The pharmaceutical product of embodiment 42, wherein the subject achieves at least about a 90% reduction in Psoriasis Area and Severity Index score compared to the baseline.
44. The pharmaceutical product of embodiment 42 or 43, wherein the subject achieves about 100% reduction in Psoriasis Area and Severity Index score compared to the baseline.
45. The pharmaceutical product of any one of embodiments 42 to 44, wherein the patient achieves an Investigator's Global Assessment (IGA) Score of 2 or lower after treatment.
46. The pharmaceutical product of any one of embodiments 42 to 45, wherein the compound of Formula (I) or pharmaceutically acceptable salt or solvate thereof is in an amount of about 25 mg to about 800 mg.
47. The pharmaceutical product of any one of embodiments 42 to 46, wherein the compound of Formula (I) or pharmaceutically acceptable salt or solvate thereof is in an amount of about 50 mg to about 800 mg.
48. The pharmaceutical product of any one of embodiments 42 to 47, wherein the compound of Formula (I) or pharmaceutically acceptable salt or solvate thereof is in an amount of about 50 mg to about 500 mg.
49. The pharmaceutical product of any one of embodiments 42 to 46, wherein the compound of Formula (I) or pharmaceutically acceptable salt or solvate thereof is in an amount of about 25 mg.
50. The pharmaceutical product of any one of embodiments 42 to 48, wherein the compound of Formula (I) or pharmaceutically acceptable salt or solvate thereof is in an amount of about 50 mg.
51. The pharmaceutical product of any one of embodiments 42 to 48, wherein the compound of Formula (I) or pharmaceutically acceptable salt or solvate thereof is in an amount of about 100 mg.
52. The pharmaceutical product of any one of embodiments 42 to 48, wherein the compound of Formula (I) or pharmaceutically acceptable salt or solvate thereof is in an amount of about 200 mg.
53. A method for treating psoriasis in a subject in need thereof, comprising administering a compound of Formula (I):

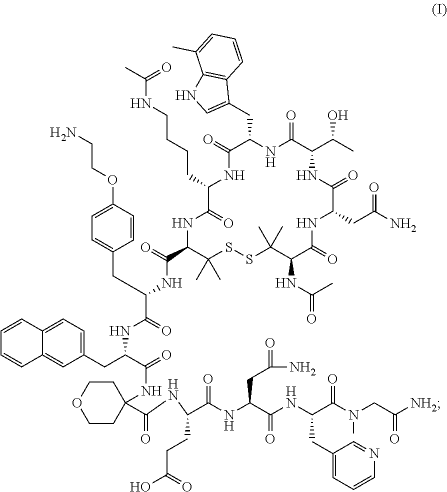 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to a patient in need thereof in an amount of about 200 mg.
54. The method of embodiment 53, wherein the method comprises administering the compound of Formula (I) or a pharmaceutically acceptable salt or solvate thereof in an amount of about 200 mg once daily.
55. The method of embodiment 53 or 54, wherein the method further comprises a step of achieving one or more selected from the group consisting of:
   (i) a 75% or higher reduction in Psoriasis Area and Severity Index (PASI) compared to a baseline PASI;
   (ii) an Investigator's Global Assessment (IGA) Score of 2 or lower after treatment;
   (iii) reduction from baseline in Psoriasis Symptoms and Signs Diary (PSSD) Symptoms Score by 1 or higher; and
   (iv) a reduction of Dermatological Life Quality Index (DLQI) score.
56. The method of embodiment 53 to 55 wherein the psoriasis is plaque psoriasis.
57. The method of embodiment 56, wherein the psoriasis is moderate to severe plaque psoriasis.
58. The method of any one of embodiments 53 to 57, wherein the subject is a candidate for phototherapy or systematic therapy.
59. The method of any one of embodiments 53 to 58, wherein the compound of Formula (I) or pharmaceutically acceptable salt or solvate thereof is a hydrochloride salt of the compound of Formula (I) or solvate thereof.
60. The method of any one of embodiments 53 to 59, comprising orally administering the compound of Formula (I) or pharmaceutically acceptable salt or solvate thereof.
61. The method of any one of embodiments 53 to 60, comprising administering the compound of Formula (I) or pharmaceutically acceptable salt or solvate thereof daily.
62. The method of any one of embodiments 53 to 61, wherein the patient has a Body Surface Area (BSA) of at least 10%/prior to treatment.
63. The method of any one of embodiments 53 to 62, wherein the patient has a PASI score of about 12 to 72 prior to treatment.
64. The method of any one of embodiments 53 to 63, wherein the subject has an Investigator's Global Assessment (IGA) of at least 3 prior to treatment.
65. The method of any one of embodiments 53 to 64, wherein the subject has a Psoriasis Symptom and Sign Diary (PSSD) symptoms score of at least 1 prior to treatment.
66. The method of any one of embodiments 53 to 65, wherein the subject has a PSSD signs score of at least 1 prior to treatment.
67. The method of any one of embodiments 53 to 66, wherein the subject has a Dermatological Life Quality Index (DLQI) score of greater than 1 prior to treatment.
68. The method of any one of embodiments 53 to 67, wherein, before the start of treatment with the compound of Formula (I) or pharmaceutically acceptable salt or solvate thereof, the subject has not been treated with a biologic agent for psoriasis.
69. The method of any one of embodiments 53 to 68, wherein, before the start of treatment with the compound of Formula (I) or pharmaceutically acceptable salt or solvate thereof, the subject has not been treated with any IL-23 receptor antagonist for psoriasis.
70. A method of treating a patient population diagnosed with psoriasis, comprising administering a compound of Formula (I):

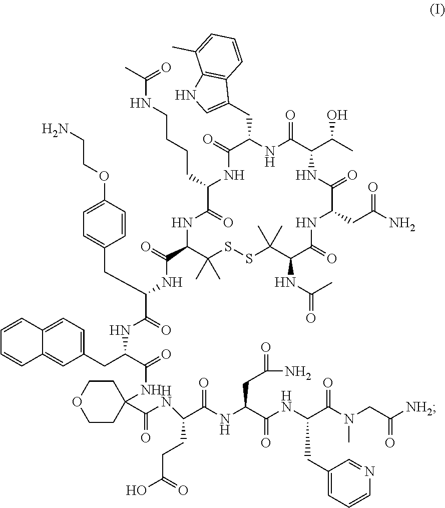 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, to said patient population in an amount of about 200 mg.
71. The method of embodiment 66, wherein the method comprises administering the compound of Formula (I) or a pharmaceutically acceptable salt or solvate thereof in an amount of about 200 mg daily.
72. The method of embodiment 70 or 71, wherein the method further comprises a step of achieving one or more selected from the group consisting of: (i) achieving an at least about 75% decrease in the Psoriasis Area and Severity Index (PASI) relative to baseline in at least about 70% of the patient population; (ii) achieving an at least about 90% decrease in the Psoriasis Area and Severity Index (PASI) relative to baseline in at least about 60% of the patient population; (iii) achieving an about 100% decrease in the Psoriasis Area and Severity Index (PASI) relative to baseline in at least about 40% of the patient population; (iv) achieving at least an average about 14-point decrease in PASI score relative to baseline in at least half of the patient population; (v) achieving an average decrease of at least about 40 points in Psoriasis Symptom and Sign Diary (PSSD) symptoms score; (vi) achieving an average decrease of at least about 50 points in PSSD signs score of 0; (vii) achieving an Investigator's Global Assessment (IGA) score of 0 or 1 in at least about 60% of the patient population; (viii) achieving an IGA score of 0 in at least about 40% of the patient population.
73. The method of embodiment 70 to 72, wherein the psoriasis is plaque psoriasis.
74. The method of embodiment 73, wherein the psoriasis is moderate to severe plaque psoriasis.
75. The method of any one of embodiments 70 to 74, wherein the compound of Formula (I) or pharmaceutically acceptable salt or solvate thereof is a hydrochloride salt of the compound of Formula (I) or solvate thereof.
76. The method of any one of embodiments 70 to 75, comprising orally administering the compound of Formula (I) or pharmaceutically acceptable salt or solvate thereof.
77. The method of any one of embodiments 70 to 76, comprising administering the compound of Formula (I) or pharmaceutically acceptable salt or solvate thereof daily.
78. The method of any one of embodiments 70 to 77, wherein a patient in the patient population has a Body Surface Area (BSA) of at least 10% prior to treatment.
79. The method of any one of embodiments 70 to 78, wherein a patient in the patient population has a PASI score of about 12 to 72 prior to treatment.
80. The method of any one of embodiments 70 to 79, wherein a patient in the patient population has an Investigator's Global Assessment (IGA) of at least 3 prior to treatment.
81. The method of any one of embodiments 70 to 80, wherein a patient in the patient population has a Psoriasis Symptom and Sign Diary (PSSD) symptoms score of at least 1 prior to treatment.
82. The method of any one of embodiments 70 to 81, wherein, before the start of treatment with the compound of Formula (I) or pharmaceutically acceptable salt or solvate thereof, a patient in the patient population has not been treated with a biologic agent for psoriasis.
83. The method of any one of embodiments 70 to 82, wherein, before the start of treatment with the compound of Formula (I) or pharmaceutically acceptable salt or solvate thereof, a patient in the patient population has not been treated with any IL-23 receptor antagonist for psoriasis.
84. The method of any one of embodiments 70 to 83, wherein the subject is a patient.
85. The method of embodiment 84, wherein the subject is an adult or adolescent patient.
86. The method of embodiments 70 to 85, wherein the compound of formula (I) a pharmaceutically acceptable salt or solvate thereof is a hydrochloride salt.
87. The method of embodiments 70 to 86, wherein the compound of formula (I) a pharmaceutically acceptable salt or solvate thereof is a hydrochloride salt in a crystalline form.
88. A pharmaceutical product for treating a subject diagnosed with psoriasis, comprising a compound of Formula (I):

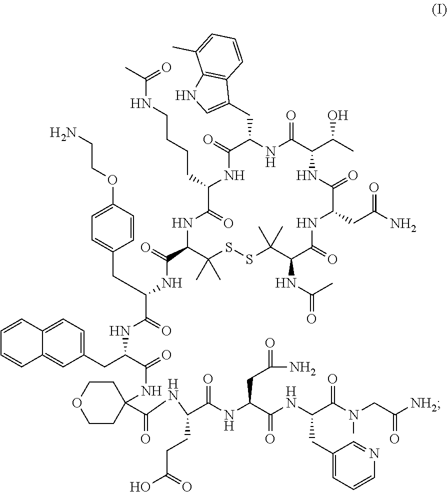 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

or
a pharmaceutically acceptable salt or solvate thereof, wherein the compound of Formula (I) or pharmaceutically acceptable salt or solvate thereof is in an amount of about 200 mg.

### EXAMPLES

### Example 1. Daily Administration for 16 Weeks in Adult Human Subjects with Moderate to Severe Plaque Psoriasis

IL-23 receptor antagonist peptide as used in this example refers to a compound of Formula (I) or a pharmaceutically acceptable salt or solvate thereof, specifically a hydrochloride salt of compound of Formula (I) in a crystalline form.

Described below was a multicenter, randomized, placebo-controlled, dose-ranging study to evaluate the efficacy and safety of the hydrochloride salt of the compound of Formula (i) for the treatment of moderate-to-severe plaque psoriasis.

Interleukin-23 is responsible for activation and maintenance of T helper 17 (Th17) in order to secrete pro-inflammatory cytokines involved in the progression of psoriasis. Due to its high potency, IL-23 receptor antagonist peptide can antagonize IL-23R systemically, providing activity in skin and/or joint tissue and addressing unmet needs for a broad range of diseases.

| Objectives | Endpoints |
|---|---|
| Primary | |
| To evaluate the dose response of IL- | Proportion of subjects achieving |
| 23 receptor antagonist peptide at Week 16 | PASI 75 response (≥75% improvement |
| in subjects with moderate-to-severe plaque | from baseline in PASI) at Week 16 |
| psoriasis | |
| Secondary | |
| To characterize additional efficacy | Change from baseline in PASI total |
| of IL-23 receptor antagonist peptide versus | score at Week 16 |
| placebo in subjects with moderate-to-severe | Proportion of subjects achieving |
| plaque psoriasis | PASI 90 response (≥90% improvement |
| | from baseline in PASI) at Week 16 |
| | Proportion of subjects achieving |
| | PASI 100 response (100% improvement |
| | from baseline in PASI) at Week 16 |
| | Proportion of subjects achieving an |
| | IGA score of cleared (0) or minimal (1) at |
| | Week 16 |
| | Proportion of subjects achieving an |
| | IGA score of cleared (0) at Week 16 |
| | Change from baseline in BSA at |
| | Week 16 |
| To evaluate the effect of IL-23 | Change from baseline in Psoriasis |
| receptor antagonist peptide treatment on | Symptoms and Signs Diary (PSSD) |
| patient-reported psoriasis severity versus | symptoms scores at Week 16 |
| placebo in subjects with moderate-to-severe | Change from baseline in PSSD |
| plaque psoriasis | signs score at Week 16 |
| | Proportion of subjects achieving |
| | PSSD symptoms score = 0 at Week 16 |
| | among subjects with a baseline symptoms |
| | score ≥1 |
| | Proportion of subjects achieving |
| | PSSD signs score = 0 at Week 16 among |
| | subjects with a baseline signs score ≥1 |
| To evaluate the effect of IL-23 | Proportion of subjects achieving a |
| receptor antagonist peptide treatment on | DLQI of 0 or 1 at Week 16 among subjects |
| dermatology-specific health-related quality | with baseline DLQI score >1 |
| of life versus placebo in subjects with | |
| moderate-to-severe plaque psoriasis | |
| To evaluate the effect of IL-23 | Change from baseline in domain |
| receptor antagonist peptide treatment on | scores of the Patient-Reported Outcomes |
| general health-related quality of life versus | Measurement Information System |
| placebo in subjects with moderate-to-severe | (PROMIS-29) at Week 16 |
| plaque psoriasis | Proportion of subjects who achieve |
| | at least a 5-point improvement from |
| | baseline in each PROMIS-29 domain at |
| | Week 16 |
| To assess the safety and tolerability of IL-23 | Frequency and type of AEs and SAEs |
| receptor antagonist peptide in subjects with | |
| moderate-to-severe plaque psoriasis | |
| Exploratory | |
| To evaluate the PK and | PK parameters (i.e., plasma |
| immunogenicity of IL-23 receptor | concentration just prior to the beginning or |
| antagonist peptide and explore the PK/PD | at the end of the dosing interval [Ctrough], |
| relationship of IL-23 receptor antagonist | Cmax, tmax, and AUC) |
| peptide for biomarkers, efficacy, and safety | The relationship between PK |
| in participants with moderate-to-severe | parameters and PD (i.e., skin, blood cellular |
| plaque psoriasis | and molecular biomarker activity as well as |
| | clinical endpoints and safety parameters) |
| | The incidence of anti-drug antibodies |
| | to IL-23 receptor antagonist peptide |
| To characterize additional efficacy | Proportion of participants |
| of IL-23 receptor antagonist peptide versus | achieving an ss-IGA score of absence of |
| placebo for the treatment of regional | disease (0) or very mild disease (1) and at |
| psoriasis | least a 2-grade improvement from baseline |
| | at Week 16 among those participants |
| | randomized with scalp psoriasis and an ss- |
| | IGA score ≥2 at baseline |
| | Percent change from baseline in |
| | NAPSI at Week 16 among participants |
| | randomized with a NAPSI score >0 at |
| | baseline |
| | Proportion of participants |
| | achieving an f-PGA score of clear (0) or |
| | minimal (1) at Week 16 among those |
| | participants randomized with nail psoriasis |
| | and an f-PGA ≥2 score at baseline |
| | Proportion of participants who |
| | achieve an hf-PGA score of clear (0) or |
| | almost clear (1) and a reduction of at least 2 |
| | grades on the hf-PGA scale from baseline at |
| | Week 16 among those participants with |
| | hand and/or foot psoriasis and an hf-PGA |
| | score ≥2 at baseline |
| | Proportion of participants |
| | achieving a sPGA of genitalia score of clear |
| | (0) or minimal (1) at Week 16 among |
| | participants randomized with genital |
| | psoriasis and an sPGA of genitalia score ≥3 |
| | at baseline |
| | Proportion of participants |
| | achieving a GenPs-SFQ item 2 score of |
| | never (0) or rarely (1) at Week 16 among |
| | those participants with a GenPs-SFQ item 2 |
| | score ≥2 at baseline |
| To further assess the safety and | Frequency and type of related AEs, |
| tolerability of IL-23 receptor antagonist | and AEs leading to discontinuation of study |
| peptide in participants with moderate-to- | intervention |
| severe plaque psoriasis | Laboratory parameters and change |
| | from baseline in laboratory parameters |
| | (hematology and chemistry) over time |
| | Systolic and diastolic blood |
| | pressures over time |
| To explore biomarkers in participants with | Change from baseline in levels of |
| moderate-to-severe plaque psoriasis | skin and blood biomarkers |
| To explore treatment satisfaction | Assessment of treatment |
| after using IL-23 receptor antagonist peptide | satisfaction domains at Week 16 using the |
| in participants with moderate-to-severe | Treatment Satisfaction Questionnaire for |
| plaque psoriasis | Medications-9 items (TSQM-9) |

### Overall Design

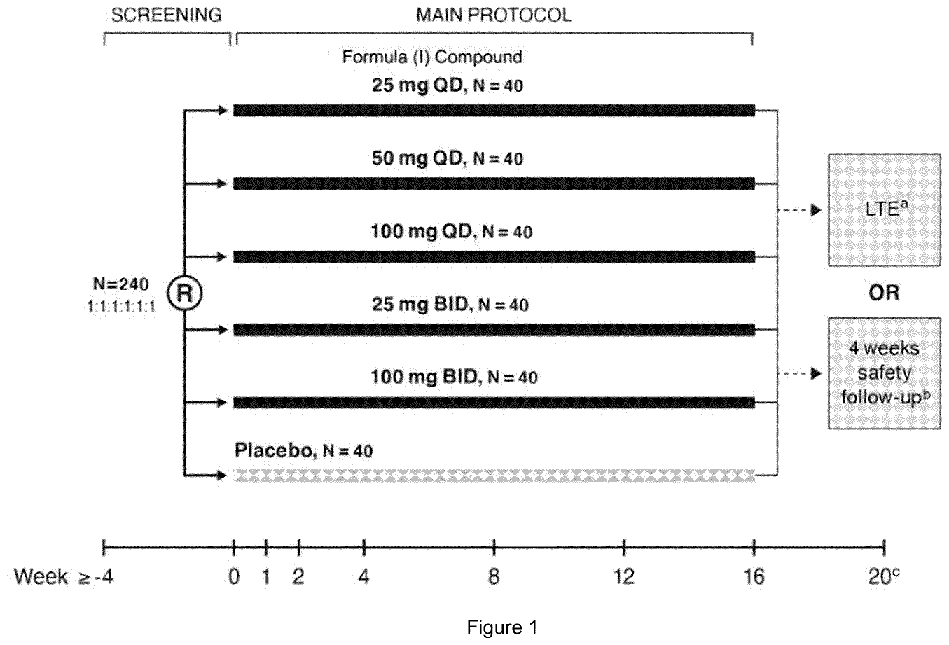

This was a randomized, double-blind, placebo-controlled, dose-ranging, parallel group, multicenter, interventional study in subjects with moderate-to-severe plaque psoriasis. A target of 240 subjects were enrolled in this study with 40 subjects planned per intervention group. A schematic view of this study is shown in FIG. 1.

Subjects were instructed to take the study intervention in the morning (AM) and in the evening (PM) on an empty stomach with approximately 240 mL of noncarbonated water at approximately the same time every day. An empty stomach is defined as abstaining from food intake for at least 2 hours before taking the study intervention and abstaining from food and liquid intake for at least 30 minutes after taking the study intervention.

Group 1: 25 mg once daily: Subjects receive IL-23 receptor antagonist peptide 25 mg once daily (QD) from Week 0 through Week 16.

Group 2: 50 mg once daily: Subjects receive IL-23 receptor antagonist peptide 50 mg QD from Week 0 through Week 16.

Group 3: 100 mg once daily: Subjects receive IL-23 receptor antagonist peptide 100 mg QD from Week 0 through Week 16.

Group 4: 25 mg twice daily: Subjects receive IL-23 receptor antagonist peptide 25 mg twice daily (BID) from Week 0 through Week 16.

Group 5: 100 mg twice daily: Subjects receive IL-23 receptor antagonist peptide 100 mg BID from Week 0 through Week 16.

Group 6: Placebo: Subjects receive placebo BID from Week 0 through Week 16.

At Week 16, eligible subjects have the option to enroll in a 36-week treatment long term extension (LTE) study; all subjects will be treated with active study intervention in the LTE.

The total duration of this study was 24 weeks: a screening period of ≤4 weeks, a 16-week treatment period, and a 4-week safety follow-up period after the last study intervention administration for subjects who were ineligible or decided to not enroll in the LTE study at Week 16. All eligible subjects who enrolled in the LTE continue treatment after the Week 16 visited under the LTE protocol.

Efficacy, safety, PK, immunogenicity, and biomarkers were assessed. In addition, there were four optional substudy collections for subjects who consented (where local regulations permit): a pharmacogenomic blood sample, ex vivo cytokine release blood sample, skin biopsy, and photograph collection (lesional or lesional and full body).

### Inclusion Criteria

Each subject was ≥18 years old, and satisfied all of the following criteria:
(a) had a diagnosis of plaque psoriasis, with or without PsA, for at least 6 months prior to the first administration of study intervention;
(b) had a total BSA≥10% at screening and baseline;
(c) had a total PASI≥12 at screening and baseline;
(d) had a total IGA≥3 at screening and baseline;
(e) was a candidate for phototherapy or systemic treatment for plaque psoriasis;
(f) agreed to avoid prolonged sun exposure and avoid use of tanning booths or other ultraviolet (UV) light sources during the study; and was
(g) otherwise healthy on the basis of physical examination, medical history, and vital signs, and 12-lead triplicate electrocardiogram (ECG) performed at screening.

### Exclusion Criteria

A potential subject was excluded if the subject has any of the following disease characteristics:
(a) had a nonplaque form of psoriasis (e.g., erythrodermic, guttate, or pustular); or
(b) had current drug-induced psoriasis (e.g., a new onset of psoriasis or an exacerbation of psoriasis from beta blockers, calcium channel blockers, or lithium).

A subject was also excluded if the subject:
(a) had previously received any other therapeutic agent directly targeted to IL-23;
(b) had received any therapeutic agent directly targeted to IL-17 or IL-12/23 or had received anti-TNFα biologic therapy within 12 weeks or 5 half-lives, whichever is longer, of the first administration of study intervention;
(c) had received agents that deplete B cells (including, but not limited to, rituximab, or alemtuzumab) within 26 weeks of the first administration of study intervention;
(d) had received natalizumab, belimumab, or agents that modulate T cells (including, but not limited to abatacept or visilizumab) 12 weeks or 5 half-lives, whichever is longer, of the first administration of study intervention;
(e) had received JAK inhibitor therapy within 4 weeks of the first administration of study intervention;
(f) had received phosphodiesterase 4 (PDE4) inhibitor therapy (including but not limited to apremilast) within 4 weeks of the first administration of study intervention;
(g) had received any systemic immunosuppressants (including, but not limited to, methotrexate (MTX), azathioprine, cyclosporine, 6-thioguanine, mercaptopurine, mycophenolate mofetil, and tacrolimus) within 4 weeks of the first administration of study intervention;
(h) had received, or was expected to receive, any live virus or bacterial vaccination within 12 weeks before the first administration of study intervention, with the exception of the Bacille Calmette Guerin (BCG) vaccine below;
(i) had received the BCG vaccine within 12 months of the first administration of study intervention;
(j) had received phototherapy or any systemic medications that could affect psoriasis or the PASI or IGA evaluations (including, but not limited to, oral or injectable corticosteroids, acitretin, retinoids, 1,25-dihydroxy vitamin D3 and analogues, herbal treatments or traditional Taiwanese, Korean, or Chinese medicines) within 4 weeks of the first administration of study intervention; or
(k) had received topical therapies that could affect psoriasis or the PASI or IGA evaluation (including, but not limited to corticosteroids, tar, anthralin, calcipotriene, tazarotene, methoxsalen, pimecrolimus, tacrolimus, and traditional Taiwanese, Korean, or Chinese medicines) within 2 weeks of the first administration of study intervention.

### Intervention Groups

Each subject in the groups was administered a film-coated tablet containing IL-23 receptor antagonist peptide or placebo twice daily according to Table 1. Subjects took the tablet with approximately 240 mL of noncarbonated water and on an empty stomach consistently throughout the study (including site visit days). An empty stomach is defined as abstaining from food intake for at least 2 hours before taking the study intervention and abstaining from food and liquid intake for at least 30 minutes after taking the study intervention.

TABLE 1
Study Interventions Administered on an Empty Stomach

| Group | 1 | 2 | 3 | 4 | 5 | 6 |
|---|---|---|---|---|---|---|
| Dosage and | 25 mg QD | 50 mg QD | 100 mg QD | 25 mg BID | 100 mg BID | placebo |
| Frequency | (AM) and | (AM) and | (AM) and | (AM and PM) | (AM and PM) | (AM and PM) |
| | placebo (PM) | placebo (PM) | placebo (PM) | | | |

### Study Evaluations

Efficacy, PK, immunogenicity, biomarker, pharmacogenomic, and safety criteria were measured during the treatment. Subjects were provided with an electronic device to enter patient-reported outcome (PRO) data at each study visit. All visit-specific PRO assessments were conducted/completed before any tests, procedures or other clinical assessments, with the exception of the urine pregnancy test and urinalysis, to prevent influencing subject perceptions.

Electrocardiograms (ECGs) precede vital signs and both procedures were completed prior to any invasive procedures. Vital signs were recorded from the opposite arm from which blood samples are being taken.

All samples (including safety, efficacy, PK, and biomarkers) were obtained after the PRO and ECG assessments.

Clinician-reported outcomes including Psoriasis Area and Severity Index (PASI), Body Surface Area (BSA), Investigator's Global Assessment (IGA), Nail Psoriasis Area and Severity Index (NAPSI), Fingernail Physician's Global Assessment (f-PGA), Static Physician's Global Assessment of Genitalia (s-PGA-G), Physician's Global Assessment of Hands and/or Feet (hf-PGA), and Scalp Specific Investigator Global Assessment (ss-IGA) were obtained at Week 0, 8, and 16. PASI and IGA were also obtained at Weeks 1, 2, 4, and 12, and at Week 20, four weeks after the administration of the tablets has stopped.

Adverse events were reported and followed by the investigator. Any clinically relevant changes occurring during the study were recorded. Any clinically significant abnormalities persisting at the end of the study/early withdrawal were followed by the investigator until resolution or until a clinically stable condition was reached if possible. The evaluations of safety and tolerability include: physical examinations, vital signs, electrocardiograms, clinical safety laboratory assessments, review of concomitant medication, pregnancy testing, suicidal ideation evaluation via the Columbia—Suicide Severity Rating Scale, and tuberculosis evaluation.

### Results Summary

A total of 337 participants were screened of which 255 participants were randomized into 6 treatment groups (Placebo (n=43), 25 mg QD (n=43), 50 mg QD (n=43), 25 mg BID (n=41), 100 mg QD (n=43), 100 mg BID (n=43)). The study was conducted at 65 sites across 10 countries with 49.4% of participants from Europe, and the remaining participants distributed across Asia (17.3%), and North America (33.3%). Most participants were White (74.5%) and male (69.0%). The mean age (SD) was 44.3 (12.65) years and mean baseline weight was 88.9 (20.87) kg.

Disposition and baseline characteristics for the primary efficacy analysis set (n=255): In total, twenty-four (9.4%) participants discontinued study agent through Week 16. The proportion of participants discontinuing study agent was highest in the placebo group and 25 mg QD group (16.3% [n=7] each). A total of 17 participants (8.0%) in the combined groups discontinued study agent, with some variability across dose groups. No dose-dependent trends were observed for any reason of discontinuation.

Baseline disease characteristics were generally comparable between the treatment groups. Overall, the mean psoriasis disease duration (SD) was 18.2 (12.79) years. The mean PASI total score (SD) was 19.05 (5.831). A higher proportion of patients in the 25 mg QD arm (30.2%) and the 100 mg BID dose (28.6%) had severe psoriasis based on IGA score of 4 at baseline compared to placebo (IGA of 4 in 11.6%) and other treatment arms (range 16.3-19.5%). Variability in the baseline data were observed between treatment groups, but arms were mostly balanced.

The proportions of participants receiving previous therapies were comparable across treatment groups. Overall, 43.1% previously received phototherapy, 47.8% previously received conventional non-biologic systemic therapy, 7.1% previously received novel non-biologic systemics, and 22.0% previously received biologic therapy.

### Study Results Analysis

The primary efficacy endpoint was the proportion of participants achieving a PASI 75 response at Week 16, defined as at least a 75% reduction from baseline in PASI total score.

For the primary analysis, composite strategy (non-responder imputation) was applied to address intercurrent event #1 (Discontinuation of study intervention due to lack of efficacy or due to an AE of worsening of psoriasis) and #2 (initiation of a protocol-prohibited medication or therapy that could improve psoriasis); and treatment policy strategy (observed data) was applied to intercurrent event #3 (Discontinuation of study intervention due to other reasons). Participants with missing data after application of ICEs are also considered as non-responders.

The MCP Mod was used to test if there is a positive overall treatment effect based on candidate response models. A response signal was detected (p-value<0.001) for the primary endpoint.

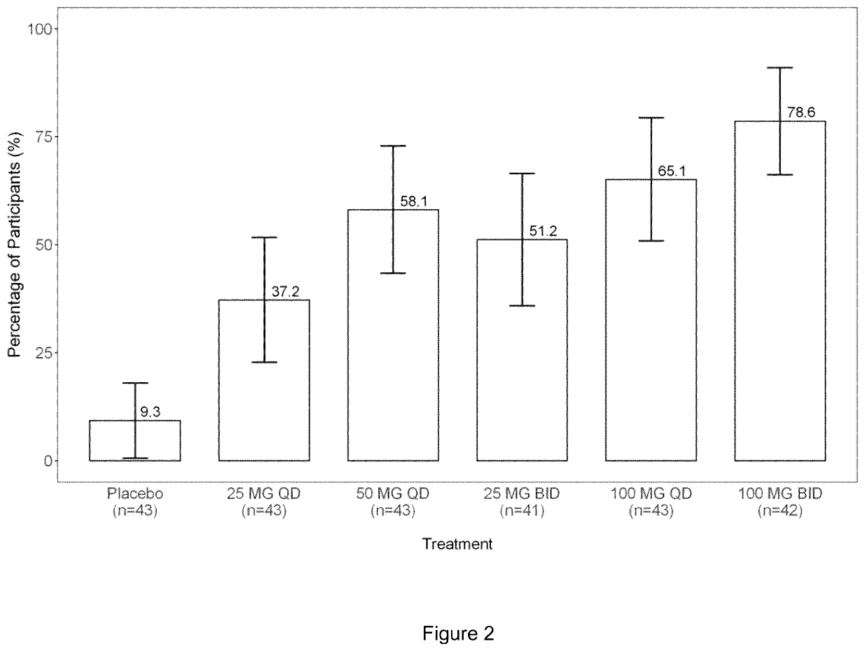

In addition to the response analysis, pairwise comparisons of each treatment group versus the placebo group were performed for the primary endpoint of PASI 75 at Week 16. Pairwise comparisons were not adjusted for multiplicity. The Cochran-Mantel-Haenszel chi-square statistic stratified by baseline weight category (≤90 kg, >90 kg) at a 2-sided significance level of 0.05 was used (Table 2). PASI 75 response at Week 16 is also shown in FIG. 2. A significantly higher proportion of participants in each dose group achieved a PASI 75 response at Week 16 than the placebo group.

As shown in the response signal, a significantly higher proportion of participants in each dose group achieved a PASI 75 response at Week 16 (37.2% for 25 mg QD, 58.1% for 50 mg QD, 51.2% for 25 mg BID, 65.1% for 100 mg QD, 78.6% for 100 mg BID; p=0.002 for 25 mg QD; p<0.001 for all other dose groups) than the placebo group (9.3%).

A significant response signal was detected for the primary endpoint (PASI 75 response at Week 16) as shown in FIG. 2.

TABLE 2
Proportion of Subjects Achieving PASI 75 Response at Week 16

| Treatment Regimen | Placebo | 25 mg QD | 50 mg QD | 25 mg BID | 100 mg QD | 100 mg BID |
|---|---|---|---|---|---|---|
| Analysis set: Full analysis set | 43 | 43 | 43 | 41 | 43 | 42 |
| Subjects achieving ≥75% | 4 | 16 | 25 | 21 | 28 | 33 |
| Improvement at Week 16^{a} | (9.3%) | (37.2%) | (58.1%) | (51.2%) | (65.1%) | (78.6%) |
| Treatment difference (95% | N/A | 27.9% | 48.8% | 41.9% | 55.8% | 69.4% |
| CI)^{b} | | (11.2%, | (31.9%, | (24.5%, | (39.3%, | (54.7%, |
| | | 44.6%) | 65.8%) | 59.3%) | 72.3%) | 84.1%) |
| p-value^{c} | N/A | 0.002 | <0.001 | <0.001 | <0.001 | <0.001 |

^{a}Subjects with ICE 1-2 were assumed to be non-responders after the event. Observed data were used for subjects with ICE 3. After accounting for the ICEs, subjects with missing data were considered as non-responders.
^{b}Treatment difference and 95% CI were calculated adjusting for baseline weight category (≤90 kg, >90 kg) using Mantel-Haenszel (MH) weights.
^{c}The p-values are based on Cochran-Mantel-Haenszel (CMH) chi-square test stratified by baseline weight category (≤90 kg, >90 kg).

### Secondary Efficacy Endpoints:

No multiplicity adjustment was made for the secondary endpoints for this study. All statistical testing was performed at the 2-sided 0.05 significance level. Nominal p-values are presented.

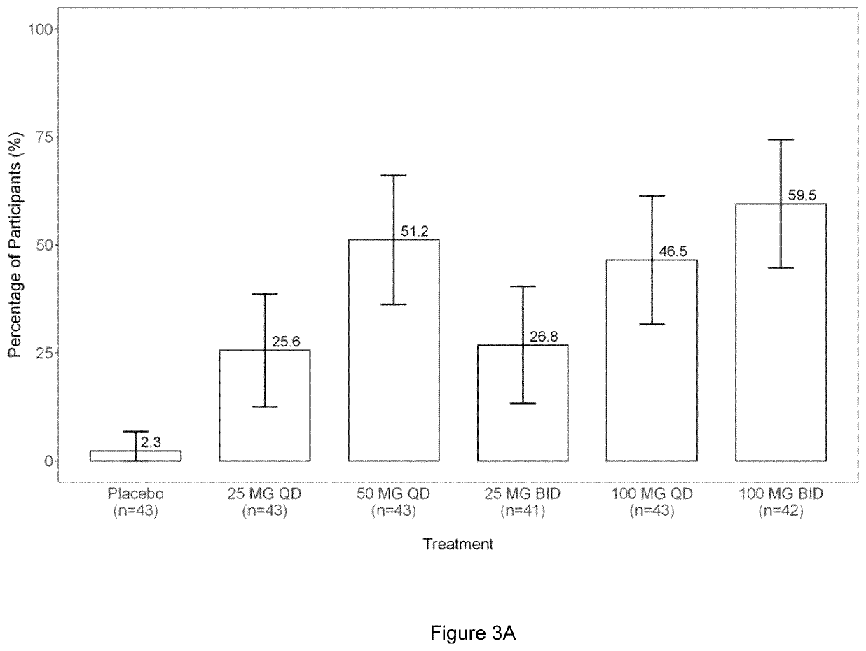

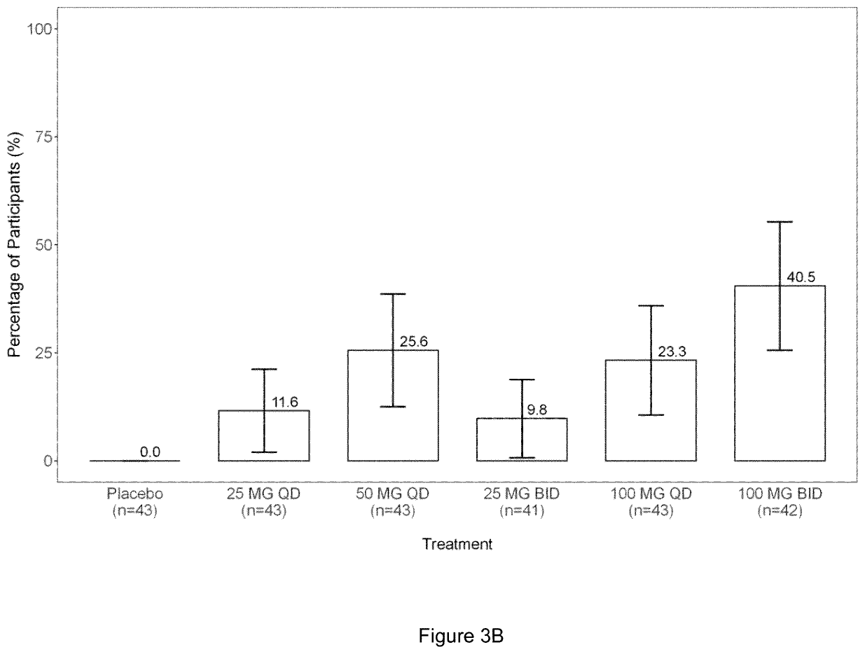

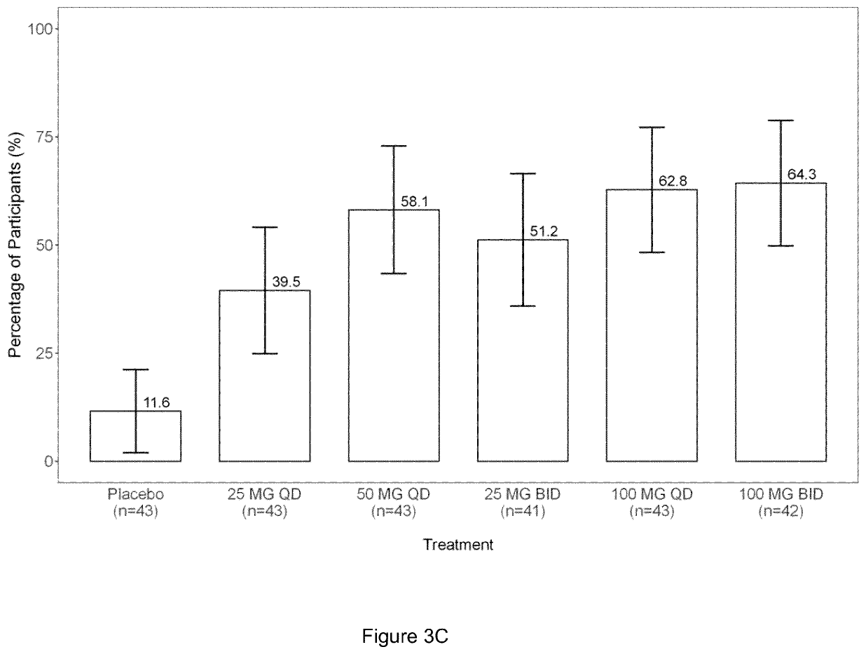

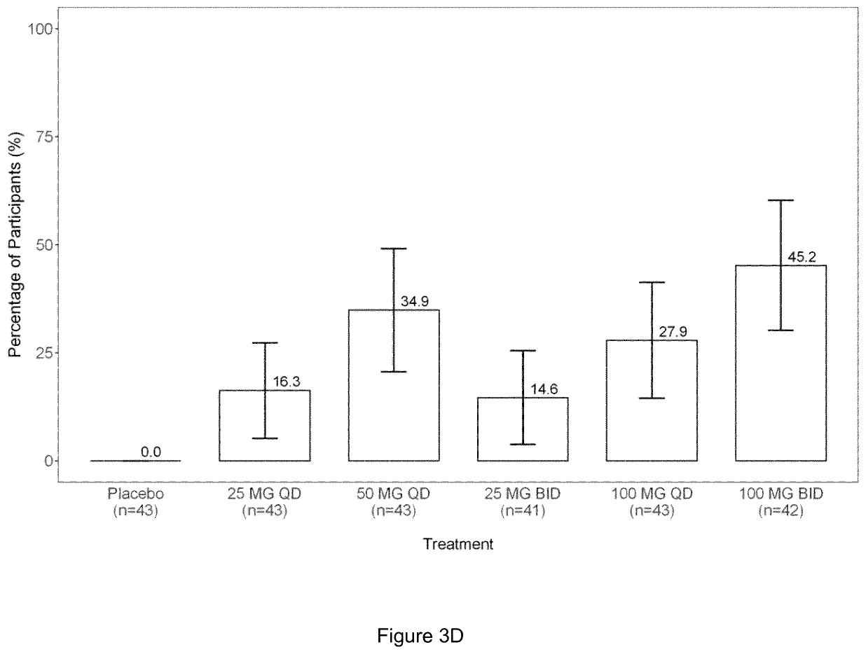

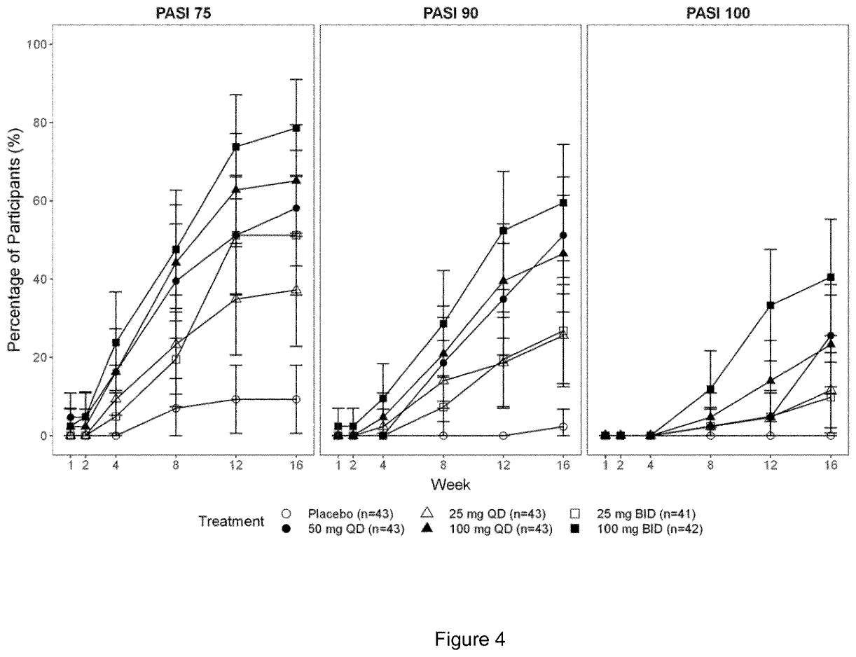

All selected secondary endpoints shown achieved statistical significance at a nominal significance level of 0.05 (2-sided) at Week 16. At Week 16, the proportions of participants achieving an IGA score of cleared (0) or minimal (1), IGA score of cleared (0), PASI 90 response, PASI 100 response (FIG. 3) or DLQI score of 0 or 1 were significantly higher for participants in each peptide dose group compared with placebo. At Week 16, participants in each peptide dose groups had a significantly greater improvement (reduction) as measured by PASI total score, BSA, PSSD symptoms score, and PSSD signs score from baseline than participants treated with placebo. In overtime analyses, PASI and IGA response rates showed clear separation between peptide doses from placebo as early as Week 4 and generally continued to increase through Week 16. PASI 75, PASI 90, and PASI 100 response rates in most dose groups continued on an upward trend without obvious plateaus through Week 16 (FIG. 4).

The proportions of participants who achieved an IGA score of cleared (0), an IGA score of cleared (0) or minimal (1), a PASI 100, and a PASI 90 response at Week 16 are summarized in Table 3 and FIG. 3.

TABLE 3
PASI and IGA Responses at Week 16

| Treatment Regimen | Placebo | | 25 mg QD | | 50 mg QD | 25 mg BID | | 100 mg QD | 100 mg BID | |
|---|---|---|---|---|---|---|---|---|---|---|
| Analysis set: Full analysis set | 43 | | 43 | | 43 | 41 | | 43 | 42 | |
| Subjects achieving ≥90% | 1 | (2.3%) | 11 | (25.6%) | 22 (51.2%) | 11 | (26.8%) | 20 (46.5%) | 25 | (59.5%) |
| Improvement at Week 16^{aa} | | | | | | | | | | |
| Treatment difference (95% CI)^{b} | N/A | | 23.3% (9.6%, | | 48.8% (33.3%, | 24.5% (10.4%, | | 44.2% (28.6%, | 57.3% (42.0%, | |
| | | | 36.9%) | | 64.4%) | 38.5%) | | 59.7%) | 72.6%) | |
| p-value^{c} | N/A | | 0.002 | | <0.001 | 0.001 | | <0.001 | <0.001 | |
| Subjects achieving 100% | 0 | | 5 | (11.6%) | 11 (25.6%) | 4 | (9.8%) | 10 (23.3%) | 17 | (40.5%) |
| Improvement at Week 16^{a} | | | | | | | | | | |
| Treatment difference (95% CI)^{b} | N/A | | 11.6% (2.4%, | | 25.6% (12.7%, | 9.7% (0.7%, | | 23.3% (10.7%, | 40.5% (25.8%, | |
| | | | 20.8%) | | 38.5%) | 18.8%) | | 35.8%) | 55.3%) | |
| p-value^{c} | N/A | | 0.021 | | <0.001 | 0.038 | | <0.001 | <0.001 | |
| Subjects achieving IGA Score = | 5 | (11.6%) | 17 | (39.5%) | 25 (58.1%) | 21 | (51.2%) | 27 (62.8%) | 27 | (64.3%) |

| 0 or 1 at Week 16^{a} | | | | | | | | | |
|---|---|---|---|---|---|---|---|---|---|
| Treatment difference (95% CI)^{b} | N/A | 27.9% (10.5%, | | 46.5% (29.1%, | 39.5% (21.7%, | | 51.2% (34.0%, | 52.8% (36.0%, | |
| | | 45.3%) | | 63.9%) | 57.3%) | | 68.3%) | 69.7%) | |
| p-value^{c} | N/A | 0.003 | | <0.001 | <0.001 | | <0.001 | <0.001 | |
| Subjects achieving IGA Score = | 0 | 7 | (16.3%) | 15 (34.9%) | 6 | (14.6%) | 12 (27.9%) | 19 | (45.2%) |

| | | | | | | |
|---|---|---|---|---|---|---|
| 0 at Week 16^{a} | | | | | | |
| Treatment difference (95% CI)^{b} | N/A | 16.3% (5.3%, | 34.9% (20.8%, | 14.6% (4.0%, | 27.9% (14.6%, | 45.3% (30.6%, |
| | | 27.2%) | 48.9%) | 25.2%) | 41.2%) | 60.1%) |
| p-valuc^{c} | N/A | 0.006 | <0.001 | 0.009 | <0.001 | <0.001 |

^{a}Subjects with ICE 1-2 were assumed to be non-responders after the event. Observed data were used for subjects with ICE 3. After accounting for the ICEs, subjects with missing data were considered as non-responders.
^{b}Treatment difference and 95% CI were calculated adjusting for baseline weight category (≤90 kg, >90 kg) using Mantel-Haenszel (MH) weights.
^{c}The p-values are based on Cochran-Mantel-Haenszel (CMH) chi-square test stratified by baseline weight category (≤90 kg, >90 kg).

### Over Time Through Week 16

### PASI75, PASI90, PASI100, IGA0/1 and IGA0 Over Time:

The proportions and the corresponding 95% CIs of participants achieving PASI75, PASI90, PASI100, IGA0/1 and IGA0 over time from Week 1 through Week 16 are summarized in FIG. 4. A clear separation between peptide doses and placebo (0.0%) in the proportion of participants achieved PASI 75 responses started at Week 4 for 50 mg QD (16.3%), 100 mg QD (16.3%) and 100 mg BID group (23.8%). After week 4, PASI 75 response continued to improve through Week 16 for the treatment groups. The PASI 75 response was generally stable or increased from Week 12 through Week 16 for all of the groups. Similar response pattern was observed for PASI 90 and IGA 0/1 responses respectively. For PASI 100 response, a clear separation between peptide doses and placebo (0.0%) in the proportion of participants achieved PASI 100 responses started at Week 8 for 100 mg BID group (11.9%), and PASI 100 response rate for 100 mg BID group continue increase to Week 16 (40.5%). Similar response pattern was observed for the endpoint of proportion of participants achieving IGA score of (0).

### Safety:

Safety was assessed among all randomized and treated participants who received at least 1 dose of study agent (partial or complete) according to the assigned treatment received during the study. This is also referred to as the safety analysis set.

Overall, IL-23 receptor antagonist peptide was well-tolerated by participants in all IL-23 receptor antagonist peptide treatment groups during the study and the proportion of subjects experiencing 1 or more AEs was comparable between IL-23 receptor antagonist peptide groups and the placebo group. There was no evidence of dose-dependent increase in the occurrence of AEs across the peptide treatment groups. There were three SAEs that occurred in the study (n=1 each: suicide attempt, COVID-19, infected cyst). No dose-dependent relationship was observed.

A low number of laboratory abnormalities occurred during the study and were comparable between placebo and treatment group. There was no evidence of a dose-dependent increase in the occurrence of abnormalities. There were no deaths, MACE or malignancies during the study.

Table 4 provides an overview of the key safety results through the end of the study.

TABLE 4
Key Safety Results

| Treatment Regimen | Placebo | | 25 mg QD | | 50 mg QD | | 25 mg BID | | 100 mg QD | | 100 mg BID | | Combined^{a} | |
|---|---|---|---|---|---|---|---|---|---|---|---|---|---|---|
| Analysis set: Safety analysis set | 43 | | 43 | | 43 | | 41 | | 43 | | 42 | | 212 | |
| Avg duration of follow-up | 15.03 | | 15.70 | | 15.75 | | 16.20 | | 16.07 | | 15.81 | | 15.90 | |
| (weeks) | | | | | | | | | | | | | | |
| Subjects who discontinued study | 1 | (2.4%) | 3 | (7.0%) | 1 | (2.3%) | 1 | (2.3%) | 0 | | 0 | | 5 | (2.3%) |
| agent because of 1 or more | | | | | | | | | | | | | | |
| adverse events | | | | | | | | | | | | | | |
| Subjects with 1 or more: | | | | | | | | | | | | | | |
| Adverse events | 22 | (51.2%) | 20 | (46.5%) | 26 | (60.5%) | 20 | (48.8%) | 19 | (44.2%) | 26 | (61.9%) | 111 | (52.4%) |
| Serious adverse events (3 events | — | | — | | — | | — | | — | | — | | — | |
| total) | | | | | | | | | | | | | | |
| Serious infections (2 events total) | — | | — | | — | | — | | — | | — | | — | |
| AEs occurring with frequence of | | | | | | | | | | | | | | |
| at least 5% | | | | | | | | | | | | | | |
| Infections and infestations | 12 | (27.9%) | 15 | (34.9%) | 17 | (39.5%) | 14 | (34.1%) | 7 | (16.3%) | 11 | (26.2%) | 64 | (30.2%) |
| COVID-19 | 5 | (11.6%) | 5 | (11.6%) | 3 | (7.0%) | 8 | (19.5%) | 3 | (7.0%) | 4 | (9.5%) | 23 | (10.8%) |
| Nasopharyngitis | 2 | (4.7%) | 1 | (2.3%) | 8 | (18.6%) | 3 | (7.3%) | 1 | (2.3%) | 2 | (4.8%) | 15 | (7.1%) |
| Upper respiratory tract infection | 1 | (2.3%) | 3 | (7.0%) | 0 | | 0 | | 0 | | 2 | (4.8%) | 5 | (2.4%) |
| Gastrointestinal disorders | 5 | (11.6%) | 3 | (7.0%) | 6 | (14.0%) | 4 | (9.8%) | 4 | (9.3%) | 7 | (16.7%) | 24 | (11.3%) |
| Diarrhea | 1 | (2.3%) | 2 | (4.7%) | 4 | (9.3%) | 2 | (4.9%) | 1 | (2.3%) | 1 | (2.4%) | 10 | (4.7%) |

| | | | | | | | | | | | | | |
|---|---|---|---|---|---|---|---|---|---|---|---|---|---|
| Nervous system disorders | 1 | (2.3%) | 0 | 3 | (7.0%) | 2 | (4.9%) | 3 | (7.0%) | 2 | (4.8%) | 10 | (4.7%) |
| Headache | 1 | (2.3%) | 0 | 1 | (2.3%) | 1 | (2.4%) | 3 | (7.0%) | 1 | (2.4%) | 6 | (2.8%) |

| | | | | | | | | | | | | | |
|---|---|---|---|---|---|---|---|---|---|---|---|---|---|
| Respiratory, thoracic and | 1 | (2.3%) | 1 | (2.3%) | 1 | (2.3%) | 0 | 3 | (7.0%) | 2 | (4.8%) | 7 | (3.3%) |

| | | | | | | | |
|---|---|---|---|---|---|---|---|
| mediastinal disorders | | | | | | | |
| Cough (6 events total) | — | — | — | — | — | — | — |

^{a}Includes all IL-23 receptor antagonist peptide treatment columns (25 mg QD, 50 mg QD, 25 mg BID, 100 mg QD, and 100 mg BID). Subjects are counted only once for any given event, regardless of the number of times they actually experienced the event. Adverse events are coded using MedDRA Version 25.1.
*MACE: myocardial infarction (MI), stroke or CV death.

### Pharmacokinetics:

The evaluable pharmacokinetics (PK) analysis set included all randomized subjects who received at least 1 complete dose of the IL-23 receptor antagonist peptide and had at least 1 valid blood sample drawn for PK analysis after their first dose of the IL-23 receptor antagonist peptide. Plasma concentrations of the IL-23 receptor antagonist peptide from Week 1 through Week 16 are summarized in Table 5. The number of participants with plasma the IL-23 receptor antagonist peptide concentration below the LLOQ (0.02 ng/mL) from Week 1 through Week 16 is summarized in Table 6.

TABLE 5
Plasma IL-23 Receptor Peptide Antagonist Concentrations (ng/mL) through Week 16

| Treatment Regimen | 25 mg QD | | 50 mg QD | | 25 mg BID | | 100 mg QD | | 100 mg BID | |
|---|---|---|---|---|---|---|---|---|---|---|
| Analysis set: | 42 | | 43 | | 41 | | 42 | | 42 | |
| Pharmacokinetics | | | | | | | | | | |
| Evaluable^{a} | | | | | | | | | | |
| Week 1 | | | | | | | | | | |
| Trough^{b, c} | | | | | | | | | | |
| N | 23 | | 22 | | 23 | | 21 | | 30 | |
| Mean (SD) | 0.051 | (0.0291) | 0.087 | (0.0372) | 0.146 | (0.1030) | 0.159 | (0.0972) | 0.742 | (0.3416) |
| Median | 0.057 | | 0.074 | | 0.116 | | 0.132 | | 0.625 | |
| Geometric Mean | 0.055 | | 0.080 | | 0.125 | | 0.134 | | 0.665 | |
| Range | (0.00; 0.11) | | (0.04; 0.17) | | (0.06; 0.54) | | (0.04; 0.42) | | (0.17; 1.57) | |
| IQ range | (0.034; 0.069) | | (0.056; 0.109) | | (0.090; 0.168) | | (0.092; 0.200) | | (0.503; 1.010) | |
| Intermediate^{b, c} | | | | | | | | | | |
| N | 3 | | 3 | | 1 | | 5 | | 5 | |
| Mean (SD) | 0.131 | (0.0387) | 0.349 | (0.1932) | 0.077 | (—) | 0.598 | (0.6217) | 1.551 | (1.0516) |
| Median | 0.128 | | 0.360 | | 0.077 | | 0.480 | | 1.410 | |
| Geometric Mean | 0.127 | | 0.307 | | 0.077 | | 0.378 | | 1.257 | |
| Range | (0.09; 0.17) | | (0.15; 0.54) | | (0.08; 0.08) | | (0.12; 1.64) | | (0.45; 3.11) | |
| IQ range | (0.094; 0.171) | | (0.150; 0.536) | | (0.077; 0.077) | | (0.135; 0.619) | | (0.793; 1.990) | |
| Peak^{b, c} | | | | | | | | | | |
| N | 24 | | 26 | | 29 | | 23 | | 19 | |
| Mean (SD) | 0.528 | (0.3970) | 0.900 | (0.8287) | 0.572 | (0.5240) | 1.482 | (0.9745) | 1.980 | (1.9157) |
| Median | 0.453 | | 0.663 | | 0.447 | | 1.270 | | 1.740 | |
| Geometric Mean | 0.397 | | 0.621 | | 0.409 | | 1.245 | | 1.501 | |
| Range | (0.04; 1.59) | | (0.07; 3.83) | | (0.05; 2.71) | | (0.28; 4.97) | | (0.32; 9.19) | |
| IQ range | (0.210; 0.669) | | (0.465; 1.190) | | (0.311; 0.678) | | (0.880; 1.790) | | (1.020; 2.230) | |
| Week 2 | | | | | | | | | | |
| Trough | | | | | | | | | | |
| N | 16 | | 20 | | 19 | | 15 | | 24 | |
| Mean (SD) | 0.069 | (0.0596) | 0.124 | (0.1331) | 0.228 | (0.2260) | 0.252 | (0.1854) | 0.670 | (0.3715) |
| Median | 0.044 | | 0.089 | | 0.165 | | 0.148 | | 0.636 | |
| Geometric Mean | 0.056 | | 0.096 | | 0.181 | | 0.191 | | 0.575 | |
| Range | (0.02; 0.26) | | (0.03; 0.66) | | (0.00; 0.98) | | (0.04; 0.61) | | (0.16; 1.75) | |
| IQ range | (0.042; 0.068) | | (0.071; 0.134) | | (0.088; 0.264) | | (0.113; 0.437) | | (0.399; 0.822) | |
| Intermediate | | | | | | | | | | |
| N | 2 | | 3 | | 2 | | 5 | | 1 | |
| Mean (SD) | 0.210 | (0.0474) | 0.271 | (0.0967) | 0.658 | (0.5127) | 0.628 | (0.1063) | 0.912 | (—) |
| Median | 0.210 | | 0.295 | | 0.658 | | 0.649 | | 0.912 | |
| Geometric Mean | 0.207 | | 0.258 | | 0.549 | | 0.621 | | 0.912 | |
| Range | (0.18; 0.24) | | (0.17; 0.35) | | (0.30; 1.02) | | (0.50; 0.78) | | (0.91; 0.91) | |
| IQ range | (0.176; 0.243) | | (0.165; 0.354) | | (0.295; 1.020) | | (0.560; 0.655) | | (0.912; 0.912) | |
| Peak | | | | | | | | | | |
| N | 18 | | 18 | | 21 | | 21 | | 17 | |
| Mean (SD) | 0.251 | (0.3176) | 0.943 | (1.2772) | 0.529 | (0.5460) | 0.845 | (0.8036) | 2.318 | (2.8265) |
| Median | 0.125 | | 0.573 | | 0.368 | | 0.627 | | 1.530 | |
| Geometric Mean | 0.171 | | 0.457 | | 0.355 | | 0.487 | | 1.492 | |
| Range | (0.00; 1.26) | | (0.00; 5.20) | | (0.06; 2.49) | | (0.06; 2.67) | | (0.42; 12.20) | |
| IQ range | (0.032; 0.400) | | (0.112; 1.330) | | (0.248; 0.578) | | (0.136; 1.350) | | (0.748; 2.710) | |
| Week 4 | | | | | | | | | | |
| Trough | | | | | | | | | | |
| N | 18 | | 27 | | 27 | | 19 | | 23 | |
| Mean (SD) | 0.067 | (0.1037) | 0.106 | (0.0471) | 0.185 | (0.1338) | 0.238 | (0.1950) | 1.008 | (2.2383) |
| Median | 0.041 | | 0.106 | | 0.147 | | 0.183 | | 0.452 | |
| Geometric Mean | 0.055 | | 0.096 | | 0.155 | | 0.190 | | 0.473 | |
| Range | (0.00; 0.47) | | (0.02; 0.25) | | (0.00; 0.52) | | (0.08; 0.89) | | (0.05; 10.90) | |
| IQ range | (0.024; 0.065) | | (0.078; 0.127) | | (0.091; 0.254) | | (0.119; 0.287) | | (0.303; 0.542) | |
| Intermediate | | | | | | | | | | |
| N | 3 | | 3 | | 1 | | 4 | | 3 | |
| Mean (SD) | 0.178 | (0.0760) | 0.280 | (0.1926) | 0.083 | (—) | 0.701 | (0.1981) | 0.804 | (0.3280) |
| Median | 0.177 | | 0.185 | | 0.083 | | 0.626 | | 0.808 | |
| Geometric Mean | 0.166 | | 0.243 | | 0.083 | | 0.683 | | 0.756 | |
| Range | (0.10; 0.25) | | (0.15; 0.50) | | (0.08; 0.08) | | (0.56; 0.99) | | (0.47; 1.13) | |
| IQ range | (0.102; 0.254) | | (0.154; 0.502) | | (0.083; 0.083) | | (0.584; 0.819) | | (0.474; 1.130) | |
| Peak | | | | | | | | | | |
| N | 16 | | 15 | | 12 | | 14 | | 11 | |
| Mean (SD) | 0.591 | (0.4048) | 0.564 | (0.3035) | 0.655 | (0.4122) | 1.310 | (0.8150) | 2.048 | (1.3703) |
| Median | 0.425 | | 0.539 | | 0.520 | | 1.345 | | 1.840 | |
| Geometric Mean | 0.469 | | 0.481 | | 0.539 | | 0.968 | | 1.696 | |
| Range | (0.09; 1.51) | | (0.16; 1.22) | | (0.18; 1.37) | | (0.11; 2.69) | | (0.49; 5.53) | |
| IQ range | (0.290; 0.838) | | (0.332; 0.773) | | (0.341; 0.997) | | (0.675; 1.980) | | (1.300; 2.550) | |
| Week 8 | | | | | | | | | | |
| Trough | | | | | | | | | | |
| N | 24 | | 28 | | 29 | | 30 | | 22 | |
| Mean (SD) | 0.068 | (0.0601) | 0.147 | (0.1368) | 0.184 | (0.1514) | 0.229 | (0.2226) | 0.961 | (1.0516) |
| Median | 0.060 | | 0.112 | | 0.143 | | 0.149 | | 0.744 | |
| Geometric Mean | 0.061 | | 0.111 | | 0.151 | | 0.175 | | 0.664 | |
| Range | (0.00; 0.29) | | (0.03; 0.70) | | (0.00; 0.61) | | (0.05; 1.20) | | (0.11; 5.14) | |
| IQ range | (0.037; 0.073) | | (0.063; 0.171) | | (0.089; 0.268) | | (0.121; 0.250) | | (0.388; 1.060) | |
| Intermediate | | | | | | | | | | |
| N | 4 | | 1 | | 1 | | 1 | | 4 | |
| Mean (SD) | 0.211 | (0.1584) | 0.441 | (—) | 0.092 | (—) | 0.235 | (—) | 1.041 | (0.9418) |
| Median | 0.177 | | 0.441 | | 0.092 | | 0.235 | | 0.840 | |
| Geometric Mean | 0.168 | | 0.441 | | 0.092 | | 0.235 | | 0.675 | |
| Range | (0.06; 0.43) | | (0.44; 0.44) | | (0.09; 0.09) | | (0.24; 0.24) | | (0.13; 2.36) | |
| IQ range | (0.110; 0.313) | | (0.441; 0.441) | | (0.092; 0.092) | | (0.235; 0.235) | | (0.472; 1.610) | |
| Peak | | | | | | | | | | |
| N | 12 | | 15 | | 13 | | 12 | | 15 | |
| Mean (SD) | 0.443 | (0.4220) | 1.104 | (0.9460) | 0.672 | (0.8784) | 1.145 | (1.1575) | 3.551 | (4.8202) |
| Median | 0.325 | | 0.780 | | 0.545 | | 0.873 | | 1.440 | |
| Geometric Mean | 0.284 | | 0.853 | | 0.483 | | 0.892 | | 1.861 | |
| Range | (0.03; 1.60) | | (0.25; 4.06) | | (0.00; 3.48) | | (0.44; 4.67) | | (0.20; 15.10) | |
| IQ range | (0.179; 0.570) | | (0.504; 1.380) | | (0.265; 0.689) | | (0.610; 1.008) | | (1.210; 2.280) | |
| Week 12 | | | | | | | | | | |
| Trough | | | | | | | | | | |
| N | 23 | | 20 | | 24 | | 25 | | 24 | |
| Mean (SD) | 0.052 | (0.0668) | 0.085 | (0.0517) | 0.202 | (0.1595) | 0.355 | (0.4482) | 1.096 | (0.9754) |
| Median | 0.038 | | 0.076 | | 0.151 | | 0.138 | | 0.834 | |
| Geometric Mean | 0.056 | | 0.086 | | 0.180 | | 0.235 | | 0.708 | |
| Range | (0.00; 0.33) | | (0.00; 0.19) | | (0.00; 0.59) | | (0.00; 2.13) | | (0.05; 3.96) | |
| IQ range | (0.000; 0.070) | | (0.054; 0.120) | | (0.096; 0.252) | | (0.112; 0.491) | | (0.410; 1.445) | |
| Intermediate | | | | | | | | | | |
| N | 6 | | 3 | | 3 | | 8 | | 2 | |
| Mean (SD) | 0.234 | (0.0834) | 0.498 | (0.1922) | 0.286 | (0.2725) | 0.625 | (0.4614) | 1.641 | (1.4701) |
| Median | 0.237 | | 0.558 | | 0.151 | | 0.688 | | 1.641 | |
| Geometric Mean | 0.222 | | 0.469 | | 0.214 | | 0.521 | | 1.269 | |
| Range | (0.14; 0.36) | | (0.28; 0.65) | | (0.11; 0.60) | | (0.00; 1.12) | | (0.60; 2.68) | |
| IQ range | (0.155; 0.270) | | (0.283; 0.653) | | (0.108; 0.600) | | (0.220; 1.032) | | (0.601; 2.680) | |
| Peak | | | | | | | | | | |
| N | 13 | | 15 | | 16 | | 16 | | 16 | |
| Mean (SD) | 0.397 | (0.2700) | 0.948 | (0.7210) | 0.502 | (0.4297) | 1.148 | (1.0773) | 2.983 | (5.4797) |
| Median | 0.353 | | 0.766 | | 0.383 | | 0.995 | | 1.260 | |
| Geometric Mean | 0.317 | | 0.718 | | 0.365 | | 0.860 | | 1.354 | |
| Range | (0.08; 0.98) | | (0.21; 2.64) | | (0.04; 1.80) | | (0.00; 4.47) | | (0.15; 22.60) | |
| IQ range | (0.261; 0.412) | | (0.372; 1.670) | | (0.237; 0.555) | | (0.517; 1.500) | | (0.965; 1.955) | |
| Week 16 | | | | | | | | | | |
| Trough | | | | | | | | | | |
| N | 22 | | 20 | | 24 | | 20 | | 19 | |
| Mean (SD) | 0.104 | (0.1685) | 0.154 | (0.2566) | 0.194 | (0.1685) | 0.195 | (0.2602) | 0.699 | (0.5547) |
| Median | 0.057 | | 0.089 | | 0.134 | | 0.132 | | 0.451 | |
| Geometric Mean | 0.071 | | 0.114 | | 0.157 | | 0.153 | | 0.564 | |
| Range | (0.00; 0.81) | | (0.00; 1.18) | | (0.00; 0.77) | | (0.00; 1.22) | | (0.00; 2.18) | |
| IQ range | (0.034; 0.090) | | (0.053; 0.129) | | (0.087; 0.277) | | (0.076; 0.203) | | (0.398; 1.030) | |
| Intermediate | | | | | | | | | | |
| N | 3 | | 5 | | 4 | | 4 | | 5 | |
| Mean (SD) | 0.185 | (0.1199) | 0.377 | (0.2732) | 1.144 | (1.4882) | 0.288 | (0.2125) | 1.176 | (0.5652) |
| Median | 0.165 | | 0.365 | | 0.523 | | 0.333 | | 0.946 | |
| Geometric Mean | 0.157 | | 0.301 | | 0.614 | | 0.373 | | 1.073 | |
| Range | (0.08; 0.31) | | (0.12; 0.80) | | (0.18; 3.35) | | (0.00; 0.49) | | (0.67; 1.94) | |
| IQ range | (0.076; 0.313) | | (0.153; 0.446) | | (0.253; 2.036) | | (0.134; 0.443) | | (0.724; 1.600) | |
| Peak | | | | | | | | | | |
| N | 8 | | 8 | | 8 | | 8 | | 10 | |
| Mean (SD) | 0.702 | (1.4128) | 0.867 | (0.4308) | 0.696 | (0.4207) | 1.429 | (1.1046) | 2.077 | (1.0284) |

| Median | 0.295 | 0.917 | 0.796 | 1.165 | 1.970 |
|---|---|---|---|---|---|
| Geometric Mean | 0.347 | 0.762 | 0.546 | 1.137 | 1.832 |
| Range | (0.00; 4.17) | (0.33; 1.60) | (0.15; 1.25) | (0.41; 3.85) | (0.72; 3.65) |
| IQ range | (0.015; 0.413) | (0.471; 1.115) | (0.293; 0.998) | (0.647; 1.775) | (1.060; 2.740) |

^{a}All randomized subjects who received at least 1 complete dose of and had at least 1 valid blood sample drawn for PK analysis after their first dose.
^{b}For QD dosing, peak is defined as any sample between 0.25 to <6 h since last AM dose; intermediate is 6 to <20 h since last AM dose; trough is at least 20 h since last AM dose.
^{c}For BID dosing, peak is defined as any sample between 0.25 to <4 h since last dose; intermediate 4 to <8 h since last dose; trough at least 8 h since last dose.
Note:
Subjects could have multiple samples collected at the same time window, and N is the number of the samples included in the summary.
Note:
The data from a participant who discontinued study agent will be excluded from that point onwards. In addition, the data from a participant who received an incomplete/incorrect or skipped dose(s) based on 2 previous doses prior to the PK sample collection or data from a participant who skip dose prior to PK sample collection will be excluded for that visit.

TABLE 6
Proportion of Subjects with Plasma Concentrations (ng/mL) Below
the Lowest Level of Quantitation (LLOQ) through Week 16

| Treatment Regimen | 25 mg QD | 50 mg QD | 25 mg BID | 100 mg QD | 100 mg BID |
|---|---|---|---|---|---|
| Analysis set: | 42 | 43 | 41 | 42 | 42 |
| Pharmacokinetics Evaluable^{a} | | | | | |
| Week 1 | | | | | |
| N | 3 | 0 | 0 | 0 | 0 |
| Trough^{b, c} | 3 (100.0%) | — | — | — | — |
| Intermediate^{b, c} | 0 | — | — | — | — |
| Peak^{b, c} | 0 | — | — | — | — |
| Week 2 | | | | | |
| N | 3 | 1 | 1 | 0 | 0 |
| Trough | 0 | 0 | 1 (100.0%) | — | — |
| Intermediate | 0 | 0 | 0 | — | — |
| Peak | 3 (100.0%) | 1 (100.0%) | 0 | — | — |
| Week 4 | | | | | |
| N | 3 | 0 | 1 | 0 | 0 |
| Trough | 3 (100.0%) | — | 1 (100.0%) | — | — |
| Intermediate | 0 | — | 0 | — | — |
| Peak | 0 | — | 0 | — | — |
| Week 8 | | | | | |
| N | 2 | 0 | 2 | 0 | 0 |
| Trough | 2 (100.0%) | — | 2 (100.0%) | — | — |
| Intermediate | 0 | — | 0 | — | — |
| Peak | 0) | — | 1 (50.0%) | — | — |
| Week 12 | | | | | |
| N | 6 | 2 | 2 | 3 | 0 |
| Trough | 6 (100.0%) | 2 (100.0%) | 2 (100.0%) | 1 (33.3%) | — |
| Intermediate | 0 | 0 | 0 | 1 (33.3%) | — |
| Peak | 0 | 0 | 0 | 1 (33.3%) | — |
| Week 16 | | | | | |
| N | 4 | 3 | 1 | 3 | 1 |
| Trough | 2 (50.0%) | 3 (100.0%) | 1 (100.0%) | 2 (66.7%) | 1 (100.0%) |
| Intermediate | 0 | 0 | 0 | 1 (33.3%) | 0 |
| Peak | 2 (50.0%) | 0 | 0 | 0 | 0 |

^{a}All randomized subjects who received at least 1 complete dose of and had at least 1 valid blood sample drawn for PK analysis after their first dose.
^{b}For QD dosing, peak is defined as any sample between 0.25 to <6 h since last AM dose; intermediate is 6 to <20 h since last AM dose; trough is at least 20 h since last AM dose.
^{c}For BID dosing, peak is defined as any sample between 0.25 to <4 h since last dose; intermediate 4 to <8 h since last dose; trough at least 8 h since last dose.
Note:
The data from a participant who discontinued study agent will be excluded from that point onwards. In addition, the data from a participant who received an incomplete/incorrect or skipped dose(s) based on 2 previous doses prior to the PK sample collection or data from a participant who skip dose prior to PK sample collection will be excluded for that visit.
Key: LLOQ = lowest level of quantification (0.02 ng/mL)

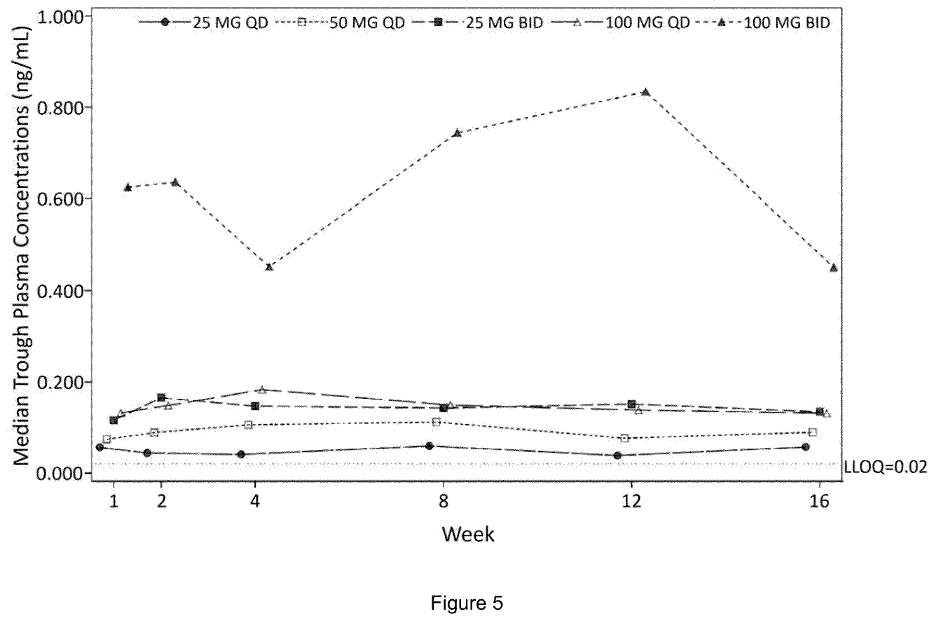

Following oral administration of the IL-23 receptor antagonist peptide, median plasma drug concentrations increased in a dose related manner. Median trough concentrations of the IL-23 receptor antagonist peptide over time were generally similar, with no apparent drug accumulation between Week 1 and 16, consistent with the terminal elimination half-life of the IL-23 receptor antagonist peptide of approximately 9 to 12 hours and attainment of steady state conditions by Week 1 of treatment. The median trough plasma concentrations of the IL-23 receptor antagonist peptide over time are presented graphically in FIG. 5.

Median peak plasma concentrations of the IL-23 receptor antagonist peptide at Week 16 were 0.295 ng/mL for the 25 mg QD, 0.917 ng/mL for the 50 mg QD, 0.796 ng/mL for the 25 mg BID, 1.165 ng/mL for the 100 mg QD, and 1.970 ng/mL for the 100 mg BID dose (Table 5). Median trough plasma concentrations of the IL-23 receptor antagonist peptide at Week 16 were 0.057 ng/mL for the 25 mg QD, 0.089 ng/mL for the 50 mg QD, 0.134 ng/mL for the 25 mg BID, 0.132 ng/mL for the 100 mg QD, and 0.451 ng/mL for the 100 mg BID dose (Table 5). Median trough plasma concentrations of the IL-23 receptor antagonist peptide are much higher for the BID dosing regimens versus the same total daily dose given QD, as illustrated by in FIG. 5, where trough concentrations for the 25 mg BID are comparable to 100 mg QD. For QD dosing, peak concentration was defined as any PK sample taken between 0.25 to <6 hours after the last morning dose and trough concentration was at least 20 hours after the last morning dose. For BID dosing, peak concentration was defined as any PK sample taken between 0.25 to <4 hours after the last dose and trough concentration was at least 8 hours after the last dose.

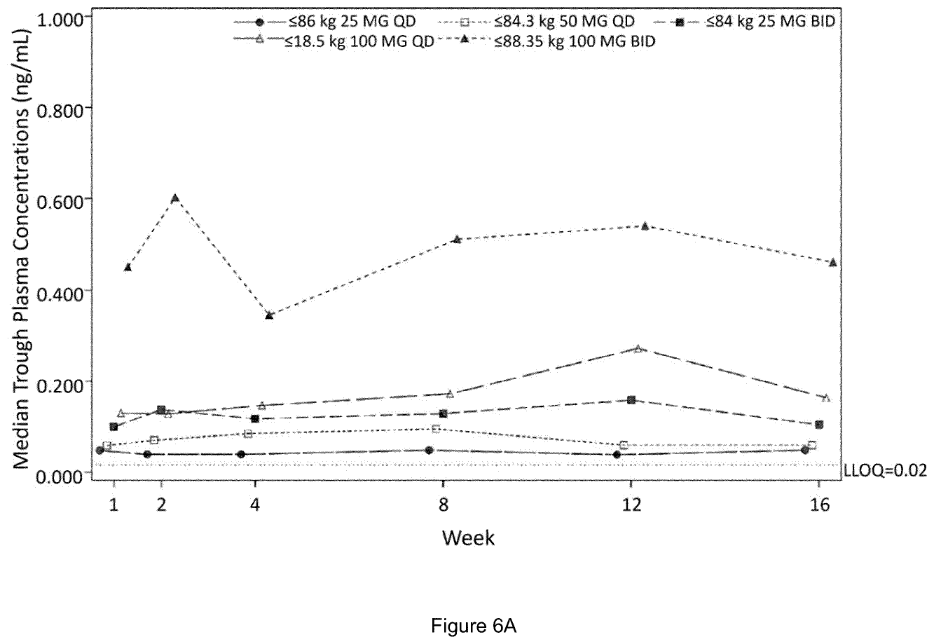

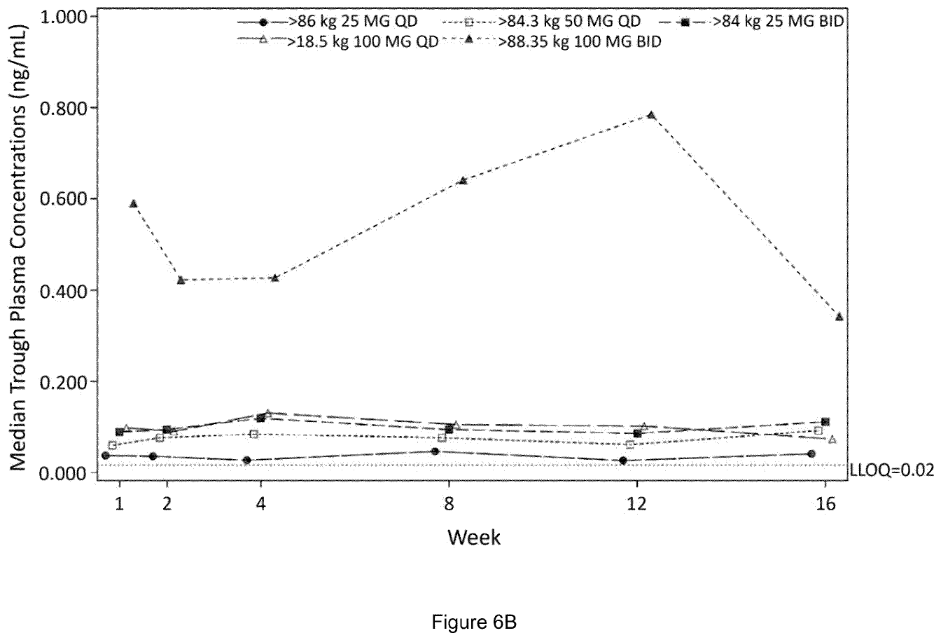

To assess the effect of body weight on the PK of the IL-23 receptor antagonist peptide, plasma concentrations of the IL-23 receptor antagonist peptide through Week 16 were compared between 2 subgroups based on baseline body weight of ≤90 kg (Table 7) and >90 kg Table 8. Line plots of ≤median and >median body weight at baseline for trough concentrations of the IL-23 receptor antagonist peptide for each treatment group through Week 16 were generated to assess the effect of body weight on the PK of the IL-23 receptor antagonist peptide (FIG. 6). Based on the tabular and graphical presentation of the IL-23 receptor antagonist peptide plasma concentration data by median baseline body weight, it is not consistently evident that body weight has a major impact on systemic exposure of the drug.

TABLE 7
Plasma Concentrations (ng/mL) through Week 16 Among Subjects with Baseline Weight ≤90 kg

| Treatment Regimen | 25 mg QD | | 50 mg QD | | 25 mg BID | | 100 mg QD | | 100 mg BID | |
|---|---|---|---|---|---|---|---|---|---|---|
| Analysis set: | 42 | | 43 | | 41 | | 42 | | 42 | |
| Pharmacokinetics | | | | | | | | | | |
| Evaluable^{a} | | | | | | | | | | |
| Subjects with | 24 | | 25 | | 24 | | 26 | | 24 | |
| Baseline Weight ≤90 kg | | | | | | | | | | |
| Week 1 | | | | | | | | | | |
| Trough^{b, c} | | | | | | | | | | |
| N | 14 | | 15 | | 12 | | 11 | | 15 | |
| Mean (SD) | 0.056 | (0.0298) | 0.093 | (0.0336) | 0.131 | (0.0647) | 0.188 | (0.0931) | 0.740 | (0.3024) |
| Median | 0.058 | | 0.079 | | 0.110 | | 0.177 | | 0.581 | |
| Geometric Mean | 0.055 | | 0.088 | | 0.119 | | 0.171 | | 0.689 | |
| Range | (0.00; 0.11) | | (0.05; 0.15) | | (0.06; 0.30) | | (0.09; 0.42) | | (0.39; 1.37) | |
| IQ range | (0.036; 0.068) | | (0.067; 0.120) | | (0.091; 0.163) | | (0.123; 0.215) | | (0.506; 0.901) | |
| Intermediate^{b, c} | | | | | | | | | | |
| N | 2 | | 1 | | 1 | | 4 | | 3 | |
| Mean (SD) | 0.111 | (0.0242) | 0.360 | (—) | 0.077 | (—) | 0.628 | (0.7138) | 1.658 | (1.3457) |
| Median | 0.111 | | 0.360 | | 0.077 | | 0.377 | | 1.410 | |
| Geometric Mean | 0.110 | | 0.360 | | 0.077 | | 0.356 | | 1.257 | |
| Range | (0.09; 0.13) | | (0.36; 0.36) | | (0.08; 0.08) | | (0.12; 1.64) | | (0.45; 3.11) | |
| IQ range | (0.094; 0.128) | | (0.360; 0.360) | | (0.077; 0.077) | | (0.126; 1.130) | | (0.453; 3.110) | |
| Peak^{b, c} | | | | | | | | | | |
| N | 14 | | 15 | | 18 | | 13 | | 13 | |
| Mean (SD) | 0.511 | (0.3237) | 1.094 | (1.0219) | 0.683 | (0.6169) | 1.579 | (1.2121) | 2.343 | (2.1961) |
| Median | 0.471 | | 0.656 | | 0.494 | | 1.190 | | 1.820 | |
| Geometric Mean | 0.388 | | 0.705 | | 0.476 | | 1.248 | | 1.828 | |
| Range | (0.04; 1.10) | | (0.07; 3.83) | | (0.05; 2.71) | | (0.28; 4.97) | | (0.56; 9.19) | |
| IQ range | (0.211; 0.698) | | (0.491; 1.350) | | (0.348; 0.919) | | (0.880; 1.790) | | (1.220; 2.420) | |
| Week 2 | | | | | | | | | | |
| Trough | | | | | | | | | | |
| N | 12 | | 14 | | 11 | | 9 | | 11 | |
| Mean (SD) | 0.075 | (0.0667) | 0.136 | (0.1567) | 0.261 | (0.2643) | 0.260 | (0.1516) | 0.662 | (0.2708) |
| Median | 0.044 | | 0.089 | | 0.167 | | 0.172 | | 0.722 | |
| Geometric Mean | 0.060 | | 0.100 | | 0.196 | | 0.224 | | 0.595 | |
| Range | (0.04; 0.26) | | (0.04; 0.66) | | (0.09; 0.98) | | (0.12; 0.53) | | (0.17; 1.10) | |
| IQ range | (0.043; 0.068) | | (0.076; 0.131) | | (0.130; 0.239) | | (0.143; 0.365) | | (0.393; 0.844) | |
| Intermediate | | | | | | | | | | |
| N | 0 | | 1 | | 2 | | 4 | | 1 | |
| Mean (SD) | — | | 0.295 | (—) | 0.658 | (0.5127) | 0.623 | (0.1220) | 0.912 | (—) |
| Median | — | | 0.295 | | 0.658 | | 0.608 | | 0.912 | |
| Geometric Mean | — | | 0.295 | | 0.549 | | 0.614 | | 0.912 | |
| Range | — | | (0.30; 0.30) | | (0.30; 1.02) | | (0.50; 0.78) | | (0.91; 0.91) | |
| IQ range | — | | (0.295; 0.295) | | (0.295; 1.020) | | (0.529; 0.717) | | (0.912; 0.912) | |
| Peak | | | | | | | | | | |
| N | 11 | | 11 | | 10 | | 11 | | 14 | |
| Mean (SD) | 0.318 | (0.3794) | 1.261 | (1.5440) | 0.624 | (0.7207) | 1.135 | (0.9971) | 2.610 | (3.0443) |
| Median | 0.157 | | 0.762 | | 0.349 | | 1.350 | | 1.575 | |
| Geometric Mean | 0.188 | | 0.652 | | 0.386 | | 0.609 | | 1.685 | |
| Range | (0.00; 1.26) | | (0.00; 5.20) | | (0.07; 2.49) | | (0.10; 2.67) | | (0.42; 12.20) | |
| IQ range | (0.049; 0.574) | | (0.112; 1.640) | | (0.209; 0.893) | | (0.135; 1.950) | | (0.748; 2.880) | |
| Week 4 | | | | | | | | | | |
| Trough | | | | | | | | | | |
| N | 12 | | 15 | | 15 | | 14 | | 12 | |
| Mean (SD) | 0.082 | (0.1249) | 0.102 | (0.0390) | 0.200 | (0.1282) | 0.260 | (0.2071) | 0.592 | (0.8121) |
| Median | 0.050 | | 0.104 | | 0.155 | | 0.202 | | 0.432 | |
| Geometric Mean | 0.064 | | 0.095 | | 0.169 | | 0.212 | | 0.373 | |
| Range | (0.00; 0.47) | | (0.03; 0.21) | | (0.06; 0.49) | | (0.08; 0.89) | | (0.05; 3.13) | |
| IQ range | (0.028; 0.080) | | (0.078; 0.119) | | (0.097; 0.260) | | (0.128; 0.287) | | (0.300; 0.480) | |
| Intermediate | | | | | | | | | | |
| N | 1 | | 1 | | 0 | | 2 | | 2 | |
| Mean (SD) | 0.102 | (—) | 0.502 | (—) | — | | 0.602 | (0.0587) | 0.641 | (0.2362) |
| Median | 0.102 | | 0.502 | | — | | 0.602 | | 0.641 | |
| Geometric Mean | 0.102 | | 0.502 | | — | | 0.600 | | 0.619 | |
| Range | (0.10; 0.10) | | (0.50; 0.50) | | — | | (0.56; 0.64) | | (0.47; 0.81) | |
| IQ range | (0.102; 0.102) | | (0.502; 0.502) | | — | | (0.560; 0.643) | | (0.474; 0.808) | |
| Peak | | | | | | | | | | |
| N | 10 | | 8 | | 5 | | 9 | | 8 | |
| Mean (SD) | 0.573 | (0.3641) | 0.591 | (0.3058) | 0.988 | (0.3968) | 1.405 | (0.8733) | 2.087 | (1.5637) |
| Median | 0.471 | | 0.544 | | 1.180 | | 1.410 | | 1.950 | |
| Geometric Mean | 0.459 | | 0.521 | | 0.913 | | 1.002 | | 1.651 | |
| Range | (0.09; 1.22) | | (0.17; 1.22) | | (0.48; 1.37) | | (0.11; 2.69) | | (0.49; 5.53) | |
| IQ range | (0.319; 0.764) | | (0.429; 0.693) | | (0.646; 1.260) | | (0.881; 2.020) | | (1.020; 2.365) | |
| Week 8 | | | | | | | | | | |
| Trough | | | | | | | | | | |
| N | 15 | | 17 | | 19 | | 19 | | 12 | |
| Mean (SD) | 0.065 | (0.0404) | 0.164 | (0.1535) | 0.195 | (0.1534) | 0.274 | (0.2630) | 1.083 | (1.3285) |
| Median | 0.061 | | 0.119 | | 0.164 | | 0.194 | | 0.732 | |
| Geometric Mean | 0.063 | | 0.129 | | 0.157 | | 0.205 | | 0.739 | |
| Range | (0.00; 0.19) | | (0.04; 0.70) | | (0.00; 0.61) | | (0.05; 1.20) | | (0.18; 5.14) | |
| IQ range | (0.038; 0.077) | | (0.093; 0.171) | | (0.093; 0.268) | | (0.122; 0.347) | | (0.444; 0.980) | |
| Intermediate | | | | | | | | | | |
| N | 1 | | 0 | | 0 | | 1 | | 3 | |
| Mean (SD) | 0.059 | (—) | — | | — | | 0.235 | (—) | 1.346 | (0.8781) |
| Median | 0.059 | | — | | — | | 0.235 | | 0.860 | |
| Geometric Mean | 0.059 | | — | | — | | 0.235 | | 1.185 | |
| Range | (0.06; 0.06) | | — | | — | | (0.24; 0.24) | | (0.82; 2.36) | |
| IQ range | (0.059; 0.059) | | — | | — | | (0.235; 0.235) | | (0.819; 2.360) | |
| Peak | | | | | | | | | | |
| N | 5 | | 8 | | 5 | | 7 | | 10 | |
| Mean (SD) | 0.620 | (0.6126) | 0.885 | (0.5402) | 1.248 | (1.2488) | 1.499 | (1.4395) | 1.872 | (1.9712) |
| Median | 0.589 | | 0.837 | | 0.724 | | 0.886 | | 1.295 | |
| Geometric Mean | 0.316 | | 0.723 | | 0.951 | | 1.158 | | 1.280 | |
| Range | (0.03; 1.60) | | (0.25; 1.62) | | (0.61; 3.48) | | (0.61; 4.67) | | (0.20; 7.17) | |
| IQ range | (0.195; 0.690) | | (0.409; 1.360) | | (0.689; 0.736) | | (0.679; 1.640) | | (0.741; 2.060) | |
| Week 12 | | | | | | | | | | |
| Trough | | | | | | | | | | |
| N | 11 | | 12 | | 14 | | 16 | | 12 | |
| Mean (SD) | 0.045 | (0.0337) | 0.083 | (0.0483) | 0.234 | (0.1753) | 0.428 | (0.5343) | 0.954 | (1.0368) |
| Median | 0.048 | | 0.076 | | 0.198 | | 0.188 | | 0.745 | |
| Geometric Mean | 0.059 | | 0.082 | | 0.209 | | 0.288 | | 0.591 | |
| Range | (0.00; 0.09) | | (0.00; 0.19) | | (0.00; 0.59) | | (0.00; 2.13) | | (0.05; 3.96) | |
| IQ range | (0.000; 0.084) | | (0.052; 0.112) | | (0.106; 0.276) | | (0.130; 0.535) | | (0.410; 0.978) | |
| Intermediate | | | | | | | | | | |
| N | 4 | | 1 | | 0 | | 5 | | 1 | |
| Mean (SD) | 0.208 | (0.0695) | 0.653 | (—) | — | | 0.502 | (0.4958) | 2.680 | (—) |
| Median | 0.211 | | 0.653 | | — | | 0.433 | | 2.680 | |
| Geometric Mean | 0.199 | | 0.653 | | — | | 0.396 | | 2.680 | |
| Range | (0.14; 0.27) | | (0.65; 0.65) | | — | | (0.00; 1.08) | | (2.68; 2.68) | |
| IQ range | (0.148; 0.268) | | (0.653; 0.653) | | — | | (0.056; 0.942) | | (2.680; 2.680) | |
| Peak | | | | | | | | | | |
| N | 9 | | 9 | | 11 | | 11 | | 11 | |
| Mean (SD) | 0.449 | (0.2997) | 1.055 | (0.7420) | 0.552 | (0.4843) | 1.197 | (1.3142) | 1.426 | (0.9187) |
| Median | 0.378 | | 0.872 | | 0.395 | | 0.618 | | 1.230 | |
| Geometric Mean | 0.362 | | 0.856 | | 0.387 | | 0.783 | | 1.101 | |
| Range | (0.08; 0.98) | | (0.29; 2.64) | | (0.04; 1.80) | | (0.00; 4.47) | | (0.15; 3.55) | |
| IQ range | (0.284; 0.412) | | (0.528; 1.230) | | (0.269; 0.633) | | (0.145; 1.880) | | (0.973; 1.760) | |
| Week 16 | | | | | | | | | | |
| Trough | | | | | | | | | | |
| N | 12 | | 13 | | 14 | | 13 | | 11 | |
| Mean (SD) | 0.136 | (0.2189) | 0.185 | (0.3155) | 0.224 | (0.1953) | 0.241 | (0.3158) | 0.658 | (0.4590) |
| Median | 0.061 | | 0.077 | | 0.131 | | 0.150 | | 0.576 | |
| Geometric Mean | 0.077 | | 0.119 | | 0.165 | | 0.199 | | 0.600 | |
| Range | (0.03; 0.81) | | (0.00; 1.18) | | (0.05; 0.77) | | (0.00; 1.22) | | (0.00; 1.44) | |
| IQ range | (0.038; 0.113) | | (0.043; 0.134) | | (0.097; 0.323) | | (0.091; 0.241) | | (0.416; 1.030) | |
| Intermediate | | | | | | | | | | |
| N | 2 | | 2 | | 3 | | 2 | | 4 | |
| Mean (SD) | 0.194 | (0.1679) | 0.462 | (0.4787) | 1.465 | (1.6443) | 0.199 | (0.2814) | 1.289 | (0.5838) |
| Median | 0.194 | | 0.462 | | 0.722 | | 0.199 | | 1.273 | |
| Geometric Mean | 0.154 | | 0.314 | | 0.922 | | 0.398 | | 1.184 | |
| Range | (0.08; 0.31) | | (0.12; 0.80) | | (0.32; 3.35) | | (0.00; 0.40) | | (0.67; 1.94) | |
| IQ range | (0.076; 0.313) | | (0.123; 0.800) | | (0.324; 3.350) | | (0.000; 0.398) | | (0.808; 1.770) | |
| Peak | | | | | | | | | | |
| N | 5 | | 2 | | 4 | | 7 | | 6 | |
| Mean (SD) | 0.973 | (1.7958) | 1.255 | (0.4879) | 0.818 | (0.3405) | 1.397 | (1.1892) | 2.161 | (1.1942) |

| Median | 0.333 | 1.255 | 0.803 | 1.130 | 1.825 |
|---|---|---|---|---|---|
| Geometric Mean | 0.794 | 1.207 | 0.761 | 1.078 | 1.894 |
| Range | (0.00; 4.17) | (0.91; 1.60) | (0.42; 1.25) | (0.41; 3.85) | (1.00; 3.65) |
| IQ range | (0.000; 0.360) | (0.910; 1.600) | (0.609; 1.028) | (0.628; 1.900) | (1.060; 3.610) |

^{a}All randomized subjects who received at least 1 complete dose of and had at least 1 valid blood sample drawn for PK analysis after their first dosc.
^{b}For QD dosing, peak is defined as any sample between 0.25 to <6 h since last AM dose; intermediate is 6 to <20 h since last AM dose; trough is at least 20 h since last AM dose.
^{c}For BID dosing, peak is defined as any sample between 0.25 to <4 h since last dose; intermediate 4 to <8 h since last dose; trough at least 8 h since last dose.
Note:
Subjects could have multiple samples collected at the same time window, and N is the number of the samples included in the summary.
Note:
The data from a participant who discontinued study agent will be excluded from that point onwards. In addition, the data from a participant who received an incomplete/incorrect or skipped dose(s) based on 2 previous doses prior to the PK sample collection or data from a participant who skip dose prior to PK sample collection will be excluded for that visit.

TABLE 8
Plasma Concentrations (ng/mL) through Week 16 Among Subjects with Baseline Weight >90 kg

| Treatment Regimen | 25 mg QD | | 50 mg QD | | 25 mg BID | | 100 mg QD | | 100 mg BID | |
|---|---|---|---|---|---|---|---|---|---|---|
| Analysis set: | 42 | | 43 | | 41 | | 42 | | 42 | |
| Pharmacokinetics Evaluable^{a} | | | | | | | | | | |
| Participants with Baseline | 18 | | 18 | | 17 | | 16 | | 18 | |
| Weight >90 kg | | | | | | | | | | |
| Week 1 | | | | | | | | | | |
| Trough^{b, c} | | | | | | | | | | |
| N | 9 | | 7 | | 11 | | 10 | | 15 | |
| Mean (SD) | 0.044 | (0.0282) | 0.073 | (0.0435) | 0.162 | (0.1350) | 0.127 | (0.0959) | 0.745 | (0.3878) |
| Median | 0.049 | | 0.051 | | 0.133 | | 0.102 | | 0.738 | |
| Geometric Mean | 0.055 | | 0.065 | | 0.132 | | 0.102 | | 0.642 | |
| Range | (0.00; 0.07) | | (0.04; 0.17) | | (0.06; 0.54) | | (0.04; 0.36) | | (0.17; 1.57) | |
| IQ range | (0.034; 0.069) | | (0.044; 0.086) | | (0.081; 0.168) | | (0.072; 0.152) | | (0.472; 1.030) | |
| Intermediate^{b, c} | | | | | | | | | | |
| N | 1 | | 2 | | 0 | | 1 | | 2 | |
| Mean (SD) | 0.171 | (—) | 0.343 | (0.2729) | — | | 0.480 | (—) | 1.392 | (0.8464) |
| Median | 0.171 | | 0.343 | | — | | 0.480 | | 1.392 | |
| Geometric Mean | 0.171 | | 0.284 | | — | | 0.480 | | 1.256 | |
| Range | (0.17; 0.17) | | (0.15; 0.54) | | — | | (0.48; 0.48) | | (0.79; 1.99) | |
| IQ range | (0.171; 0.171) | | (0.150; 0.536) | | — | | (0.480; 0.480) | | (0.793; 1.990) | |
| Peak^{b, c} | | | | | | | | | | |
| N | 10 | | 11 | | 11 | | 10 | | 6 | |
| Mean (SD) | 0.552 | (0.5003) | 0.634 | (0.3470) | 0.389 | (0.2499) | 1.356 | (0.5758) | 1.195 | (0.7439) |
| Median | 0.330 | | 0.669 | | 0.346 | | 1.305 | | 1.068 | |
| Geometric Mean | 0.409 | | 0.523 | | 0.319 | | 1.241 | | 0.979 | |
| Range | (0.18; 1.59) | | (0.10; 1.19) | | (0.10; 0.92) | | (0.56; 2.46) | | (0.32; 2.23) | |
| IQ range | (0.209; 0.599) | | (0.327; 0.787) | | (0.179; 0.543) | | (1.070; 1.700) | | (0.635; 1.850) | |
| Week 2 | | | | | | | | | | |
| Trough | | | | | | | | | | |
| N | 4 | | 6 | | 8 | | 6 | | 13 | |
| Mean (SD) | 0.050 | (0.0274) | 0.097 | (0.0472) | 0.183 | (0.1660) | 0.239 | (0.2433) | 0.678 | (0.4508) |
| Median | 0.043 | | 0.092 | | 0.124 | | 0.113 | | 0.565 | |
| Geometric Mean | 0.044 | | 0.086 | | 0.158 | | 0.150 | | 0.558 | |
| Range | (0.02; 0.09) | | (0.03; 0.16) | | (0.00; 0.49) | | (0.04; 0.61) | | (0.16; 1.75) | |
| IQ range | (0.033; 0.067) | | (0.065; 0.140) | | (0.068; 0.294) | | (0.073; 0.491) | | (0.404; 0.720) | |
| Intermediate | | | | | | | | | | |
| N | 2 | | 2 | | 0 | | 1 | | 0 | |
| Mean (SD) | 0.210 | (0.0474) | 0.260 | (0.1336) | — | | 0.649 | (—) | — | |
| Median | 0.210 | | 0.260 | | — | | 0.649 | | — | |
| Geometric Mean | 0.207 | | 0.242 | | — | | 0.649 | | — | |
| Range | (0.18; 0.24) | | (0.17; 0.35) | | — | | (0.65; 0.65) | | — | |
| IQ range | (0.176; 0.243) | | (0.165; 0.354) | | — | | (0.649; 0.649) | | — | |
| Peak | | | | | | | | | | |
| N | 7 | | 7 | | 11 | | 10 | | 3 | |
| Mean (SD) | 0.147 | (0.1587) | 0.444 | (0.4157) | 0.442 | (0.3339) | 0.526 | (0.3381) | 0.958 | (0.5492) |
| Median | 0.093 | | 0.267 | | 0.375 | | 0.624 | | 0.908 | |
| Geometric Mean | 0.142 | | 0.275 | | 0.330 | | 0.381 | | 0.845 | |
| Range | (0.00; 0.40) | | (0.07; 1.10) | | (0.06; 1.28) | | (0.06; 1.00) | | (0.44; 1.53) | |
| IQ range | (0.000; 0.288) | | (0.090; 0.889) | | (0.248; 0.578) | | (0.136; 0.834) | | (0.435; 1.530) | |
| Week 4 | | | | | | | | | | |
| Trough | | | | | | | | | | |
| N | 6 | | 12 | | 12 | | 5 | | 11 | |
| Mean (SD) | 0.037 | (0.0253) | 0.112 | (0.0571) | 0.167 | (0.1440) | 0.176 | (0.1592) | 1.462 | (3.1404) |
| Median | 0.034 | | 0.106 | | 0.137 | | 0.119 | | 0.533 | |
| Geometric Mean | 0.041 | | 0.097 | | 0.139 | | 0.140 | | 0.611 | |
| Range | (0.00; 0.07) | | (0.02; 0.25) | | (0.00; 0.52) | | (0.08; 0.46) | | (0.19; 10.90) | |
| IQ range | (0.024; 0.057) | | (0.077; 0.134) | | (0.066; 0.216) | | (0.107; 0.119) | | (0.303; 0.752) | |
| Intermediate | | | | | | | | | | |
| N | 2 | | 2 | | 1 | | 2 | | 1 | |
| Mean (SD) | 0.216 | (0.0544) | 0.170 | (0.0219) | 0.083 | (—) | 0.801 | (0.2729) | 1.130 | (—) |
| Median | 0.216 | | 0.170 | | 0.083 | | 0.801 | | 1.130 | |
| Geometric Mean | 0.212 | | 0.169 | | 0.083 | | 0.777 | | 1.130 | |
| Range | (0.18; 0.25) | | (0.15; 0.19) | | (0.08; 0.08) | | (0.61; 0.99) | | (1.13; 1.13) | |
| IQ range | (0.177; 0.254) | | (0.154; 0.185) | | (0.083; 0.083) | | (0.608; 0.994) | | (1.130; 1.130) | |
| Peak | | | | | | | | | | |
| N | 6 | | 7 | | 7 | | 5 | | 3 | |
| Mean (SD) | 0.621 | (0.5013) | 0.535 | (0.3224) | 0.418 | (0.2203) | 1.140 | (0.7593) | 1.947 | (0.8994) |
| Median | 0.405 | | 0.481 | | 0.361 | | 0.856 | | 1.520 | |
| Geometric Mean | 0.487 | | 0.439 | | 0.370 | | 0.911 | | 1.824 | |
| Range | (0.23; 1.51) | | (0.16; 0.94) | | (0.18; 0.81) | | (0.29; 1.98) | | (1.34; 2.98) | |
| IQ range | (0.261; 0.911) | | (0.195; 0.855) | | (0.209; 0.538) | | (0.675; 1.900) | | (1.340; 2.980) | |
| Week 8 | | | | | | | | | | |
| Trough | | | | | | | | | | |
| N | 9 | | 11 | | 10 | | 11 | | 10 | |
| Mean (SD) | 0.073 | (0.0865) | 0.120 | (0.1073) | 0.165 | (0.1536) | 0.152 | (0.0939) | 0.814 | (0.6158) |
| Median | 0.058 | | 0.082 | | 0.105 | | 0.131 | | 0.744 | |
| Geometric Mean | 0.058 | | 0.088 | | 0.138 | | 0.133 | | 0.585 | |
| Range | (0.00; 0.29) | | (0.03; 0.38) | | (0.00; 0.49) | | (0.06; 0.40) | | (0.11; 2.01) | |
| IQ range | (0.021; 0.069) | | (0.047; 0.171) | | (0.077; 0.273) | | (0.078; 0.175) | | (0.260; 1.220) | |
| Intermediate | | | | | | | | | | |
| N | 3 | | 1 | | 1 | | 0 | | 1 | |
| Mean (SD) | 0.262 | (0.1490) | 0.441 | (—) | 0.092 | (—) | — | | 0.125 | (—) |
| Median | 0.193 | | 0.441 | | 0.092 | | — | | 0.125 | |
| Geometric Mean | 0.237 | | 0.441 | | 0.092 | | — | | 0.125 | |
| Range | (0.16; 0.43) | | (0.44; 0.44) | | (0.09; 0.09) | | — | | (0.13; 0.13) | |
| IQ range | (0.160; 0.433) | | (0.441; 0.441) | | (0.092; 0.092) | | — | | (0.125; 0.125) | |
| Peak | | | | | | | | | | |
| N | 7 | | 7 | | 8 | | 5 | | 5 | |
| Mean (SD) | 0.318 | (0.1785) | 1.354 | (1.2690) | 0.312 | (0.2165) | 0.648 | (0.2175) | 6.908 | (7.1726) |
| Median | 0.289 | | 0.780 | | 0.279 | | 0.613 | | 2.280 | |
| Geometric Mean | 0.263 | | 1.029 | | 0.298 | | 0.618 | | 3.934 | |
| Range | (0.07; 0.55) | | (0.50; 4.06) | | (0.00; 0.60) | | (0.44; 0.88) | | (1.32; 15.10) | |
| IQ range | (0.162; 0.530) | | (0.535; 1.770) | | (0.158; 0.515) | | (0.440; 0.863) | | (1.440; 14.400) | |
| Week 12 | | | | | | | | | | |
| Trough | | | | | | | | | | |
| N | 12 | | 8 | | 10 | | 9 | | 12 | |
| Mean (SD) | 0.058 | (0.0884) | 0.089 | (0.0596) | 0.157 | (0.1294) | 0.225 | (0.1944) | 1.238 | (0.9329) |
| Median | 0.033 | | 0.075 | | 0.108 | | 0.122 | | 1.016 | |
| Geometric Mean | 0.053 | | 0.092 | | 0.146 | | 0.167 | | 0.849 | |
| Range | (0.00; 0.33) | | (0.00; 0.18) | | (0.00; 0.45) | | (0.08; 0.55) | | (0.12; 2.79) | |
| IQ range | (0.013; 0.068) | | (0.055; 0.139) | | (0.096; 0.200) | | (0.091; 0.327) | | (0.490; 1.970) | |
| Intermediate | | | | | | | | | | |
| N | 2 | | 2 | | 3 | | 3 | | 1 | |
| Mean (SD) | 0.286 | (0.1103) | 0.421 | (0.1945) | 0.286 | (0.2725) | 0.829 | (0.3920) | 0.601 | (—) |
| Median | 0.286 | | 0.421 | | 0.151 | | 0.983 | | 0.601 | |
| Geometric Mean | 0.275 | | 0.397 | | 0.214 | | 0.750 | | 0.601 | |
| Range | (0.21; 0.36) | | (0.28; 0.56) | | (0.11; 0.60) | | (0.38; 1.12) | | (0.60; 0.60) | |
| IQ range | (0.208; 0.364) | | (0.283; 0.558) | | (0.108; 0.600) | | (0.383; 1.120) | | (0.601; 0.601) | |
| Peak | | | | | | | | | | |
| N | 4 | | 6 | | 5 | | 5 | | 5 | |
| Mean (SD) | 0.281 | (0.1617) | 0.786 | (0.7229) | 0.391 | (0.2896) | 1.042 | (0.1155) | 6.409 | (9.4420) |
| Median | 0.291 | | 0.388 | | 0.365 | | 1.090 | | 1.290 | |
| Geometric Mean | 0.236 | | 0.551 | | 0.320 | | 1.036 | | 2.133 | |
| Range | (0.08; 0.46) | | (0.21; 1.76) | | (0.14; 0.88) | | (0.90; 1.16) | | (0.23; 22.60) | |
| IQ range | (0.156; 0.406) | | (0.302; 1.670) | | (0.202; 0.369) | | (0.939; 1.120) | | (0.957; 6.970) | |
| Week 16 | | | | | | | | | | |
| Trough | | | | | | | | | | |
| N | 10 | | 7 | | 10 | | 7 | | 8 | |
| Mean (SD) | 0.066 | (0.0688) | 0.096 | (0.0582) | 0.152 | (0.1186) | 0.110 | (0.0440) | 0.755 | (0.6958) |
| Median | 0.047 | | 0.115 | | 0.138 | | 0.129 | | 0.428 | |
| Geometric Mean | 0.063 | | 0.105 | | 0.144 | | 0.102 | | 0.522 | |
| Range | (0.00; 0.23) | | (0.00; 0.18) | | (0.00; 0.44) | | (0.05; 0.18) | | (0.10; 2.18) | |
| IQ range | (0.021; 0.090) | | (0.063; 0.124) | | (0.078; 0.173) | | (0.064; 0.133) | | (0.352; 1.101) | |
| Intermediate | | | | | | | | | | |
| N | 1 | | 3 | | 1 | | 2 | | 1 | |
| Mean (SD) | 0.165 | (—) | 0.321 | (0.1513) | 0.181 | (—) | 0.378 | (0.1563) | 0.724 | (—) |
| Median | 0.165 | | 0.365 | | 0.181 | | 0.378 | | 0.724 | |
| Geometric Mean | 0.165 | | 0.292 | | 0.181 | | 0.361 | | 0.724 | |
| Range | (0.17; 0.17) | | (0.15; 0.45) | | (0.18; 0.18) | | (0.27; 0.49) | | (0.72; 0.72) | |
| IQ range | (0.165; 0.165) | | (0.153; 0.446) | | (0.181; 0.181) | | (0.267; 0.488) | | (0.724; 0.724) | |
| Peak | | | | | | | | | | |
| N | 3 | | 6 | | 4 | | 1 | | 4 | |
| Mean (SD) | 0.250 | (0.2180) | 0.738 | (0.3632) | 0.575 | (0.5073) | 1.650 | (—) | 1.950 | (0.8720) |

| Median | 0.257 | 0.760 | 0.480 | 1.650 | 2.170 |
|---|---|---|---|---|---|
| Geometric Mean | 0.152 | 0.654 | 0.392 | 1.650 | 1.743 |
| Range | (0.03; 0.47) | (0.33; 1.14) | (0.15; 1.19) | (1.65; 1.65) | (0.72; 2.74) |
| IQ range | (0.029; 0.465) | (0.345; 1.090) | (0.159; 0.991) | (1.650; 1.650) | (1.370; 2.530) |

^{a}All randomized subjects who received at least 1 complete dose of and had at least 1 valid blood sample drawn for PK analysis after their first dose.
^{b}For QD dosing, peak is defined as any sample between 0.25 to <6 h since last AM dose; intermediate is 6 to <20 h since last AM dose; trough is at least 20 h since last AM dose.
^{c}For BID dosing, peak is defined as any sample between 0.25 to <4 h since last dose; intermediate 4 to <8 h since last dose; trough at least 8 h since last dose.
Note:
Subjects could have multiple samples collected at the same time window, and N is the number of the samples included in the summary.
Note:
The data from a participant who discontinued study agent will be excluded from that point onwards. In addition, the data from a participant who received an incomplete/incorrect or skipped dose(s) based on 2 previous doses prior to the PK sample collection or data from a participant who skip dose prior to PK sample collection will be excluded for that visit.

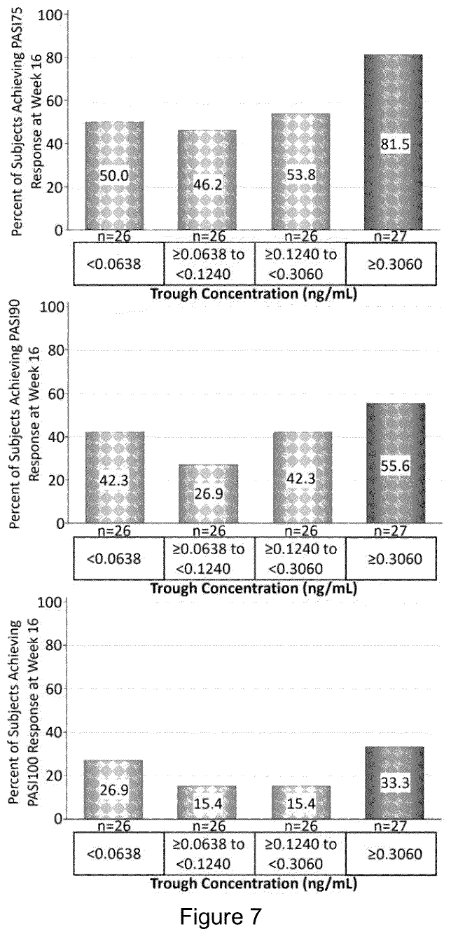

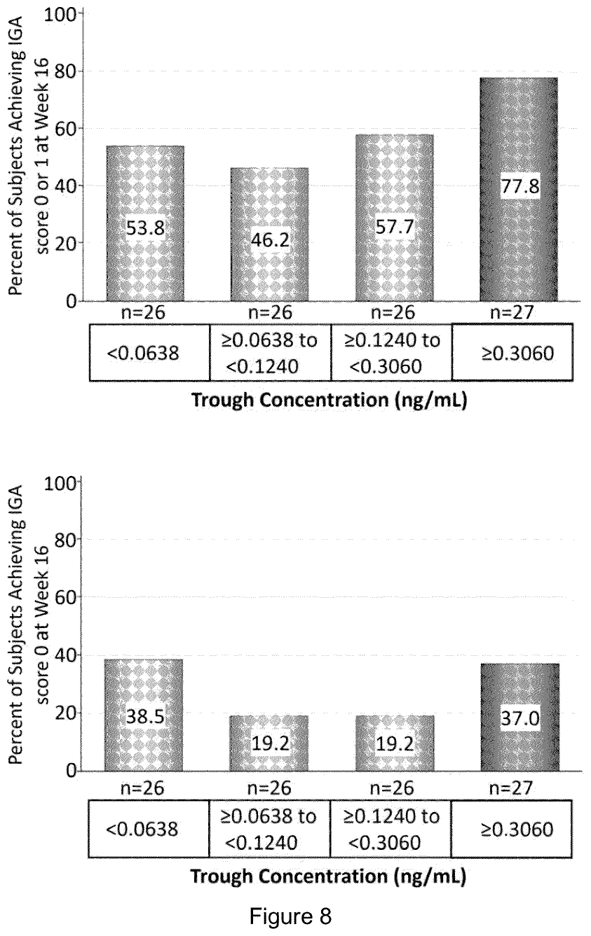

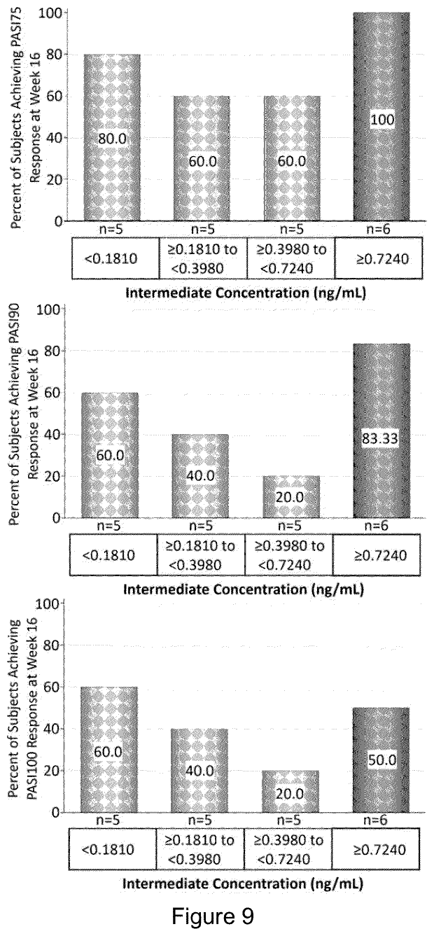

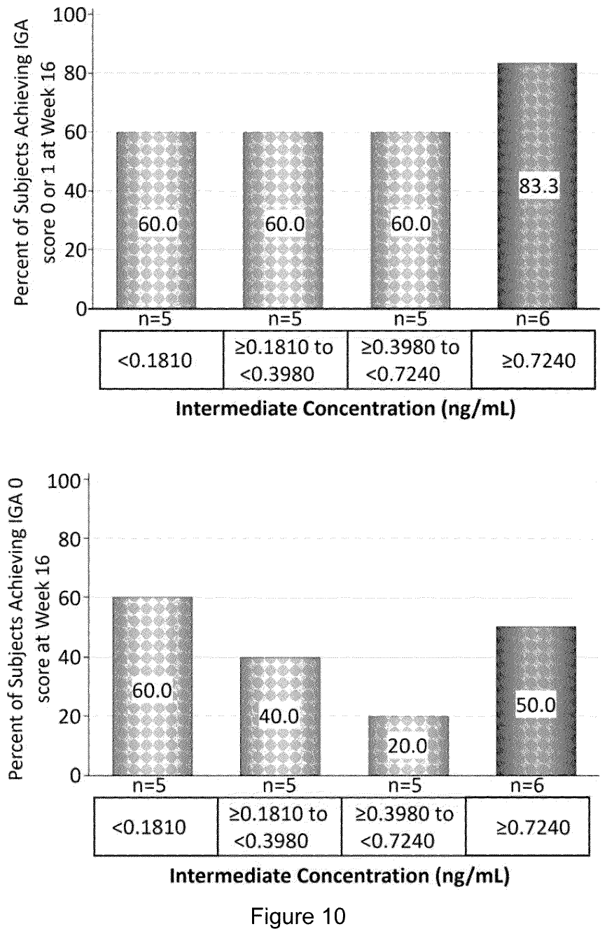

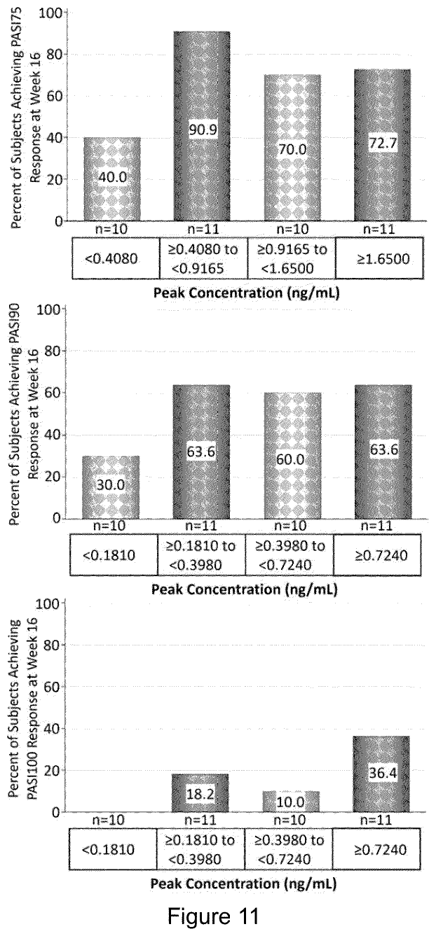

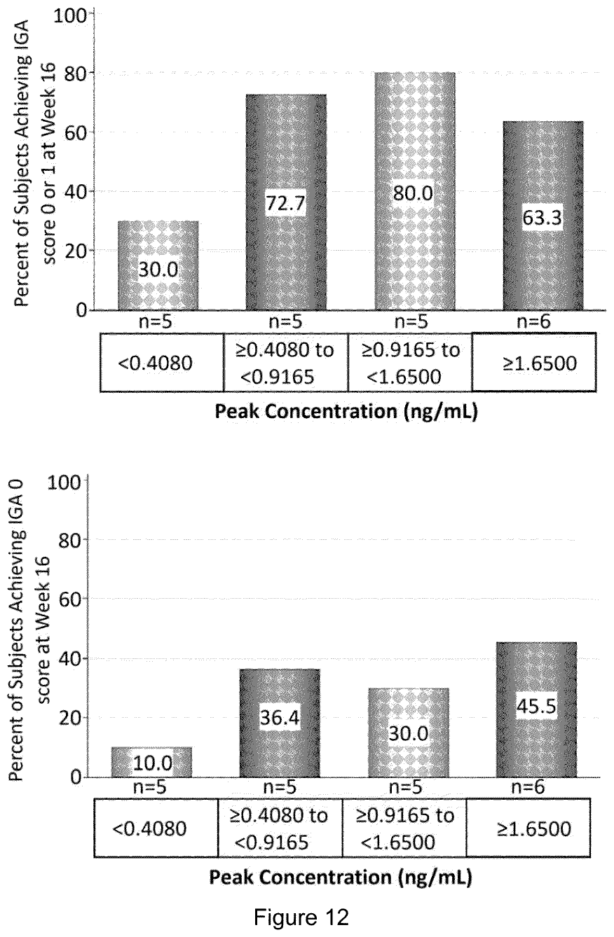

A summary of clinical response rate at Week 16 by plasma the IL-23 receptor antagonist peptide concentration quartiles at Week 16 is presented in Table 9 (trough concentrations), Table 10 (intermediate concentrations) and Table 11 (peak concentrations). Bar plots of the percent of participants achieving PASI75, PASI90 and PASI100 response or IGA score of 0/1 or 0 at Week 16 by trough plasma the IL-23 receptor antagonist peptide concentrations at Week 16 is presented in FIG. 7 and FIG. 8, respectively. Bar plots of the percent of participants achieving PASI75, PASI90 and PASI100 response or IGA score of 0/1 or 0 at Week 16 by intermediate plasma the IL-23 receptor antagonist peptide concentrations at Week 16 is presented in FIG. 9 and FIG. 10 respectively. Bar plots of the percent of participants achieving PASI75, PASI90 and PASI100 response or IGA score of 0/1 or 0 at Week 16 by the peak plasma IL-23 receptor antagonist peptide concentrations at Week 16 is presented in FIG. 11 and FIG. 12 respectively.

TABLE 9
Clinical Response at Week 16 by Trough Plasma Concentrations (ng/mL)

| | Trough Plasma Concentration Quartiles | | | |
|---|---|---|---|---|
| | <Q1 | Q1 to <Q2 | Q2 to <Q3 | ≥Q3 |
| Analysis set: | 25 | 27 | 26 | 27 |
| Pharmacokinetics Evaluable^{a} | | | | |
| Subjects treated | 5 | 6 | 5 | 6 |
| with 25 mg QD | | | | |
| IGA | | | | |
| N | 5 | 6 | 5 | 6 |
| IGA Score = 0 or 1 | 2 (40.0%) | 3 (50.0%) | 1 (20.0%) | 4 (66.7%) |
| IGA Score = 0 | 1 (20.0%) | 2 (33.3%) | 0 | 1 (16.7%) |
| PASI | | | | |
| N | 5 | 6 | 5 | 6 |
| ≥75% Improvement | 2 (40.0%) | 2 (33.3%) | 2 (40.0%) | 3 (50.0%) |
| ≥90% Improvement | 2 (40.0%) | 2 (33.3%) | 0 | 1 (16.7%) |
| 100% Improvement | 1 (20.0%) | 1 (16.7%) | 0) | 1 (16.7%) |
| Subjects treated | 5 | 5 | 5 | 5 |
| with 50 mg QD | | | | |
| IGA | | | | |
| N | 5 | 5 | 5 | 5 |
| IGA Score = 0 or 1 | 2 (40.0%) | 4 (80.0%) | 2 (40.0%) | 3 (60.0%) |
| IGA Score = 0 | 1 (20.0%) | 3 (60.0%) | 2 (40.0%) | 2 (40.0%) |
| PASI | | | | |
| N | 5 | 5 | 5 | 5 |
| ≥75% Improvement | 2 (40.0%) | 3 (60.0%) | 3 (60.0%) | 3 (60.0%) |
| ≥90% Improvement | 1 (20.0%) | 3 (60.0%) | 2 (40.0%) | 3 (60.0%) |
| 100% Improvement | 1 (20.0%) | 2 (40.0%) | 2 (40.0%) | 1 (20.0%) |
| Subjects treated | 6 | 6 | 6 | 6 |
| with 25 mg BID | | | | |
| IGA | | | | |
| N | 6 | 6 | 6 | 6 |
| IGA Score = 0 or 1 | 4 (66.7%) | 1 (16.7%) | 4 (66.7%) | 6 (100.0%) |
| IGA Score = 0 | 2 (33.3%) | 1 (16.7%) | 0 | 2 (33.3%) |
| PASI | | | | |
| N | 6 | 6 | 6 | 6 |
| ≥75% Improvement | 3 (50.0%) | 1 (16.7%) | 3 (50.0%) | 6 (100.0%) |
| ≥90% Improvement | 2 (33.3%) | 1 (16.7%) | 2 (33.3%) | 3 (50.0%) |
| 100% Improvement | 1 (16.7%) | 1 (16.7%) | 0 | 2 (33.3%) |
| Subjects treated | 5 | 5 | 5 | 5 |
| with 100 mg QD | | | | |
| IGA | | | | |
| N | 5 | 5 | 5 | 5 |
| IGA Score = 0 or 1 | 3 (60.0%) | 3 (60.0%) | 3 (60.0%) | 3 (60.0%) |
| IGA Score = 0 | 3 (60.0%) | 0 | 1 (20.0%) | 1 (20.0%) |
| PASI | | | | |
| N | 5 | 5 | 5 | 5 |
| ≥75% Improvement | 3 (60.0%) | 3 (60.0%) | 3 (60.0%) | 3 (60.0%) |
| ≥90% Improvement | 3 (60.0%) | 3 (60.0%) | 2 (40.0%) | 2 (40.0%) |
| 100% Improvement | 2 (40.0%) | 0 | 1 (20.0%) | 1 (20.0%) |
| Subjects treated | 4 | 5 | 5 | 5 |
| with 100 mg BID | | | | |
| IGA | | | | |
| N | 4 | 5 | 5 | 5 |
| IGA Score = 0 or 1 | 3 (75.0%) | 4 (80.0%) | 3 (60.0%) | 4 (80.0%) |
| IGA Score = 0 | 1 (25.0%) | 2 (40.0%) | 2 (40.0%) | 3 (60.0%) |
| PASI | | | | |
| N | 4 | 5 | 5 | 5 |
| ≥75% Improvement | 3 (75.0%) | 4 (80.0%) | 5 (100.0%) | 4 (80.0%) |
| ≥90% Improvement | 2 (50.0%) | 3 (60.0%) | 3 (60.0%) | 4 (80.0%) |
| 100% Improvement | 1 (25.0%) | 2 (40.0%) | 2 (40.0%) | 2 (40.0%) |
| Combined | 26 | 26 | 26 | 27 |
| IGA | | | | |
| N | 26 | 26 | 26 | 27 |
| IGA Score = 0 or 1 | 14 (53.8%) | 12 (46.2%) | 15 (57.7%) | 21 (77.8%) |
| IGA Score = 0 | 10 (38.5%) | 5 (19.2%) | 5 (19.2%) | 10 (37.0%) |
| PASI | | | | |
| N | 26 | 26 | 26 | 27 |
| ≥75% Improvement | 13 (50.0%) | 12 (46.2%) | 14 (53.8%) | 22 (81.5%) |
| ≥90% Improvement | 11 (42.3%) | 7 (26.9%) | 11 (42.3%) | 15 (55.6%) |
| 100% Improvement | 7 (26.9%) | 4 (15.4%) | 4 (15.4%) | 9 (33.3%) |

^{a}All randomized subjects who received at least 1 complete dose of and had at least 1 valid blood sample drawn for PK analysis after their first dose.
Note:
For QD dosing, trough is at least 20 h since last AM dose. For BID dosing, trough at least 8 h since last dose.
Note:
The sample result from a participant who discontinued study agent will be excluded from that point onwards. In addition, the sample result from a participant who received an incomplete/incorrect or skipped dose(s) based on 2 previous doses prior to the PK sample collection or data from a participant who skip dose prior to PK sample collection will be excluded for that visit.

TABLE 10
Clinical Response at Week 16 by Intermediate Plasma Concentrations (ng/mL)

| | Intermediate Plasma Concentration Quartiles | | | |
|---|---|---|---|---|
| | <Q1 | Q1 to <Q2 | Q2 to <Q3 | ≥Q3 |
| Analysis set: | 4 | 5 | 5 | 7 |
| Pharmacokinetics Evaluable^{a} | | | | |
| Subjects treated | 0 | 1 | 1 | 1 |
| with 25 mg QD | | | | |
| IGA | | | | |
| N | 0 | 1 | 1 | 1 |
| IGA Score = 0 or 1 | 0 | 0 | 1 (100.0%) | 0 |
| IGA Score = 0 | 0 | 0 | 1 (100.0%) | 0 |
| PASI | | | | |
| N | 0 | 1 | 1 | 1 |
| ≥75% Improvement | 0 | 0 | 1 (100.0%) | 0 |
| ≥90% Improvement | 0 | 0 | 1 (100.0%) | 0 |
| 100% Improvement | 0 | 0 | 1 (100.0%) | 0 |
| Subjects treated | 1 | 1 | 1 | 2 |
| with 50 mg QD | | | | |
| IGA | | | | |
| N | 1 | 1 | 1 | 2 |
| IGA Score = 0 or 1 | 0 | 1 (100.0%) | 1 (100.0%) | 1 (50.0%) |
| IGA Score = 0 | 0 | 1 (100.0%) | 1 (100.0%) | 0 |
| PASI | | | | |
| N | 1 | 1 | 1 | 2 |
| ≥75% Improvement | 1 (100.0%) | 1 (100.0%) | 1 (100.0%) | 1 (50.0%) |
| ≥90% Improvement | 0 | 1 (100.0%) | 1 (100.0%) | 1 (50.0%) |
| 100% Improvement | 0 | 1 (100.0%) | 1 (100.0%) | 0 |
| Subjects treated | 1 | 1 | 1 | 1 |
| with 25 mg BID | | | | |
| IGA | | | | |
| N | 1 | 1 | 1 | 1 |
| IGA Score = 0 or 1 | 0 | 1 (100.0%) | 0 | 1 (100.0%) |
| IGA Score = 0 | 0 | 0 | 0 | 0 |
| PASI | | | | |
| N | 1 | 1 | 1 | 1 |
| ≥75% Improvement | 0 | 1 (100.0%) | 0 | 1 (100.0%) |
| ≥90% Improvement | 0 | 0 | 0 | 1 (100.0%) |
| 100% Improvement | 0 | 0 | 0 | 0 |
| Subjects treated | 1 | 1 | 1 | 1 |
| with 100 mg QD | | | | |
| IGA | | | | |
| N | 1 | 1 | 1 | 1 |
| IGA Score = 0 or 1 | 1 (100.0%) | 1 (100.0%) | 1 (100.0%) | 1 (100.0%) |
| IGA Score = 0 | 1 (100.0%) | 1 (100.0%) | 0 | 0 |
| PASI | | | | |
| N | 1 | 1 | 1 | 1 |
| ≥75% Improvement | 1 (100.0%) | 1 (100.0%) | 1 (100.0%) | 1 (100.0%) |
| ≥90% Improvement | 1 (100.0%) | 1 (100.0%) | 0 | 0) |
| 100% Improvement | 1 (100.0%) | 1 (100.0%) | 0 | 0 |
| Subjects treated | 1 | 1 | 1 | 2 |
| with 100 mg BID | | | | |
| IGA | | | | |
| N | 1 | 1 | 1 | 2 |
| IGA Score = 0 or 1 | 1 (100.0%) | 1 (100.0%) | 0 | 2 (100.0%) |
| IGA Score = 0 | 1 (100.0%) | 1 (100.0%) | 0 | 2 (100.0%) |
| PASI | | | | |
| N | 1 | 1 | 1 | 2 |
| ≥75% Improvement | 1 (100.0%) | 1 (100.0%) | 1 (100.0%) | 2 (100.0%) |
| ≥90% Improvement | 1 (100.0%) | 1 (100.0%) | 0 | 2 (100.0%) |
| 100% Improvement | 1 (100.0%) | 1 (100.0%) | 0 | 2 (100.0%) |
| Combined | 5 | 5 | 5 | 6 |
| IGA | | | | |
| N | 5 | 5 | 5 | 6 |
| IGA Score = 0 or 1 | 3 (60.0%) | 3 (60.0%) | 3 (60.0%) | 5 (83.3%) |
| IGA Score = 0 | 3 (60.0%) | 2 (40.0%) | 1 (20.0%) | 3 (50.0%) |
| PASI | | | | |
| N | 5 | 5 | 5 | 6 |
| ≥75% Improvement | 4 (80.0%) | 3 (60.0%) | 3 (60.0%) | 6 (100.0%) |
| ≥90% Improvement | 3 (60.0%) | 2 (40.0%) | 1 (20.0%) | 5 (83.3%) |
| 100% Improvement | 3 (60.0%) | 2 (40.0%) | 1 (20.0%) | 3 (50.0%) |

^{a}All randomized subjects who received at least 1 complete dose of and had at least 1 valid blood sample drawn for PK analysis after their first dose.
Note:
For QD dosing, intermediate is 6 to <20 h since last AM dose. For BID dosing, intermediate is 4 to <8 h since last dose.
Note:
The sample result from a participant who discontinued study agent will be excluded from that point onwards. In addition, the sample result from a participant who received an incomplete/incorrect or skipped dose(s) based on 2 previous doses prior to the PK sample collection or data from a participant who skip dose prior to PK sample collection will be excluded for that visit.

TABLE 11
Clinical Response at Week 16 by Peak Plasma Concentrations (ng/mL)

| | Peak Plasma Concentration Quartiles | | | | | | | |
|---|---|---|---|---|---|---|---|---|
| | <Q1 | | Q1 to <Q2 | | Q2 to <Q3 | | ≥Q3 | |
| Analysis set: | 10 | | 11 | | 10 | | 11 | |
| Pharmacokinetics | | | | | | | | |
| Evaluable^{a} | | | | | | | | |
| Subjects treated | 2 | | 2 | | 2 | | 2 | |
| with 25 mg QD | | | | | | | | |
| IGA | | | | | | | | |
| N | 2 | | 2 | | 2 | | 2 | |
| IGA Score = 0 or 1 | 1 | (50.0%) | 1 | (50.0%) | 1 | (50.0%) | 1 | (50.0%) |
| IGA Score = 0 | 0 | | 0 | | 1 | (50.0%) | 0 | |
| PASI | | | | | | | | |
| N | 2 | | 2 | | 2 | | 2 | |
| ≥75% Improvement | 1 | (50.0%) | 1 | (50.0%) | 1 | (50.0%) | 1 | (50.0%) |
| ≥90% Improvement | 1 | (50.0%) | 1 | (50.0%) | 1 | (50.0%) | 1 | (50.0%) |
| 100% Improvement | 0 | | 0 | | 0 | | 0 | |
| Subjects treated | 2 | | 2 | | 2 | | 2 | |
| with 50 mg QD | | | | | | | | |
| IGA | | | | | | | | |
| N | 2 | | 2 | | 2 | | 2 | |
| IGA Score = 0 or 1 | 0 | | 2 | (100.0%) | 2 | (100.0%) | 2 | (100.0%) |
| IGA Score = 0 | 0 | | 2 | (100.0%) | 0 | | 0 | |
| PASI | | | | | | | | |
| N | 2 | | 2 | | 2 | | 2 | |
| ≥75% Improvement | 1 | (50.0%) | 2 | (100.0%) | 1 | (50.0%) | 2 | (100.0%) |
| ≥90% Improvement | 0 | | 2 | (100.0%) | 1 | (50.0%) | 2 | (100.0%) |
| 100% Improvement | 0 | | 1 | (50.0%) | 0 | | 0 | |
| Subjects treated | 2 | | 2 | | 2 | | 2 | |
| with 25 mg BID | | | | | | | | |
| IGA | | | | | | | | |
| N | 2 | | 2 | | 2 | | 2 | |
| IGA Score = 0 or 1 | 0 | | 1 | (50.0%) | 1 | (50.0%) | 1 | (50.0%) |
| IGA Score = 0 | 0 | | 0 | | 0 | | 1 | (50.0%) |
| PASI | | | | | | | | |
| N | 2 | | 2 | | 2 | | 2 | |
| ≥75% Improvement | 0 | | 2 | (100.0%) | 1 | (50.0%) | 1 | (50.0%) |
| ≥90% Improvement | 0 | | 0 | | 1 | (50.0%) | 1 | (50.0%) |
| 100% Improvement | 0 | | 0 | | 0 | | 0 | |
| Subjects treated | 2 | | 2 | | 2 | | 2 | |
| with 100 mg QD | | | | | | | | |
| IGA | | | | | | | | |
| N | 2 | | 2 | | 2 | | 2 | |
| IGA Score = 0 or 1 | 1 | (50.0%) | 2 | (100.0%) | 1 | (50.0%) | 1 | (50.0%) |
| IGA Score = 0 | 1 | (50.0%) | 1 | (50.0%) | 1 | (50.0%) | 0 | |
| PASI | | | | | | | | |
| N | 2 | | 2 | | 2 | | 2 | |
| ≥75% Improvement | 2 | (100.0%) | 2 | (100.0%) | 1 | (50.0%) | 1 | (50.0%) |
| ≥90% Improvement | 1 | (50.0%) | 1 | (50.0%) | 1 | (50.0%) | 1 | (50.0%) |
| 100% Improvement | 0 | | 1 | (50.0%) | 0 | | 0 | |
| Subjects treated | 2 | | 3 | | 2 | | 3 | |
| with 100 mg BID | | | | | | | | |
| IGA | | | | | | | | |
| N | 2 | | 3 | | 2 | | 3 | |
| IGA Score = 0 or 1 | 2 | (100.0%) | 2 | (66.7%) | 1 | (50.0%) | 3 | (100.0%) |
| IGA Score = 0 | 1 | (50.0%) | 2 | (66.7%) | 1 | (50.0%) | 2 | (66.7%) |
| PASI | | | | | | | | |
| N | 2 | | 3 | | 2 | | 3 | |
| ≥75% Improvement | 2 | (100.0%) | 3 | (100.0%) | 1 | (50.0%) | 3 | (100.0%) |
| ≥90% Improvement | 2 | (100.0%) | 3 | (100.0%) | 1 | (50.0%) | 2 | (66.7%) |
| 100% Improvement | 1 | (50.0%) | 1 | (33.3%) | 1 | (50.0%) | 2 | (66.7%) |
| Combined | 10 | | 11 | | 10 | | 11 | |
| IGA | | | | | | | | |
| N | 10 | | 11 | | 10 | | 11 | |
| IGA Score = 0 or 1 | 3 | (30.0%) | 8 | (72.7%) | 8 | (80.0%) | 7 | (63.6%) |
| IGA Score = 0 | 1 | (10.0%) | 4 | (36.4%) | 3 | (30.0%) | 5 | (45.5%) |
| PASI | | | | | | | | |
| N | 10 | | 11 | | 10 | | 11 | |
| ≥75% Improvement | 4 | (40.0%) | 10 | (90.9%) | 7 | (70.0%) | 8 | (72.7%) |
| ≥90% Improvement | 3 | (30.0%) | 7 | (63.6%) | 6 | (60.0%) | 7 | (63.6%) |

| | | | | | | | |
|---|---|---|---|---|---|---|---|
| 100% Improvement | 0 | 2 | (18.2%) | 1 | (10.0%) | 4 | (36.4%) |

^{a}All randomized subjects who received at least 1 complete dose of and had at least 1 valid blood sample drawn for PK analysis after their first dose.
Note:
For QD dosing, peak is defined as any sample between 0.25 to <6 h since last AM dose. For BID dosing, peak is defined as any sample between 0.25 to <4 h since last dose.
Note:
The sample result from a participant who discontinued study agent will be excluded from that point onwards. In addition, the sample result from a participant who received an incomplete/incorrect or skipped dose(s) based on 2 previous doses prior to the PK sample collection or data from a participant who skip dose prior to PK sample collection will be excluded for that visit.

### Pharmacodynamics

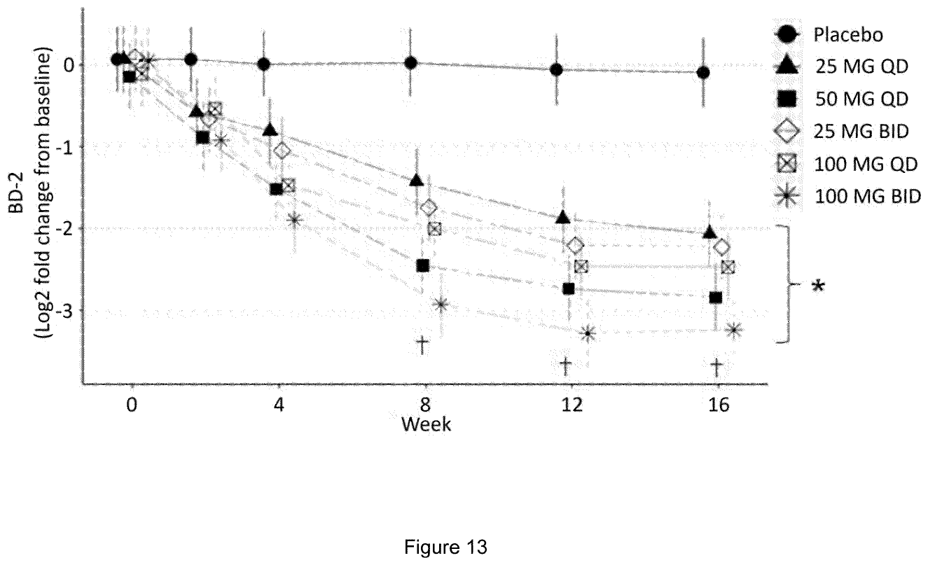

The evaluable pharmacodynamics (PD) analysis set included all randomized subjects. In all doses of the IL-23 receptor antagonist peptide, strong systemic PD responses were observed that significantly distinguished the treatment from the placebo. The IL-23 receptor antagonist peptide significantly dampened serum levels of psoriasis-associated biomarkers BD-2 (FIG. 13). Statistically significant reduction of BD-2 serum levels was observable in all treatment groups as early as Week 4, with 100 mg BID dosage resulting in a statistically significant reduction in BD-2 relative to all other dosages from Week 8 on.

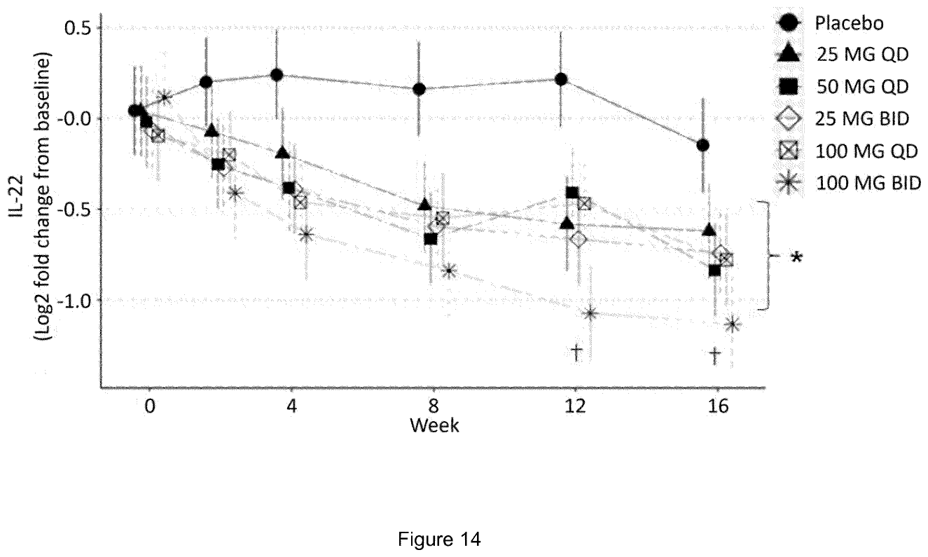

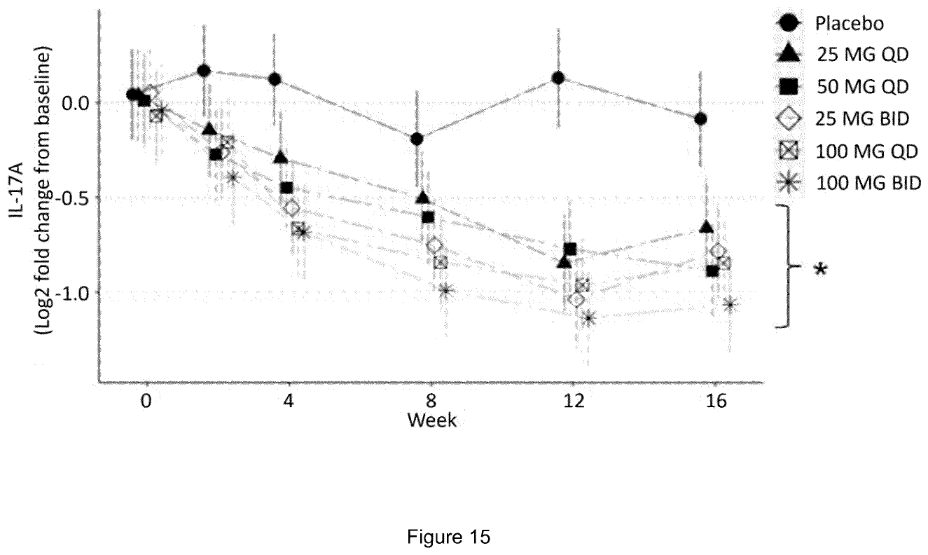

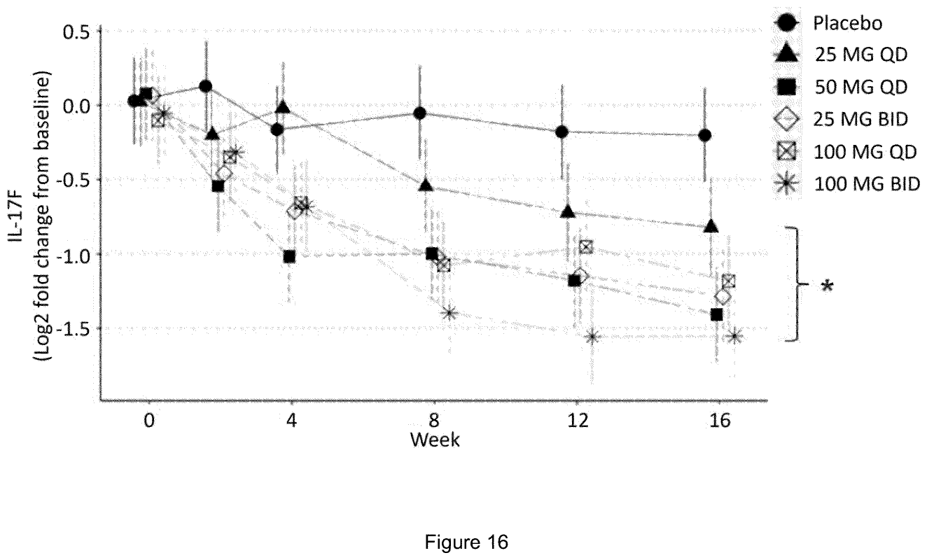

The statistically significant reduction in IL-22 serum levels was similarly observed in all treatment groups relative to placebo, with the 100 mg BID treatment group again producing a statistically significant reduction relative to all other dosages from Week 12 on (FIG. 14). Statistically significant reductions in serum levels of IL-17A (FIG. 15) and IL-17F (FIG. 16) were also observed, with all treatment groups relative to placebo.

To evaluate psoriasis relevant systemic biomarkers (Beta-defensin 2 (BD-2), IL-17A, IL-17F, and IL-22), protein concentrations were measured in serum using validated immunoassays. The IL-17A (Catalog #03-0159-00) and IL-17F (Catalog #03-0164-00) assays used are commercially available SMC™-based high sensitivity immunoassays produced by the EMD Millipore Corporation. The manufacturer's protocols were followed and analyzed on the Erenna® instrument platform except for the use of 11 μL Elution Buffer B. While the IL-22 assay is also based on the SMC™ platform and run on the Erenna instrument, the assay was developed internally as a coated plate-based assay. The development and optimization of the BD-2 assay was done on the Meso Scale Discovery on the electrochemiluminescence-based platform, a division of Meso Scale Diagnostics LLC (MSD). For all assays, concentration adjustments were made to values measuring below empirically defined LLOQ or empirically defined ULOQ values. Assay QC verified performance of generated standard curves (average concentration recovery & CV of technical replicates) and control samples (inter-assay plate agreement (CV)).

### Immunogenicity:

A summary of the incidence of anti-drug antibodies (ADAs) to the IL-23 receptor antagonist peptide through Week 16 is presented in Table 12. The overall incidence of ADA was low, with 3 of 209 (1.4%) participants testing positive for ADA prior to the first dose of the IL-23 receptor antagonist peptide (baseline) who were negative thereafter. All 3 participants were randomized to the IL-23 receptor antagonist peptide 50 mg QD treatment group. Eight of 209 (3.8u) additional participants had one sample positive for ADA post-dose. Antibody responses to the IL-23 receptor antagonist peptide were low titer (1:50, the minimum detectable titer in the assay) and were transient, with ADA-positive results observed at a single timepoint during the study for all participants with reported ADA-positive results. There was no clear association between positive ADA results and dose level or time on treatment.

TABLE 12
Summary of Immunogenicity

| Treatment Regimen | 25 mg QD | 50 mg QD | 25 mg BID | 100 mg QD | 100 mg BID | Combined^{a} | |
|---|---|---|---|---|---|---|---|
| Total Set | 42 | 43 | 41 | 42 | 41 | 209 | |
| Subjects positive for anti-IL-23 | 0 | 3 (7.0%) | 0 | 0 | 0 | 3 | (1.4%) |
| receptor antagonist peptide | | | | | | | |
| antibodies at baseline^{b} | | | | | | | |
| Baseline titers 1:50 | 0 | 3 | 0 | 0 | 0 | 3 | |
| Subjects positive for anti-IL-23 | 0 | 0 | 0 | 0 | 0 | 0 | |
| receptor antagonist peptide | | | | | | | |
| antibodies at baseline who were | | | | | | | |
| treatment-boosted for antibodie^{c} | | | | | | | |
| Baseline titers 1:50 | 0 | 0 | 0 | 0 | 0 | 0 | |
| Subjects positive for anti-IL-23 | 0 | 3 (7.0%) | 0 | 0 | 0 | 3 | (1.4%) |
| receptor antagonist peptide | | | | | | | |
| antibodies at baseline who were | | | | | | | |
| treatment-boosted for antibodies^{d} | | | | | | | |
| Baseline titers 1:50 | 0 | 3 | 0 | 0 | 0 | 3 | |
| Subjects positive for treatment- | 1 (2.4%) | 1 (2.3%) | 0 | 3 (7.1%) | 3 (7.3%) | 8 | (3.8%) |
| emergent anti-IL-23 receptor | | | | | | | |
| antagonist peptide antibodies^{e} | | | | | | | |
| Peak titers 1:50 | 1 | 1 | 0 | 3 | 3 | 8 | |
| Peak titer group | | | | | | | |
| <50 | 1 | 1 | 0 | 3 | 3 | 8 | |
| >50-<100 | 0 | 0 | 0 | 0 | 0 | 0 | |
| ≥100-<1000 | 0 | 0 | 0 | 0 | 0 | 0 | |
| ≥1000 | 0 | 0 | 0 | 0 | 0 | 0 | |
| Subjects negative for treatment- | 41 (97.6%) | 42 (97.7%) | 41 (100%) | 39 (92.9%) | 38 (92.7%) | 201 | (96.2%) |

emergent anti-IL-23 receptor
antagonist peptide antibodies^{f}

^{a}Includes all treatment columns (25 mg QD, 50 mg QD, 25 mg BID, 100 mg QD, and 100 mg BID).
^{b}Subjects positive for antibodies at baseline, regardless of status after first administration.
^{c}Subjects positive for treatment-boosted antibodies includes subjects who were positive at baseline and whose titers increased 2-fold at any time. Subjects with baseline samples and without 2-fold increased titer after treatment are not considered treatment-boosted.
^{d}Includes subjects positive for antibodies at baseline but whose titers did not increase 2-fold after their first administration, remained the same after treatment or ADA titers were reduced or disappeared after administration.
^{e}Subjects positive for treatment-emergent antibodies includes all subjects who were positive (treatment-boosted or treatment-induced at any time after their first administration through Week 16 or Follow-up Weck 20 visit. Subjects with baseline positive samples and without 2-fold increased titer after treatment are not considered treatment-boosted.
^{f}Excludes subjects who were treatment-emergent positive at any time through Week 16 or Follow-up Week 20 visit.

A summary of the incidence of neutralizing antibodies (NAbs) to the IL-23 receptor antagonist peptide through Week 16 is presented in Table 13. All ADA-positive samples were negative in the NAb assay. There was no apparent correlation between ADA positivity and efficacy.

TABLE 13
Summary of Treatment-emergent Neutralizing Antibodies through Week 16

| Treatment Group | 25 mg QD | | 50 mg QD | | 25 mg BID | 100 mg QD | | 100 mg BID | | Combined^{a} | |
|---|---|---|---|---|---|---|---|---|---|---|---|
| Total Set | 42 | | 43 | | 41 | 42 | | 41 | | 209 | |
| Subjects positive for treatment-emergent | 1 | (2.4%) | 1 | (2.3%) | 0 | 3 | (7.1%) | 3 | (7.3%) | 8 | (3.8%) |
| anti-IL-23 receptor antagonist peptide | | | | | | | | | | | |
| antibodies at any time^{b} | | | | | | | | | | | |
| Subjects evaluable for neutralizing | 1 | (2.4%) | 1 | (2.3%) | 0 | 3 | (7.1%) | 3 | (7.3%) | 8 | (3.8%) |
| antibodies^{c} | | | | | | | | | | | |
| Subjects positive for neutralizing | — | | — | | — | — | | 1 | | — | |
| antibodies at baseline^{d} | | | | | | | | | | | |
| Subjects positive for treatment-emergent | 0 | | 0 | | — | 0 | | 0 | | 0 | |
| neutralizing antibodies^{e} | | | | | | | | | | | |
| Subjects negative for neutralizing | 1 | (100%) | 1 | (100%) | — | 3 | (100%) | 3 | (100%) | 8 | (100%) |

antibodies^{f}

^{a}Includes all treatment columns (25 mg QD, 50 mg QD, 25 mg BID, 100 mg QD, and 100 mg BID).
^{b}Subjects positive for treatment-emergent antibodies includes all subjects who were positive (treatment-boosted or treatment-induced) at any time after their first administration through Week 16 or Follow-up Week 20. Subjects with baseline positive samples and without 2-fold increased titer after treatment are not considered treatment-boosted.
^{c}An evaluable subject is a subject positive for treatment-emergent antibodies with no detectable interference in the neutralizing antibody assay.
^{d}Subjects positive for neutralizing antibodies at baseline regardless of antibody status after first administration.
^{e}Subjects positive for neutralizing antibodies from positive treatment-emergent antibody subjects.
^{f}Excludes subjects for neutralizing antibodies at any time.

### Baseline Disease Characteristics

The baseline psoriasis disease characteristics of participants who received at least one study agent (Full Analysis Set) are consistent with a population with moderate to severe psoriasis (Table 14). Baseline disease characteristics were generally comparable across the treatment groups, with some variability observed across treatment groups. The mean duration of disease of psoriasis was 18.2 (12.79) years. The mean percent of body surface area (BSA) involved was 22.8 (12.99), with a mean total PASI score of 19.05 (5.831). In addition, 79.2% participants presented with an IGA=3, and 20.8% of participants had severe disease as defined by their baseline IGA score of 4.

The mean (SD) baseline DLQI, PSSD symptoms and signs scores were 13.5 (7.08), 51.8 (23.84), and 64.6 (18.25) respectively (Table 14, Table 15, and Table 16).

TABLE 14
Psoriasis Disease Characteristics at Baseline

| Treatment | | | | | | | | |
|---|---|---|---|---|---|---|---|---|
| Regimen | Placebo | 25 mg QD | 50 mg QD | 25 mg BID | 100 mg QD | 100 mg BID | Combined^{a} | Total |
| Analysis set: Full | 43 | 43 | 43 | 41 | 43 | 42 | 212 | 255 |
| Analysis set | | | | | | | | |
| Mean Disease | 17.9 | 15.5 | 21.5 | 18.1 | 19.5 | 16.7 | 18.3 | 18.2 |
| Duration in | (14.37) | (11.76) | (11.16) | (11.82) | (13.34) | (13.78) | (12.48) | (12.79) |
| Years(SD) | | | | | | | | |

Age at diagnosis (years)

| | | | | | | | | |
|---|---|---|---|---|---|---|---|---|
| Mean (SD) | 26.1 | 29.1 | 23.7 | 27.7 | 25.3 | 25.5 | 26.2 | 26.2 |
| | (15.55) | (15.56) | (11.57) | (13.73) | (15.08) | (15.26) | (14.31) | (14.50) |

PASI total score

| | | | | | | | | |
|---|---|---|---|---|---|---|---|---|
| Mean (SD) | 18.99 | 18.90 | 19.23 | 18.46 | 18.42 | 20.33 | 19.07 | 19.05 |
| | (5.341) | (5.272) | (5.082) | (5.383) | (6.873) | (6.509) | (5.938) | (5.831) |

IAG Score

| | | | | | | | | |
|---|---|---|---|---|---|---|---|---|
| Severe (4) | 5 | 13 | 7 | 8 | 8 | 12 | 48 | 53 |
| | (11.5%) | (30.2%) | (16.3%) | (19.5%) | (18.6%) | (28.6%) | (22.6%) | (20.8%) |
| Moderate (3) | 38 | 30 | 36 | 33 | 35 | 30 | 164 | 202 |
| | (88.4%) | (69.8%) | (83.7%) | (80.5%) | (81.4%) | (71.4%) | (77.4%) | (79.2%) |

BSA (%)

| | | | | | | | | |
|---|---|---|---|---|---|---|---|---|
| Mean (SD) | 26.1 | 21.1 | 23.9 | 20.9 | 20.5 | 24.2 | 22.1 | 22.8 |
| | (15.72) | (9.28) | (13.59) | (11.93) | (13.69) | (12.55) | (12.30) | (12.99) |

^{a}Includes all peptide treatment columns (25 mg QD, 50 mg QD, 25 mg BID, 100 mg QD, and 100 mg BID)

TABLE 15
Summary of Patient Reported Outcomes at Baseline

| Treatment | | | | | | | | |
|---|---|---|---|---|---|---|---|---|
| Regimen | Placebo | 25 mg QD | 50 mg QD | 25 mg BID | 100 mg QD | 100 mg BID | Combined^{a} | Total |
| Analysis set: Full | 43 | 43 | 43 | 41 | 43 | 42 | 212 | 255 |
| Analysis set | | | | | | | | |

PSSD symptom score (0-100)

| | | | | | | | | |
|---|---|---|---|---|---|---|---|---|
| Mean (SD) | 47.3 | 59.0 | 53.9 | 51.9 | 43.0 | 55.9 | 52.7 | 51.8 |
| | (20.67) | (23.63) | (24.49) | (24.05) | (21.27) | (26.27) | (24.37) | (23.84) |

PSSSD sign score (0-100)

| | | | | | | | | |
|---|---|---|---|---|---|---|---|---|
| Mean (SD) | 62.9 | 69.5 | 64.7 | 64.1 | 60.4 | 66.3 | 65.0 | 64.6 |
| | (16.64) | (16.50) | (19.41) | (18.90) | (18.56) | (19.09) | (18.58) | (18.25) |

DLQI (0-30)

| | | | | | | | | |
|---|---|---|---|---|---|---|---|---|
| Mean (SD) | 12.4 | 13.8 | 14.1 | 14.0 | 11.1 | 15.6 | 13.7 | 13.5 |
| | (7.56) | (7.55) | (5.53) | (7.50) | (6.15) | (7.50) | (6.97) | (7.08) |

^{a}Includes all peptide treatment columns (25 mg QD, 50 mg QD, 25 mg BID, 100 mg QD, and 100 mg BID)

TABLE 16
Summary of Previous Psoriasis Medications and Therapies

| Treatment | | | | | | | | |
|---|---|---|---|---|---|---|---|---|
| Regimen | Placebo | 25 mg QD | 50 mg QD | 25 mg BID | 100 mg QD | 100 mg BID | Combined^{a} | Total |
| Analysis set: Full | 43 | 43 | 43 | 41 | 43 | 42 | 212 | 255 |
| Analysis set | | | | | | | | |
| Phototherapy | 19 | 17 | 24 | 15 | 21 | 14 | 91 | 110 |
| (PUVA or UVB) | (44.2%) | (39.5%) | (55.8%) | (36.6%) | (48.8%) | (33.3%) | (42.9%) | (43.1%) |
| Conventional | 17 | 20 | 21 | 20 | 24 | 20 | 105 | 122 |
| non-biologic | (39.5%) | (46.5%) | (48.8%) | (48.8%) | (55.8%) | (47.6%) | (49.5%) | (47.8%) |
| systemic | | | | | | | | |
| Novel non- | 4 | 5 | 2 | 2 | 2 | 3 | 14 | 18 |
| biologic systemic | (9.3%) | (11.6%) | (4.7%) | (4.9%) | (4.7%) | (7.1%) | (6.6%) | (7.1%) |
| Biologics | 7 | 7 | 11 | 13 | 9 | 9 | 49 | 56 |
| | (16.3%) | (16.3%) | (25.6%) | (31.7%) | (20.9%) | (21.4%) | (23.1%) | (22.0%) |
| Systemics | 34 | 33 | 35 | 33 | 34 | 31 | 166 | 200 |
| | (79.1%) | (76.7%) | (81.4%) | (80.5%) | (79.1%) | (73.8%) | (78.3%) | (78.4%) |

Conventional non-biologic systemic
Novel non-biologic systemic
Biologics
Systemics (conventional nonbiologic systemics, novel nonbiologic systemics, 1,25- vitamin D3 and analogues, phototherapy, biologics)
^{a}Includes all peptide treatment columns (25 mg QD, 50 mg QD, 25 mg BID, 100 mg QD, and 100 mg BID)

### Scalp Psoriasis Response

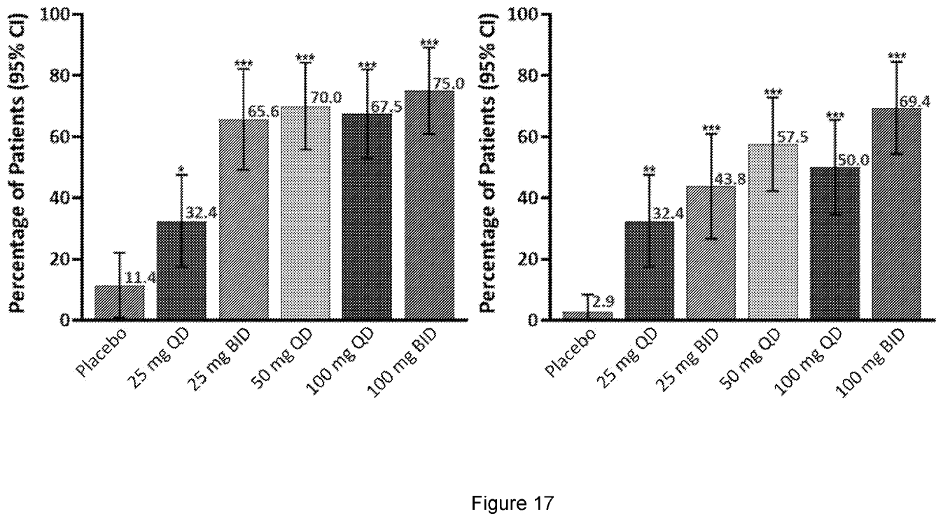

A significant dose response was observed for the primary endpoint of PASI 75 in the study described above. Response rates (PASI 75, PASI 90, PASI 100, IGA score 0/1, IGA score 0, and ss-IGA score 0/1 with ≥2-grade improvement from baseline) for all doses were significantly higher than placebo (nominal p<0.05 for all comparisons) at W16 (Table 17). The compound of Formula (I) demonstrated significantly greater efficacy compared with placebo in patients with moderate-to-severe plaque psoriasis, including scalp psoriasis, and was well-tolerated in all treatment groups. Proportion of Patients Achieving scalp-specific (ss)-IGA score 0/1 and ss-IGA score 0 and With at Least a 2-Grade Improvement from baseline at Week 16. Response rates for scalp-specific (ss)-IGA scores of 0/1 and 0 and with at least a 2-grade improvement from baseline for all doses were significantly higher than placebo at Week 16 as shown in FIG. 17.

TABLE 17
Proportions of Patients Achieving Overall and Scalp Psoriasis Efficacy Endpoints at Week 16

| | | | 25 mg QD | | 50 mg QD | 25 mg BID | | 100 mg QD | 100 mg BID |
|---|---|---|---|---|---|---|---|---|---|
| Treatment Regimen | Placebo | | (N = 43) | | (N = 43) | (N = 41) | | (N = 43) | (N = 42) |
| PASI 75, n (%) | 4 | (9.3) | 16 | (37.2) | 25 (58.1) | 21 | (51.2) | 28 (65.1) | 33 (78.6) |
| PASI 90, n (%) | 1 | (2.3) | 11 | (25.6) | 22 (51.2) | 11 | (26.8) | 20 (46.5) | 25 (59.5) |
| PASI 100, n (%) | 0 | | 5 | (11.6) | 11 (25.6) | 4 | (9.8) | 10 (23.3) | 17 (40.5) |
| IGA score 0/1, n (%) | 5 | (11.6) | 17 | (39.5) | 25 (58.1) | 21 | (51.2) | 27 (62.8) | 27 (64.3) |
| IGA score 0, n (%) | 0 | | 7 | (16.3) | 15 (34.9) | 6 | (14.6) | 12 (27.9) | 19 (45.2) |
| Baseline ss-IGA score ≥2, n | 35 | | 37 | | 40 | 32 | | 40 | 36 |
| ss-IGA score 0/1 and ≥2- | 4 | (11.4) | 12 | (32.4) | 28 (70.0) | 21 | (65.6) | 27 (67.5) | 27 (75.0) |

grade improvement from
baseline, n (%)

IGA = Investigator's Global Assessment;
PASI = Psoriasis Area and Severity Index;
ss-IGA = Scalp-specific Investigator's Global Assessment

### Serum Levels of Psoriasis Disease Biomarkers BD-2, IL-22, IL-17A, and IL-17F

Serum levels of psoriasis disease biomarkers BD-2, IL-22, IL-17A, and IL-17F relative to baseline were analyzed in patients who received placebo or the compound of Formula (I) and compared to clinical response. A linear mixed effect model was used to analyze the treatment over time interaction, baseline serum protein levels, and patient random effect.

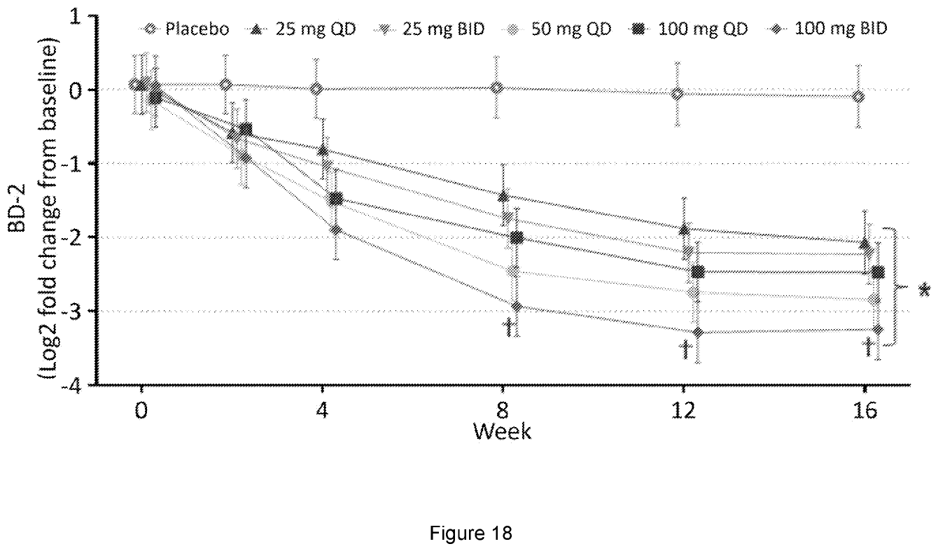

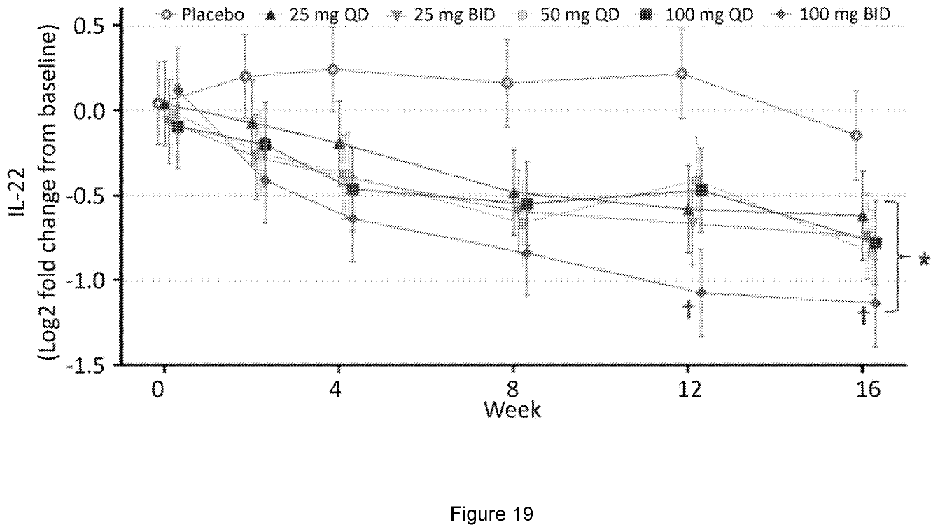

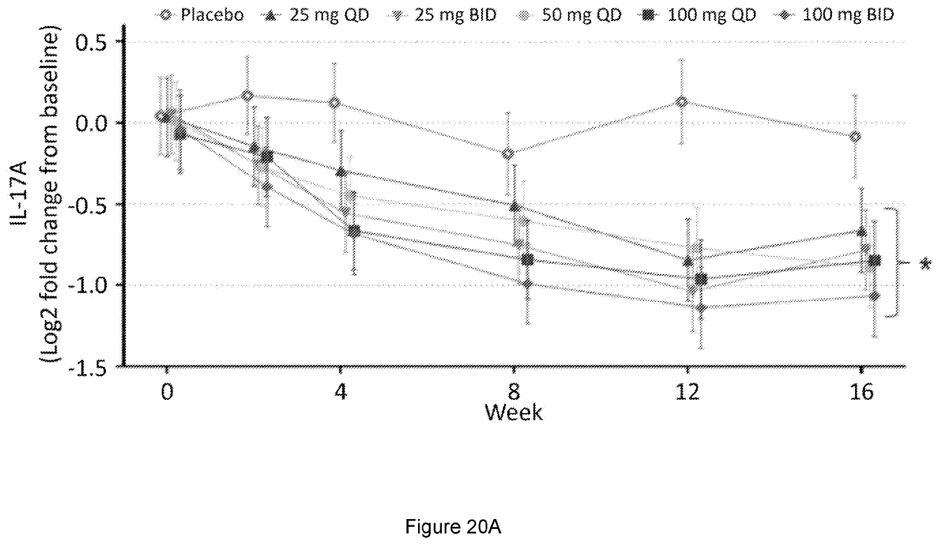

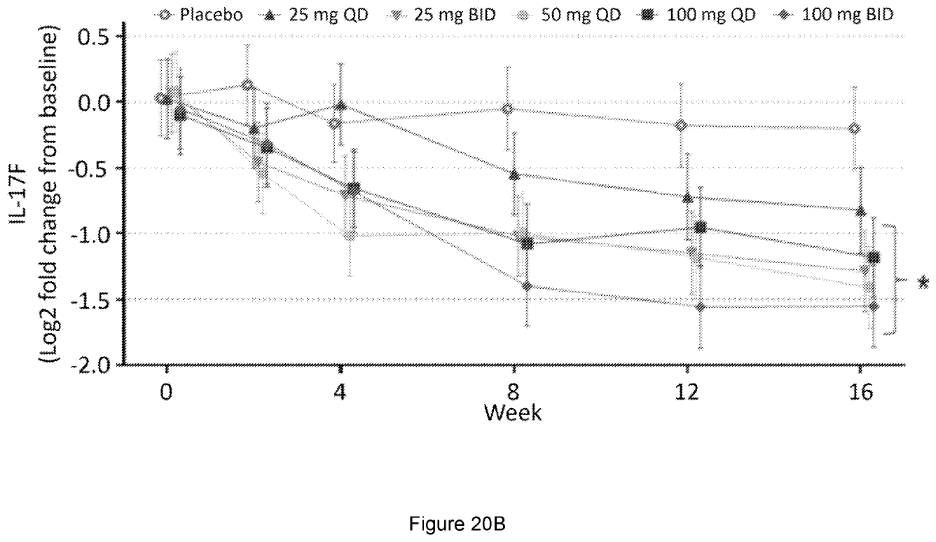

Systemic pharmacodynamic changes demonstrated by changes in the serum levels of biomarkers like BD-2, which is an antimicrobial peptide, have been shown to correlate with PASI responses following therapeutic treatment responses. Serum levels of these biomarkers relevant to psoriasis disease pathophysiology were analyzed and it was found that the fold reduction of BD 2 by all doses was significantly differentiated from placebo starting from Week 4 as shown in FIG. 18. The fold change in BD-2 was greater with increasing clinical response. Regardless of time point or dose, a progressive increase in the fold reduction in serum levels of BD-2 was observed with increase in clinical response indicating a correlation between dampening of serum BD-2 and PASI response. The treatment of compound of formula (I) drove significant dampening of serum IL-22 that was distinguished from placebo at all doses as shown in FIG. 19, statistically significant relative to placebo starting from Week 4. The pharmacodynamic response on serum IL-22 levels correlated with clinical outcomes. The isoform of the IL17 cytokines with significant dampening of serum IL-17A and IL-17F levels with higher reduction statistically significant relative to placebo as shown in FIG. 20. The treatment did not increase serum levels of IL-23 in psoriasis patients.

### Example 2. Extended Daily Administration for 52 Weeks in Adult Human Subjects with Moderate to Severe Plaque Psoriasis

IL-23 receptor antagonist peptide as used in this example refers to a compound of Formula (I) or a pharmaceutically acceptable salt or solvate thereof, specifically a hydrochloride salt of compound of Formula (I) in a crystalline form.

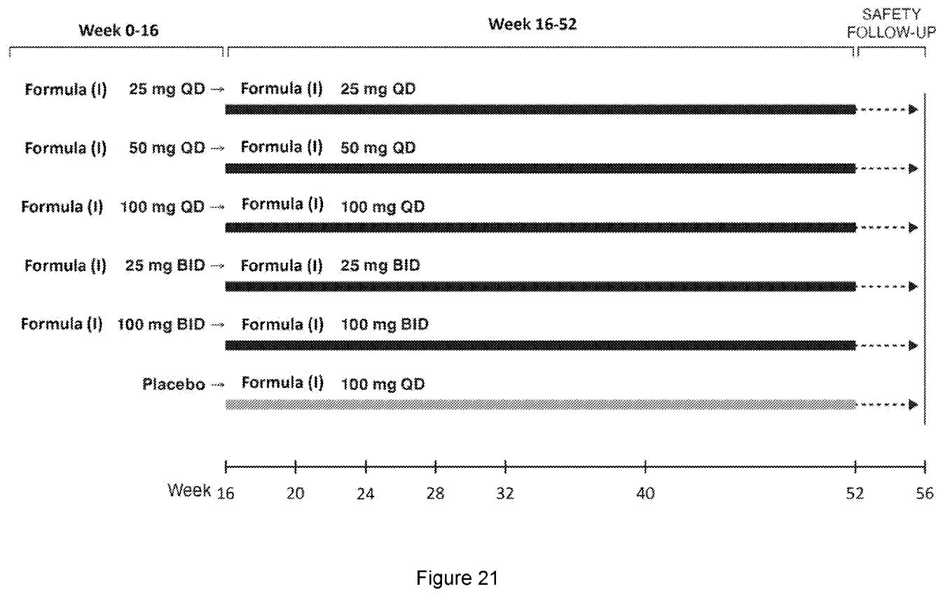

Described below is a multicenter, long-term extension (LTE), double-blind, dose-ranging, parallel group interventional study in participants from Example 1 to evaluate the long-term efficacy and safety of the hydrochloride salt of the compound of Formula (I) for the treatment of moderate-to-severe plaque psoriasis. Patients from Example 1 continued to receive the same dose of the compound of Formula (I) for an additional 36-week treatment period with a 4-week safety follow up period after the last administration of the compound of Formula (I). Patients randomized to placebo in Example 1 received the compound of Formula (I) at a dose of 100 mg QD starting at Week 16 of Example 1 (FIG. 21). A summary of treatment groups is shown in Table 18.

TABLE 18
Summary of Treatment through Week 52

| | | Placebo → | | | | | | | | | |
|---|---|---|---|---|---|---|---|---|---|---|---|
| Treatment Regimen | Placebo | 100 mg QD^{a} | 25 mg QD | 50 mg QD | 25 mg BID | | 100 mg QD | | 100 mg BID | | Combined^{b} |
| Analysis set: Safety | 43 | 35 | 43 | 43 | 41 | | 43 | | 42 | | 247 |
| analysis set | | | | | | | | | | | |
| Total duration of | | | | | | | | | | | |
| exposure (weeks)^{c} | | | | | | | | | | | |
| N | 43 | 35 | 43 | 43 | 41 | | 43 | | 42 | | 247 |
| Mean (SD) | 14.1 (4.74) | 32.6 (8.32) | 40.6 (18.09) | 46.2 (14.47) | 44.9 | (12.53) | 44.7 | (14.82) | 46.0 | (15.72) | 42.8 (15.04) |
| Median | 16.0 | 36.0 | 52.0 | 52.1 | 52.0 | | 52.1 | | 52.1 | | 52.0 |
| Range | (2; 18) | (3; 37) | (0; 55) | (0; 54) | (8; 55) | | (0; 54) | | (1; 56) | | (0; 56) |
| IQ range | (15.6; 16.1) | (36.0; 36.1) | (24.1; 52.1) | (51.9; 52.7) | (40.1; 52.6) | | (51.0; 52.3) | | (52.0; 53.0) | | (36.0; 52.3) |
| Average daily dose | | | | | | | | | | | |
| (mg/day)^{d} | | | | | | | | | | | |
| N | 0 | 35 | 43 | 43 | 41 | | 43 | | 42 | | 247 |
| Mean (SD) | — | 106.9 (24.39) | 30.7 (26.97) | 53.1 (15.47) | 50.5 | (6.73) | 103.4 | (17.33) | 199.2 | (20.23) | 90.0 (59.96) |

| | | | | | | | | |
|---|---|---|---|---|---|---|---|---|
| Median | — | 99.6 | 25.0 | 50.0 | 49.5 | 100.0 | 199.4 | 72.6 |
| Range | — | (91; 222) | (22; 200) | (46; 150) | (36; 79) | (81; 200) | (140; 291) | (22; 291) |
| IQ range | — | (98.4; 100.4) | (24.6; 25.2) | (49.4; 50.4) | (48.3; 50.0) | (98.4; 100.3) | (196.4; 200.0) | (48.8; 100.4) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
^{c}Total duration of treatment is defined as (date of last dose of study medication − date of first dose of study medication) + 1.
^{d}Number of the total active tablets taken × tablet's strength (25 mg or 100 mg)/(the last dose date − the first dose date + 1).

Example 1 began with 255 participants randomized into six treatment groups: Placebo (n=43), 25 mg QD (n=43), 25 mg BID (n=41), 50 mg QD (n=43), 100 mg QD (n=43), and 100 mg BID (n=43). At week 16, 227 participants continued treatment and received at least one additional dose: Placebo→100 mg QD (n=25), 25 mg QD (n=35), 25 mg BID (n=40), 50 mg QD (n=39), 100 mg QD (n=40), and 100 mg BID (n=38). The proportion of participants discontinuing treatment was highest in the 25 mg BID group (n=10, 25.0%). The most common reason for discontinuing treatment was lack of efficacy (n=19, 8.4%), which tended to be higher in the 25 mg QD (n=4, 11.4%) and 25 mg BID (n=5, 12.50%) groups (Table 19).

TABLE 19
Treatment Disposition from Week 16 to Week 52

| | Placebo → | | | | | | | | | | | | |
|---|---|---|---|---|---|---|---|---|---|---|---|---|---|
| Treatment Regimen | 100 mg QD | | 25 mg QD | | 50 mg QD | | 25 mg BID | | 100 mg QD | | 100 mg BID | Combined^{a} | |
| Analysis set: LTE Full | 35 | | 35 | | 39 | | 40 | | 40 | | 38 | 227 | |
| analysis set | | | | | | | | | | | | | |
| Discontinued study | 6 | (17.1%) | 6 | (17.1%) | 4 | (10.3%) | 10 | (25.0%) | 7 | (17.5%) | 3 (7.9%) | 36 | (15.9%) |
| treatment | | | | | | | | | | | | | |
| Reason for Discontinuation | | | | | | | | | | | | | |
| Adverse Event | 0 | | 1 | (2.9%) | 0 | | 1 | (2.5%) | 1 | (2.5%) | 0 | 3 | (1.3%) |
| Worsening of Psoriasis | 0 | | 0 | | 0 | | 0 | | 0 | | 0 | 0 | |
| Other Adverse Event | 0 | | 1 | (2.9%) | 0 | | 1 | (2.5%) | 1 | (2.5%) | 0 | 3 | (1.3%) |
| Death | 0 | | 0 | | 0 | | 0 | | 0 | | 0 | 0 | |
| Lack of Efficacy | 4 | (11.4%) | 4 | (11.4%) | 3 | (7.7%) | 5 | (12.5%) | 2 | (5.0%) | 1 (2.6%) | 19 | (8.4%) |

| | | | | | | | | | | | |
|---|---|---|---|---|---|---|---|---|---|---|---|
| Lost to Follow-up | 0 | 1 | (2.9%) | 0 | 1 | (2.5%) | 1 | (2.5%) | 1 (2.6%) | 4 | (1.8%) |

| | | | | | | | | | | |
|---|---|---|---|---|---|---|---|---|---|---|
| Non-Compliance with | 0 | 0 | 0 | 1 | (2.5%) | 1 | (2.5%) | 0 | 2 | (0.9%) |

| Study Drug | | | | | | | | | |
|---|---|---|---|---|---|---|---|---|---|
| Physician Decision | 0 | 0 | 0 | | 0 | 0 | 1 (2.6%) | 1 | (0.4%) |
| Pregnancy | 0 | 0 | 1 | (2.6%) | 0 | 0 | 0 | 1 | (0.4%) |

| Product Quality Complaint | 0 | | 0 | 0 | 0 | | 0 | | 0 | 0 | |
|---|---|---|---|---|---|---|---|---|---|---|---|
| Protocol Deviation | 0 | | 0 | 0 | 0 | | 0 | | 0 | 0 | |
| Site Terminated by | 0 | | 0 | 0 | 0 | | 0 | | 0 | 0 | |
| Sponsor | | | | | | | | | | | |
| Study Terminated by | 0 | | 0 | 0 | 0 | | 0 | | 0 | 0 | |
| Sponsor | | | | | | | | | | | |
| Withdrawal by Subject | 1 | (2.9%) | 0 | 0 | 2 | (5.0%) | 2 | (5.0%) | 0 | 5 | (2.2%) |

| Non-Compliance With | 0 | | 0 | 0 | 0 | 0 | 0 | 0 | |
|---|---|---|---|---|---|---|---|---|---|
| Study Schedule | | | | | | | | | |
| Other | 1 | (2.9%) | 0 | 0 | 0) | 0 | 0 | 1 | (0.4%) |

^{a}Includes all columns of treatment with the compound of Formula (I) (Placebo → 100 mg QD, 25 mg QD, 50 mg QD, 25 mg BID, 100 mg QD, and 100 mg BID).
The full analysis set consists of randomized participants in Example 1 who continued into Example 2 and received at least one dose of study intervention (including a partial dose) during long-term extension period.

Among participants who received the compound of Formula (I), a total of 21.5% (53/247) participants discontinued study intervention through Week 52. The most common reason for discontinuing was lack of efficacy (8.1% a, n=20; Table 20).

TABLE 20
Treatment Disposition through Week 52

| Treatment | | | Placebo → | | | | | | | | | | | | | | | |
|---|---|---|---|---|---|---|---|---|---|---|---|---|---|---|---|---|---|---|
| Regimen | Placebo | | 100 mg QD^{a} | | 25 mg QD | | 50 mg QD | | 25 mg BID | | 100 mg QD | | 100 mg BID | | Combined^{b} | | Total | |
| Analysis set: Full | 43 | | 35 | | 43 | | 43 | | 41 | | 43 | | 42 | | 247 | | 255 | |
| analysis set | | | | | | | | | | | | | | | | | | |
| Discontinued | 7 | (16.3%) | 6 | (17.1%) | 13 | (30.2%) | 7 | (16.3%) | 11 | (26.8%) | 9 | (20.9%) | 7 | (16.7%) | 53 | (21.5%) | 60 | (23.5%) |
| study treatment | | | | | | | | | | | | | | | | | | |
| Reason for | | | | | | | | | | | | | | | | | | |
| discontinuation | | | | | | | | | | | | | | | | | | |
| Adverse Event | 0 | | 0 | | 3 | (7.0%) | 2 | (4.7%) | 1 | (2.4%) | 2 | (4.7%) | 1 | (2.4%) | 9 | (3.6%) | 9 | (3.5%) |
| Worsening of | 0 | | 0 | | 0 | | 0 | | 0 | | 0 | | 0 | | 0 | | 0 | |
| Psoriasis | | | | | | | | | | | | | | | | | | |
| Other Adverse | 0 | | 0 | | 3 | (7.0%) | 2 | (4.7%) | 1 | (2.4%) | 2 | (4.7%) | 1 | (2.4%) | 9 | (3.6%) | 9 | (3.5%) |
| Event | | | | | | | | | | | | | | | | | | |
| Death | 0 | | 0 | | 0 | | 0 | | 0 | | 0 | | 0 | | 0 | | 0 | |
| Lack of Efficacy | 4 | (9.3%) | 4 | (11.4%) | 5 | (11.6%) | 3 | (7.0%) | 5 | (12.2%) | 2 | (4.7%) | 1 | (2.4%) | 20 | (8.1%) | 24 | (9.4%) |
| Lost to Follow-up | 1 | (2.3%) | 0 | | 4 | (9.3%) | 0 | | 1 | (2.4%) | 1 | (2.3%) | 2 | (4.8%) | 8 | (3.2%) | 9 | (3.5%) |
| Non-Compliance | 0 | | 0 | | 0 | | 0 | | 1 | (2.4%) | 1 | (2.3%) | 0 | | 2 | (0.8%) | 2 | (0.8%) |
| with Study Drug | | | | | | | | | | | | | | | | | | |
| Physician | 0 | | 0 | | 0 | | 0 | | 0 | | 0 | | 1 | (2.4%) | 1 | (0.4%) | 1 | (0.4%) |
| Decision | | | | | | | | | | | | | | | | | | |
| Pregnancy | 0 | | 0 | | 0 | | 1 | (2.3%) | 0 | | 0 | | 0 | | 1 | (0.4%) | 1 | (0.4%) |
| Product Quality | 0 | | 0 | | 0 | | 0 | | 0 | | 0 | | 0 | | 0 | | 0 | |
| Complaint | | | | | | | | | | | | | | | | | | |
| Protocol | 0 | | 0 | | 0 | | 0 | | 0 | | 0 | | 0 | | 0 | | 0 | |
| Deviation | | | | | | | | | | | | | | | | | | |
| Site Terminated | 0 | | 0 | | 0 | | 0 | | 0 | | 0 | | 0 | | 0 | | 0 | |
| by Sponsor | | | | | | | | | | | | | | | | | | |
| Study Terminated | 0 | | 0 | | 0 | | 0 | | 0 | | 0 | | 0 | | 0 | | 0 | |
| by Sponsor | | | | | | | | | | | | | | | | | | |
| Withdrawal by | 2 | (4.7%) | 1 | (2.9%) | 1 | (2.3%) | 1 | (2.3%) | 3 | (7.3%) | 3 | (7.0%) | 2 | (4.8%) | 11 | (4.5%) | 13 | (5.1%) |

| Subject | | | | | | | | | | | | |
|---|---|---|---|---|---|---|---|---|---|---|---|---|
| Non-Compliance | 0 | 0 | | 0 | 0 | 0 | 0 | 0 | 0 | | 0 | |
| With Study | | | | | | | | | | | | |
| Schedule | | | | | | | | | | | | |
| Other | 0 | 1 | (2.9%) | 0 | 0 | 0 | 0 | 0 | 1 | (0.4%) | 1 | (0.4%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).

### Primary Endpoint

The primary efficacy endpoint is the proportion of participants achieving a PASI 75 response at Week 52, defined as at least a 750% reduction from baseline PASI of Example 1. For primary analysis, composite strategy (non-responder imputation) was applied to address intercurrent event #1 (Discontinuation of study intervention due to lack of efficacy or due to an AE of worsening of psoriasis) and 32 (initiation of a protocol-prohibited medication or therapy that could improve psoriasis); treatment policy strategy (observed data) was applied to intercurrent event #3 (Discontinuation of study intervention due to other reasons). Participants with missing data after application of ICEs are considered as non-responders.

Since there was no control arm (after week 16), in this primary analysis, only the proportion of participants who achieved a PASI75 response and its 950% confidence interval at Week 52 were summarized for each intervention group.

The proportion of subjects achieving a PASI 75 response through Week 52 is summarized in Table 21. In addition, the proportion of participants achieving a PASI 75 response over time from Week 1 of study through Week 52 is summarized in FIG. 22, Table 22, and Table 23.

TABLE 21
Proportion of Subjects Achieving PASI 75 Response at Week 52

| Treatment Regimen | 25 mg QD | 50 mg QD | 25 mg BID | 100 mg QD | 100 mg BID |
|---|---|---|---|---|---|
| Number of Subjects | 43 | 43 | 41 | 43 | 42 |
| Subjects achieving ≥75% | 21 (48.8%) | 30 (69.8%) | 24 (28.5%) | 28 (65.1%) | 32 (76.2%) |
| Improvement at Week 52 | | | | | |
| 95% CI^{a} | (33.9%, 63.8%) | (56.0%, 83.5%) | (43.5%, 73.6%) | (50.9%, 79.4%) | (63.3%, 89.1%) |

^{a}95% CIDs were calculated based on Wald method.
Subjects with ICE 1-2 were assumed to be non-responders after the event. Observed data were used for subjects with ICE 3. After accounting for the ICEs, subjects with missing data were considered as non-responders.

TABLE 22
Change from Baseline in PASI Total Score through Week 52

| | Placebo → | | | | | | | | | | | |
|---|---|---|---|---|---|---|---|---|---|---|---|---|
| Treatment Regimen | 100 mg QD | | 25 mg QD | | 50 mg QD | | 25 mg BID | | 100 mg QD | | 100 mg BID | |
| Analysis set: Full analysis set | 43 | | 43 | | 43 | | 41 | | 43 | | 42 | |
| Baseline | | | | | | | | | | | | |
| N | 43 | | 43 | | 43 | | 41 | | 43 | | 42 | |
| Mean (SD) | 18.99 | (5.341) | 18.90 | (5.272) | 19.23 | (5.082) | 18.46 | (5.838) | 18.42 | (6.873) | 20.33 | (6.509) |

| | | | | | | |
|---|---|---|---|---|---|---|
| Median | 17.60 | 16.90 | 18.00 | 16.80 | 16.60 | 18.65 |
| Range | (12.1; 34.2) | (12.0; 30.3) | (13.6; 40.8) | (12.2; 39.9) | (10.6; 50.9) | (12.0; 38.1) |
| IQ range | (14.40; 21.80) | (15.00; 23.40) | (15.80; 20.20) | (14.30; 20.00) | (14.80; 20.70) | (15.10; 24.90) |

Change from Baseline in PASI Total Score

| Week 1 | | | | | | | | | | | | |
|---|---|---|---|---|---|---|---|---|---|---|---|---|
| N | 42 | | 42 | | 43 | | 41 | | 40 | | 42 | |
| Mean (SD) | −2.21 | (3.651) | −2.25 | (3.497) | −3.10 | (5.621) | −1.20 | (2.719) | −2.60 | (5.577) | −3.25 | (5.683) |
| Median | −1.05 | | −0.80 | | −1.80 | | −0.80 | | −1.10 | | −1.20 | |
| Range | (−11.0; 4.9) | | (−11.3; 4.0) | | (−23.2: 12.2) | | (−7.0; 6.9) | | (−27.6; 6.8) | | (−24.0; 9.3) | |
| IQ range | (−4.70; 0.00) | | (−5.10; 0.00) | | (−4.20; 0.00) | | (−3.00; 0.00) | | (−3.95; 0.00) | | (−5.70; 0.00) | |
| LSMean (95% CI)^{a} | −2.2 | | −2.4 | | −3.1 | | −1.4 | | −2.8 | | −2.9 | |
| | (−3.56, −0.92) | | (−3.69, −1.04) | | (−4.40, −1.79) | | (−2.75, −0.07) | | (−4.17, −1.48) | | (−4.26, −1.61) | |
| Week 2 | | | | | | | | | | | | |
| N | 43 | | 41 | | 41 | | 41 | | 42 | | 40 | |
| Mean (SD) | −3.10 | (3.730) | −4.50 | (4.739) | −5.74 | (5.654) | −3.65 | (3.409) | −5.13 | (6.253) | 6.07 | (6.084) |
| Median | −2.40 | | −4.00 | | −4.30 | | −3.20 | | −3.25 | | −4.85 | |
| Range | (−11.6; 7.2) | | (−15.5; 4.1) | | (−21.2; 1.8) | | (−11.0; 4.8) | | (−31.6; 2.0) | | (−24.1; 4.0) | |
| IQ range | (−5.00; −0.40) | | (−6.60; −0.60) | | (−9.20; −1.00) | | (−6.50; −1.20) | | (−6.90; −0.90) | | (−9.20; −0.90) | |
| LSMean (95% CI)^{a} | −3.1 | | −4.5 | | −5.7 | | −3.8 | | −5.3 | | −5.7 | |
| | (−4.52, −1.71) | | (−5.92, −3.06) | | (−7.10, −4.25) | | (−5.28, −2.40) | | (−6.70, −3.85) | | (−7.14, −4.26) | |
| Week 4 | | | | | | | | | | | | |
| N | 43 | | 42 | | 42 | | 41 | | 42 | | 41 | |
| Mean (SD) | −2.84 | (5.366) | −6.50 | (6.727) | −8.25 | (5.809) | −6.26 | (4.806) | −8.39 | (8.029) | −10.71 | (7.925) |
| Median | −3.50 | | −5.60 | | −7.85 | | −6.30 | | −7.30 | | −9.70 | |
| Range | (−14.5; 16.5) | | (−20.8; 3.5) | | (−21.5; 0.0) | | (−18.1; 1.2) | | (−46.2; 0.0) | | (−35.9; 1.2) | |
| IQ range | (−6.30; 0.00) | | (−10.30; −0.90) | | (−12.40; −3.90) | | (−9.00; −2.00) | | (−11.00; −3.10) | | (−14.20; −5.60) | |
| LSMean (95% CI)^{a} | −2.9 | | −6.7 | | −8.2 | | −6.7 | | −8.7 | | −10.0 | |
| | (−4.64, −1.24) | | (−8.45, −5.01) | | (−9.93, −6.51) | | (−8.41, −4.93) | | (−10.46, −7.02) | | (−11.76, −8.28) | |
| Week 8 | | | | | | | | | | | | |
| N | 41 | | 41 | | 42 | | 41 | | 41 | | 41 | |
| Mean (SD) | −4.38 | (7.103) | −9.39 | (7.590) | −11.41 | (5.873) | −9.72 | (7.370) | −12.04 | (8.892) | −14.66 | (8.018) |
| Median | −3.30 | | −7.70 | | −11.85 | | −9.90 | | −11.30 | | −13.30 | |
| Range | (−23.9; 14.5) | | (−30.2; 3.2) | | (−23.7; 0.0) | | (−26.1; 7.0) | | (−49.6; 0.0) | | (−38.1; 0.0) | |
| IQ range | (−7.50; 0.00) | | (−14.30; −5.30) | | (−15.90; −7.00) | | (−14.70; −5.70) | | (−14.70; −7.20) | | (−19.30; −9.20) | |
| LSMean (95% CI)^{a} | −4.3 | | −9.5 | | −11.3 | | −10.2 | | −12.5 | | −13.8 | |
| | (−6.25, −2.40) | | (−11.43, −7.56) | | (−13.26, −9.42) | | (−12.10, −8.21) | | (−14.41, −10.55) | | (−15.75, −11.85) | |
| Week 12 | | | | | | | | | | | | |
| N | 40 | | 41 | | 42 | | 41 | | 42 | | 40 | |
| Mean (SD) | −3.61 | (8.615) | −10.71 | (8.570) | −12.96 | (6.939) | −11.46 | (8.051) | −13.70 | (8.773) | −16.77 | (8.404) |
| Median | −2.25 | | −9.20 | | −14.25 | | −12.30 | | −13.60 | | −17.05 | |
| Range | (−25.3; 18.1) | | (−29.6; 7.9) | | (−27.9; 8.4) | | (−36.3; 4.7) | | (−50.9; 0.0) | | (−38.1; 4.6) | |
| IQ range | (−8.80; 0.00) | | (−16.80; −3.80) | | (−17.60; −9.10) | | (−15.20; −5.60) | | (−16.50; −9.30) | | (−22.25; −11.95) | |
| LSMean (95% CI)^{a} | −3.7 | | −10.8 | | −12.8 | | −11.9 | | −14.0 | | −15.9 | |
| | (−5.75, −1.58) | | (−12.88, −8.71) | | (−14.91, −10.77) | | (−14.00, −9.81) | | (−16.12, −11.97) | | (−17.97, −13.76) | |
| Week 16 | | | | | | | | | | | | |
| N | 40 | | 37 | | 41 | | 40 | | 42 | | 39 | |
| Mean (SD) | −3.59 | (9.436) | −12.76 | (8.050) | −14.56 | (6.528) | −12.73 | (8.021) | −13.99 | (8.653) | −17.44 | (8.356) |
| Median | −2.55 | | −12.20 | | −14.90 | | −12.70 | | −14.75 | | −17.40 | |
| Range | (−28.1; 25.4) | | (−29.5; 3.1) | | (−27.9; 0.6) | | (−38.1; 3.0) | | (−50.9; − 0.2) | | (−38.1; 0.8) | |
| IQ range | (−10.10; 0.00) | | (−18.40; −7.80) | | (−18.60; −12.30) | | (−16.80; −8.65) | | (−16.60; −7.50) | | (−22.80; −12.40) | |
| LSMean (95% CI)^{a} | −3.7 | | −12.0 | | −14.4 | | −13.0 | | −14.4 | | −16.5 | |
| | (−5.81, −1.65) | | (−14.08, −9.89) | | (−16.42, −12.30) | | (−15.07, −10.89) | | (−16.45, −12.32) | | (−18.65, −14.44) | |
| Week 20^{b} | | | | | | | | | | | | |
| N | 34 | | 36 | | 40 | | 39 | | 39 | | 37 | |
| Mean (SD) | −10.44 | (7.013) | −14.01 | (7.415) | −14.92 | (6.224) | −13.50 | (7.967) | −14.57 | (8.694) | −17.83 | (7.954) |
| Median | −10.35 | | −13.70 | | −15.10 | | −13.60 | | −14.10 | | −17.40 | |
| Range | (−29.6; 3.1) | | (−30.2; 0.0) | | (−27.0; 0.0) | | (−39.0; 0.0) | | (−50.3; − 0.2) | | (−38.1; 0.0) | |
| IQ range | (−15.00; −6.20) | | (−18.50; −10.10) | | (−18.90; −11.80) | | (−16.80; −8.00) | | (−18.80; −9.50) | | (−21.30; −13.00) | |
| LSMean (95% CI)^{a} | −10.3 | | −13.2 | | −14.7 | | −13.7 | | −15.1 | | −17.0 | |
| | (−12.13, −8.49) | | (−14.97, −11.35) | | (−16.51, −12.97) | | (−15.52, −11.93) | | (−16.89, −13.33) | | (−18.83, −15.19) | |
| Week 24^{b} | | | | | | | | | | | | |
| N | 35 | | 36 | | 40 | | 38 | | 38 | | 37 | |
| Mean (SD) | −13.26 | (6.661) | −13.56 | (7.856) | −15.10 | (6.245) | −13.64 | (8.250) | −15.27 | (8.266) | −17.99 | (8.175) |
| Median | −14.10 | | −14.30 | | −15.70 | | −13.55 | | −14.90 | | −17.80 | |
| Range | (−29.9; 4.0) | | (−29.4; 2.9) | | (−29.7; 0.0) | | (−39.9; 2.2) | | (−48.8; −1.0) | | (−38.1; 2.8) | |
| IQ range | (−18.10; −7.50) | | (−18.95; −8.85) | | (−18.60; −11.75) | | (−16.80; −9.40) | | (−19.50; −11.20) | | (−23.20; −13.50) | |
| LSMean (95% CI)^{a} | −13.2 | | −12.8 | | −14.9 | | −13.5 | | −15.6 | | −17.1 | |
| | (−15.01, −11.34) | | (−14.59, −10.94) | | (−16.68, −13.14) | | (−15.31, −11.71) | | (−17.42, −13.84) | | (−18.97, −15.32) | |
| Week 28^{b} | | | | | | | | | | | | |
| N | 35 | | 34 | | 40 | | 37 | | 35 | | 36 | |
| Mean (SD) | −13.77 | (6.733) | −13.35 | (7.214) | −14.75 | (6.912) | −14.33 | (8.317) | −16.11 | (8.109) | −17.96 | (8.027) |
| Median | −14.10 | | −14.60 | | −15.50 | | −14.40 | | −15.00 | | −17.75 | |
| Range | (−29.9; 2.7) | | (−29.4; 0.0) | | (−29.1; 4.2) | | (−39.9; 0.0) | | (−48.8; −0.8) | | (−38.1; 0.0) | |
| IQ range | (−17.60; −9.80) | | (−18.40; −8.10) | | (−18.20; −12.50) | | (−16.80; −10.80) | | (−20.10; −12.20) | | (−22.90; −13.10) | |
| LSMean (95% CI)^{a} | −13.7 | | −13.3 | | −14.6 | | −14.5 | | −15.8 | | −17.2 | |
| | (−15.56, −11.86) | | (−15.10, −11.42) | | (−16.33, −12.78) | | (−16.34, −12.73) | | (−17.61, −13.99) | | (−19.00, −15.32) | |
| Week 32^{b} | | | | | | | | | | | | |
| N | 34 | | 35 | | 39 | | 37 | | 36 | | 36 | |
| Mean (SD) | −14.14 | (6.660) | −14.06 | (7.882) | −14.88 | (6.787) | −14.36 | (8.183) | −16.21 | (8.330) | −18.37 | (7.971) |
| Median | −14.75 | | −15.50 | | −15.80 | | −14.30 | | −15.10 | | −18.20 | |
| Range | (−29.9; 0.0) | | (−30.2; 0.0) | | (−27.9; 1.5) | | (−39.9; 0.0) | | (−49.7; 0.0) | | (−38.1; 0.0) | |
| IQ range | (−17.60; −9.70) | | (−18.80; −9.00) | | (−18.80; −12.90) | | (−16.80; −11.70) | | (−20.25; −12.70) | | (−23.00; −13.25) | |
| LSMean (95% CI)^{a} | −13.7 | | −13.6 | | −14.6 | | −14.6 | | −16.2 | | −17.3 | |
| | (−15.60, −11.88) | | (−15.42, −11.72) | | (−16.35, −12.79) | | (−16.45, −12.83) | | (−17.99, −14.35) | | (−19.16, −15.47) | |
| Week 40^{b} | | | | | | | | | | | | |
| N | 34 | | 34 | | 40 | | 36 | | 35 | | 36 | |
| Mean (SD) | −13.73 | (7.585) | −13.70 | (8.022) | −14.33 | (6.822) | −13.58 | (8.625) | −15.95 | (8.603) | −18.30 | (7.965) |
| Median | −14.85 | | −15.25 | | −15.45 | | −14.10 | | −15.00 | | −18.30 | |
| Range | (−29.9; 0.3) | | (−29.4; 0.0) | | (−27.9; 0.0) | | (−39.9; 0.0) | | (−50.0; 1.4) | | (−38.1; 0.0) | |
| IQ range | (−18.80; −10.20) | | (−18.40; −9.00) | | (−18.00; −12.20) | | (−17.30; −8.25) | | (−19.80; −12.80) | | (−22.85; −13.05) | |
| LSMean (95% CI)^{a} | −13.6 | | −13.1 | | −14.2 | | −13.9 | | −15.7 | | −17.4 | |
| | (−15.63, −11.55) | | (−15.12, −11.05) | | (−16.12, −12.24) | | (−15.93, −11.95) | | (−17.68, −13.69) | | (−19.38, −15.34) | |
| Week 52^{b} | | | | | | | | | | | | |
| N | 34 | | 34 | | 40 | | 36 | | 35 | | 35 | |
| Mean (SD) | −14.15 | (8.068) | −13.55 | (8.232) | −14.45 | (6.878) | −13.24 | (8.981) | −15.81 | (8.908) | −18.46 | (7.892) |

| | | | | | | |
|---|---|---|---|---|---|---|
| Median | −15.30 | −14.55 | −15.80 | −13.75 | −15.00 | −18.60 |
| Range | (−29.9; 4.5) | (−30.2; 0.0) | (−28.7; 0.0) | (−39.9; 0.0) | (−50.3; 0.0) | (−38.1; 0.0) |
| IQ range | (−19.40; −10.90) | (−18.30; −8.00) | (−18.20; −12.30) | (−17.15; −8.65) | (−20.40; −11.40) | (−22.50; −13.50) |
| LSMean (95% CI)^{a} | −14.0 | −12.9 | −14.3 | −13.6 | −15.5 | −17.4 |
| | (−16.12, −11.89) | (−15.01, −10.79) | (−16.28, −12.28) | (−15.62, −11.50) | (−17.61, −13.46) | (−19.51, −15.33) |

^{a}LS Means are based on the MMRM model with treatment group, visit, treatment group by visit interaction, baseline weight category (≤90 kg, >90 kg), baseline weight category by visit interaction, baseline PASI total score, and baseline PASI total score by visit interaction as covariates.
^{b}For subjects who were randomized to placebo at Week 0, only include those subjects who crossed over to treatment with the compound of Formula (I) at or after Week 16.
Subjects who have ICE 1-2 have zero change from baseline after the event. Observed data were used for subjects with ICE 3.

TABLE 23
Proportion of Subjects Achieving PASI 75 Response at Week 52

| | Placebo → | | | | | |
|---|---|---|---|---|---|---|
| Treatment Regimen | 100 mg QD | 25 mg QD | 50 mg QD | 25 mg BID | 100 mg QD | 100 mg BID |
| Analysis set: Full analysis set | 35 | 43 | 43 | 41 | 43 | 42 |
| Subjects achieving ≥75% | 23 (65.7%) | 21 (48.8%) | 30 (69.8%) | 24 (58.5%) | 28 (65.1%) | 32 (76.2%) |
| Improvement at Week 52^{a} | | | | | | |
| 95% CI^{b} | (50.0%, 81.4%) | (33.9%, 63.8%) | (56.0%, 83.5%) | (43.5%, 73.6%) | (50.9%, 79.4%) | (63.3%, 89.1%) |

^{a}For subjects who were randomized to placebo at Week 0, only include those subjects who crossed over to treatment with the compound of Formula (I) at or after Week 16.
^{b}95% CIs were calculated based on Wald method.
Subjects with ICE 1-2 were assumed to be non-responders after the event. Observed data were used for subjects with ICE 3. After accounting for the ICEs, subjects with missing data were considered as non-responders.

### Secondary Endpoints

Secondary endpoints included additional change from baseline of Example 1 in PASI, IGA, and PSSD at Week 52, including the proportions of subjects that achieved PAST 90, PAST 100, an IGA score of cleared (0) or minimal (1), PSSD symptoms score=0, and PSSD signs score=0.

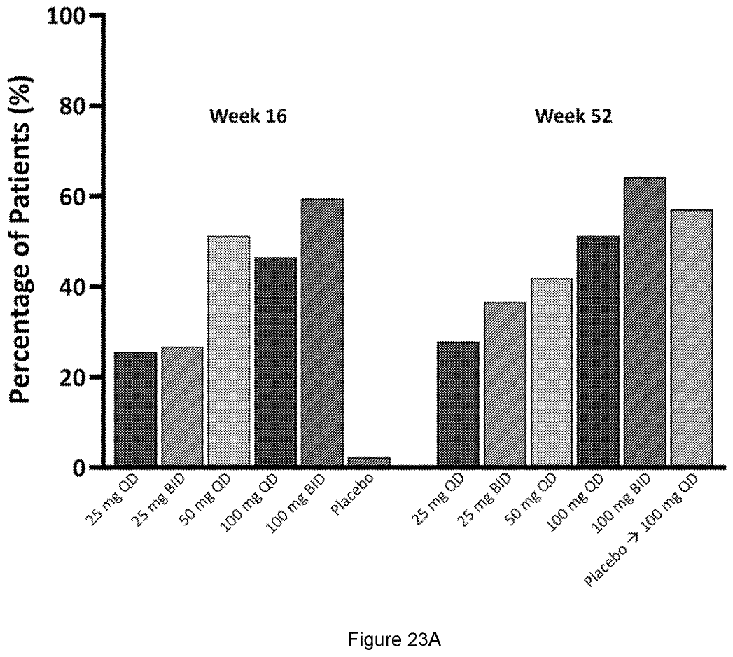

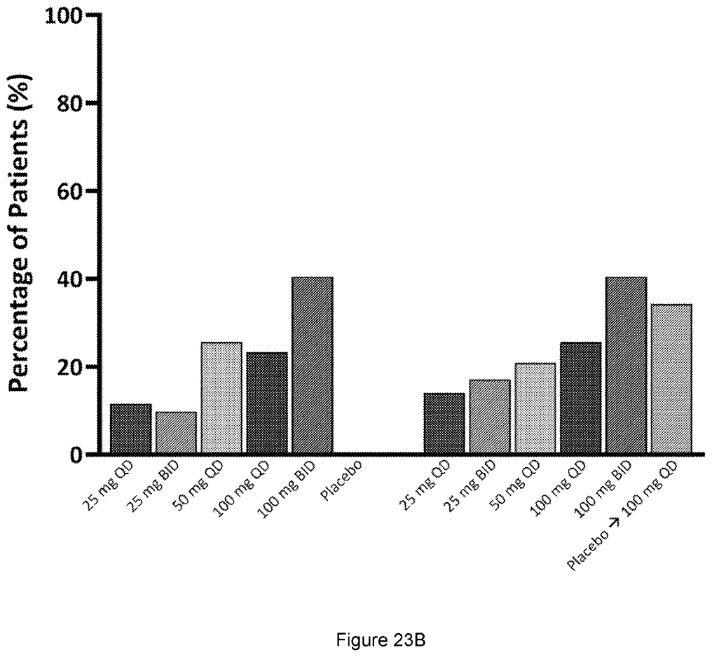

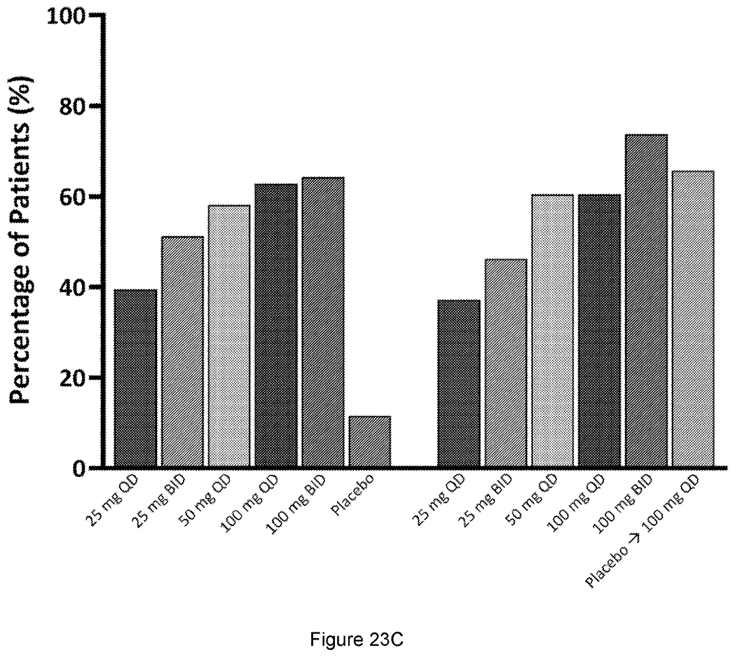

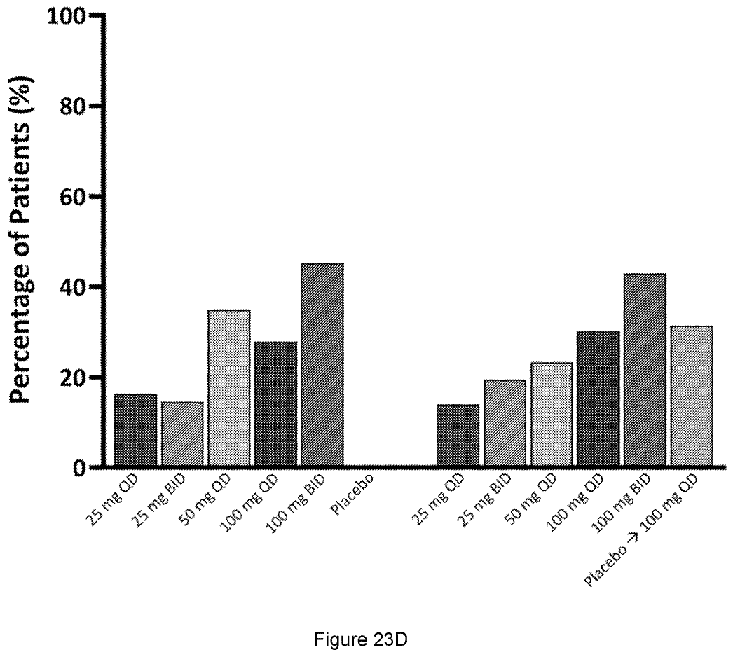

Similar results were observed for PAS and IGA binary secondary endpoints at Week 52 (Table 24 and Table 25). Efficacy results based on other PAST and IGA binary endpoints were comparable to PAST 75 response over time (FIG. 22). PAST and IGA response rates were generally maintained from Week 16 to Week 52; and the highest responses rates were observed in the 100 mg BID group (FIG. 23).

TABLE 24
PASI and IGA Responses at Week 52

| Treatment Regimen | 25 mg QD | | 50 mg QD | | 25 mg BID | | 100 mg QD | | 100 mg BID | |
|---|---|---|---|---|---|---|---|---|---|---|
| Analysis set: Full | 43 | | 43 | | 41 | | 43 | | 42 | |
| analysis set | | | | | | | | | | |
| Subjects achieving ≥90% | 12 | (27.9%) | 18 | (41.9%) | 15 | (36.6%) | 22 | (51.2%) | 27 | (64.3%) |
| Improvement at Week 52 | | | | | | | | | | |
| 95% CI^{a} | (14.5%, 41.3%) | | (27.1%, 56.6%) | | (21.8%, 51.3%) | | (36.2%, 66.1%) | | (49.8%, 78.8%) | |
| Subjects achieving 100% | 6 | (14.0%) | 9 | (20.9%) | 7 | (17.1%) | 11 | (25.6%) | 17 | (40.5%) |
| Improvement at Week 52 | | | | | | | | | | |
| 95% CI^{a} | (3.6%, 24.3%) | | (8.8%, 33.1%) | | (5.6%, 28.6%) | | (12.5%, 38.6%) | | (25.6%, 55.3%) | |
| Subjects achieving IGA | 16 | (37.2%) | 26 | (60.5%) | 19 | (46.3%) | 26 | (60.5%) | 31 | (73.8%) |
| Score = 0 or 1 at Week 52 | | | | | | | | | | |
| 95% CI^{a} | (22.8%, 51.7%) | | (45.9%, 75.1%) | | (31.1%, 61.6%) | | (45.9%, 75.1%) | | (60.5%, 87.1%) | |
| Subjects achieving IGA | 6 | (14.0%) | 10 | (23.3%) | 8 | (19.5%) | 13 | (30.2%) | 18 | (42.9%) |

| | | | | | |
|---|---|---|---|---|---|
| Score = 0 at Week 52 | | | | | |
| 95% CI^{a} | (3.6%, 24.3%) | (10.6%, 35.9%) | (7.4%, 31.6%) | (16.5%, 44.0%) | (27.9%, 57.8%) |

^{a}95% CIs were calculated based on Wald method.
Subjects with ICE 1-2 were assumed to be non-responders after the event. Observed data were used for subjects with ICE 3. After accounting for the ICEs, subjects with missing data were considered as non-responders.

TABLE 25
PASI and IGA Responses at Week 52

| | Placebo → | | | | | | | | |
|---|---|---|---|---|---|---|---|---|---|
| Treatment Regimen | 100 mg QD | 25 mg QD | | 50 mg QD | | 25 mg BID | | 100 mg QD | 100 mg BID |
| Analysis set: Full analysis set | 39 | 43 | | 43 | | 43 | | 42 | 42 |
| Subjects achieving ≥90% | 20 (57.1%) | 12 | (27.9%) | 18 | (41.9%) | 15 | (36.6%) | 22 (51.2%) | 27 (64.3%) |
| Improvement at Week 52^{a} | | | | | | | | | |
| 95% CI^{b} | (40.7%, 73.5%) | (14.5%, 41.3%) | | (27.1%, 56.6%) | | (21.8%, 51.3%) | | (36.2%, 66.1%) | (49.8%, 78.8%) |
| Subjects achieving 100% | 12 (34.3%) | 6 | (14.0%) | 9 | (20.9%) | 7 | (17.1%) | 11 (25.6%) | 17 (40.5%) |
| Improvement at Week 52^{a} | | | | | | | | | |
| 95% CI^{b} | (18.6%, 50.0%) | (3.6%, 24.3%) | | (8.8%, 33.1%) | | (5.6%, 28.6%) | | (12.5%, 38.6%) | (25.6%, 55.3%) |
| Subjects achieving IGA | 23 (65.7%) | 16 | (37.2%) | 26 | (60.5%) | 19 | (46.3%) | 26 (60.5%) | 31 (73.8%) |
| Score = 0 or 1 at Week 52^{a} | | | | | | | | | |
| 95% CI^{b} | (50.0%, 81.4%) | (22.8%, 51.7%) | | (45.9%, 75.1%) | | (31.1%, 61.6%) | | (45.9%, 75.1%) | (60.5%, 87.1%) |
| Subjects achieving IGA | 11 (31.4%) | 6 | (14.0%) | 10 | (23.3%) | 8 | (19.5%) | 13 (30.2%) | 18 (42.9%) |

| | | | | | | |
|---|---|---|---|---|---|---|
| Score = 0 at Week 52^{a} | | | | | | |
| 95% CI^{b} | (16.0%, 46.8%) | (3.6%, 24.3%) | (10.6%, 35.9%) | (7.4%, 31.6%) | (16.5%, 44.0%) | (27.9%, 57.8%) |

^{a}For subjects who were randomized to placebo at Week 0, only include those subjects who crossed over to treatment with the compound of Formula (I) at or after Week 16.
^{b}95% CIs were calculated based on Wald method.
Subjects with ICE 1-2 were assumed to be non-responders after the event. Observed data were used for subjects with ICE 3. After accounting for the ICEs, subjects with missing data were considered as non-responders.

The proportions of participants who had a PSSD symptom score of 0 and PSSD sign score of 0 at Week 52 among participants with original baselines≥1 in Example 1 are summarized in Table 26 and Table 27.

TABLE 26
Proportion of Subjects Achieving PSSD Symptom Score 0 and PSSD Sign Score 0 at Week 52

| Treatment Regimen | 25 mg QD | 50 mg QD | 25 mg BID | 100 mg QD | 100 mg BID |
|---|---|---|---|---|---|
| Analysis set: Full | 43 | 43 | 41 | 43 | 42 |
| analysis set | | | | | |
| Subjects w/Baseline | 43 | 43 | 41 | 43 | 42 |
| PSSD Symptom | | | | | |
| Score ≥1 | | | | | |
| Subjects Achieving | 8 (18.6%) | 9 (21.4%) | 7 (17.1%) | 13 (30.2%) | 11 (26.2%) |
| PSSD Symptom Score | | | | | |
| 0 at Week 52 | | | | | |
| 95% CI^{a} | (7.0%, 30.2%) | (9.0%, 33.8%) | (5.6%, 28.6%) | (16.5%, 44.0%) | (12.9%, 39.5%) |
| Subjects w/Baseline | 43 | 43 | 41 | 43 | 42 |
| PSSD Sign Score ≥1 | | | | | |
| Subjects Achieving | 7 (16.3%) | 5 (11.6%) | 5 (12.2%) | 6 (14.0%) | 7 (16.7%) |
| PSSD Symptom Score | | | | | |
| 0 at Week 52 | | | | | |
| 95% CI^{a} | (5.2%, 27.3%) | (2.0%, 21.2%) | (2.2%, 22.2%) | (3.6%, 24.3%) | (5.4%, 27.9%) |

^{a}95% CIs were calculated based on Wald method.
Subjects with ICE 1-2 were assumed to be non-responders after the event. Observed data were used for subjects with ICE 3. After accounting for the ICEs, subjects with missing data were considered as non-responders.

TABLE 27
Proportion of Subjects Achieving PSSD Symptom Score 0 and PSSD Sign Score 0 at Week 52

| | Placebo → | | | | | | | | |
|---|---|---|---|---|---|---|---|---|---|
| Treatment Regimen | 100 mg QD | | 25 mg QD | 50 mg QD | 25 mg BID | 100 mg QD | | 100 mg BID | |
| Analysis set: Full analysis | 35 | | 43 | 43 | 41 | 43 | | 42 | |
| set | | | | | | | | | |
| Subjects with Baseline | 35 | | 43 | 42 | 41 | 43 | | 42 | |
| PSSD Symptom | | | | | | | | | |
| Score ≥1 | | | | | | | | | |
| Subjects achieving PSSD | 12 | (34.3%) | 8 (18.6%) | 9 (21.4%) | 7 (17.1%) | 13 | (30.2%) | 11 | (26.2%) |

| Symptom | | | | | | | |
|---|---|---|---|---|---|---|---|
| Score = 0 at Week 52^{a} | | | | | | | |
| 95% CI^{b} | (18.6% ,50.0%) | | (7.0%, 30.2%) | (9.0%, 33.8%) | (5.6%, 28.6%) | (16.5%, 44.0%) | (12.9%, 39.5%) |
| Subjects with Baseline | 35 | | 43 | 43 | 41 | 43 | 42 |
| PSSD Sign Score ≥1 | | | | | | | |
| Subjects achieving PSSD | 8 | (22.9%) | 7 (16.3%) | 5 (11.6%) | 5 (12.2%) | 6 (14.0%) | 7 (16.7%) |

| | | | | | | |
|---|---|---|---|---|---|---|
| Sign Score = 0 at Week 52^{a} | | | | | | |
| 95% CI^{b} | (8.9%, 36.8%) | (5.2%, 27.3%) | (2.0%, 21.2%) | (2.2%, 22.2%) | (3.6%, 24.3%) | (5.4%, 27.9%) |

^{a}For subjects who were randomized to placebo at Week 0, only include those subjects who crossed over to treatment with the compound of Formula (I) at or after Week 16.
^{b}95% CIs were calculated based on Wald method.
Subjects with ICE 1-2 were assumed to be non-responders after the event. Observed data were used for subjects with ICE 3. After accounting for the ICEs, subjects with missing data were considered as non-responders.

The change from baseline in total PASI score, PSSD symptom score, and PSSD sign score at Week 52 are summarized in Table 28 through Table 30.

TABLE 28
Change from Baseline in PASI Total Score at Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 35 | 43 | 43 | 41 | 43 | 42 |
| Full analysis set | | | | | | |
| Baseline | | | | | | |
| N | 35 | 43 | 43 | 41 | 43 | 42 |
| Mean (SD) | 19.12 | 18.90 | 19.23 | 18.46 | 18.42 | 20.33 |
| | (5.298) | (5.272) | (5.082) | (5.838) | (6.873) | (6.509) |
| Median | 18.60 | 16.90 | 18.00 | 16.80 | 16.60 | 18.65 |
| Range | (12.1; 34.2) | (12.0; 30.3) | (13.6; 40.8) | (12.2; 39.9) | (10.6; 50.9) | (12.0; 38.1) |
| IQ range | (14.70; 21.80) | (15.00; 23.40) | (15.80; 20.20) | (14.30; 20.00) | (14.80; 20.70) | (15.10; 24.90) |
| Change from Baseline | | | | | | |
| in PASI Total Score at | | | | | | |
| Week 52^{a} | | | | | | |
| N | 34 | 34 | 40 | 36 | 35 | 35 |
| Mean (SD) | −14.15 | −13.55 | −14.45 | −13.24 | −15.81 | −18.46 |
| | (8.068) | (8.232) | (6.878) | (8.981) | (8.908) | (7.892) |
| Median | −15.30 | −14.55 | −15.80 | −13.75 | −15.00 | −18.60 |
| Range | (−29.9; 4.5) | (−30.2; 0.0) | (−28.7; 0.0) | (−39.9; 0.0) | (−50.3; 0.0) | (−38.1; 0.0) |
| IQ range | (−19.40; −10.90) | (−18.30; −8.00) | (−18.20; −12.30) | (−17.15; −8.65) | (−20.40; −11.40) | (−22.50; −13.50) |
| LSMean (95% CI)^{b} | −14.0 | −12.9 | −14.3 | −13.6 | −15.5 | −17.4 |
| | (−16.12, −11.89) | (−15.01, −10.79) | (−16.28, −12.28) | (−15.62, −11.50) | (−17.61, −13.46) | (−19.51, −15.33) |

^{a}For subjects who were randomized to placebo at Week 0, only include those subjects who crossed over to treatment with the compound of Formula (I) at or after Week 16.
^{b}LS Means are based on the MMRM model with treatment group, visit, treatment group by visit interaction, baseline weight category (≤90 kg, >90 kg), baseline weight category by visit interaction, baseline PASI total score, and baseline PASI total score by visit interaction as covariates.
Subjects who have ICE 1-2 have zero change from baseline after the event. Observed data were used for subjects with ICE 3.

TABLE 29
Change from Baseline in PSSD Symptom Score at Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 35 | 43 | 43 | 41 | 43 | 42 |
| Full analysis set | | | | | | |
| Baseline | | | | | | |
| N | 35 | 43 | 43 | 41 | 43 | 42 |
| Mean (SD) | 49.4 | 59.0 | 53.9 | 51.9 | 43.0 | 55.9 |
| | (21.05) | (23.63) | (24.49) | (24.05) | (21.27) | (26.27) |
| Median | 46.0 | 56.0 | 56.0 | 50.0 | 44.0 | 56.0 |
| Range | (12; 98) | (6; 98) | (0; 100) | (4; 100) | (6; 96) | (6; 100) |
| IQ range | (36.0; 64.0) | (42.0; 76.0) | (34.0; 68.0) | (36.0; 70.0) | (28.0; 56.0) | (40.0; 76.0) |
| Change from Baseline in | | | | | | |
| PSSD Symptom Score at | | | | | | |
| Week 52^{a} | | | | | | |
| N | 34 | 34 | 40 | 36 | 35 | 36 |
| Mean (SD) | −29.5 | −30.1 | −35.2 | −31.2 | −29.4 | −47.7 |
| | (25.59) | (28.09) | (30.81) | (28.48) | (27.41) | (28.04) |
| Median | −30.0 | −35.0 | −34.0 | −27.0 | −28.0 | −53.0 |
| Range | (−98; 24) | (−96; 16) | (−100; 30) | (−80; 22) | (−90; 16) | (−98; 0) |
| IQ range | (−44.0; −12.0) | (−46.0; 0.0) | (−58.0; −12.0) | (−55.0; −7.0) | (−50.0; 0.0) | (−69.0; −22.0) |
| LSMean (95% CI)^{b} | −32.4 | −22.3 | −33.0 | −31.4 | −36.5 | −44.0 |
| | (−39.70, −25.02) | (−29.65, −14.90) | (−39.94, −26.02) | (−38.57, −24.16) | (−43.86, −29.14) | (−51.28, −36.77) |

^{a}For subjects who were randomized to placebo at Week 0, only include those subjects who crossed over to treatment with the compound of Formula (I) at or after Week 16.
^{b}LS Means are based on the MMRM model with treatment group, visit, treatment group by visit interaction, baseline weight category (≤90 kg, >90 kg), baseline weight category by visit interaction, baseline PSSD symptom score, and baseline PSSD symptom score by visit interaction as covariates.
Subjects who have ICE 1-2 have zero change from baseline after the event. Observed data were used for subjects with ICE 3.

TABLE 30
Change from Baseline in PSSD Sign Score at Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 35 | 43 | 43 | 41 | 43 | 42 |
| Full analysis set | | | | | | |
| Baseline | | | | | | |
| N | 35 | 43 | 43 | 41 | 43 | 42 |
| Mean (SD) | 65.8 | 69.5 | 64.7 | 64.1 | 60.4 | 66.3 |
| | (14.59) | (16.50) | (19.41) | (18.90) | (18.56) | (19.09) |
| Median | 68.0 | 70.0 | 65.0 | 62.0 | 63.0 | 70.0 |
| Range | (35; 97) | (28; 100) | (5; 100) | (20; 100) | (13; 95) | (23; 100) |
| IQ range | (55.0; 73.0) | (58.0; 82.0) | (55.0; 77.0) | (50.0; 78.0) | (48.0; 73.0) | (53.0; 82.0) |
| Change from Baseline | | | | | | |
| in PSSD Sign Score at | | | | | | |
| Week 52^{a} | | | | | | |
| N | 34 | 34 | 40 | 36 | 35 | 36 |
| Mean (SD) | −42.8 | −35.2 | −39.2 | −36.6 | −43.1 | −53.1 |
| | (28.65) | (29.02) | (31.64) | (29.16) | (26.87) | (22.03) |
| Median | −48.5 | −32.5 | −45.0 | −38.5 | −43.0 | −55.0 |
| Range | (−94; 11) | (−98; 12) | (−100; 22) | (−92; 15) | (−87; 9) | (−85; 0) |
| IQ range | (−65.0; −23.0) | (−60.0; −15.0) | (−63.5; −14.0) | (−57.0; −14.0) | (−61.0; −29.0) | (−70.5; −40.0) |
| LSMean (95% CI)^{b} | −43.1 | −27.9 | −39.0 | −37.2 | −45.8 | −51.9 |
| | (−51.27, −34.99) | (−36.11, −19.71) | (−46.71, −31.23) | (−45.15, −29.15) | (−53.88, −37.68) | (−59.97, −43.87) |

^{a}For subjects who were randomized to placebo at Week 0, only include those subjects who crossed over to treatment with the compound of Formula (I) at or after Week 16.
^{b}LS Means are based on the MMRM model with treatment group, visit, treatment group by visit interaction, baseline weight category (≤90 kg, >90 kg), baseline weight category by visit interaction, baseline PSSD sign score, and baseline PSSD sign score by visit interaction as covariates.
Subjects who have ICE 1-2 have zero change from baseline after the event. Observed data were used for subjects with ICE 3.

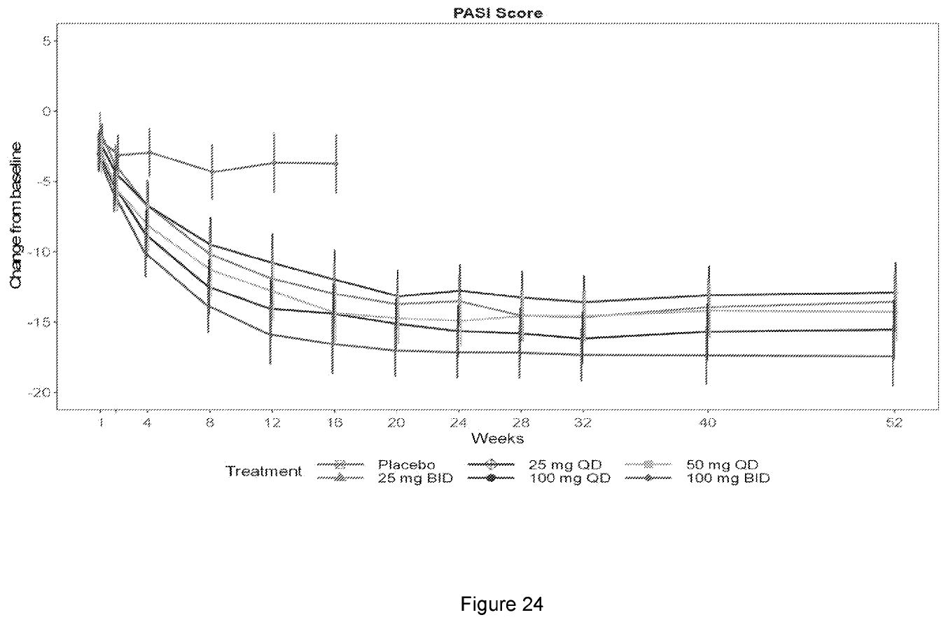

The proportions and the corresponding 9500 CIs of participants achieving PASI 75, PASI 90, PASI 100, IGA 0/1 and IGA 0 over time from Week 1 through Week 52 are summarized in FIG. 22, FIG. 23, Table 31, and Table 32. LSMeans and the corresponding 95% CIs for change from baseline in PASI total score are presented in FIG. 24 and Table 22.

TABLE 31
IGA Response through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Week 1 | | | | | | |
| N | 43 | 43 | 43 | 41 | 43 | 42 |
| IGA Score ≤2 | 7 | 6 | 7 | 3 | 4 | 10 |
| (‘Mild’or better) | (16.3%) | (14.0%) | (16.3%) | (7.3%) | (9.3%) | (23.8%) |
| IGA Score = 0 | 0 | 0 | 1 | 0 | 2 | 1 |
| (‘Cleared’) or | | | (2.3%) | | (4.7%) | (2.4%) |
| 1 (‘Minimal’) | | | | | | |
| IGA Score = 0 | 0 | 0 | 0 | 0 | 0 | 0 |
| (‘Cleared’) | | | | | | |
| Week 2 | | | | | | |
| N | 43 | 43 | 43 | 41 | 43 | 42 |
| IGA Score ≤2 | 11 | 9 | 15 | 9 | 15 | 14 |
| (‘Mild’or better) | (25.6%) | (20.9%) | (34.9%) | (22.0%) | (34.9%) | (33.3%) |
| IGA Score = 0 | 1 | 0 | 3 | 1 | 2 | 4 |
| (‘Cleared’) or | (2.3%) | | (7.0%) | (2.4%) | (4.7%) | (9.5%) |
| 1 (‘Minimal’) | | | | | | |
| IGA Score = 0 | 0 | 0 | 0 | 0 | 0 | 0 |
| (‘Cleared’) | | | | | | |
| Week 4 | | | | | | |
| N | 43 | 43 | 43 | 41 | 43 | 42 |
| IGA Score ≤2 | 16 | 19 | 21 | 21 | 28 | 29 |
| (‘Mild’or better) | (37.2%) | (44.2%) | (48.8%) | (51.2%) | (65.1%) | (69.0%) |
| IGA Score = 0 | 1 | 4 | 7 | 7 | 9 | 16 |
| (‘Cleared’) or | (2.3%) | (9.3%) | (16.3%) | (17.1%) | (20.9%) | (38.1%) |
| 1 (‘Minimal’) | | | | | | |
| IGA Score = 0 | 0 | 0 | 0 | 0 | 0 | 0 |
| (‘Cleared’) | | | | | | |
| Week 8 | | | | | | |
| N | 43 | 43 | 43 | 41 | 43 | 42 |
| IGA Scorc ≤2 | 14 | 24 | 32 | 30 | 31 | 33 |
| (‘Mild’or better) | (32.6%) | (55.8%) | (74.4%) | (73.2%) | (72.1%) | (78.6%) |
| IGA Score = 0 | 3 | 12 | 19 | 11 | 20 | 27 |
| (‘Cleared’) or 1 | (7.0%) | (27.9%) | (44.2%) | (26.8%) | (46.5%) | (64.3%) |
| (‘Minimal’) | | | | | | |
| IGA Score = 0 | 0 | 1 | 2 | 2 | 4 | 8 |
| (‘Cleared’) | | (2.3%) | (4.7%) | (4.9%) | (9.3%) | (19.0%) |
| Week 12 | | | | | | |
| N | 43 | 43 | 43 | 41 | 43 | 42 |
| IGA Score ≤2 | 11 | 31 | 35 | 32 | 35 | 36 |
| (‘Mild’or better) | (25.6%) | (72.1%) | (81.4%) | (78.0%) | (81.4%) | (85.7%) |
| IGA Score = 0 | 4 | 11 | 22 | 18 | 23 | 28 |
| (‘Cleared’) or 1 | (9.3%) | (25.6%) | (51.2%) | (43.9%) | (53.5%) | (66.7%) |
| (‘Minimal’) | | | | | | |
| IGA Score = 0 | 0 | 3 | 4 | 3 | 8 | 17 |
| (‘Cleared’) | | (7.0%) | (9.3%) | (7.3%) | (18.6%) | (40.5%) |
| Week 16 | | | | | | |
| N | 43 | 43 | 43 | 41 | 43 | 42 |
| IGA Score ≤2 | 13 | 29 | 35 | 30 | 34 | 34 |
| (‘Mild’or better) | (30.2%) | (67.4%) | (81.4%) | (73.2%) | (79.1%) | (81.0%) |
| IGA Score = 0 | 5 | 17 | 25 | 21 | 27 | 27 |
| (‘Cleared’) or 1 | (11.6%) | (39.5%) | (58.1%) | (51.2%) | (62.8%) | (64.3%) |
| (‘Minimal’) | | | | | | |
| IGA Score = 0 | 0 | 7 | 15 | 6 | 12 | 19 |
| (‘Cleared’) | | (16.3%) | (34.9%) | (14.6%) | (27.9%) | (45.2%) |
| Week 20^{a} | | | | | | |
| N | 35 | 43 | 43 | 41 | 43 | 42 |
| IGA Score ≤2 | 25 | 28 | 35 | 31 | 31 | 34 |
| (‘Mild’or better) | (71.4%) | (65.1%) | (81.4%) | (75.6%) | (72.1%) | (81.0%) |
| IGA Score = 0 | 10 | 19 | 24 | 20 | 25 | 26 |
| (‘Cleared’) or 1 | (28.6%) | (44.2%) | (55.8%) | (48.8%) | (58.1%) | (61.9%) |
| (‘Minimal’) | | | | | | |
| IGA Score = 0 | 2 | 6 | 9 | 8 | 7 | 18 |
| (‘Cleared’) | (5.7%) | (14.0%) | (20.9%) | (19.5%) | (16.3%) | (42.9%) |
| Week 24^{a} | | | | | | |
| N | 35 | 43 | 43 | 41 | 43 | 42 |
| IGA Score ≤2 | 30 | 29 | 35 | 31 | 33 | 33 |
| (‘Mild’or better) | (85.7%) | (67.4%) | (81.4%) | (75.6%) | (76.7%) | (78.6%) |
| IGA Score = 0 | 19 | 16 | 26 | 16 | 28 | 31 |
| (‘Cleared’) or 1 | (54.3%) | (37.2%) | (60.5%) | (39.0%) | (65.1%) | (73.8%) |
| (‘Minimal’) | | | | | | |
| IGA Score = 0 | 5 | 7 | 14 | 8 | 10 | 16 |
| (‘Cleared’) | (14.3%) | (16.3%) | (32.6%) | (19.5%) | (23.3%) | (38.1%) |
| Week 28^{a} | | | | | | |
| N | 35 | 43 | 43 | 41 | 43 | 42 |
| IGA Score ≤2 | 30 | 28 | 33 | 30 | 32 | 33 |
| (‘Mild’or better) | (85.7%) | (65.1%) | (76.7%) | (73.2%) | (74.4%) | (78.6%) |
| IGA Score = 0 | 21 | 16 | 27 | 17 | 27 | 27 |
| (‘Cleared’) or 1 | (60.0%) | (37.2%) | (62.8%) | (41.5%) | (62.8%) | (64.3%) |
| (‘Minimal’) | | | | | | |
| IGA Score = 0 | 6 | 8 | 7 | 8 | 11 | 18 |
| (‘Cleared’) | (17.1%) | (18.6%) | (16.3%) | (19.5%) | (25.6%) | (42.9%) |
| Week 32^{a} | | | | | | |
| N | 35 | 43 | 43 | 41 | 43 | 42 |
| IGA Score ≤2 | 28 | 26 | 34 | 30 | 33 | 33 |
| (‘Mild’or better) | (80.0%) | (60.5%) | (79.1%) | (73.2%) | (76.7%) | (78.6%) |
| IGA Score = 0 | 23 | 17 | 25 | 19 | 29 | 28 |
| (‘Cleared’) or 1 | (65.7%) | (39.5%) | (58.1%) | (46.3%) | (67.4%) | (66.7%) |
| (‘Minimal’) | | | | | | |
| IGA Score = 0 | 7 | 8 | 13 | 9 | 11 | 17 |
| (‘Cleared’) | (20.0%) | (18.6%) | (30.2%) | (22.0%) | (25.6%) | (40.5%) |
| Week 40^{a} | | | | | | |
| N | 35 | 43 | 43 | 41 | 43 | 42 |
| IGA Score ≤2 | 26 | 29 | 34 | 28 | 30 | 33 |
| (‘Mild’or better) | (74.3%) | (67.4%) | (79.1%) | (68.3%) | (69.8%) | (78.6%) |
| IGA Score = 0 | 20 | 18 | 25 | 13 | 28 | 28 |
| (‘Cleared’) or 1 | (57.1%) | (41.9%) | (58.1%) | (31.7%) | (65.1%) | (66.7%) |
| (‘Minimal’) | | | | | | |
| IGA Score = 0 | 11 | 5 | 10 | 7 | 12 | 17 |
| (‘Cleared’) | (31.4%) | (11.6%) | (23.3%) | (17.1%) | (27.9%) | (40.5%) |
| Week 52^{a} | | | | | | |
| N | 35 | 43 | 43 | 41 | 43 | 42 |
| IGA Score ≤2 | 27 | 24 | 34 | 26 | 29 | 32 |
| (‘Mild’or better) | (77.1%) | (55.8%) | (79.1%) | (63.4%) | (67.4%) | (76.2%) |
| IGA Score = 0 | 23 | 16 | 26 | 19 | 26 | 31 |
| (‘Cleared’) or 1 | (65.7%) | (37.2%) | (60.5%) | (46.3%) | (60.5%) | (73.8%) |
| (‘Minimal’) | | | | | | |
| IGA Score = 0 | 11 | 6 | 10 | 8 | 13 | 18 |
| (‘Cleared’) | (31.4%) | (14.0%) | (23.3%) | (19.5%) | (30.2%) | (42.9%) |

^{a}For subjects who were randomized to placebo at Weck 0, only include those subjects who crossed over to treatment with the compound of Formula (I) at or after Week 16.
Subjects with ICE 1-2 were assumed to be non-responders after the event. Observed data were used for subjects with ICE 3. After accounting for the ICEs, subjects with missing data were considered as non-responders.

TABLE 32
PASI Response through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Week 1 | | | | | | |
| N | 43 | 43 | 43 | 41 | 43 | 42 |
| ≥75% Improvement | 0 | 0 | 2 | 0 | 1 | 1 |
| | | | (4.7%) | | (2.3%) | (2.4%) |
| ≥90% Improvement | 0 | 0 | 0 | 0 | 0 | 1 |
| | | | | | | (2.4%) |
| 100% Improvement | 0 | 0 | 0 | 0 | 0 | 0 |
| Week 2 | | | | | | |
| N | 43 | 43 | 43 | 41 | 43 | 42 |
| ≥75% Improvement | 0 | 0 | 2 | 0 | 1 | 2 |
| | | | (4.7%) | | (2.3%) | (4.8%) |
| ≥90% Improvement | 0 | 0 | 0 | 0 | 0 | 1 |
| | | | | | | (2.4%) |
| 100% Improvement | 0 | 0 | 0 | 0 | 0 | 0 |
| Week 4 | | | | | | |
| N | 43 | 43 | 43 | 41 | 43 | 42 |
| ≥75% Improvement | 0 | 4 | 7 | 2 | 7 | 10 |
| | | (9.3%) | (16.3%) | (4.9%) | (16.3%) | (23.8%) |
| ≥90% Improvement | 0 | 1 | 0 | 0 | 2 | 4 |
| | | (2.3%) | | | (4.7%) | (9.5%) |
| 100% Improvement | 0 | 0 | 0 | 0 | 0 | 0 |
| Week 8 | | | | | | |
| N | 43 | 43 | 43 | 41 | 43 | 42 |
| ≥75% Improvement | 3 | 10 | 17 | 8 | 19 | 20 |
| | (7.0%) | (23.3%) | (39.5%) | (19.5%) | (44.2%) | (47.6%) |
| ≥90% Improvement | 0 | 6 | 8 | 3 | 9 | 12 |
| | | (14.0%) | (18.6%) | (7.3%) | (20.9%) | (28.6%) |
| 100% Improvement | 0 | 1 | 1 | 1 | 2 | 5 |
| | | (2.3%) | (2.3%) | (2.4%) | (4.7%) | (11.9%) |
| Week 12 | | | | | | |
| N | 43 | 43 | 43 | 41 | 43 | 42 |
| ≥75% Improvement | 4 | 15 | 22 | 21 | 27 | 31 |
| | (9.3%) | (34.9%) | (51.2%) | (51.2%) | (62.8%) | (73.8%) |
| ≥90% Improvement | 0 | 8 | 15 | 8 | 17 | 22 |
| | | (18.6%) | (34.9%) | (19.5%) | (39.5%) | (52.4%) |
| 100% Improvement | 0 | 2 | 2 | 2 | 6 | 14 |
| | | (4.7%) | (4.7%) | (4.9%) | (14.0%) | (33.3%) |
| Week 16 | | | | | | |
| N | 43 | 43 | 43 | 41 | 43 | 42 |
| ≥75% Improvement | 4 | 16 | 25 | 21 | 28 | 33 |
| | (9.3%) | (37.2%) | (58.1%) | (51.2%) | (65.1%) | (78.6%) |
| ≥90% Improvement | 1 | 11 | 22 | 11 | 20 | 25 |
| | (2.3%) | (25.6%) | (51.2%) | (26.8%) | (46.5%) | (59.5%) |
| 100% Improvement | 0 | 5 | 11 | 4 | 10 | 17 |
| | | (11.6%) | (25.6%) | (9.8%) | (23.3%) | (40.5%) |
| Week 20^{a} | | | | | | |
| N | 35 | 43 | 43 | 41 | 43 | 42 |
| ≥75% Improvement | 14 | 21 | 27 | 23 | 26 | 34 |
| | (40.0%) | (48.8%) | (62.8%) | (56.1%) | (60.5%) | (81.0%) |
| ≥90% Improvement | 5 | 13 | 21 | 14 | 19 | 26 |
| | (14.3%) | (30.2%) | (48.8%) | (34.1%) | (44.2%) | (61.9%) |
| 100% Improvement | 1 | 6 | 8 | 8 | 6 | 17 |
| | (2.9%) | (14.0%) | (18.6%) | (19.5%) | (14.0%) | (40.5%) |
| Week 24^{a} | | | | | | |
| N | 35 | 43 | 43 | 41 | 43 | 42 |
| ≥75% Improvement | 21 | 20 | 26 | 25 | 30 | 34 |
| | (60.0%) | (46.5%) | (60.5%) | (61.0%) | (69.8%) | (81.0%) |
| ≥90% Improvement | 8 | 14 | 23 | 12 | 24 | 29 |
| | (22.9%) | (32.6%) | (53.5%) | (29.3%) | (55.8%) | (69.0%) |
| 100% Improvement | 3 | 7 | 12 | 8 | 9 | 15 |
| | (8.6%) | (16.3%) | (27.9%) | (19.5%) | (20.9%) | (35.7%) |
| Week 28^{a} | | | | | | |
| N | 35 | 43 | 43 | 41 | 43 | 42 |
| ≥75% Improvement | 22 | 17 | 30 | 25 | 28 | 33 |
| | (62.9%) | (39.5%) | (69.8%) | (61.0%) | (65.1%) | (78.6%) |
| ≥90% Improvement | 8 | 12 | 21 | 15 | 22 | 26 |
| | (22.9%) | (27.9%) | (48.8%) | (36.6%) | (51.2%) | (61.9%) |
| 100% Improvement | 5 | 8 | 6 | 7 | 11 | 16 |
| | (14.3%) | (18.6%) | (14.0%) | (17.1%) | (25.6%) | (38.1%) |
| Week 32^{a} | | | | | | |
| N | 35 | 43 | 43 | 41 | 43 | 42 |
| ≥75% Improvement | 23 | 21 | 28 | 22 | 30 | 33 |
| | (65.7%) | (48.8%) | (65.1%) | (53.7%) | (69.8%) | (78.6%) |
| ≥90% Improvement | 14 | 13 | 22 | 16 | 27 | 28 |
| | (40.0%) | (30.2%) | (51.2%) | (39.0%) | (62.8%) | (66.7%) |
| 100% Improvement | 7 | 8 | 11 | 7 | 11 | 14 |
| | (20.0%) | (18.6%) | (25.6%) | (17.1%) | (25.6%) | (33.3%) |
| Week 40^{a} | | | | | | |
| N | 35 | 43 | 43 | 41 | 43 | 42 |
| ≥75% Improvement | 21 | 21 | 29 | 22 | 30 | 33 |
| | (60.0%) | (48.8%) | (67.4%) | (53.7%) | (69.8%) | (78.6%) |
| ≥90% Improvement | 16 | 14 | 20 | 13 | 24 | 27 |
| | (45.7%) | (32.6%) | (46.5%) | (31.7%) | (55.8%) | (64.3%) |
| 100% Improvement | 10 | 5 | 9 | 6 | 11 | 15 |
| | (28.6%) | (11.6%) | (20.9%) | (14.6%) | (25.6%) | (35.7%) |
| Week 52^{a} | | | | | | |
| N | 35 | 43 | 43 | 41 | 43 | 42 |
| ≥75% Improvement | 23 | 21 | 30 | 24 | 28 | 32 |
| | (65.7%) | (48.8%) | (69.8%) | (58.5%) | (65.1%) | (76.2%) |
| ≥90% Improvement | 20 | 12 | 18 | 15 | 22 | 27 |
| | (57.1%) | (27.9%) | (41.9%) | (36.6%) | (51.2%) | (64.3%) |
| 100% Improvement | 12 | 6 | 9 | 7 | 11 | 17 |
| | (34.3%) | (14.0%) | (20.9%) | (17.1%) | (25.6%) | (40.5%) |

^{a}For subjects who were randomized to placebo at Week 0, only include those subjects who crossed over to treatment with the compound of Formula (I) at or after Week 16.
Subjects with ICE 1-2 were assumed to be non-responders after the event. Observed data were used for subjects with ICE 3. After accounting for the ICEs, subjects with missing data were considered as non-responders.

Overall, response rates were maintained for the 100 mg BID dose over the 52-week treatment period, with some endpoints showing slight improvement after Week 16 (most notably PASI 90 and IGA 0/1). Change from baseline endpoints (e.g., change in PASI score overtime) showed continued improvement through Week 52.

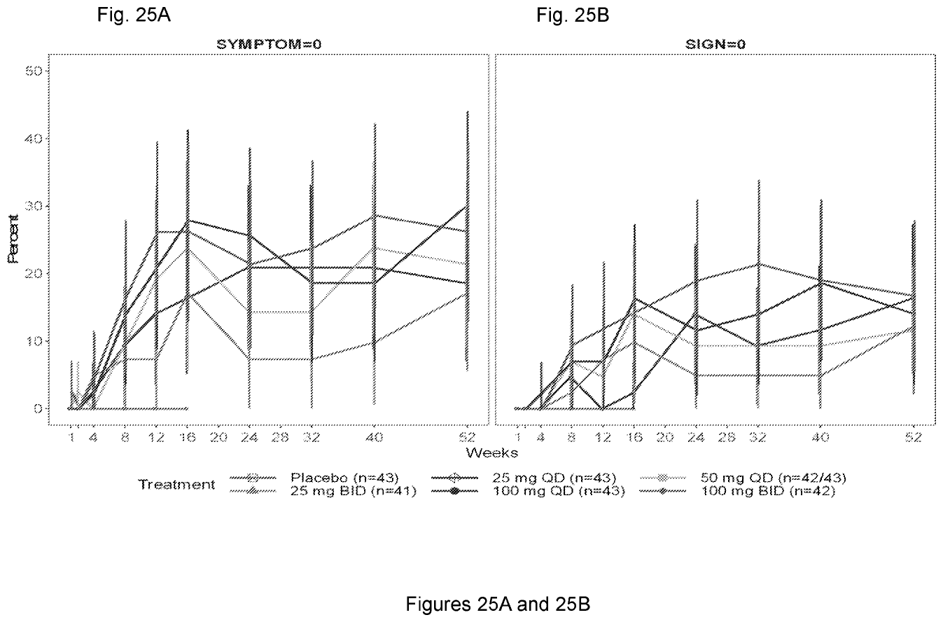

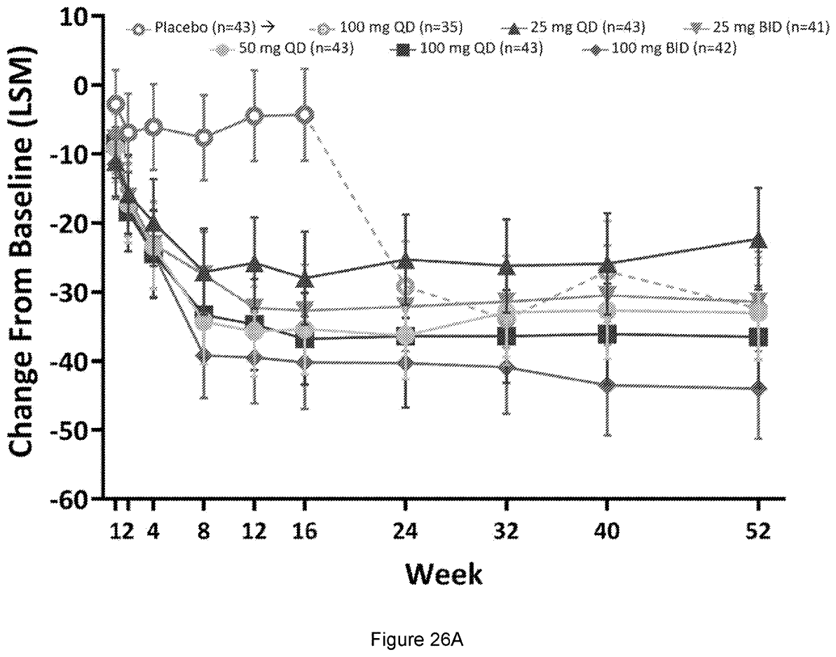

The proportion of participants achieving PSSD symptom score of absent (0) over time among participants with a baseline symptom score≥1 and the proportion of participants achieving a PSSD sign score of absent (0) among participants with a baseline sign score≥1 over time are summarized in FIG. 25, Table 33, and Table 34.

TABLE 33
Proportion of Subjects Achieving PSSD Symptom Score 0 through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Subjects with Baseline | 43 | 43 | 42 | 41 | 43 | 42 |
| Symptom Score ≥1 | | | | | | |
| Week 1 | 0 | 0 | 0 | 0 | 0 | 1 |
| | | | | | | (2.4%) |
| Week 2 | 0 | 0 | 1 | 0 | 0 | 0 |
| | | | (2.4%) | | | |
| Week 4 | 0 | 1 | 0 | 2 | 1 | 2 |
| | | (2.3%) | | (4.9%) | (2.3%) | (4.8%) |
| Week 8 | 0 | 4 | 4 | 3 | 6 | 7 |
| | | (9.3%) | (9.5%) | (7.3%) | (14.0%) | (16.7%) |
| Week 12 | 0 | 6 | 8 | 3 | 9 | 11 |
| | | (14.0%) | (19.0%) | (7.3%) | (20.9%) | (26.2%) |
| Week 16 | 0 | 7 | 10 | 7 | 12 | 11 |
| | | (16.3%) | (23.8%) | (17.1%) | (27.9%) | (26.2%) |
| Week 24^{a} | 4 | 9 | 6 | 3 | 11 | 9 |
| | (11.4%) | (20.9%) | (14.3%) | (7.3%) | (25.6%) | (21.4%) |
| Week 32^{a} | 7 | 9 | 6 | 3 | 8 | 10 |
| | (20.0%) | (20.9%) | (14.3%) | (7.3%) | (18.6%) | (23.8%) |
| Week 40^{a} | 9 | 9 | 10 | 4 | 8 | 12 |
| | (25.7%) | (20.9%) | (23.8%) | (9.8%) | (18.6%) | (28.6%) |
| Week 52^{a} | 12 | 8 | 9 | 7 | 13 | 11 |
| | (34.3%) | (18.6%) | (21.4%) | (17.1%) | (30.2%) | (26.2%) |

^{a}For subjects who were randomized to placebo at Week 0, only include those subjects who crossed over to treatment with the compound of Formula (I) at or after Week 16.
Subjects with ICE 1-2 were assumed to be non-responders after the event. Observed data were used for subjects with ICE 3. After accounting for the ICEs, subjects with missing data were considered as non-responders.

TABLE 34
Proportion of Subjects Achieving PSSD Sign Score 0 through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Subjects with Baseline | 43 | 43 | 43 | 41 | 43 | 42 |
| Sign Score ≥1 | | | | | | |
| Week 1 | 0 | 0 | 0 | 0 | 0 | 0 |
| Week 2 | 0 | 0 | 0 | 0 | 0 | 0 |
| Week 4 | 0 | 0 | 0 | 0 | 1 | 0 |
| | | | | | (2.3%) | |
| Week 8 | 0 | 2 | 3 | 1 | 3 | 4 |
| | | (4.7%) | (7.0%) | (2.4%) | (7.0%) | (9.5%) |
| Week 12 | 0 | 0 | 2 | 3 | 3 | 5 |
| | | | (4.7%) | (7.3%) | (7.0%) | (11.9%) |
| Week 16 | 0 | 1 | 6 | 4 | 7 | 6 |
| | | (2.3%) | (14.0%) | (9.8%) | (16.3%) | (14.3%) |
| Week 24^{a} | 3 | 6 | 4 | 2 | 5 | 8 |
| | (8.6%) | (14.0%) | (9.3%) | (4.9%) | (11.6%) | (19.0%) |
| Week 32^{a} | 6 | 4 | 4 | 2 | 6 | 9 |
| | (17.1%) | (9.3%) | (9.3%) | (4.9%) | (14.0%) | (21.4%) |
| Week 40^{a} | 5 | 5 | 4 | 2 | 8 | 8 |
| | (14.3%) | (11.6%) | (9.3%) | (4.9%) | (18.6%) | (19.0%) |
| Week 52^{a} | 8 | 7 | 5 | 5 | 6 | 7 |
| | (22.9%) | (16.3%) | (11.6%) | (12.2%) | (14.0%) | (16.7%) |

^{a}For subjects who were randomized to placebo at Week 0, only include those subjects who crossed over to treatment with the compound of Formula (I) at or after Week 16.
Subjects with ICE 1-2 were assumed to be non-responders after the event. Observed data were used for subjects with ICE 3. After accounting for the ICEs, subjects with missing data were considered as non-responders.

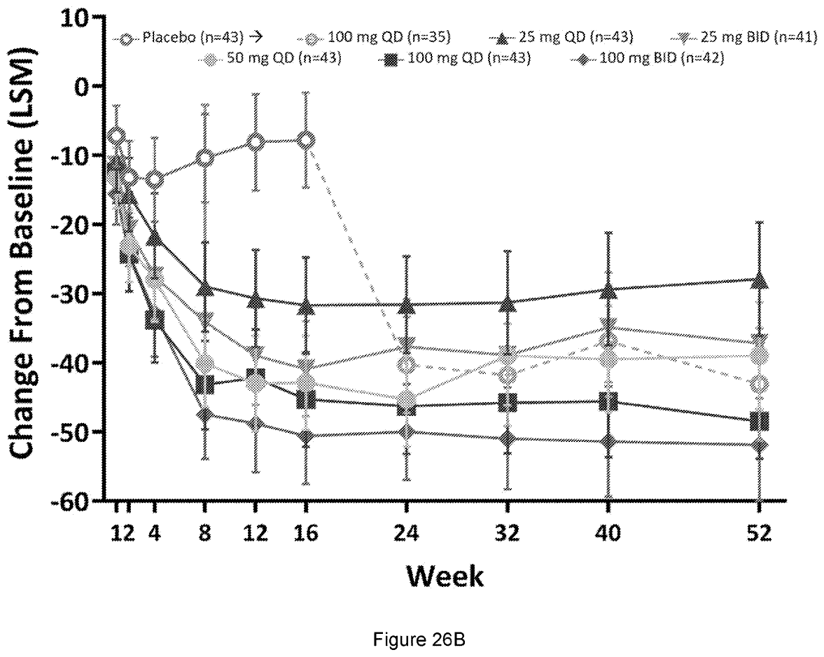

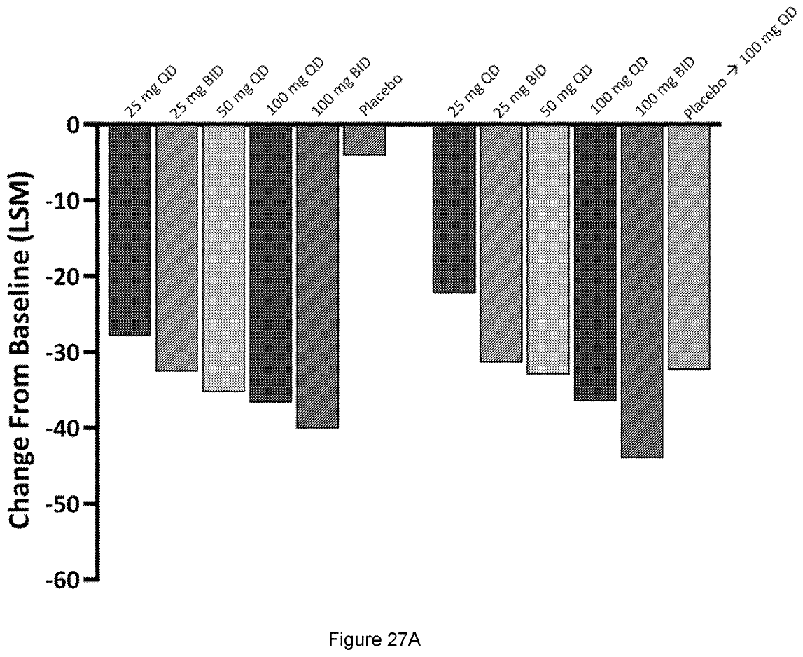

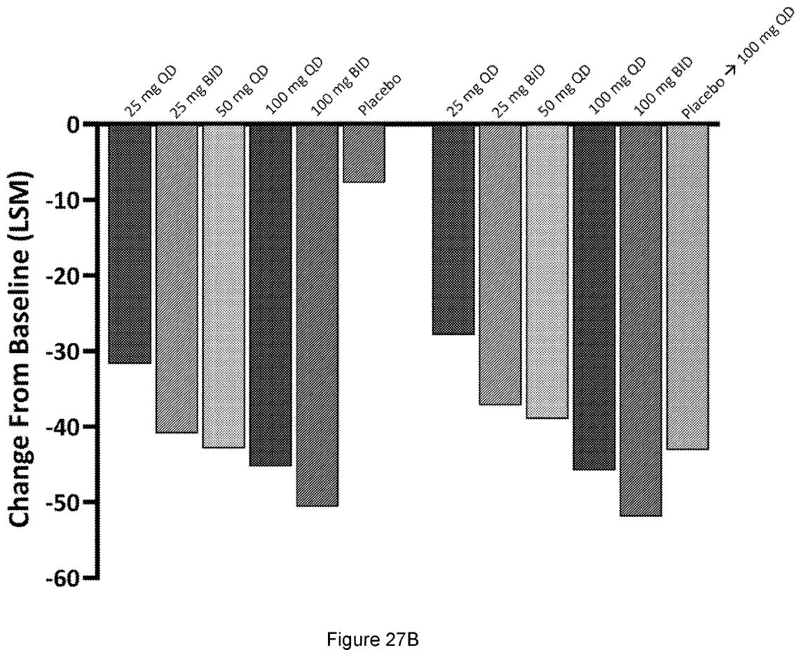

LSMeans and the corresponding 950 CIs for change from baseline in PSSD symptom score, and PSSD sign score respectively are presented in FIG. 26, FIG. 27, Table 35, and Table 36. In general, the response pattern was similar to the response pattern observed in PASI through Week 52.

TABLE 35
Change from Baseline in PSSD Symptom Score Through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Baseline | | | | | | |
| N | 43 | 43 | 43 | 41 | 43 | 42 |
| Mean (SD) | 47.3 | 59.0 | 53.9 | 51.9 | 43.0 | 55.9 |
| | (20.67) | (23.63) | (24.49) | (24.05) | (21.27) | (26.27) |
| Median | 44.0 | 56.0 | 56.0 | 50.0 | 44.0 | 56.0 |
| Range | (10; 98) | (6; 98) | (0; 100) | (4; 100) | (6; 96) | (6; 100) |
| IQ range | (34.0; 64.0) | (42.0; 76.0) | (34.0; 68.0) | (36.0; 70.0) | (28.0; 56.0) | (40.0; 76.0) |

Change from Baseline in PSSD Symptom Score

| | | | | | | |
|---|---|---|---|---|---|---|
| Week 1 | | | | | | |
| N | 42 | 42 | 43 | 41 | 40 | 42 |
| Mean (SD) | −1.5 | −12.9 | −9.6 | −7.6 | −6.2 | −12.5 |
| | (18.68) | (15.64) | (16.88) | (15.96) | (18.03) | (20.11) |
| Median | 2.0 | −12.0 | −12.0 | −8.0 | −7.0 | −8.0 |
| Range | (−52; 42) | (−50; 16) | (−46; 38) | (−50; 30) | (−44; 46) | (−80; 24) |
| IQ range | (−12.0; 10.0) | (−22.0; −2.0) | (−18.0; 0.0) | (−14.0; 0.0) | (−17.0; 4.0) | (−22.0; 0.0) |
| LSMean (95% CI)^{a} | −2.8 | −11.1 | −9.1 | −7.8 | −8.4 | −11.5 |
| | (−7.79, 2.18) | (−16.14, −6.10) | (−14.07, −4.20) | (−12.81, −2.71) | (−13.48, −3.23) | (−16.53, −6.54) |
| Week 2 | | | | | | |
| N | 43 | 42 | 42 | 41 | 42 | 40 |
| Mean (SD) | −5.0 | −18.1 | −17.3 | −16.0 | −15.2 | −18.4 |
| | (23.54) | (16.49) | (20.84) | (21.84) | (17.85) | (21.55) |
| Median | −2.0 | −17.0 | −16.0 | −12.0 | −14.0 | −14.0 |
| Range | (−60; 56) | (−56; 10) | (−56; 42) | (−70; 28) | (−58; 46) | (−84; 24) |
| IQ range | (−20.0; 8.0) | (−30.0; −8.0) | (−36.0; −6.0) | (−28.0; −4.0) | (−28.0; −6.0) | (−33.0; −4.0) |
| LSMean (95% CI)^{a} | −6.9 | −15.8 | −17.2 | −16.2 | −18.4 | −17.1 |
| | (−12.52, −1.26) | (−21.54, −10.12) | (−22.85, −11.54) | (−21.93, −10.44) | (−24.12, −12.64) | (−22.86, −11.33) |
| Week 4 | | | | | | |
| N | 43 | 42 | 42 | 41 | 42 | 41 |
| Mean (SD) | −3.0 | −23.6 | −23.6 | −22.8 | −19.4 | −26.1 |
| | (25.69) | (23.13) | (22.67) | (27.28) | (21.33) | (27.49) |
| Median | −2.0 | −23.0 | −23.0 | −20.0 | −17.0 | −22.0 |
| Range | (−58; 48) | (−66; 26) | (−64; 34) | (−78; 38) | (−64; 34) | (−88; 44) |
| IQ range | (−18.0; 16.0) | (−40.0; −8.0) | (−43.0; −4.0) | (−34.0; −10.0) | (−40.0; 0.0) | (−46.0; −8.0) |
| LSMean (95% CI)^{a} | −6.1 | −19.9 | −23.4 | −23.1 | −24.5 | −24.4 |
| | (−12.29, 0.12) | (−26.23, −13.64) | (−29.61, −17.14) | (−29.48, −16.82) | (−30.86, −18.21) | (−30.73, −18.09) |
| Week 8 | | | | | | |
| N | 41 | 41 | 42 | 41 | 41 | 41 |
| Mean (SD) | −5.3 | −32.3 | −35.0 | −27.5 | −27.8 | −41.7 |
| | (24.19) | (27.18) | (26.53) | (29.35) | (23.16) | (26.45) |
| Median | 0.0 | −32.0 | −36.0 | −22.0 | −26.0 | −42.0 |
| Range | (−84; 36) | (−92; 42) | (−86; 40) | (−84; 32) | (−76; 20) | (−96; 6) |
| IQ range | (−20.0; 6.0) | (−50.0; −14.0) | (−50.0; −16.0) | (−52.0; −8.0) | (−44.0; −12.0) | (−62.0; −18.0) |
| LSMean (95% CI)^{a} | −7.6 | −27.1 | −34.3 | −27.5 | −33.3 | −39.2 |
| | (−13.80, −1.49) | (−33.26, −20.84) | (−40.46, −28.21) | (−33.69, −21.27) | (−39.52, −27.07) | (−45.38, −32.96) |
| Week 12 | | | | | | |
| N | 40 | 41 | 42 | 41 | 42 | 40 |
| Mean (SD) | −1.1 | −31.5 | −36.3 | −32.0 | −27.9 | −42.3 |
| | (27.52) | (28.73) | (29.26) | (29.86) | (27.40) | (28.29) |
| Median | 0.0 | −34.0 | −37.0 | −28.0 | −27.0 | −41.0 |
| Range | (−56; 66) | (−94; 34) | (−90; 38) | (−90; 34) | (−78; 46) | (−98; 24) |
| IQ range | (−22.0; 19.0) | (−50.0; −14.0) | (−56.0; −18.0) | (−54.0; −14.0) | (−52.0; −10.0) | (−63.0; −21.0) |
| LSMean (95% CI)^{a} | −4.5 | −25.8 | −35.7 | −32.3 | −34.7 | −39.5 |
| | (−11.02, 2.12) | (−32.41, −19.21) | (−42.24, −29.23) | (−38.88, −25.68) | (−41.32, −28.13) | (−46.15, −32.90) |
| Week 16 | | | | | | |
| N | 40 | 37 | 41 | 40 | 42 | 39 |
| Mean (SD) | −0.8 | −35.8 | −36.7 | −34.0 | −29.4 | −44.0 |
| | (29.59) | (29.22) | (29.95) | (29.19) | (28.28) | (31.22) |
| Median | 0.0 | −34.0 | −36.0 | −28.0 | −31.0 | −42.0 |
| Range | (−66; 64) | (−96; 22) | (−90; 34) | (−100; 24) | (−96; 22) | (−98; 34) |
| IQ range | (−20.0; 21.0) | (−56.0; −20.0) | (−58.0; −20.0) | (−55.0; −16.0) | (−50.0; −6.0) | (−68.0; −20.0) |
| LSMean (95% CI)^{a} | −4.3 | −28.0 | −35.4 | −32.7 | −36.8 | −40.2 |
| | (−10.96, 2.34) | (−34.74, −21.26) | (−41.95, −28.79) | (−39.39, −26.05) | (−43.42, −30.11) | (−46.94, −33.51) |
| Week 24^{b} | | | | | | |
| N | 35 | 36 | 40 | 38 | 38 | 37 |
| Mean (SD) | −26.6 | −32.9 | −38.8 | −33.3 | −28.4 | −44.8 |
| | (28.22) | (31.58) | (28.54) | (26.90) | (25.37) | (28.01) |
| Median | −26.0 | −33.0 | −40.0 | −29.0 | −26.0 | −44.0 |
| Range | (−98; 46) | (−96; 34) | (−100; 44) | (−90; 22) | (−76; 18) | (−98; 12) |
| IQ range | (−42.0; −12.0) | (−55.0; −9.0) | (−56.0; −22.0) | (−54.0; −14.0) | (−44.0; −12.0) | (−66.0; −24.0) |
| LSMean (95% CI)^{a} | −29.2 | −25.3 | −36.3 | −32.1 | −36.4 | −40.3 |
| | (−35.73, −22.67) | (−31.87, −18.79) | (−42.62, −29.96) | (−38.56, −25.70) | (−42.83, −29.88) | (−46.76, −33.75) |
| Week 32^{b} | | | | | | |
| N | 34 | 35 | 39 | 37 | 36 | 36 |
| Mean (SD) | −31.9 | −32.5 | −35.9 | −31.0 | −28.0 | −45.5 |
| | (27.12) | (30.06) | (29.11) | (28.29) | (26.52) | (29.64) |
| Median | −36.0 | −32.0 | −32.0 | −28.0 | −25.0 | −47.0 |
| Range | (−90; 42) | (−96; 46) | (−100; 32) | (−90; 10) | (−94; 16) | (−98; 8) |
| IQ range | (−50.0; −14.0) | (−54.0; −12.0) | (−58.0; −14.0) | (−54.0; −8.0) | (−46.0; −11.0) | (−70.0; −17.0) |
| LSMean (95% CI)^{a} | −33.9 | −26.2 | −32.9 | −31.4 | −36.4 | −40.9 |
| | (−40.62, −27.09) | (−33.01, −19.45) | (−39.39, −26.33) | (−38.07, −24.78) | (−43.16, −29.69) | (−47.63, −34.17) |
| Week 40^{b} | | | | | | |
| N | 34 | 34 | 40 | 36 | 35 | 36 |
| Mean (SD) | −23.8 | −33.9 | −34.9 | −30.0 | −28.2 | −47.5 |
| | (30.22) | (26.66) | (29.25) | (29.27) | (27.77) | (27.89) |
| Median | −24.0 | −35.0 | −32.0 | −32.0 | −24.0 | −49.5 |
| Range | (−88; 40) | (−96; 2) | (−100; 30) | (−80; 42) | (−94; 22) | (−100; 0) |
| IQ range | (−42.0; 0.0) | (−56.0; −12.0) | (−55.0; −14.0) | (−53.0; −4.0) | (−48.0; −12.0) | (−72.0; −23.0) |
| LSMean (95% CI)^{a} | −27.0 | −25.9 | −32.7 | −30.5 | −36.1 | −43.5 |
| | (−34.28, −19.68) | (−33.27, −18.59) | (−39.68, −25.72) | (−37.65, −23.26) | (−43.42, −28.78) | (−50.78, −36.30) |
| Week 52^{b} | | | | | | |
| N | 34 | 34 | 40 | 36 | 35 | 36 |
| Mean (SD) | −29.5 | −30.1 | −35.2 | −31.2 | −29.4 | −47.7 |
| | (25.59) | (28.09) | (30.81) | (28.48) | (27.41) | (28.04) |
| Median | −30.0 | −35.0 | −34.0 | −27.0 | −28.0 | −53.0 |
| Range | (−98; 24) | (−96; 16) | (−100; 30) | (−80; 22) | (−90; 16) | (−98; 0) |
| IQ range | (−44.0; −12.0) | (−46.0; 0.0) | (−58.0; −12.0) | (−55.0; −7.0) | (−50.0; 0.0) | (−69.0; −22.0) |
| LSMean (95% CI)^{a} | −32.4 | −22.3 | −33.0 | −31.4 | −36.5 | −44.0 |
| | (−39.70, −25.02) | (−29.65, −14.90) | (−39.94, −26.02) | (−38.57, −24.16) | (−43.86, −29.14) | (−51.28, −36.77) |

^{a}LS Means are based on the MMRM model with treatment group, visit, treatment group by visit interaction, baseline weight category (≤90 kg, >90 kg), baseline weight category by visit interaction, baseline PSSD symptom score, and baseline PSSD symptom score by visit interaction as covariates.
^{b}For subjects who were randomized to placebo at Week 0, only include those subjects who crossed over to treatment with the compound of Formula (I) at or after Week 16.
Subjects who have ICE 1-2 have zero change from baseline after the event. Observed data were used for subjects with ICE 3.

TABLE 36
Change from Baseline in PSSD Sign Score through Week 52

| | Placebo → | 25 mg | 50 mg | 25 mg | 100 mg | 100 mg |
|---|---|---|---|---|---|---|
| Treatment Regimen | 100 mg QD | QD | QD | BID | QD | BID |
| Analysis set: | 43 | 43 | 43 | 41 | 43 | 42 |
| Full analysis set | | | | | | |
| Baseline | | | | | | |
| N | 43 | 43 | 43 | 41 | 43 | 42 |
| Mean (SD) | 62.9 | 69.5 | 64.7 | 64.1 | 60.4 | 66.3 |
| | (16.64) | (16.50) | (19.41) | (18.90) | (18.56) | (19.09) |
| Median | 67.0 | 70.0 | 65.0 | 62.0 | 63.0 | 70.0 |
| Range | (12; 97) | (28; 100) | (5; 100) | (20; 100) | (13; 95) | (23; 100) |
| IQ range | (52.0; 72.0) | (58.0; 82.0) | (55.0; 77.0) | (50.0; 78.0) | (48.0; 73.0) | (53.0; 82.0) |

Change from Baseline in PSSD Sign Score

| | | | | | | |
|---|---|---|---|---|---|---|
| Week 1 | | | | | | |
| N | 42 | 42 | 43 | 41 | 40 | 42 |
| Mean (SD) | −7.0 | −11.9 | −13.3 | −11.3 | −12.0 | −15.9 |
| | (16.35) | (15.32) | (13.05) | (15.01) | (14.60) | (15.08) |
| Median | −4.5 | −10.5 | −11.0 | −11.0 | −9.0 | −16.0 |
| Range | (−52; 30) | (−48; 16) | (−41; 17) | (−45; 20) | (−53; 14) | (−52; 11) |
| IQ range | (−15.0; 2.0) | (−20.0; 0.0) | (−22.0; −6.0) | (−22.0; −1.0) | (−22.0; −1.0) | (−24.0; −6.0) |
| LSMean (95% CI)^{a} | −7.2 | −10.9 | −13.3 | −11.4 | −12.4 | −15.6 |
| | (−11.63, −2.83) | (−15.36, −6.48) | (−17.65, −8.92) | (−15.92, −6.97) | (−16.89, −7.87) | (−20.01, −11.17) |
| Week 2 | | | | | | |
| N | 43 | 42 | 42 | 41 | 42 | 40 |
| Mean (SD) | −12.3 | −17.6 | −22.7 | −20.3 | −22.6 | −24.7 |
| | (22.14) | (14.54) | (19.58) | (19.73) | (17.49) | (19.36) |
| Median | −10.0 | −17.5 | −22.0 | −15.0 | −20.0 | −28.0 |
| Range | (−77; 40) | (−55; 10) | (−54; 28) | (−68; 15) | (−68; 7) | (−65; 15) |
| IQ range | (−31.0; 0.0) | (−27.0; −7.0) | (−42.0; −10.0) | (−36.0; −8.0) | (−38.0; −7.0) | (−37.0; −10.5) |
| LSMean (95% CI)^{a} | −13.2 | −15.7 | −23.1 | −20.7 | −24.3 | −24.3 |
| | (−18.38, −7.94) | (−21.01, −10.39) | (−28.33, −17.82) | (−26.02, −15.32) | (−29.65, −19.04) | (−29.70, −19.00) |
| Week 4 | | | | | | |
| N | 43 | 42 | 42 | 41 | 42 | 41 |
| Mean (SD) | −12.1 | −24.5 | −27.4 | −26.9 | −31.1 | −33.3 |
| | (21.74) | (22.48) | (23.35) | (25.35) | (23.61) | (23.10) |
| Median | −10.0 | −29.5 | −29.0 | −25.0 | −32.5 | −32.0 |
| Range | (−63; 27) | (−68; 22) | (−68; 35) | (−84; 33) | (−75; 8) | (−79; 22) |
| IQ range | (−32.0; 2.0) | (−42.0; −3.0) | (−47.0; −12.0) | (−43.0; −8.0) | (−45.0; −11.0) | (−50.0; −20.0) |
| LSMean (95% CI)^{a} | −13.5 | −21.7 | −28.0 | −27.5 | −33.8 | −32.9 |
| | (−19.58, −7.47) | (−27.81, −15.49) | (−34.11, −21.91) | (−33.72, −21.32) | (−39.97, −27.67) | (−39.11, −26.74) |
| Week 8 | | | | | | |
| N | 41 | 41 | 42 | 41 | 41 | 41 |
| Mean (SD) | −10.0 | −33.0 | −39.7 | −33.8 | −40.2 | −48.3 |
| | (20.70) | (25.13) | (25.56) | (28.70) | (24.50) | (23.10) |
| Median | −5.0 | −40.0 | −40.5 | −38.0 | −43.0 | −52.0 |
| Range | (−53; 24) | (−75; 24) | (−81; 9) | (−92; 25) | (−83; 15) | (−84; 0) |
| IQ range | (−22.0; 2.0) | (−50.0; −20.0) | (−62.0; −23.0) | (−52.0; −17.0) | (−50.0; −28.0) | (−69.0; −29.0) |
| LSMean (95% CI)^{a} | −10.4 | −29.0 | −40.1 | −34.1 | −43.2 | −47.5 |
| | (−16.79, −4.06) | (−35.47, −22.60) | (−46.44, −33.74) | (−40.57, −27.69) | (−49.65, −36.84) | (−53.91, −41.04) |
| Week 12 | | | | | | |
| N | 40 | 41 | 42 | 41 | 42 | 40 |
| Mean (SD) | −6.6 | −34.8 | −42.5 | −38.5 | −39.1 | −49.9 |
| | (23.03) | (29.11) | (25.88) | (30.08) | (29.02) | (22.93) |
| Median | 0.0 | −40.0 | −46.0 | −40.0 | −45.0 | −53.0 |
| Range | (−55; 27) | (−84; 25) | (−87; 16) | (−95; 24) | (−85; 32) | (−87; 5) |
| IQ range | (−25.0; 11.0) | (−55.0; −16.0) | (−60.0; −27.0) | (−59.0; −23.0) | (−58.0; −17.0) | (−69.0; −36.5) |
| LSMean (95% CI)^{a} | −8.1 | −30.7 | −43.0 | −39.0 | −42.2 | −48.8 |
| | (−15.11, −1.16) | (−37.73, −23.69) | (−49.93, −36.08) | (−46.07, −32.02) | (−49.13, −35.19) | (−55.83, −41.74) |
| Week 16 | | | | | | |
| N | 40 | 37 | 41 | 40 | 42 | 39 |
| Mean (SD) | −6.2 | −38.6 | −42.7 | −41.8 | 41.9 | −51.1 |
| | (22.38) | (27.55) | (28.70) | (27.78) | (28.65) | (26.01) |
| Median | 0.0 | −43.0 | −43.0 | −36.5 | −45.0 | −55.0 |
| Range | (−52; 28) | (−91; 17) | (−93; 18) | (−98; 15) | (−93; 38) | (−90; 28) |
| IQ range | (−24.5; 8.0) | (−60.0; −15.0) | (−70.0; −20.0) | (−61.5; −21.0) | (−62.0; −20.0) | (−73.0; −37.0) |
| LSMean (95% CI)^{a} | −7.8 | −31.7 | −42.9 | −40.9 | −45.3 | −50.6 |
| | (−14.65, −0.93) | (−38.70, −24.77) | (−49.72, −36.12) | (−47.76, −33.97) | (−52.14, −38.50) | (−57.54, −43.69) |
| Week 24^{b} | | | | | | |
| N | 35 | 36 | 40 | 38 | 38 | 37 |
| Mean (SD) | −40.2 | −38.6 | −45.7 | −39.8 | −41.9 | −51.6 |
| | (24.93) | (30.65) | (26.26) | (28.07) | (25.80) | (24.24) |
| Median | −40.0 | −44.5 | −47.5 | −41.0 | −44.0 | −53.0 |
| Range | (−90; 20) | (−98; 17) | (−97; 10) | (−90; 8) | (−95; 18) | (−89; 12) |
| IQ range | (−52.0; −28.0) | (−62.0; −11.0) | (−66.5; −25.5) | (−58.0; −15.0) | (−60.0; −23.0) | (−72.0; −40.0) |
| LSMean (95% CI)^{a} | −40.3 | −31.6 | −45.3 | −37.7 | −46.3 | −50.0 |
| | (−47.28, −33.35) | (−38.62, −24.64) | (−52.11, −38.56) | (−44.62, −30.85) | (−53.20, −39.44) | (−56.96, −43.08) |
| Week 32^{b} | | | | | | |
| N | 34 | 35 | 39 | 37 | 36 | 36 |
| Mean (SD) | −42.3 | −37.2 | −39.7 | −38.3 | −42.3 | −52.3 |
| | (27.58) | (30.49) | (30.08) | (26.06) | (25.49) | (23.33) |
| Median | −45.0 | −39.0 | −45.0 | −38.0 | −43.0 | −54.0 |
| Range | (−83; 18) | (−96; 33) | (−98; 20) | (−90; 0) | (−92; 9) | (−85; 0) |
| IQ range | (−62.0; −25.0) | (−61.0; −9.0) | (−65.0; −15.0) | (−56.0; −17.0) | (−60.0; −23.0) | (−73.0; −37.5) |
| LSMean (95% CI)^{a} | −41.8 | −31.3 | −39.0 | −38.9 | −45.8 | −51.0 |
| | (−49.17, −34.36) | (−38.78, −23.89) | (−46.14, −31.81) | (−46.20, −31.61) | (−53.16, −38.49) | (−58.32, −43.58) |
| Week 40^{b} | | | | | | |
| N | 34 | 34 | 40 | 36 | 35 | 36 |
| Mean (SD) | −36.6 | −36.9 | −39.6 | −34.1 | −42.4 | −52.4 |
| | (32.25) | (28.17) | (29.84) | (27.50) | (28.11) | (22.33) |
| Median | −48.5 | −36.0 | −42.0 | −34.0 | −43.0 | −54.5 |
| Range | (−85; 25) | (−96; 0) | (−100; 12) | (−92; 15) | (−95; 15) | (−86; 0) |
| IQ range | (−65.0; −5.0) | (−60.0; −10.0) | (−65.5; −17.5) | (−49.0; −9.0) | (−65.0; −26.0) | (−71.0; −40.0) |
| LSMean (95% CI)^{a} | −36.9 | −29.4 | −39.5 | −34.9 | −45.6 | −51.4 |
| | (−44.92, −28.81) | (−37.47, −21.24) | (−47.22, −31.78) | (−42.84, −26.94) | (−53.65, −37.61) | (−59.41, −43.43) |
| Week 52^{b} | | | | | | |
| N | 34 | 34 | 40 | 36 | 35 | 36 |
| Mean (SD) | −42.8 | −35.2 | −39.2 | −36.6 | −43.1 | −53.1 |
| | (28.65) | (29.02) | (31.64) | (29.16) | (26.87) | (22.03) |
| Median | −48.5 | −32.5 | −45.0 | −38.5 | −43.0 | −55.0 |
| Range | (−94; 11) | (−98; 12) | (−100; 22) | (−92; 15) | (−87; 9) | (−85; 0) |
| IQ range | (−65.0; −23.0) | (−60.0; −15.0) | (−63.5; −14.0) | (−57.0; −14.0) | (−61.0; −29.0) | (−70.5; −40.0) |
| LSMean (95% CI)^{a} | −43.1 | −27.9 | −39.0 | −37.2 | −45.8 | −51.9 |
| | (−51.27, −34.99) | (−36.11, −19.71) | (−46.71, −31.23) | (−45.15, −29.15) | (−53.88, −37.68) | (−59.97, −43.87) |

^{a}LS Means are based on the MMRM model with treatment group, visit, treatment group by visit interaction, baseline weight category (≤90 kg, >90 kg), baseline weight category by visit interaction, baseline PSSD sign score, and baseline PSSD sign score by visit interaction as covariates.
^{b}For subjects who were randomized to placebo at Week 0, only include those subjects who crossed over to treatment with the compound of Formula (I) at or after Week 16.
Subjects who have ICE 1-2 have zero change from baseline after the event. Observed data were used for subjects with ICE 3.

### Safety

As with Example 1, the compound of Formula (I) was well-tolerated by participants in all treatment groups during the study, and the proportion of subjects experiencing 1 or more AEs was comparable between groups receiving the compound of Formula (I) and the placebo group. In addition, there was no observed dose-dependent increase in the occurrence of AEs across the treatment groups.

Cumulative 56-week safety data, including Example 1, are presented in Table 37 through Table 47. The LTE safety analysis was based on randomized participants in Example who entered the present long-term extension study and received at least one dose of study intervention (including a partial dose) during the study period.

TABLE 37
Key Safety Events through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: | 43 | 35 | 43 | 43 | 41 | 43 | 42 | 247 |
| Safety analysis set | | | | | | | | |
| Avg duration of | 15.03 | 37.81 | 45.36 | 50.48 | 50.15 | 49.31 | 50.63 | 47.56 |
| follow-up (weeks) | | | | | | | | |
| Subjects with 1 or more | 0 | 0 | 3 | 2 | 2 | 2 | 1 | 10 |
| treatment-emergent | | | (7.0%) | (4.7%) | (4.9%) | (4.7%) | (2.4%) | (4.0%) |
| adverse events leading | | | | | | | | |
| to discontinuation of | | | | | | | | |
| study agent | | | | | | | | |
| Subjects with 1 or more | | | | | | | | |
| Adverse events | 22 | 23 | 24 | 32 | 32 | 31 | 32 | 174 |
| | (51.2%) | (65.7%) | (55.8%) | (74.4%) | (78.0%) | (72.1%) | (76.2%) | (70.4%) |
| Adverse events (with at least | | | | | | | | |
| 5%) by preferred Term | | | | | | | | |
| Nasopharyngitis | 2 | 9 | 3 | 13 | 8 | 11 | 5 | 49 |
| | (4.7%) | (25.7%) | (7.0%) | (30.2%) | (19.5%) | (25.6%) | (11.9%) | (19.8%) |
| COVID-19 | 5 | 2 | 6 | 6 | 9 | 5 | 7 | 35 |
| | (11.6%) | (5.7%) | (14.0%) | (14.0%) | (22.0%) | (11.6%) | (16.7%) | (14.2%) |
| Upper respiratory tract | 1 | 4 | 7 | 3 | 3 | 2 | 5 | 24 |
| infection | (2.3%) | (11.4%) | (16.3%) | (7.0%) | (7.3%) | (4.7%) | (11.9%) | (9.7%) |
| Bronchitis | 0 | 1 | 2 | 4 | 2 | 0 | 0 | 9 |
| | | (2.9%) | (4.7%) | (9.3%) | (4.9%) | | | (3.6%) |
| Urinary tract infection | 0 | 2 | 2 | 1 | 1 | 0 | 2 | 8 |
| | | (5.7%) | (4.7%) | (2.3%) | (2.4%) | | (4.8%) | (3.2%) |
| Influenza | 1 | 1 | 0 | 1 | 3 | 1 | 1 | 7 |
| | (2.3%) | (2.9%) | | (2.3%) | (7.3%) | (2.3%) | (2.4%) | (2.8%) |
| Diarrhea | 1 | 1 | 2 | 4 | 2 | 1 | 1 | 11 |
| | (2.3%) | (2.9%) | (4.7%) | (9.3%) | (4.9%) | (2.3%) | (2.4%) | (4.5%) |
| Alanine aminotransferase | 0 | 2 | 1 | 1 | 1 | 0 | 4 | 9 |
| increased | | (5.7%) | (2.3%) | (2.3%) | (2.4%) | | (9.5%) | (3.6%) |
| Aspartate aminotransferase | 0 | 1 | 1 | 1 | 1 | 0 | 3 | 7 |
| increased | | (2.9%) | (2.3%) | (2.3%) | (2.4%) | | (7.1%) | (2.8%) |
| Arthralgia | 0 | 1 | 1 | 1 | 0 | 3 | 2 | 8 |
| | | (2.9%) | (2.3%) | (2.3%) | | (7.0%) | (4.8%) | (3.2%) |
| Headache | 1 | 0 | 2 | 1 | 3 | 5 | 1 | 12 |
| | (2.3%) | | (4.7%) | (2.3%) | (7.3%) | (11.6%) | (2.4%) | (4.9%) |
| Cough | 0 | 0 | 1 | 2 | 0 | 3 | 2 | 8 |
| | | | (2.3%) | (4.7%) | | (7.0%) | (4.8%) | (3.2%) |
| Serious adverse events | 0 | 1 | 0 | 3 | 3 | 3 | 1 | 11 |
| (by preferred term) | | (2.9%) | | (7.0%) | (7.3%) | (7.0%) | (2.4%) | (4.5%) |
| Coronary artery disease | 0 | 0 | 0 | 1 | 0 | 0 | 0 | 1 |
| | | | | (2.3%) | | | | (0.4%) |
| Ventricular dysfunction | 0 | 0 | 0 | 0 | 1 | 0 | 0 | 1 |
| | | | | | (2.4%) | | | (0.4%) |
| COVID-19 | 0 | 0 | 0 | 0 | 0 | 1 | 0 | 1 |
| | | | | | | (2.3%) | | (0.4%) |
| Diverticulitis | 0 | 0 | 0 | 0 | 0 | 1 | 0 | 1 |
| | | | | | | (2.3%) | | (0.4%) |
| Infected cyst | 0 | 0 | 0 | 1 | 0 | 0 | 0 | 1 |
| | | | | (2.3%) | | | | (0.4%) |
| Foot deformity | 0 | 0 | 0 | 1 | 0 | 0 | 0 | 1 |
| | | | | (2.3%) | | | | (0.4%) |
| Intervertebral disc protrusion | 0 | 0 | 0 | 0 | 0 | 1 | 0 | 1 |
| | | | | | | (2.3%) | | (0.4%) |
| Non-cardiac chest pain | 0 | 0 | 0 | 1 | 0 | 0 | 0 | 1 |
| | | | | (2.3%) | | | | (0.4%) |
| Ligament injury | 0 | 0 | 0 | 0 | 1 | 0 | 0 | 1 |
| | | | | | (2.4%) | | | (0.4%) |
| Uterine leiomyoma | 0 | 0 | 0 | 0 | 0 | 0 | 1 | 1 |
| | | | | | | | (2.4%) | (0.4%) |
| Cerebrovascular accident | 0 | 1 | 0 | 0 | 0 | 0 | 0 | 1 |
| | | (2.9%) | | | | | | (0.4%) |
| Suicide attempt | 0 | 0 | 0 | 0 | 0 | 1 | 0 | 1 |
| | | | | | | (2.3%) | | (0.4%) |
| Tonsillar hypertrophy | 0 | 0 | 0 | 0 | 1 | 0 | 0 | 1 |
| | | | | | (2.4%) | | | (0.4%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 38
Number of Subjects with 1 or More Treatment-Emergent AEs Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: | 43 | 35 | 43 | 43 | 41 | 43 | 42 | 247 |
| Safety analysis set | | | | | | | | |
| Avg duration of | 15.03 | 37.81 | 45.36 | 50.48 | 50.15 | 49.31 | 50.63 | 47.56 |
| follow-up (weeks) | | | | | | | | |
| Subjects with 1 or more | 22 | 23 | 24 | 32 | 32 | 31 | 32 | 174 |
| treatment-emergent | (51.2%) | (65.7%) | (55.8%) | (74.4%) | (78.0%) | (72.1%) | (76.2%) | (70.4%) |
| adverse events | | | | | | | | |
| System organ class | | | | | | | | |
| Preferred term | | | | | | | | |
| Infections and infestations | 12 | 16 | 18 | 23 | 27 | 23 | 20 | 127 |
| | (27.9%) | (45.7%) | (41.9%) | (53.5%) | (65.9%) | (53.5%) | (47.6%) | (51.4%) |
| Nasopharyngitis | 2 | 9 | 3 | 13 | 8 | 11 | 5 | 49 |
| | (4.7%) | (25.7%) | (7.0%) | (30.2%) | (19.5%) | (25.6%) | (11.9%) | (19.8%) |
| COVID-19 | 5 | 2 | 6 | 6 | 9 | 5 | 7 | 35 |
| | (11.6%) | (5.7%) | (14.0%) | (14.0%) | (22.0%) | (11.6%) | (16.7%) | (14.2%) |
| Upper respiratory tract | 1 | 4 | 7 | 3 | 3 | 2 | 5 | 24 |
| infection | (2.3%) | (11.4%) | (16.3%) | (7.0%) | (7.3%) | (4.7%) | (11.9%) | (9.7%) |
| Bronchitis | 0 | 1 | 2 | 4 | 2 | 0 | 0 | 9 |
| | | (2.9%) | (4.7%) | (9.3%) | (4.9%) | | | (3.6%) |
| Urinary tract infection | 0 | 2 | 2 | 1 | 1 | 0 | 2 | 8 |
| | | (5.7%) | (4.7%) | (2.3%) | (2.4%) | | (4.8%) | (3.2%) |
| Influenza | 1 | 1 | 0 | 1 | 3 | 1 | 1 | 7 |
| | (2.3%) | (2.9%) | | (2.3%) | (7.3%) | (2.3%) | (2.4%) | (2.8%) |
| Sinusitis | 0 | 0 | 0 | 2 | 2 | 1 | 0 | 5 |
| | | | | (4.7%) | (4.9%) | (2.3%) | | (2.0%) |
| Tooth infection | 0 | 0 | 0 | 1 | 2 | 1 | 0 | 4 |
| | | | | (2.3%) | (4.9%) | (2.3%) | | (1.6%) |
| Cellulitis | 0 | 0 | 2 | 0 | 0 | 1 | 0 | 3 |
| | | | (4.7%) | | | (2.3%) | | (1.2%) |
| Gastroenteritis | 0 | 1 | 0 | 1 | 1 | 0 | 0 | 3 |
| | | (2.9%) | | (2.3%) | (2.4%) | | | (1.2%) |
| Pharyngitis | 0 | 0 | 0 | 2 | 0 | 1 | 0 | 3 |
| | | | | (4.7%) | | (2.3%) | | (1.2%) |
| Respiratory tract infection | 0 | 0 | 1 | 0 | 1 | 0 | 1 | 3 |
| | | | (2.3%) | | (2.4%) | | (2.4%) | (1.2%) |
| Acute sinusitis | 0 | 0 | 0 | 0 | 0 | 0 | 2 | 2 |
| | | | | | | | (4.8%) | (0.8%) |
| Conjunctivitis | 0 | 0 | 1 | 0 | 0 | 1 | 0 | 2 |
| | | | (2.3%) | | | (2.3%) | | (0.8%) |
| Hordcolum | 0 | 0 | 1 | 0 | 0 | 1 | 0 | 2 |
| | | | (2.3%) | | | (2.3%) | | (0.8%) |
| Oral herpes | 0 | 1 | 0 | 1 | 0 | 0 | 0 | 2 |
| | | (2.9%) | | (2.3%) | | | | (0.8%) |
| Otitis media | 0 | 0 | 1 | 0 | 1 | 0 | 0 | 2 |
| | | | (2.3%) | | (2.4%) | | | (0.8%) |
| Paronychia | 0 | 0 | 1 | 0 | 0 | 0 | 1 | 2 |
| | | | (2.3%) | | | | (2.4%) | (0.8%) |
| Pulpitis dental | 0 | 0 | 0 | 0 | 0 | 1 | 1 | 2 |
| | | | | | | (2.3%) | (2.4%) | (0.8%) |
| Skin candida | 0 | 0 | 0 | 2 | 0 | 0 | 0 | 2 |
| | | | | (4.7%) | | | | (0.8%) |
| Tooth abscess | 0 | 0 | 0 | 0 | 0 | 0 | 2 | 2 |
| | | | | | | | (4.8%) | (0.8%) |
| Balanitis candida | 0 | 0 | 0 | 0 | 0 | 0 | 1 | 1 |
| | | | | | | | (2.4%) | (0.4%) |
| Chlamydial infection | 0 | 0 | 0 | 0 | 0 | 1 | 0 | 1 |
| | | | | | | (2.3%) | | (0.4%) |
| Cystitis | 0 | 0 | 0 | 0 | 0 | 0 | 1 | 1 |
| | | | | | | | (2.4%) | (0.4%) |
| Cystitis bacterial | 0 | 0 | 1 | 0 | 0 | 0 | 0 | 1 |
| | | | (2.3%) | | | | | (0.4%) |
| Diverticulitis | 0 | 0 | 0 | 0 | 0 | 1 | 0 | 1 |
| | | | | | | (2.3%) | | (0.4%) |
| Folliculitis | 0 | 0 | 1 | 0 | 0 | 0 | 0 | 1 |
| | | | (2.3%) | | | | | (0.4%) |
| Furuncle | 0 | 1 | 0 | 0 | 0 | 0 | 0 | 1 |
| | | (2.9%) | | | | | | (0.4%) |
| Gastroenteritis viral | 1 | 0 | 0 | 1 | 0 | 0 | 0 | 1 |
| | (2.3%) | | | (2.3%) | | | | (0.4%) |
| Gastrointestinal viral infection | 0 | 0 | 0 | 1 | 0 | 0 | 0 | 1 |
| | | | | (2.3%) | | | | (0.4%) |
| Gingivitis | 0 | 0 | 0 | 1 | 0 | 0 | 0 | 1 |
| | | | | (2.3%) | | | | (0.4%) |
| Hand-foot-and-mouth disease | 0 | 0 | 0 | 1 | 0 | 0 | 0 | 1 |
| | | | | (2.3%) | | | | (0.4%) |
| Helicobacter infection | 0 | 0 | 0 | 0 | 1 | 0 | 0 | 1 |
| | | | | | (2.4%) | | | (0.4%) |
| Herpes zoster | 0 | 0 | 0 | 0 | 0 | 1 | 0 | 1 |
| | | | | | | (2.3%) | | (0.4%) |
| Impetigo | 0 | 0 | 1 | 0 | 0 | 0 | 0 | 1 |
| | | | (2.3%) | | | | | (0.4%) |
| Infected cyst | 0 | 0 | 0 | 1 | 0 | 0 | 0 | 1 |
| | | | | (2.3%) | | | | (0.4%) |
| Otitis media acute | 0 | 0 | 1 | 0 | 0 | 0 | 0 | 1 |
| | | | (2.3%) | | | | | (0.4%) |
| Papilloma viral infection | 0 | 0 | 0 | 1 | 0 | 0 | 0 | 1 |
| | | | | (2.3%) | | | | (0.4%) |
| Pneumonia mycoplasmal | 0 | 0 | 0 | 0 | 0 | 1 | 0 | 1 |
| | | | | | | (2.3%) | | (0.4%) |
| Rhinitis | 0 | 0 | 0 | 0 | 1 | 0 | 0 | 1 |
| | | | | | (2.4%) | | | (0.4%) |
| Subcutaneous abscess | 0 | 0 | 1 | 0 | 0 | 0 | 0 | 1 |
| | | | (2.3%) | | | | | (0.4%) |
| Tonsillitis | 0 | 0 | 0 | 1 | 0 | 0 | 0 | 1 |
| | | | | (2.3%) | | | | (0.4%) |
| Viral upper respiratory tract | 0 | 0 | 1 | 0 | 0 | 0 | 0 | 1 |
| infection | | | (2.3%) | | | | | (0.4%) |
| Abscess limb | 1 | 0 | 0 | 0 | 0 | 0 | 0 | 0 |
| | (2.3%) | | | | | | | |
| Bronchitis bacterial | 1 | 0 | 0 | 0 | 0 | 0 | 0 | 0 |
| | (2.3%) | | | | | | | |
| Gastrointestinal disorders | 5 | 1 | 3 | 9 | 7 | 7 | 7 | 34 |
| | (11.6%) | (2.9%) | (7.0%) | (20.9%) | (17.1%) | (16.3%) | (16.7%) | (13.8%) |
| Diarrhea | 1 | 1 | 2 | 4 | 2 | 1 | 1 | 11 |
| | (2.3%) | (2.9%) | (4.7%) | (9.3%) | (4.9%) | (2.3%) | (2.4%) | (4.5%) |
| Nausea | 0 | 0 | 0 | 1 | 1 | 1 | 1 | 4 |
| | | | | (2.3%) | (2.4%) | (2.3%) | (2.4%) | (1.6%) |
| Abdominal pain | 0 | 0 | 0 | 1 | 0 | 1 | 1 | 3 |
| | | | | (2.3%) | | (2.3%) | (2.4%) | (1.2%) |
| Dyspepsia | 0 | 0 | 0 | 0 | 1 | 2 | 0 | 3 |
| | | | | | (2.4%) | (4.7%) | | (1.2%) |
| Vomiting | 0 | 0 | 0 | 0 | 0 | 2 | 1 | 3 |
| | | | | | | (4.7%) | (2.4%) | (1.2%) |
| Abdominal discomfort | 0 | 0 | 0 | 0 | 0 | 1 | 1 | 2 |
| | | | | | | (2.3%) | (2.4%) | (0.8%) |
| Gastroesophageal reflux | 0 | 0 | 0 | 0 | 1 | 1 | 0 | 2 |
| disease | | | | | (2.4%) | (2.3%) | | (0.8%) |
| Hemorrhoids | 0 | 0 | 1 | 0 | 0 | 1 | 0 | 2 |
| | | | (2.3%) | | | (2.3%) | | (0.8%) |
| Abdominal pain lower | 0 | 0 | 0 | 0 | 0 | 0 | 1 | 1 |
| | | | | | | | (2.4%) | (0.4%) |
| Abdominal pain upper | 1 | 0 | 0 | 0 | 0 | 0 | 1 | 1 |
| | (2.3%) | | | | | | (2.4%) | (0.4%) |
| Dental caries | 0 | 0 | 0 | 0 | 0 | 0 | 1 | 1 |
| | | | | | | | (2.4%) | (0.4%) |
| Diverticulum | 0 | 0 | 1 | 0 | 0 | 0 | 0 | 1 |
| | | | (2.3%) | | | | | (0.4%) |
| Flatulence | 0 | 0 | 0 | 1 | 0 | 0 | 0 | 1 |
| | | | | (2.3%) | | | | (0.4%) |
| Gastritis | 0 | 0 | 0 | 0 | 1 | 0 | 0 | 1 |
| | | | | | (2.4%) | | | (0.4%) |
| Large intestine polyp | 0 | 0 | 0 | 1 | 0 | 0 | 0 | 1 |
| | | | | (2.3%) | | | | (0.4%) |
| Lip pain | 0 | 0 | 0 | 1 | 0 | 0 | 0 | 1 |
| | | | | (2.3%) | | | | (0.4%) |
| Proctalgia | 0 | 0 | 0 | 0 | 1 | 0 | 0 | 1 |
| | | | | | (2.4%) | | | (0.4%) |
| Toothache | 0 | 0 | 0 | 0 | 0 | 1 | 0 | 1 |
| | | | | | | (2.3%) | | (0.4%) |
| Abdominal distension | 1 | 0 | 0 | 0 | 0 | 0 | 0 | 0 |
| | (2.3%) | | | | | | | |
| Constipation | 1 | 0 | 0 | 0 | 0 | 0 | 0 | 0 |
| | (2.3%) | | | | | | | |
| Mouth ulceration | 1 | 0 | 0 | 0 | 0 | 0 | 0 | 0 |
| | (2.3%) | | | | | | | |
| Investigations | 1 | 4 | 4 | 3 | 4 | 4 | 8 | 27 |
| | (2.3%) | (11.4%) | (9.3%) | (7.0%) | (9.8%) | (9.3%) | (19.0%) | (10.9%) |
| Alanine aminotransferase | 0 | 2 | 1 | 1 | 1 | 0 | 4 | 9 |
| increased | | (5.7%) | (2.3%) | (2.3%) | (2.4%) | | (9.5%) | (3.6%) |
| Aspartate aminotransferase | 0 | 1 | 1 | 1 | 1 | 0 | 3 | 7 |
| increased | | (2.9%) | (2.3%) | (2.3%) | (2.4%) | | (7.1%) | (2.8%) |
| Blood creatine phosphokinase | 0 | 0 | 0 | 1 | 2 | 1 | 1 | 5 |
| increased | | | | (2.3%) | (4.9%) | (2.3%) | (2.4%) | (2.0%) |
| Blood pressure increased | 0 | 0 | 0 | 0 | 1 | 1 | 2 | 4 |
| | | | | | (2.4%) | (2.3%) | (4.8%) | (1.6%) |
| Hepatic enzyme increased | 0 | 0 | 1 | 0 | 1 | 1 | 1 | 4 |
| | | | (2.3%) | | (2.4%) | (2.3%) | (2.4%) | (1.6%) |
| Gamma-glutamyltransferase | 0 | 1 | 1 | 0 | 0 | 0 | 1 | 3 |
| increased | | (2.9%) | (2.3%) | | | | (2.4%) | (1.2%) |
| Blood glucose increased | 1 | 0 | 0 | 0 | 1 | 1 | 0 | 2 |
| | (2.3%) | | | | (2.4%) | (2.3%) | | (0.8%) |
| SARS-CoV-2 test positive | 0 | 1 | 0 | 0 | 0 | 0 | 1 | 2 |
| | | (2.9%) | | | | | (2.4%) | (0.8%) |
| Blood cholesterol increased | 0 | 0 | 0 | 0 | 0 | 0 | 1 | 1 |
| | | | | | | | (2.4%) | (0.4%) |
| Blood pressure decreased | 0 | 0 | 0 | 1 | 0 | 0 | 0 | 1 |
| | | | | (2.3%) | | | | (0.4%) |
| Blood triglycerides increased | 0 | 0 | 0 | 0 | 0 | 0 | 1 | 1 |
| | | | | | | | (2.4%) | (0.4%) |
| C-reactive protein increased | 0 | 0 | 0 | 0 | 0 | 1 | 0 | 1 |
| | | | | | | (2.3%) | | (0.4%) |
| Liver function test increased | 0 | 1 | 0 | 0 | 0 | 0 | 0 | 1 |
| | | (2.9%) | | | | | | (0.4%) |
| Neutrophil count decreased | 0 | 0 | 0 | 0 | 0 | 0 | 1 | 1 |
| | | | | | | | (2.4%) | (0.4%) |
| Red blood cells urine positive | 0 | 0 | 0 | 0 | 0 | 0 | 1 | 1 |
| | | | | | | | (2.4%) | (0.4%) |
| Transaminases increased | 0 | 0 | 1 | 0 | 0 | 0 | 0 | 1 |
| | | | (2.3%) | | | | | (0.4%) |
| Urine analysis | 0 | 0 | 0 | 1 | 0 | 0 | 0 | 1 |
| | | | | (2.3%) | | | | (0.4%) |
| Urine leukocyte esterase | 0 | 0 | 0 | 0 | 0 | 0 | 1 | 1 |
| positive | | | | | | | (2.4%) | (0.4%) |
| Weight increased | 0 | 0 | 1 | 0 | 0 | 0 | 0 | 1 |
| | | | (2.3%) | | | | | (0.4%) |
| White blood cells urine positive | 0 | 1 | 0 | 0 | 0 | 0 | 0 | 1 |
| | | (2.9%) | | | | | | (0.4%) |
| Musculoskeletal and | 2 | 2 | 1 | 4 | 2 | 9 | 3 | 21 |
| connective tissue disorders | (4.7%) | (5.7%) | (2.3%) | (9.3%) | (4.9%) | (20.9%) | (7.1%) | (8.5%) |
| Arthralgia | 0 | 1 | 1 | 1 | 0 | 3 | 2 | 8 |
| | | (2.9%) | (2.3%) | (2.3%) | | (7.0%) | (4.8%) | (3.2%) |
| Back pain | 0 | 1 | 0 | 0 | 1 | 1 | 1 | 4 |
| | | (2.9%) | | | (2.4%) | (2.3%) | (2.4%) | (1.6%) |
| Myalgia | 1 | 0 | 0 | 0 | 0 | 2 | 0 | 2 |
| | (2.3%) | | | | | (4.7%) | | (0.8%) |
| Axillary mass | 0 | 0 | 0 | 0 | 0 | 0 | 1 | 1 |
| | | | | | | | (2.4%) | (0.4%) |
| Chondromalacia | 0 | 0 | 0 | 0 | 1 | 0 | 0 | 1 |
| | | | | | (2.4%) | | | (0.4%) |
| Foot deformity | 0 | 0 | 0 | 1 | 0 | 0 | 0 | 1 |
| | | | | (2.3%) | | | | (0.4%) |
| Intervertebral disc degeneration | 0 | 0 | 0 | 0 | 0 | 1 | 0 | 1 |
| | | | | | | (2.3%) | | (0.4%) |
| Intervertebral disc protrusion | 0 | 0 | 0 | 0 | 0 | 1 | 0 | 1 |
| | | | | | | (2.3%) | | (0.4%) |
| Muscle contracture | 0 | 0 | 0 | 0 | 0 | 1 | 0 | 1 |
| | | | | | | (2.3%) | | (0.4%) |
| Neck pain | 0 | 0 | 0 | 1 | 0 | 0 | 0 | 1 |
| | | | | (2.3%) | | | | (0.4%) |
| Osteoarthritis | 0 | 0 | 0 | 0 | 0 | 1 | 0 | 1 |
| | | | | | | (2.3%) | | (0.4%) |
| Pain in extremity | 0 | 0 | 0 | 0 | 0 | 0 | 1 | 1 |
| | | | | | | | (2.4%) | (0.4%) |
| Periarthritis | 0 | 0 | 0 | 0 | 0 | 1 | 0 | 1 |
| | | | | | | (2.3%) | | (0.4%) |
| Rotator cuff syndrome | 0 | 0 | 0 | 1 | 0 | 0 | 0 | 1 |
| | | | | (2.3%) | | | | (0.4%) |
| Spinal pain | 0 | 0 | 0 | 0 | 0 | 1 | 0 | 1 |
| | | | | | | (2.3%) | | (0.4%) |
| Tenosynovitis | 0 | 0 | 0 | 1 | 0 | 0 | 0 | 1 |
| | | | | (2.3%) | | | | (0.4%) |
| Trigger finger | 0 | 0 | 0 | 0 | 0 | 1 | 0 | 1 |
| | | | | | | (2.3%) | | (0.4%) |
| Tendonitis | 1 | 0 | 0 | 0 | 0 | 0 | 0 | 0 |
| | (2.3%) | | | | | | | |
| Nervous system disorders | 1 | 1 | 3 | 3 | 5 | 6 | 3 | 21 |
| | (2.3%) | (2.9%) | (7.0%) | (7.0%) | (12.2%) | (14.0%) | (7.1%) | (8.5%) |
| Headache | 1 | 0 | 2 | 1 | 3 | 5 | 1 | 12 |
| | (2.3%) | | (4.7%) | (2.3%) | (7.3%) | (11.6%) | (2.4%) | (4.9%) |
| Sciatica | 0 | 0 | 0 | 1 | 1 | 1 | 0 | 3 |
| | | | | (2.3%) | (2.4%) | (2.3%) | | (1.2%) |
| Migraine | 0 | 0 | 0 | 1 | 0 | 1 | 0 | 2 |
| | | | | (2.3%) | | (2.3%) | | (0.8%) |
| Burning sensation | 0 | 0 | 0 | 0 | 0 | 0 | 1 | 1 |
| | | | | | | | (2.4%) | (0.4%) |
| Carpal tunnel syndrome | 0 | 0 | 0 | 0 | 0 | 1 | 0 | 1 |
| | | | | | | (2.3%) | | (0.4%) |
| Cerebrovascular accident | 0 | 1 | 0 | 0 | 0 | 0 | 0 | 1 |
| | | (2.9%) | | | | | | (0.4%) |
| Dizziness postural | 0 | 0 | 0 | 0 | 0 | 0 | 1 | 1 |
| | | | | | | | (2.4%) | (0.4%) |
| Dysgeusia | 0 | 0 | 1 | 0 | 0 | 0 | 0 | 1 |
| | | | (2.3%) | | | | | (0.4%) |
| Memory impairment | 0 | 0 | 0 | 0 | 0 | 1 | 0 | 1 |
| | | | | | | (2.3%) | | (0.4%) |
| Paresthesia | 0 | 0 | 0 | 0 | 1 | 0 | 0 | 1 |
| | | | | | (2.4%) | | | (0.4%) |
| Injury, poisoning and | 0 | 3 | 2 | 2 | 4 | 2 | 4 | 17 |
| procedural complications | | (8.6%) | (4.7%) | (4.7%) | (9.8%) | (4.7%) | (9.5%) | (6.9%) |
| Meniscus injury | 0 | 0 | 1 | 0 | 2 | 0 | 0 | 3 |
| | | | (2.3%) | | (4.9%) | | | (1.2%) |
| Chest injury | 0 | 0 | 0 | 0 | 0 | 1 | 0 | 1 |
| | | | | | | (2.3%) | | (0.4%) |
| Concussion | 0 | 1 | 0 | 0 | 0 | 0 | 0 | 1 |
| | | (2.9%) | | | | | | (0.4%) |
| Contusion | 0 | 0 | 0 | 0 | 1 | 0 | 0 | 1 |
| | | | | | (2.4%) | | | (0.4%) |
| Corneal abrasion | 0 | 0 | 0 | 0 | 1 | 0 | 0 | 1 |
| | | | | | (2.4%) | | | (0.4%) |
| Ear injury | 0 | 0 | 0 | 0 | 0 | 0 | 1 | 1 |
| | | | | | | | (2.4%) | (0.4%) |
| Fall | 0 | 1 | 0 | 0 | 0 | 0 | 0 | 1 |
| | | (2.9%) | | | | | | (0.4%) |
| Heat oedema | 0 | 0 | 0 | 1 | 0 | 0 | 0 | 1 |
| | | | | (2.3%) | | | | (0.4%) |
| Ligament injury | 0 | 0 | 0 | 0 | 1 | 0 | 0 | 1 |
| | | | | | (2.4%) | | | (0.4%) |
| Ligament rupture | 0 | 0 | 0 | 1 | 0 | 0 | 0 | 1 |
| | | | | (2.3%) | | | | (0.4%) |
| Ligament sprain | 0 | 0 | 1 | 0 | 0 | 0 | 0 | 1 |
| | | | (2.3%) | | | | | (0.4%) |
| Limb injury | 0 | 1 | 0 | 0 | 0 | 0 | 0 | 1 |
| | | (2.9%) | | | | | | (0.4%) |
| Muscle injury | 0 | 0 | 0 | 0 | 0 | 0 | 1 | 1 |
| | | | | | | | (2.4%) | (0.4%) |
| Procedural pain | 0 | 0 | 0 | 0 | 0 | 0 | 1 | 1 |
| | | | | | | | (2.4%) | (0.4%) |
| Skin laceration | 0 | 0 | 0 | 0 | 0 | 1 | 0 | 1 |
| | | | | | | (2.3%) | | (0.4%) |
| Sunburn | 0 | 0 | 0 | 0 | 1 | 0 | 0 | 1 |
| | | | | | (2.4%) | | | (0.4%) |
| Tooth fracture | 0 | 0 | 0 | 0 | 0 | 0 | 1 | 1 |
| | | | | | | | (2.4%) | (0.4%) |
| Skin and subcutaneous tissue | 4 | 2 | 3 | 4 | 2 | 2 | 3 | 16 |
| disorders | (9.3%) | (5.7%) | (7.0%) | (9.3%) | (4.9%) | (4.7%) | (7.1%) | (6.5%) |
| Dermatitis contact | 0 | 0 | 0 | 1 | 1 | 1 | 1 | 4 |
| | | | | (2.3%) | (2.4%) | (2.3%) | (2.4%) | (1.6%) |
| Psoriasis | 1 | 0 | 0 | 0 | 1 | 0 | 2 | 3 |
| | (2.3%) | | | | (2.4%) | | (4.8%) | (1.2%) |
| Pruritus | 1 | 0 | 0 | 1 | 0 | 1 | 0 | 2 |
| | (2.3%) | | | (2.3%) | | (2.3%) | | (0.8%) |
| Actinic keratosis | 0 | 0 | 0 | 1 | 0 | 0 | 0 | 1 |
| | | | | (2.3%) | | | | (0.4%) |
| Alopecia | 1 | 0 | 1 | 0 | 0 | 0 | 0 | 1 |
| | (2.3%) | | (2.3%) | | | | | (0.4%) |
| Dermal cyst | 0 | 0 | 1 | 0 | 0 | 0 | 0 | 1 |
| | | | (2.3%) | | | | | (0.4%) |
| Dermatitis acneiform | 0 | 1 | 0 | 0 | 0 | 0 | 0 | 1 |
| | | (2.9%) | | | | | | (0.4%) |
| Diffuse alopecia | 0 | 0 | 1 | 0 | 0 | 0 | 0 | 1 |
| | | | (2.3%) | | | | | (0.4%) |
| Hand dermatitis | 0 | 1 | 0 | 0 | 0 | 0 | 0 | 1 |
| | | (2.9%) | | | | | | (0.4%) |
| Intertrigo | 0 | 0 | 0 | 0 | 0 | 0 | 1 | 1 |
| | | | | | | | (2.4%) | (0.4%) |
| Night sweats | 1 | 0 | 0 | 1 | 0 | 0 | 0 | 1 |
| | (2.3%) | | | (2.3%) | | | | (0.4%) |
| Rash pruritic | 1 | 0 | 0 | 0 | 0 | 0 | 0 | 0 |
| | (2.3%) | | | | | | | |
| General disorders and | 2 | 0 | 1 | 3 | 2 | 3 | 4 | 13 |
| administration site conditions | (4.7%) | | (2.3%) | (7.0%) | (4.9%) | (7.0%) | (9.5%) | (5.3%) |
| Fatigue | 1 | 0 | 0 | 1 | 0 | 1 | 2 | 4 |
| | (2.3%) | | | (2.3%) | | (2.3%) | (4.8%) | (1.6%) |
| Oedema peripheral | 1 | 0 | 0 | 1 | 0 | 1 | 2 | 4 |
| | (2.3%) | | | (2.3%) | | (2.3%) | (4.8%) | (1.6%) |
| Chills | 0 | 0 | 0 | 0 | 1 | 1 | 0 | 2 |
| | | | | | (2.4%) | (2.3%) | | (0.8%) |
| Pyrexia | 0 | 0 | 1 | 0 | 0 | 0 | 1 | 2 |
| | | | (2.3%) | | | | (2.4%) | (0.8%) |
| Asthenia | 0 | 0 | 0 | 0 | 1 | 0 | 0 | 1 |
| | | | | | (2.4%) | | | (0.4%) |
| Influenza like illness | 0 | 0 | 0 | 0 | 1 | 0 | 0 | 1 |
| | | | | | (2.4%) | | | (0.4%) |
| Non-cardiac chest pain | 0 | 0 | 0 | 1 | 0 | 0 | 0 | 1 |
| | | | | (2.3%) | | | | (0.4%) |
| Respiratory, thoracic and | 1 | 1 | 1 | 2 | 1 | 4 | 4 | 13 |
| mediastinal disorders | (2.3%) | (2.9%) | (2.3%) | (4.7%) | (2.4%) | (9.3%) | (9.5%) | (5.3%) |
| Cough | 0 | 0 | 1 | 2 | 0 | 3 | 2 | 8 |
| | | | (2.3%) | (4.7%) | | (7.0%) | (4.8%) | (3.2%) |
| Oropharyngeal pain | 0 | 1 | 0 | 0 | 0 | 1 | 0 | 2 |
| | | (2.9%) | | | | (2.3%) | | (0.8%) |
| Asthma | 1 | 0 | 0 | 0 | 0 | 1 | 0 | 1 |
| | (2.3%) | | | | | (2.3%) | | (0.4%) |
| Bronchitis chronic | 0 | 0 | 0 | 0 | 0 | 1 | 0 | 1 |
| | | | | | | (2.3%) | | (0.4%) |
| Chronic obstructive pulmonary | 0 | 0 | 0 | 0 | 0 | 1 | 0 | 1 |
| disease | | | | | | (2.3%) | | (0.4%) |
| Productive cough | 0 | 0 | 0 | 0 | 0 | 0 | 1 | 1 |
| | | | | | | | (2.4%) | (0.4%) |
| Rhinorrhoea | 0 | 0 | 0 | 0 | 0 | 0 | 1 | 1 |
| | | | | | | | (2.4%) | (0.4%) |
| Tonsillar hypertrophy | 0 | 0 | 0 | 0 | 1 | 0 | 0 | 1 |
| | | | | | (2.4%) | | | (0.4%) |
| Metabolism and nutrition | 0 | 1 | 2 | 1 | 3 | 2 | 2 | 11 |
| disorders | | (2.9%) | (4.7%) | (2.3%) | (7.3%) | (4.7%) | (4.8%) | (4.5%) |
| Hyperglycemia | 0 | 0 | 0 | 1 | 0 | 1 | 2 | 4 |
| | | | | (2.3%) | | (2.3%) | (4.8%) | (1.6%) |
| Diabetes mellitus | 0 | 0 | 1 | 0 | 1 | 0 | 0 | 2 |
| | | | (2.3%) | | (2.4%) | | | (0.8%) |
| Decreased appetite | 0 | 0 | 1 | 0 | 0 | 0 | 0 | 1 |
| | | | (2.3%) | | | | | (0.4%) |
| Haemochromatosis | 0 | 1 | 0 | 0 | 0 | 0 | 0 | 1 |
| | | (2.9%) | | | | | | (0.4%) |
| Hypercholesterolemia | 0 | 0 | 0 | 0 | 0 | 1 | 0 | 1 |
| | | | | | | (2.3%) | | (0.4%) |
| Hyperlipidemia | 0 | 0 | 0 | 0 | 1 | 0 | 0 | 1 |
| | | | | | (2.4%) | | | (0.4%) |
| Hypertriglyceridemia | 0 | 0 | 1 | 0 | 0 | 0 | 0 | 1 |
| | | | (2.3%) | | | | | (0.4%) |
| Type 2 diabetes mellitus | 0 | 0 | 0 | 0 | 0 | 0 | 1 | 1 |
| | | | | | | | (2.4%) | (0.4%) |
| Vitamin D deficiency | 0 | 0 | 0 | 0 | 1 | 0 | 0 | 1 |
| | | | | | (2.4%) | | | (0.4%) |
| Renal and urinary disorders | 0 | 2 | 1 | 1 | 0 | 2 | 2 | 8 |
| | | (5.7%) | (2.3%) | (2.3%) | | (4.7%) | (4.8%) | (3.2%) |
| Hematuria | 0 | 1 | 0 | 1 | 0 | 0 | 0 | 2 |
| | | (2.9%) | | (2.3%) | | | | (0.8%) |
| Dysuria | 0 | 1 | 0 | 0 | 0 | 0 | 0 | 1 |
| | | (2.9%) | | | | | | (0.4%) |
| Ketonuria | 0 | 0 | 0 | 0 | 0 | 0 | 1 | 1 |
| | | | | | | | (2.4%) | (0.4%) |
| Leukocyturia | 0 | 0 | 0 | 0 | 0 | 1 | 0 | 1 |
| | | | | | | (2.3%) | | (0.4%) |
| Nephrolithiasis | 0 | 0 | 0 | 0 | 0 | 1 | 0 | 1 |
| | | | | | | (2.3%) | | (0.4%) |
| Proteinuria | 0 | 0 | 0 | 0 | 0 | 0 | 1 | 1 |
| | | | | | | | (2.4%) | (0.4%) |
| Renal mass | 0 | 0 | 1 | 0 | 0 | 0 | 0 | 1 |
| | | | (2.3%) | | | | | (0.4%) |
| Urinary tract disorder | 0 | 0 | 0 | 0 | 0 | 0 | 1 | 1 |
| | | | | | | | (2.4%) | (0.4%) |
| Vascular disorders | 1 | 1 | 0 | 1 | 2 | 1 | 2 | 7 |
| | (2.3%) | (2.9%) | | (2.3%) | (4.9%) | (2.3%) | (4.8%) | (2.8%) |
| Hypertension | 1 | 1 | 0 | 1 | 2 | 1 | 2 | 7 |
| | (2.3%) | (2.9%) | | (2.3%) | (4.9%) | (2.3%) | (4.8%) | (2.8%) |
| Neoplasms benign, malignant | 0 | 0 | 0 | 2 | 1 | 1 | 2 | 6 |
| and unspecified (incl cysts | | | | (4.7%) | (2.4%) | (2.3%) | (4.8%) | (2.4%) |
| and polyps) | | | | | | | | |
| Acrochordon | 0 | 0 | 0 | 0 | 1 | 1 | 0 | 2 |
| | | | | | (2.4%) | (2.3%) | | (0.8%) |
| Skin papilloma | 0 | 0 | 0 | 1 | 0 | 0 | 1 | 2 |
| | | | | (2.3%) | | | (2.4%) | (0.8%) |
| Basal cell carcinoma | 0 | 0 | 0 | 1 | 0 | 0 | 0 | 1 |
| | | | | (2.3%) | | | | (0.4%) |
| Uterine leiomyoma | 0 | 0 | 0 | 0 | 0 | 0 | 1 | 1 |
| | | | | | | | (2.4%) | (0.4%) |
| Blood and lymphatic system | 0 | 0 | 1 | 3 | 0 | 0 | 1 | 5 |
| disorders | | | (2.3%) | (7.0%) | | | (2.4%) | (2.0%) |
| Lymphopenia | 0 | 0 | 0 | 2 | 0 | 0 | 0 | 2 |
| | | | | (4.7%) | | | | (0.8%) |
| Neutropenia | 0 | 0 | 0 | 2 | 0 | 0 | 0 | 2 |
| | | | | (4.7%) | | | | (0.8%) |
| Leukopenia | 0 | 0 | 0 | 1 | 0 | 0 | 0 | 1 |
| | | | | (2.3%) | | | | (0.4%) |
| Thrombocytopenia | 0 | 0 | 1 | 0 | 0 | 0 | 0 | 1 |
| | | | (2.3%) | | | | | (0.4%) |
| Thrombocytosis | 0 | 0 | 0 | 0 | 0 | 0 | 1 | 1 |
| | | | | | | | (2.4%) | (0.4%) |
| Psychiatric disorders | 0 | 0 | 0 | 0 | 1 | 3 | 1 | 5 |
| | | | | | (2.4%) | (7.0%) | (2.4%) | (2.0%) |
| Depression | 0 | 0 | 0 | 0 | 0 | 1 | 1 | 2 |
| | | | | | | (2.3%) | (2.4%) | (0.8%) |
| Insomnia | 0 | 0 | 0 | 0 | 1 | 1 | 0 | 2 |
| | | | | | (2.4%) | (2.3%) | | (0.8%) |
| Libido decreased | 0 | 0 | 0 | 0 | 0 | 1 | 0 | 1 |
| | | | | | | (2.3%) | | (0.4%) |
| Suicide attempt | 0 | 0 | 0 | 0 | 0 | 1 | 0 | 1 |
| | | | | | | (2.3%) | | (0.4%) |
| Cardiac disorders | 0 | 0 | 0 | 1 | 1 | 1 | 1 | 4 |
| | | | | (2.3%) | (2.4%) | (2.3%) | (2.4%) | (1.6%) |
| Atrioventricular block first | 0 | 0 | 0 | 0 | 0 | 1 | 0 | 1 |
| degree | | | | | | (2.3%) | | (0.4%) |
| Bundle branch block right | 0 | 0 | 0 | 0 | 0 | 0 | 1 | 1 |
| | | | | | | | (2.4%) | (0.4%) |
| Coronary artery disease | 0 | 0 | 0 | 1 | 0 | 0 | 0 | 1 |
| | | | | (2.3%) | | | | (0.4%) |
| Ventricular dysfunction | 0 | 0 | 0 | 0 | 1 | 0 | 0 | 1 |
| | | | | | (2.4%) | | | (0.4%) |
| Ear and labyrinth disorders | 0 | 0 | 1 | 0 | 0 | 1 | 1 | 3 |
| | | | (2.3%) | | | (2.3%) | (2.4%) | (1.2%) |
| Vertigo | 0 | 0 | 1 | 0 | 0 | 1 | 0 | 2 |
| | | | (2.3%) | | | (2.3%) | | (0.8%) |
| Hypoacusis | 0 | 0 | 0 | 0 | 0 | 0 | 1 | 1 |
| | | | | | | | (2.4%) | (0.4%) |
| Eye disorders | 0 | 2 | 0 | 0 | 1 | 0 | 0 | 3 |
| | | (5.7%) | | | (2.4%) | | | (1.2%) |
| Eyelid skin dryness | 0 | 0 | 0 | 0 | 1 | 0 | 0 | 1 |
| | | | | | (2.4%) | | | (0.4%) |
| Ocular cyst | 0 | 1 | 0 | 0 | 0 | 0 | 0 | 1 |
| | | (2.9%) | | | | | | (0.4%) |
| Retinal vein occlusion | 0 | 1 | 0 | 0 | 0 | 0 | 0 | 1 |
| | | (2.9%) | | | | | | (0.4%) |
| Hepatobiliary disorders | 1 | 1 | 1 | 0 | 0 | 1 | 0 | 3 |
| | (2.3%) | (2.9%) | (2.3%) | | | (2.3%) | | (1.2%) |
| Cholelithiasis | 0 | 0 | 0 | 0 | 0 | 1 | 0 | 1 |
| | | | | | | (2.3%) | | (0.4%) |
| Hypertransaminasemia | 0 | 1 | 0 | 0 | 0 | 0 | 0 | 1 |
| | | (2.9%) | | | | | | (0.4%) |
| Steatohepatitis | 0 | 0 | 1 | 0 | 0 | 0 | 0 | 1 |
| | | | (2.3%) | | | | | (0.4%) |
| Liver disorder | 1 | 0 | 0 | 0 | 0 | 0 | 0 | 0 |
| | (2.3%) | | | | | | | |
| Immune system disorders | 0 | 0 | 0 | 1 | 0 | 0 | 1 | 2 |
| | | | | (2.3%) | | | (2.4%) | (0.8%) |
| Seasonal allergy | 0 | 0 | 0 | 1 | 0 | 0 | 1 | 2 |
| | | | | (2.3%) | | | (2.4%) | (0.8%) |
| Endocrine disorders | 0 | 0 | 0 | 0 | 1 | 0 | 0 | 1 |
| | | | | | (2.4%) | | | (0.4%) |
| Goiter | 0 | 0 | 0 | 0 | 1 | 0 | 0 | 1 |
| | | | | | (2.4%) | | | (0.4%) |
| Social circumstances | 0 | 0 | 0 | 1 | 0 | 0 | 0 | 1 |
| | | | | (2.3%) | | | | (0.4%) |
| Menopause | 0 | 0 | 0 | 1 | 0 | 0 | 0 | 1 |
| | | | | (2.3%) | | | | (0.4%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 39
Number of Subjects with 1 or more Treatment-emergent AEs with Frequency ≥5%

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: | 43 | 35 | 43 | 43 | 41 | 43 | 42 | 247 |
| Safety analysis set | | | | | | | | |
| Avg duration of | 15.03 | 37.81 | 45.36 | 50.48 | 50.15 | 49.31 | 50.63 | 47.56 |
| follow-up (weeks) | | | | | | | | |
| Subjects with 1 or more | 22 | 23 | 24 | 32 | 32 | 31 | 32 | 174 |
| treatment-emergent | (51.2%) | (65.7%) | (55.8%) | (74.4%) | (78.0%) | (72.1%) | (76.2%) | (70.4%) |
| adverse events | | | | | | | | |
| System organ class | | | | | | | | |
| Preferred term | | | | | | | | |
| Infections and infestations | 12 | 16 | 18 | 23 | 27 | 23 | 20 | 127 |
| | (27.9%) | (45.7%) | (41.9%) | (53.5%) | (65.9%) | (53.5%) | (47.6%) | (51.4%) |
| Nasopharyngitis | 2 | 9 | 3 | 13 | 8 | 11 | 5 | 49 |
| | (4.7%) | (25.7%) | (7.0%) | (30.2%) | (19.5%) | (25.6%) | (11.9%) | (19.8%) |
| COVID-19 | 5 | 2 | 6 | 6 | 9 | 5 | 7 | 35 |
| | (11.6%) | (5.7%) | (14.0%) | (14.0%) | (22.0%) | (11.6%) | (16.7%) | (14.2%) |
| Upper respiratory tract | 1 | 4 | 7 | 3 | 3 | 2 | 5 | 24 |
| infection | (2.3%) | (11.4%) | (16.3%) | (7.0%) | (7.3%) | (4.7%) | (11.9%) | (9.7%) |
| Bronchitis | 0 | 1 | 2 | 4 | 2 | 0 | 0 | 9 |
| | | (2.9%) | (4.7%) | (9.3%) | (4.9%) | | | (3.6%) |
| Urinary tract infection | 0 | 2 | 2 | 1 | 1 | 0 | 2 | 8 |
| | | (5.7%) | (4.7%) | (2.3%) | (2.4%) | | (4.8%) | (3.2%) |
| Influenza | 1 | 1 | 0 | 1 | 3 | 1 | 1 | 7 |
| | (2.3%) | (2.9%) | | (2.3%) | (7.3%) | (2.3%) | (2.4%) | (2.8%) |
| Gastrointestinal disorders | 5 | 1 | 3 | 9 | 7 | 7 | 7 | 34 |
| | (11.6%) | (2.9%) | (7.0%) | (20.9%) | (17.1%) | (16.3%) | (16.7%) | (13.8%) |
| Diarrhea | 1 | 1 | 2 | 4 | 2 | 1 | 1 | 11 |
| | (2.3%) | (2.9%) | (4.7%) | (9.3%) | (4.9%) | (2.3%) | (2.4%) | (4.5%) |
| Investigations | 1 | 4 | 4 | 3 | 4 | 4 | 8 | 27 |
| | (2.3%) | (11.4%) | (9.3%) | (7.0%) | (9.8%) | (9.3%) | (19.0%) | (10.9%) |
| Alanine aminotransferase | 0 | 2 | 1 | 1 | 1 | 0 | 4 | 9 |
| increased | | (5.7%) | (2.3%) | (2.3%) | (2.4%) | | (9.5%) | (3.6%) |
| Aspartate aminotransferase | 0 | 1 | 1 | 1 | 1 | 0 | 3 | 7 |
| increased | | (2.9%) | (2.3%) | (2.3%) | (2.4%) | | (7.1%) | (2.8%) |
| Musculoskeletal and | 2 | 2 | 1 | 4 | 2 | 9 | 3 | 21 |
| connective tissue disorders | (4.7%) | (5.7%) | (2.3%) | (9.3%) | (4.9%) | (20.9%) | (7.1%) | (8.5%) |
| Arthralgia | 0 | 1 | 1 | 1 | 0 | 3 | 2 | 8 |
| | | (2.9%) | (2.3%) | (2.3%) | | (7.0%) | (4.8%) | (3.2%) |
| Nervous system disorders | 1 | 1 | 3 | 3 | 5 | 6 | 3 | 21 |
| | (2.3%) | (2.9%) | (7.0%) | (7.0%) | (12.2%) | (14.0%) | (7.1%) | (8.5%) |
| Headache | 1 | 0 | 2 | 1 | 3 | 5 | 1 | 12 |
| | (2.3%) | | (4.7%) | (2.3%) | (7.3%) | (11.6%) | (2.4%) | (4.9%) |
| Respiratory, thoracic and | 1 | 1 | 1 | 2 | 1 | 4 | 4 | 13 |
| mediastinal disorders | (2.3%) | (2.9%) | (2.3%) | (4.7%) | (2.4%) | (9.3%) | (9.5%) | (5.3%) |
| Cough | 0 | 0 | 1 | 2 | 0 | 3 | 2 | 8 |
| | | | (2.3%) | (4.7%) | | (7.0%) | (4.8%) | (3.2%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 40
Number of Subjects with 1 or More Treatment-emergent Serious AE Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: | 43 | 35 | 43 | 43 | 41 | 43 | 42 | 247 |
| Safety analysis set | | | | | | | | |
| Avg duration of | 15.03 | 37.81 | 45.36 | 50.48 | 50.15 | 49.31 | 50.63 | 47.56 |
| follow-up (weeks) | | | | | | | | |
| Subjects with 1 or more | 0 | 1 | 0 | 3 | 3 | 3 | 1 | 11 |
| treatment-emergent serious | | (2.9%) | | (7.0%) | (7.3%) | (7.0%) | (2.4%) | (4.5%) |
| adverse events | | | | | | | | |
| System organ class | | | | | | | | |
| Preferred term | | | | | | | | |
| Cardiac disorders | 0 | 0 | 0 | 1 | 1 | 0 | 0 | 2 |
| | | | | (2.3%) | (2.4%) | | | (0.8%) |
| Coronary artery disease | 0 | 0 | 0 | 1 | 0 | 0 | 0 | 1 |
| | | | | (2.3%) | | | | (0.4%) |
| Ventricular dysfunction | 0 | 0 | 0 | 0 | 1 | 0 | 0 | 1 |
| | | | | | (2.4%) | | | (0.4%) |
| Infections and infestations | 0 | 0 | 0 | 1 | 0 | 1 | 0 | 2 |
| | | | | (2.3%) | | (2.3%) | | (0.8%) |
| COVID-19 | 0 | 0 | 0 | 0 | 0 | 1 | 0 | 1 |
| | | | | | | (2.3%) | | (0.4%) |
| Diverticulitis | 0 | 0 | 0 | 0 | 0 | 1 | 0 | 1 |
| | | | | | | (2.3%) | | (0.4%) |
| Infected cyst | 0 | 0 | 0 | 1 | 0 | 0 | 0 | 1 |
| | | | | (2.3%) | | | | (0.4%) |
| Musculoskeletal and | 0 | 0 | 0 | 1 | 0 | 1 | 0 | 2 |
| connective tissue disorders | | | | (2.3%) | | (2.3%) | | (0.8%) |
| Foot deformity | 0 | 0 | 0 | 1 | 0 | 0 | 0 | 1 |
| | | | | (2.3%) | | | | (0.4%) |
| Intervertebral disc protrusion | 0 | 0 | 0 | 0 | 0 | 1 | 0 | 1 |
| | | | | | | (2.3%) | | (0.4%) |
| General disorders and | 0 | 0 | 0 | 1 | 0 | 0 | 0 | 1 |
| administration site conditions | | | | (2.3%) | | | | (0.4%) |
| Non-cardiac chest pain | 0 | 0 | 0 | 1 | 0 | 0 | 0 | 1 |
| | | | | (2.3%) | | | | (0.4%) |
| Injury, poisoning and | 0 | 0 | 0 | 0 | 1 | 0 | 0 | 1 |
| procedural complications | | | | | (2.4%) | | | (0.4%) |
| Ligament injury | 0 | 0 | 0 | 0 | 1 | 0 | 0 | 1 |
| | | | | | (2.4%) | | | (0.4%) |
| Neoplasms benign, malignant | 0 | 0 | 0 | 0 | 0 | 0 | 1 | 1 |
| and unspecified (incl cysts | | | | | | | (2.4%) | (0.4%) |
| and polyps) | | | | | | | | |
| Uterine leiomyoma | 0 | 0 | 0 | 0 | 0 | 0 | 1 | 1 |
| | | | | | | | (2.4%) | (0.4%) |
| Nervous system disorders | 0 | 1 | 0 | 0 | 0 | 0 | 0 | 1 |
| | | (2.9%) | | | | | | (0.4%) |
| Cerebrovascular accident | 0 | 1 | 0 | 0 | 0 | 0 | 0 | 1 |
| | | (2.9%) | | | | | | (0.4%) |
| Psychiatric disorders | 0 | 0 | 0 | 0 | 0 | 1 | 0 | 1 |
| | | | | | | (2.3%) | | (0.4%) |
| Suicide attempt | 0 | 0 | 0 | 0 | 0 | 1 | 0 | 1 |
| | | | | | | (2.3%) | | (0.4%) |
| Respiratory, thoracic and | 0 | 0 | 0 | 0 | 1 | 0 | 0 | 1 |
| mediastinal disorders | | | | | (2.4%) | | | (0.4%) |
| Tonsillar hypertrophy | 0 | 0 | 0 | 0 | 1 | 0 | 0 | 1 |
| | | | | | (2.4%) | | | (0.4%) |

^{a}Only placebo crossover subjects were included in the Placebo → 100 mg QD column after crossover to treatment with the compound of Formula (I).
^{b} Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 41
Number of Subjects With 1 or More Treatment-emergent AEs Leading to Discontinuation Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety analysis set | 43 | 35 | 43 | 43 | 41 | 43 | 42 | 247 |
| Avg duration of follow-up (weeks) | 15.03 | 37.81 | 45.36 | 50.48 | 50.15 | 49.31 | 50.63 | 47.56 |
| Subjects with 1 or more treatment- | 0 | 0 | 3 | 2 | 2 | 2 | 1 | 10 |
| emergent adverse events leading to | | | (7.0%) | (4.7%) | (4.9%) | (4.7%) | (2.4%) | (4.0%) |
| discontinuation of study agent | | | | | | | | |
| System organ class | | | | | | | | |
| Preferred term | | | | | | | | |
| Gastrointestinal disorders | 0 | 0 | 0 | 2 | 1 | 1 | 1 | 5 |
| | | | | (4.7%) | (2.4%) | (2.3%) | (2.4%) | (2.0%) |
| Abdominal discomfort | 0 | 0 | 0 | 0 | 0 | 0 | 1 | 1 |
| | | | | | | | (2.4%) | (0.4%) |
| Abdominal pain | 0 | 0 | 0 | 1 | 0 | 0 | 0 | 1 |
| | | | | (2.3%) | | | | (0.4%) |
| Nausea | 0 | 0 | 0 | 1 | 0 | 0 | 0 | 1 |
| | | | | (2.3%) | | | | (0.4%) |
| Proctalgia | 0 | 0 | 0 | 0 | 1 | 0 | 0 | 1 |
| | | | | | (2.4%) | | | (0.4%) |
| Vomiting | 0 | 0 | 0 | 0 | 0 | 1 | 0 | 1 |
| | | | | | | (2.3%) | | (0.4%) |
| Investigations | 0 | 0 | 3 | 0 | 0 | 0 | 0 | 3 |
| | | | (7.0%) | | | | | (1.2%) |
| Alanine aminotransferase increased | 0 | 0 | 1 | 0 | 0 | 0 | 0 | 1 |
| | | | (2.3%) | | | | | (0.4%) |
| Aspartate aminotransferase increased | 0 | 0 | 1 | 0 | 0 | 0 | 0 | 1 |
| | | | (2.3%) | | | | | (0.4%) |
| Gamma-glutamyltransferase | 0 | 0 | 1 | 0 | 0 | 0 | 0 | 1 |
| increased | | | (2.3%) | | | | | (0.4%) |
| Transaminases increased | 0 | 0 | 1 | 0 | 0 | 0 | 0 | 1 |
| | | | (2.3%) | | | | | (0.4%) |
| Weight increased | 0 | 0 | 1 | 0 | 0 | 0 | 0 | 1 |
| | | | (2.3%) | | | | | (0.4%) |
| General disorders and | 0 | 0 | 0 | 0 | 1 | 1 | 0 | 2 |
| administration site conditions | | | | | (2.4%) | (2.3%) | | (0.8%) |
| Chills | 0 | 0 | 0 | 0 | 1 | 1 | 0 | 2 |
| | | | | | (2.4%) | (2.3%) | | (0.8%) |
| Asthenia | 0 | 0 | 0 | 0 | 1 | 0 | 0 | 1 |
| | | | | | (2.4%) | | | (0.4%) |
| Nervous system disorders | 0 | 0 | 0 | 0 | 1 | 1 | 0 | 2 |
| | | | | | (2.4%) | (2.3%) | | (0.8%) |
| Headache | 0 | 0 | 0 | 0 | 1 | 1 | 0 | 2 |
| | | | | | (2.4%) | (2.3%) | | (0.8%) |
| Psychiatric disorders | 0 | 0 | 0 | 0 | 1 | 1 | 0 | 2 |
| | | | | | (2.4%) | (2.3%) | | (0.8%) |
| Insomnia | 0 | 0 | 0 | 0 | 1 | 0 | 0 | 1 |
| | | | | | (2.4%) | | | (0.4%) |
| Suicide attempt | 0 | 0 | 0 | 0 | 0 | 1 | 0 | 1 |
| | | | | | | (2.3%) | | (0.4%) |
| Ear and labyrinth disorders | 0 | 0 | 0 | 0 | 0 | 1 | 0 | 1 |
| | | | | | | (2.3%) | | (0.4%) |
| Vertigo | 0 | 0 | 0 | 0 | 0 | 1 | 0 | 1 |
| | | | | | | (2.3%) | | (0.4%) |
| Skin and subcutaneous tissue | 0 | 0 | 0 | 0 | 1 | 0 | 0 | 1 |
| disorders | | | | | (2.4%) | | | (0.4%) |
| Psoriasis | 0 | 0 | 0 | 0 | 1 | 0 | 0 | 1 |
| | | | | | (2.4%) | | | (0.4%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 42
Number of Subjects With Selected Treatment-emergent AEs
Associated with COVID-19 Infection Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety | 43 | 35 | 43 | 43 | 41 | 43 | 42 | 247 |
| analysis set | | | | | | | | |
| Avg duration of follow-up | 15.03 | 37.81 | 45.36 | 50.48 | 50.15 | 49.31 | 50.63 | 47.56 |
| (weeks) | | | | | | | | |
| Subjects with 1 or more | 5 | 3 | 6 | 6 | 9 | 5 | 8 | 37 |
| COVID-19 associated | (11.6%) | (8.6%) | (14.0%) | (14.0%) | (22.0%) | (11.6%) | (19.0%) | (15.0%) |
| adverse events | | | | | | | | |
| Special interest category | | | | | | | | |
| Preferred term | | | | | | | | |
| COVID-19 associated | | | | | | | | |
| COVID-19 | 5 | 2 | 6 | 6 | 9 | 5 | 7 | 35 |
| | (11.6%) | (5.7%) | (14.0%) | (14.0%) | (22.0%) | (11.6%) | (16.7%) | (14.2%) |
| SARS-CoV-2 test positive | 0 | 1 | 0 | 0 | 0 | 0 | 1 | 2 |
| | | (2.9%) | | | | | (2.4%) | (0.8%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 43
Number of Subjects with Post-Baseline Hematology
Measurements by Max CTCAE Grade Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety | 43 | 35 | 43 | 43 | 41 | 43 | 42 | 247 |
| analysis set | | | | | | | | |
| Hemoglobin | | | | | | | | |
| (g/L) (increased) | | | | | | | | |
| N | 43 | 35 | 42 | 43 | 41 | 42 | 42 | 245 |
| Subjects with postbaseline | 0 | 2 | 1 | 0 | 1 | 0 | 1 | 5 |
| maximum toxicity grade >0 | | (5.7%) | (2.4%) | | (2.4%) | | (2.4%) | (2.0%) |
| Subjects with Grade 1 | 0 | 2 | 1 | 0 | 1 | 0 | 1 | 5 |
| | | (5.7%) | (2.4%) | | (2.4%) | | (2.4%) | (2.0%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Hemoglobin | | | | | | | | |
| (g/L) (decreased) | | | | | | | | |
| N | 43 | 35 | 42 | 43 | 41 | 42 | 42 | 245 |
| Subjects with postbaseline | 3 | 1 | 4 | 5 | 0 | 2 | 3 | 15 |
| maximum toxicity grade >0 | (7.0%) | (2.9%) | (9.5%) | (11.6%) | | (4.8%) | (7.1%) | (6.1%) |
| Subjects with Grade 1 | 3 | 1 | 4 | 3 | 0 | 2 | 3 | 13 |
| | (7.0%) | (2.9%) | (9.5%) | (7.0%) | | (4.8%) | (7.1%) | (5.3%) |
| Subjects with Grade 2 | 0 | 0 | 0 | 2 | 0 | 0 | 0 | 2 |
| | | | | (4.7%) | | | | (0.8%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Lymphocytes | | | | | | | | |
| (×10⁹/L) (increased) | | | | | | | | |
| N | 43 | 35 | 42 | 43 | 41 | 42 | 42 | 245 |
| Subjects with postbaseline | 0 | 0 | 0 | 0 | 2 | 2 | 0 | 4 |
| maximum toxicity grade >0 | | | | | (4.9%) | (4.8%) | | (1.6%) |
| Subjects with Grade 1 | 0 | 0 | 0 | 0 | 0 | 0 | 0 | 0 |
| Subjects with Grade 2 | 0 | 0 | 0 | 0 | 2 | 2 | 0 | 4 |
| | | | | | (4.9%) | (4.8%) | | (1.6%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Lymphocytes | | | | | | | | |
| (×10⁹/L) (decreased) | | | | | | | | |
| N | 43 | 35 | 42 | 43 | 41 | 42 | 42 | 245 |
| Subjects with postbaseline | 5 | 1 | 0 | 2 | 7 | 4 | 2 | 16 |
| maximum toxicity grade >0 | (11.6%) | (2.9%) | | (4.7%) | (17.1%) | (9.5%) | (4.8%) | (6.5%) |
| Subjects with Grade 1 | 2 | 0 | 0 | 1 | 3 | 2 | 2 | 8 |
| | (4.7%) | | | (2.3%) | (7.3%) | (4.8%) | (4.8%) | (3.3%) |
| Subjects with Grade 2 | 2 | 1 | 0 | 1 | 4 | 2 | 0 | 8 |
| | (4.7%) | (2.9%) | | (2.3%) | (9.8%) | (4.8%) | | (3.3%) |
| Subjects with Grade 3 | 1 | 0 | 0 | 0 | 0 | 0 | 0 | 0 |
| | (2.3%) | | | | | | | |
| Subjects with Grade 4 | 0 | 0 | 0 | 0 | 0 | 0 | 0 | 0 |
| Neutrophils | | | | | | | | |
| (×10⁹/L) (decreased) | | | | | | | | |
| N | 43 | 0 | 42 | 43 | 41 | 42 | 42 | 210 |
| Subjects with postbaseline | 1 | — | 6 | 3 | 3 | 4 | 3 | 19 |
| maximum toxicity grade >0 | (2.3%) | | (14.3%) | (7.0%) | (7.3%) | (9.5%) | (7.1%) | (9.0%) |
| Subjects with Grade 1 | 0 | — | 6 | 2 | 2 | 3 | 2 | 15 |
| | | | (14.3%) | (4.7%) | (4.9%) | (7.1%) | (4.8%) | (7.1%) |
| Subjects with Grade 2 | 1 | — | 0 | 1 | 1 | 1 | 1 | 4 |
| | (2.3%) | | | (2.3%) | (2.4%) | (2.4%) | (2.4%) | (1.9%) |
| Subjects with Grade 3 | 0 | — | 0 | 0 | 0 | 0 | 0 | 0 |
| Subjects with Grade 4 | 0 | — | 0 | 0 | 0 | 0 | 0 | 0 |
| Platelets | | | | | | | | |
| (×10⁹/L) (decreased) | | | | | | | | |
| N | 43 | 35 | 42 | 43 | 41 | 42 | 42 | 245 |
| Subjects with postbaseline | 1 | 2 | 5 | 0 | 1 | 2 | 1 | 11 |
| maximum toxicity grade >0 | (2.3%) | (5.7%) | (11.9%) | | (2.4%) | (4.8%) | (2.4%) | (4.5%) |
| Subjects with Grade 1 | 1 | 2 | 5 | 0 | 1 | 2 | 1 | 11 |
| | (2.3%) | (5.7%) | (11.9%) | | (2.4%) | (4.8%) | (2.4%) | (4.5%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| WBC | | | | | | | | |
| (×10⁹/L) (increased) | | | | | | | | |
| N | 43 | 35 | 42 | 43 | 41 | 42 | 42 | 245 |
| Subjects with postbaseline | 0 | 0 | 0 | 0 | 0 | 0 | 0 | 0 |
| maximum toxicity grade >0 | | | | | | | | |
| Subjects with Grade 1 | 0 | 0 | 0 | 0 | 0 | 0 | 0 | 0 |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| WBC | | | | | | | | |
| (×10⁹/L) (decreased) | | | | | | | | |
| N | 43 | 35 | 42 | 43 | 41 | 42 | 42 | 245 |
| Subjects with postbaseline | 3 | 2 | 4 | 6 | 6 | 5 | 3 | 26 |
| maximum toxicity grade >0 | (7.0%) | (5.7%) | (9.5%) | (14.0%) | (14.6%) | (11.9%) | (7.1%) | (10.6%) |
| Subjects with Grade 1 | 2 | 2 | 4 | 4 | 5 | 4 | 3 | 22 |
| | (4.7%) | (5.7%) | (9.5%) | (9.3%) | (12.2%) | (9.5%) | (7.1%) | (9.0%) |
| Subjects with Grade 2 | 1 | 0 | 0 | 2 | 1 | 1 | 0 | 4 |
| | (2.3%) | | | (4.7%) | (2.4%) | (2.4%) | | (1.6%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).

TABLE 44
Number of Subjects with Post-Baseline Chemistry Measurements by Max CTCAE Grade Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety analysis | 43 | 35 | 43 | 43 | 41 | 43 | 42 | 247 |
| set | | | | | | | | |
| Alanine Aminotransferase | | | | | | | | |
| (U/L) (increased) | | | | | | | | |
| N | 43 | 35 | 42 | 43 | 41 | 42 | 42 | 245 |
| Subjects with postbaseline | 4 | 11 | 9 | 6 | 16 | 13 | 14 | 69 |
| maximum toxicity grade >0 | (9.3%) | (31.4%) | (21.4%) | (14.0%) | (39.0%) | (31.0%) | (33.3%) | (28.2%) |
| Subjects with Grade 1 | 4 | 10 | 7 | 6 | 15 | 12 | 10 | 60 |
| | (9.3%) | (28.6%) | (16.7%) | (14.0%) | (36.6%) | (28.6%) | (23.8%) | (24.5%) |
| Subjects with Grade 2 | 0 | 1 | 1 | 0 | 1 | 1 | 4 | 8 |
| | | (2.9%) | (2.4%) | | (2.4%) | (2.4%) | (9.5%) | (3.3%) |
| Subjects with Grade 3 | 0 | 0 | 1 | 0 | 0 | 0 | 0 | 1 |
| | | | (2.4%) | | | | | (0.4%) |
| Subjects with Grade 4 | 0 | 0 | 0 | 0 | 0 | 0 | 0 | 0 |
| Albumin | | | | | | | | |
| (g/L) (decreased) | | | | | | | | |
| N | 43 | 35 | 42 | 43 | 41 | 42 | 42 | 245 |
| Subjects with postbaseline | 0 | 0 | 0 | 0 | 0 | 0 | 0 | 0 |
| maximum toxicity grade >0 | | | | | | | | |
| Subjects with Grade 1 | 0 | 0 | 0 | 0 | 0 | 0 | 0 | 0 |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Alkaline Phosphatase | | | | | | | | |
| (U/L) (increased) | | | | | | | | |
| N | 43 | 35 | 42 | 43 | 41 | 42 | 42 | 245 |
| Subjects with postbaseline | 2 | 3 | 3 | 4 | 6 | 2 | 0 | 18 |
| maximum toxicity grade >0 | (4.7%) | (8.6%) | (7.1%) | (9.3%) | (14.6%) | (4.8%) | | (7.3%) |
| Subjects with Grade 1 | 2 | 3 | 3 | 4 | 6 | 2 | 0 | 18 |
| | (4.7%) | (8.6%) | (7.1%) | (9.3%) | (14.6%) | (4.8%) | | (7.3%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Aspartate Aminotransferase | | | | | | | | |
| (U/L) (increased) | | | | | | | | |
| N | 43 | 35 | 42 | 43 | 41 | 42 | 42 | 245 |
| Subjects with postbaseline | 6 | 9 | 6 | 8 | 12 | 10 | 9 | 54 |
| maximum toxicity grade >0 | (14.0%) | (25.7%) | (14.3%) | (18.6%) | (29.3%) | (23.8%) | (21.4%) | (22.0%) |
| Subjects with Grade 1 | 6 | 7 | 5 | 8 | 11 | 9 | 7 | 47 |
| | (14.0%) | (20.0%) | (11.9%) | (18.6%) | (26.8%) | (21.4%) | (16.7%) | (19.2%) |
| Subjects with Grade 2 | 0 | 1 | 1 | 0 | 1 | 1 | 1 | 5 |
| | | (2.9%) | (2.4%) | | (2.4%) | (2.4%) | (2.4%) | (2.0%) |
| Subjects with Grade 3 | 0 | 1 | 0 | 0 | 0 | 0 | 1 | 2 |
| | | (2.9%) | | | | | (2.4%) | (0.8%) |
| Subjects with Grade 4 | 0 | 0 | 0 | 0 | 0 | 0 | 0 | 0 |
| Calcium corrected | | | | | | | | |
| (mmol/L) (increased) | | | | | | | | |
| N | 43 | 35 | 42 | 43 | 41 | 42 | 42 | 245 |
| Subjects with postbaseline | 0 | 0 | 0 | 0 | 0 | 1 | 0 | 1 |
| maximum toxicity grade >0 | | | | | | (2.4%) | | (0.4%) |
| Subjects with Grade 1 | 0 | 0 | 0 | 0 | 0 | 1 | 0 | 1 |
| | | | | | | (2.4%) | | (0.4%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Calcium corrected | | | | | | | | |
| (mmol/L) (decreased) | | | | | | | | |
| N | 43 | 35 | 42 | 43 | 41 | 42 | 42 | 245 |
| Subjects with postbaseline | 0 | 2 | 3 | 2 | 1 | 1 | 2 | 11 |
| maximum toxicity grade >0 | | (5.7%) | (7.1%) | (4.7%) | (2.4%) | (2.4%) | (4.8%) | (4.5%) |
| Subjects with Grade 1 | 0 | 1 | 3 | 2 | 1 | 1 | 1 | 9 |
| | | (2.9%) | (7.1%) | (4.7%) | (2.4%) | (2.4%) | (2.4%) | (3.7%) |
| Subjects with Grade 2 | 0 | 1 | 0 | 0 | 0 | 0 | 1 | 2 |
| | | (2.9%) | | | | | (2.4%) | (0.8%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Creatinine | | | | | | | | |
| (μmol/L) (increased) | | | | | | | | |
| N | 43 | 35 | 42 | 43 | 41 | 42 | 42 | 245 |
| Subjects with postbaseline | 0 | 1 | 2 | 1 | 0 | 1 | 3 | 8 |
| maximum toxicity grade >0 | | (2.9%) | (4.8%) | (2.3%) | | (2.4%) | (7.1%) | (3.3%) |
| Subjects with Grade 1 | 0 | 1 | 2 | 1 | 0 | 1 | 2 | 7 |
| | | (2.9%) | (4.8%) | (2.3%) | | (2.4%) | (4.8%) | (2.9%) |
| Subjects with Grade 2 | 0 | 0 | 0 | 0 | 0 | 0 | 1 | 1 |
| | | | | | | | (2.4%) | (0.4%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Cholesterol | | | | | | | | |
| (mmol/L) (increased) | | | | | | | | |
| N | 41 | 33 | 39 | 43 | 41 | 42 | 41 | 239 |
| Subjects with postbaseline | 3 | 0 | 1 | 3 | 3 | 2 | 5 | 14 |
| maximum toxicity grade >0 | (7.3%) | | (2.6%) | (7.0%) | (7.3%) | (4.8%) | (12.2%) | (5.9%) |
| Subjects with Grade 1 | 2 | 0 | 1 | 3 | 3 | 0 | 5 | 12 |
| | (4.9%) | | (2.6%) | (7.0%) | (7.3%) | | (12.2%) | (5.0%) |
| Subjects with Grade 2 | 1 | 0 | 0 | 0 | 0 | 2 | 0 | 2 |
| | (2.4%) | | | | | (4.8%) | | (0.8%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Creatine Kinase | | | | | | | | |
| (Enzyme U/L) (increased) | | | | | | | | |
| N | 43 | 35 | 42 | 43 | 41 | 42 | 42 | 245 |
| Subjects with postbaseline | 7 | 8 | 9 | 17 | 21 | 13 | 15 | 83 |
| maximum toxicity grade >0 | (16.3%) | (22.9%) | (21.4%) | (39.5%) | (51.2%) | (31.0%) | (35.7%) | (33.9%) |
| Subjects with Grade 1 | 6 | 7 | 5 | 12 | 17 | 12 | 10 | 63 |
| | (14.0%) | (20.0%) | (11.9%) | (27.9%) | (41.5%) | (28.6%) | (23.8%) | (25.7%) |
| Subjects with Grade 2 | 0 | 0 | 2 | 4 | 1 | 0 | 3 | 10 |
| | | | (4.8%) | (9.3%) | (2.4%) | | (7.1%) | (4.1%) |
| Subjects with Grade 3 | 1 | 1 | 1 | 0 | 1 | 0 | 1 | 4 |
| | (2.3%) | (2.9%) | (2.4%) | | (2.4%) | | (2.4%) | (1.6%) |
| Subjects with Grade 4 | 0 | 0 | 1 | 1 | 2 | 1 | 1 | 6 |
| | | | (2.4%) | (2.3%) | (4.9%) | (2.4%) | (2.4%) | (2.4%) |
| Gamma Glutamyl | | | | | | | | |
| Transferase | | | | | | | | |
| (U/L) (increased) | | | | | | | | |
| N | 43 | 35 | 42 | 43 | 41 | 42 | 42 | 245 |
| Subjects with postbaseline | 1 | 5 | 5 | 7 | 7 | 5 | 8 | 37 |
| maximum toxicity grade >0 | (2.3%) | (14.3%) | (11.9%) | (16.3%) | (17.1%) | (11.9%) | (19.0%) | (15.1%) |
| Subjects with Grade 1 | 1 | 3 | 5 | 7 | 7 | 5 | 8 | 35 |
| | (2.3%) | (8.6%) | (11.9%) | (16.3%) | (17.1%) | (11.9%) | (19.0%) | (14.3%) |
| Subjects with Grade 2 | 0 | 2 | 0 | 0 | 0 | 0 | 0 | 2 |
| | | (5.7%) | | | | | | (0.8%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Glucose | | | | | | | | |
| (mmol/L) (decreased) | | | | | | | | |
| N | 43 | 35 | 42 | 43 | 41 | 42 | 42 | 245 |
| Subjects with postbaseline | 4 | 2 | 2 | 1 | 4 | 4 | 3 | 16 |
| maximum toxicity grade >0 | (9.3%) | (5.7%) | (4.8%) | (2.3%) | (9.8%) | (9.5%) | (7.1%) | (6.5%) |
| Subjects with Grade 1 | 4 | 1 | 2 | 1 | 4 | 3 | 3 | 14 |
| | (9.3%) | (2.9%) | (4.8%) | (2.3%) | (9.8%) | (7.1%) | (7.1%) | (5.7%) |
| Subjects with Grade 2 | 0 | 1 | 0 | 0 | 0 | 1 | 0 | 2 |
| | | (2.9%) | | | | (2.4%) | | (0.8%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Magnesium | | | | | | | | |
| (mmol/L) (increased) | | | | | | | | |
| N | 43 | 35 | 42 | 43 | 41 | 42 | 42 | 245 |
| Subjects with postbaseline | 4 | 2 | 4 | 2 | 1 | 2 | 1 | 12 |
| maximum toxicity grade >0 | (9.3%) | (5.7%) | (9.5%) | (4.7%) | (2.4%) | (4.8%) | (2.4%) | (4.9%) |
| Subjects with Grade 1 | 4 | 2 | 4 | 2 | 1 | 2 | 1 | 12 |
| | (9.3%) | (5.7%) | (9.5%) | (4.7%) | (2.4%) | (4.8%) | (2.4%) | (4.9%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Magnesium | | | | | | | | |
| (mmol/L) (decreased) | | | | | | | | |
| N | 43 | 35 | 42 | 43 | 41 | 42 | 42 | 245 |
| Subjects with postbaseline | 1 | 1 | 0 | 0 | 0 | 1 | 0 | 2 |
| maximum toxicity grade >0 | (2.3%) | (2.9%) | | | | (2.4%) | | (0.8%) |
| Subjects with Grade 1 | 1 | 1 | 0 | 0 | 0 | 1 | 0 | 2 |
| | (2.3%) | (2.9%) | | | | (2.4%) | | (0.8%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Potassium | | | | | | | | |
| (mmol/L) (increased) | | | | | | | | |
| N | 43 | 35 | 42 | 43 | 41 | 42 | 42 | 245 |
| Subjects with postbaseline | 4 | 3 | 7 | 3 | 3 | 1 | 5 | 22 |
| maximum toxicity grade >0 | (9.3%) | (8.6%) | (16.7%) | (7.0%) | (7.3%) | (2.4%) | (11.9%) | (9.0%) |
| Subjects with Grade 1 | 4 | 1 | 6 | 2 | 3 | 0 | 5 | 17 |
| | (9.3%) | (2.9%) | (14.3%) | (4.7%) | (7.3%) | | (11.9%) | (6.9%) |
| Subjects with Grade 2 | 0 | 2 | 1 | 0 | 0 | 1 | 0 | 4 |
| | | (5.7%) | (2.4%) | | | (2.4%) | | (1.6%) |
| Subjects with Grade 3 | 0 | 0 | 0 | 1 | 0 | 0 | 0 | 1 |
| | | | | (2.3%) | | | | (0.4%) |
| Subjects with Grade 4 | 0 | 0 | 0 | 0 | 0 | 0 | 0 | 0 |
| Potassium | | | | | | | | |
| (mmol/L) (decreased) | | | | | | | | |
| N | 43 | 35 | 42 | 43 | 41 | 42 | 42 | 245 |
| Subjects with postbaseline | 0 | 1 | 0 | 0 | 0 | 2 | 0 | 3 |
| maximum toxicity grade >0 | | (2.9%) | | | | (4.8%) | | (1.2%) |
| Subjects with Grade 1 | 0 | 0 | 0 | 0 | 0 | 0 | 0 | 0 |
| Subjects with Grade 2 | 0 | 1 | 0 | 0 | 0 | 2 | 0 | 3 |
| | | (2.9%) | | | | (4.8%) | | (1.2%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Sodium | | | | | | | | |
| (mmol/L) (increased) | | | | | | | | |
| N | 43 | 35 | 42 | 43 | 41 | 42 | 42 | 245 |
| Subjects with postbaseline | 2 | 0 | 1 | 0 | 1 | 2 | 2 | 6 |
| maximum toxicity grade >0 | (4.7%) | | (2.4%) | | (2.4%) | (4.8%) | (4.8%) | (2.4%) |
| Subjects with Grade 1 | 2 | 0 | 1 | 0 | 1 | 2 | 2 | 6 |
| | (4.7%) | | (2.4%) | | (2.4%) | (4.8%) | (4.8%) | (2.4%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Sodium | | | | | | | | |
| (mmol/L) (decreased) | | | | | | | | |
| N | 43 | 35 | 42 | 43 | 41 | 42 | 42 | 245 |
| Subjects with postbaseline | 0 | 0 | 1 | 0 | 0 | 0 | 0 | 1 |
| maximum toxicity grade >0 | | | (2.4%) | | | | | (0.4%) |
| Subjects with Grade 1 | 0 | 0 | 1 | 0 | 0 | 0 | 0 | 1 |
| | | | (2.4%) | | | | | (0.4%) |
| Subjects with Grade 2 | 0 | 0 | 0 | 0 | 0 | 0 | 0 | 0 |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Total Bilirubin | | | | | | | | |
| (μmol/L) (increased) | | | | | | | | |
| N | 43 | 35 | 42 | 43 | 41 | 42 | 42 | 245 |
| Subjects with postbaseline | 5 | 4 | 1 | 5 | 2 | 7 | 3 | 22 |
| maximum toxicity grade >0 | (11.6%) | (11.4%) | (2.4%) | (11.6%) | (4.9%) | (16.7%) | (7.1%) | (9.0%) |
| Subjects with Grade 1 | 5 | 4 | 1 | 4 | 2 | 7 | 3 | 21 |
| | (11.6%) | (11.4%) | (2.4%) | (9.3%) | (4.9%) | (16.7%) | (7.1%) | (8.6%) |
| Subjects with Grade 2 | 0 | 0 | 0 | 1 | 0 | 0 | 0 | 1 |
| | | | | (2.3%) | | | | (0.4%) |
| Subjects with Grade 3 | 0 | 0 | 0 | 0 | 0 | 0 | 0 | 0 |
| Subjects with Grade 4 | 0 | 0 | 0 | 0 | 0 | 0 | 0 | 0 |
| Triglycerides | | | | | | | | |
| (mmol/L) (increased) | | | | | | | | |
| N | 41 | 33 | 39 | 43 | 41 | 42 | 41 | 239 |
| Subjects with postbaseline | 2 | 4 | 8 | 3 | 8 | 5 | 9 | 37 |
| maximum toxicity grade >0 | (4.9%) | (12.1%) | (20.5%) | (7.0%) | (19.5%) | (11.9%) | (22.0%) | (15.5%) |
| Subjects with Grade 1 | 2 | 2 | 3 | 0 | 4 | 2 | 2 | 13 |
| | (4.9%) | (6.1%) | (7.7%) | | (9.8%) | (4.8%) | (4.9%) | (5.4%) |
| Subjects with Grade 2 | 0 | 2 | 4 | 2 | 3 | 3 | 6 | 20 |
| | | (6.1%) | (10.3%) | (4.7%) | (7.3%) | (7.1%) | (14.6%) | (8.4%) |
| Subjects with Grade 3 | 0 | 0 | 1 | 1 | 0 | 0 | 1 | 3 |
| | | | (2.6%) | (2.3%) | | | (2.4%) | (1.3%) |
| Subjects with Grade 4 | 0 | 0 | 0 | 0 | 1 | 0 | 0 | 1 |
| | | | | | (2.4%) | | | (0.4%) |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).

TABLE 45
Summary of Highest Post-baseline Elevated Liver Function Tests Through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety analysis | 43 | 35 | 43 | 43 | 41 | 43 | 42 | 247 |
| set | | | | | | | | |
| ALT (U/L) | | | | | | | | |
| Subjects with at least | | | | | | | | |
| 1 post-baseline value | | | | | | | | |
| N | 10 | 33 | 42 | 43 | 41 | 42 | 42 | 243 |
| ≥20 × ULN | 0 | 0 | 0 | 0 | 0 | 0 | 0 | 0 |
| ≥10 × ULN | 0 | 0 | 0 | 0 | 0 | 0 | 0 | 0 |
| ≥5 × ULN | 0 | 0 | 1 | 0 | 0 | 0 | 0 | 1 |
| | | | (2.4%) | | | | | (0.4%) |
| ≥3 × ULN | 0 | 1 | 2 | 0 | 1 | 3 | 5 | 12 |
| | | (3.0%) | (4.8%) | | (2.4%) | (7.1%) | (11.9%) | (4.9%) |
| >1 × ULN | 2 | 13 | 7 | 7 | 18 | 14 | 11 | 70 |
| | (20.0%) | (39.4%) | (16.7%) | (16.3%) | (43.9%) | (33.3%) | (26.2%) | (28.8%) |
| Subjects with non-missing | | | | | | | | |
| baseline ≤ULN and have at | | | | | | | | |
| least 1 post-baseline value | | | | | | | | |
| N | 8 | 30 | 39 | 41 | 36 | 34 | 36 | 216 |
| ≥20 × ULN | 0 | 0 | 0 | 0 | 0 | 0 | 0 | 0 |
| ≥10 × ULN | 0 | 0 | 0 | 0 | 0 | 0 | 0 | 0 |
| ≥5 × ULN | 0 | 0 | 1 | 0 | 0 | 0 | 0 | 1 |
| | | | (2.6%) | | | | | (0.5%) |
| ≥3 × ULN | 0 | 1 | 1 | 0 | 1 | 1 | 2 | 6 |
| | | (3.3%) | (2.6%) | | (2.8%) | (2.9%) | (5.6%) | (2.8%) |
| >1 × ULN | 0 | 10 | 6 | 5 | 14 | 8 | 8 | 51 |
| | | (33.3%) | (15.4%) | (12.2%) | (38.9%) | (23.5%) | (22.2%) | (23.6%) |
| AST (U/L) | | | | | | | | |
| Subjects with at least | | | | | | | | |
| 1 post-baseline value | | | | | | | | |
| N | 11 | 32 | 42 | 43 | 41 | 42 | 42 | 242 |
| ≥20 × ULN | 0 | 0 | 0 | 0 | 0 | 0 | 0 | 0 |
| ≥10 × ULN | 0 | 0 | 0 | 0 | 0 | 0 | 0 | 0 |
| ≥5 × ULN | 0 | 1 | 0 | 0 | 0 | 0 | 1 | 2 |
| | | (3.1%) | | | | | (2.4%) | (0.8%) |
| ≥3 × ULN | 0 | 2 | 1 | 0 | 1 | 2 | 2 | 8 |
| | | (6.3%) | (2.4%) | | (2.4%) | (4.8%) | (4.8%) | (3.3%) |
| >1 × ULN | 3 | 7 | 6 | 8 | 11 | 9 | 6 | 47 |
| | (27.3%) | (21.9%) | (14.3%) | (18.6%) | (26.8%) | (21.4%) | (14.3%) | (19.4%) |
| Subjects with non-missing | | | | | | | | |
| baseline ≤ULN and have at | | | | | | | | |
| least 1 post-baseline value | | | | | | | | |
| N | 11 | 30 | 40 | 43 | 39 | 37 | 38 | 227 |
| ≥20 × ULN | 0 | 0 | 0 | 0 | 0 | 0 | 0 | 0 |
| ≥10 × ULN | 0 | 0 | 0 | 0 | 0 | 0 | 0 | 0 |
| ≥5 × ULN | 0 | 1 | 0 | 0 | 0 | 0 | 1 | 2 |
| | | (3.3%) | | | | | (2.6%) | (0.9%) |
| ≥3 × ULN | 0 | 1 | 1 | 0 | 1 | 1 | 1 | 5 |
| | | (3.3%) | (2.5%) | | (2.6%) | (2.7%) | (2.6%) | (2.2%) |
| >1 × ULN | 3 | 6 | 5 | 8 | 9 | 7 | 4 | 39 |
| | (27.3%) | (20.0%) | (12.5%) | (18.6%) | (23.1%) | (18.9%) | (10.5%) | (17.2%) |
| Combined ALT/AST | | | | | | | | |
| and total bilirubin | | | | | | | | |
| Subjects with at least one | | | | | | | | |
| post-baseline measurement | | | | | | | | |
| of total bilirubin and ALT | | | | | | | | |
| or AST | | | | | | | | |
| N | 43 | 35 | 42 | 43 | 41 | 42 | 42 | 245 |
| Total Bilirubin >2 × ULN and | 0 | 0 | 0 | 0 | 0 | 0 | 0 | 0 |
| either AST or ALT ≥3 × ULN | | | | | | | | |
| at the same timepoint | | | | | | | | |

Key:
ALT = alanine aminotransferase,
AST = aspartate aminotransferase,
ULN = upper limit of normal
^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).
Note:
The maximum post-baseline value is used to determine the elevation category.

TABLE 46
Summary of Lipid Panel Laboratory Values and Change from Baseline at Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: Safety | 35 | 43 | 43 | 41 | 43 | 42 | 247 |
| analysis set | | | | | | | |
| LDL Cholesterol | | | | | | | |
| (mmol/L) | | | | | | | |
| Baseline | | | | | | | |
| N | 35 | 42 | 43 | 39 | 43 | 40 | 242 |
| Mean (SD) | 2.976 | 3.131 | 3.187 | 3.019 | 3.009 | 3.117 | 3.076 |
| | (0.9422) | (0.7935) | (0.9624) | (0.7264) | (0.9043) | (0.7366) | (0.8444) |
| Median | 2.840 | 3.010 | 3.210 | 2.950 | 3.030 | 3.020 | 3.020 |
| Range | (1.75; | (1.50; | (1.05; | (1.86; | (1.23; | (1.45; | (1.05; |
| | 5.43) | 4.77) | 5.46) | 5.12) | 5.12) | 4.61) | 5.46) |
| IQ range | (2.250; | (2.560; | (2.510; | (2.560; | (2.280; | (2.545; | (2.510; |
| | 3.390) | 3.560) | 3.830) | 3.360) | 3.530) | 3.750) | 3.550) |
| Change from baseline | | | | | | | |
| at Week 52^{b} | | | | | | | |
| N | 30 | 29 | 36 | 31 | 34 | 35 | 195 |
| Mean (SD) | −0.138 | 0.069 | −0.146 | −0.155 | −0.147 | −0.056 | −0.098 |
| | (0.6240) | (0.6705) | (0.7819) | (0.7165) | (0.5834) | (0.6541) | (0.6721) |
| Median | −0.212 | 0.132 | −0.095 | −0.105 | −0.060 | −0.110 | −0.105 |
| Range | (−1.24; | (−1.69; | (−2.68; | (−2.38; | (−2.07; | (−1.51; | (−2.68; |
| | 1.73) | 1.32) | 0.80) | 0.94) | 0.86) | 1.69) | 1.73) |
| IQ range | (−0.573; | (−0.233; | (−0.454; | (−0.389; | (−0.390; | (−0.487; | (−0.416; |
| | 0.170) | 0.350) | 0.417) | 0.280) | 0.180) | 0.460) | 0.350) |
| HDL Cholesterol | | | | | | | |
| (mmol/L) | | | | | | | |
| Baseline | | | | | | | |
| N | 35 | 43 | 43 | 40 | 43 | 41 | 245 |
| Mean (SD) | 1.469 | 1.348 | 1.366 | 1.269 | 1.303 | 1.270 | 1.335 |
| | (0.4341) | (0.3620) | (0.4371) | (0.2844) | (0.3938) | (0.3357) | (0.3797) |
| Median | 1.470 | 1.320 | 1.330 | 1.280 | 1.230 | 1.160 | 1.270 |
| Range | (0.72; | (0.85; | (0.63; | (0.72; | (0.85; | (0.76; | (0.63; |
| | 2.45) | 2.44) | 2.41) | 2.12) | 3.18) | 2.22) | 3.18) |
| IQ range | (1.100; | (1.070; | (1.010; | (1.115; | (1.060; | (1.030; | (1.070; |
| | 1.720) | 1.510) | 1.680) | 1.355) | 1.440) | 1.490) | 1.510) |
| Change from baseline | | | | | | | |
| at Week 52^{b} | | | | | | | |
| N | 30 | 29 | 36 | 31 | 34 | 35 | 195 |
| Mean (SD) | 0.013 | −0.034 | −0.009 | 0.011 | −0.023 | −0.027 | −0.012 |
| | (0.2686) | (0.2282) | (0.2586) | (0.1538) | (0.1415) | (0.2386) | (0.2185) |
| Median | −0.035 | −0.023 | 0.014 | −0.010 | −0.027 | −0.020 | −0.020 |
| Range | (−0.53; | (−0.76; | (−0.86; | (−0.31; | (−0.32; | (−1.00; | (−1.00; |
| | 0.93) | 0.39) | 0.52) | 0.33) | 0.21) | 0.31) | 0.93) |
| IQ range | (−0.128; | (−0.130; | (−0.095; | (−0.090; | (−0.120; | (−0.090; | (−0.110; |
| | 0.100) | 0.100) | 0.135) | 0.130) | 0.110) | 0.060) | 0.110) |
| Triglycerides | | | | | | | |
| (mmol/L) | | | | | | | |
| Baseline | | | | | | | |
| N | 35 | 43 | 43 | 40 | 43 | 41 | 245 |
| Mean (SD) | 1.453 | 1.633 | 1.516 | 1.651 | 1.705 | 1.904 | 1.648 |
| | (0.6671) | (1.0231) | (0.7157) | (0.9195) | (1.0418) | (1.6130) | (1.0484) |
| Median | 1.470 | 1.300 | 1.520 | 1.355 | 1.330 | 1.340 | 1.370 |
| Range | (0.61; | (0.58; | (0.51; | (0.63; | (0.45; | (0.61; | (0.45; |
| | 3.38) | 5.22) | 3.15) | 4.31) | 4.65) | 6.99) | 6.99) |
| IQ range | (0.890; | (0.940; | (0.880; | (1.025; | (1.000; | (0.970; | (0.970; |
| | 1.800) | 1.950) | 1.980) | 2.140) | 2.230) | 1.990) | 1.980) |
| Change from baseline | | | | | | | |
| at Week 52^{b} | | | | | | | |
| N | 30 | 29 | 36 | 31 | 34 | 35 | 195 |
| Mean (SD) | 0.207 | 0.034 | 0.156 | 0.144 | 0.024 | 0.137 | 0.118 |
| | (0.6517) | (1.0772) | (0.9698) | (1.6524) | (0.9283) | (1.0467) | (1.0797) |
| Median | 0.035 | 0.040 | 0.067 | 0.040 | −0.005 | 0.010 | 0.020 |
| Range | (−0.78; | (−2.93; | (−1.10; | (−1.86; | (−2.67; | (−3.06; | (−3.06; |
| | 2.25) | 3.10) | 3.51) | 8.05) | 2.10) | 4.06) | 8.05) |
| IQ range | (−0.104; | (−0.220; | (−0.668; | (−0.550; | (−0.270; | (−0.280; | (−0.290; |
| | 0.270) | 0.540) | 0.650) | 0.360) | 0.520) | 0.420) | 0.520) |

^{a}Includes all columns of treatment with the compound of Formula (I) (Placebo → 100 mg QD, 25 mg QD, 50 mg QD, 25 mg BID, 100 mg QD, and 100 mg BID).
^{b}Only placebo crossover subjects were included in the Placebo → 100 mg QD column after crossover to treatment with the compound of Formula (I).
Note:
N for change from baseline is the number of subjects with non-missing values at both baseline and the postbaseline time point.

TABLE 47
Number of Subjects with 1 or More Post-Baseline Most Severe
Suicidal Ideation or Suicidal Behavior through Week 56

| | | Placebo → | 25 mg | 50 mg | 25 mg | 100 mg | 100 mg | |
|---|---|---|---|---|---|---|---|---|
| Treatment Regimen | Placebo | 100 mg QD^{a} | QD | QD | BID | QD | BID | Combined^{b} |
| Analysis set: Safety | 43 | 35 | 43 | 43 | 41 | 43 | 42 | 247 |
| analysis set | | | | | | | | |
| No suicidal ideation or | 43 | 35 | 42 | 43 | 41 | 41 | 42 | 244 |
| behavior | (100.0%) | (100.0%) | (97.7%) | (100.0%) | (100.0%) | (95.3%) | (100.0%) | (98.8%) |
| Suicidal ideation or | 0 | 0 | 0 | 0 | 0 | 1 | 0 | 1 |
| behavior | | | | | | (2.3%) | | (0.4%) |
| Suicidal ideation | 0 | 0 | 0 | 0 | 0 | 0 | 0 | 0 |
| 1 - Wish to be dead | 0 | 0 | 0 | 0 | 0 | 0 | 0 | 0 |
| 2 - Non-specific active | 0 | 0 | 0 | 0 | 0 | 0 | 0 | 0 |
| suicidal thoughts | | | | | | | | |
| 3 - Active suicidal | 0 | 0 | 0 | 0 | 0 | 0 | 0 | 0 |
| ideation with any | | | | | | | | |
| methods (not plan) | | | | | | | | |
| without intent to act | | | | | | | | |
| 4 - Active suicidal | 0 | 0 | 0 | 0 | 0 | 0 | 0 | 0 |
| ideation with some | | | | | | | | |
| intent to act, without | | | | | | | | |
| specific plan | | | | | | | | |
| 5 - Active suicidal | 0 | 0 | 0 | 0 | 0 | 0 | 0 | 0 |
| ideation with specific | | | | | | | | |
| plan and intent | | | | | | | | |
| Suicidal behavior | 0 | 0 | 0 | 0 | 0 | 1 | 0 | 1 |
| | | | | | | (2.3%) | | (0.4%) |
| 6 - Preparatory acts or | 0 | 0 | 0 | 0 | 0 | 0 | 0 | 0 |
| behavior | | | | | | | | |
| 7 - Aborted attempt | 0 | 0 | 0 | 0 | 0 | 0 | 0 | 0 |
| 8 - Interrupted attempt | 0 | 0 | 0 | 0 | 0 | 0 | 0 | 0 |
| 9 - Non-fatal suicide | 0 | 0 | 0 | 0 | 0 | 1 | 0 | 1 |
| attempt | | | | | | (2.3%) | | (0.4%) |
| 10 - Completed suicide | 0 | 0 | 0 | 0 | 0 | 0 | 0 | 0 |

^{a}Only placebo crossover subjects were included in the Placebo → 100 mg QD column after crossover to treatment with the compound of Formula (I).
^{b}Includes all columns of treatment with the compound of Formula (I) (Placebo → 100 mg QD, 25 mg QD, 50 mg QD, 25 mg BID, 100 mg QD, and 100 mg BID).

Key safety events are presented in Table 48 (with supporting information in Table 49 through Table 54). The proportion of participants experiencing 1 or more AEs was 58.600 in the combined group receiving the compound of Formula (I), with no evidence of a dose-dependent increase in the occurrence of specific AEs across the treatment groups (Table 33 and Table 49).

TABLE 48
Key Safety Events

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis set | 35 | 35 | 39 | 40 | 40 | 38 | 227 |
| Avg duration of follow-up (weeks) | 37.81 | 36.58 | 38.44 | 34.95 | 35.88 | 38.62 | 37.02 |
| Subjects who discontinued study | 0 | 1 | 0 | 2 | 1 | 0 | 4 |
| agent because of 1 or more adverse | | (2.9%) | | (5.0%) | (2.5%) | | (1.8%) |
| events | | | | | | | |
| Subjects with 1 or more: | | | | | | | |
| Adverse events | 23 | 18 | 19 | 27 | 27 | 19 | 133 |
| | (65.7%) | (51.4%) | (48.7%) | (67.5%) | (67.5%) | (50.0%) | (58.6%) |

Adverse events with ≥5% frequency in any treatment group

| | | | | | | | |
|---|---|---|---|---|---|---|---|
| Nasopharyngitis | 9 | 3 | 7 | 6 | 11 | 5 | 41 |
| | (25.7%) | (8.6%) | (17.9%) | (15.0%) | (27.5%) | (13.2%) | (18.1%) |
| Upper respiratory tract infection | 4 | 6 | 3 | 3 | 2 | 4 | 22 |
| | (11.4%) | (17.1%) | (7.7%) | (7.5%) | (5.0%) | (10.5%) | (9.7%) |
| COVID-19 | 2 | 1 | 3 | 1 | 2 | 3 | 12 |
| | (5.7%) | (2.9%) | (7.7%) | (2.5%) | (5.0%) | (7.9%) | (5.3%) |
| Influenza | 1 | 0 | 1 | 3 | 1 | 1 | 7 |
| | (2.9%) | | (2.6%) | (7.5%) | (2.5%) | (2.6%) | (3.1%) |
| Urinary tract infection | 2 | 1 | 1 | 1 | 0 | 2 | 7 |
| | (5.7%) | (2.9%) | (2.6%) | (2.5%) | | (5.3%) | (3.1%) |
| Bronchitis | 1 | 1 | 3 | 1 | 0 | 0 | 6 |
| | (2.9%) | (2.9%) | (7.7%) | (2.5%) | | | (2.6%) |
| Sinusitis | 0 | 0 | 1 | 2 | 0 | 0 | 3 |
| | | | (2.6%) | (5.0%) | | | (1.3%) |
| Alanine aminotransferase increased | 2 | 1 | 1 | 0 | 0 | 2 | 6 |
| | (5.7%) | (2.9%) | (2.6%) | | | (5.3%) | (2.6%) |
| Aspartate aminotransferase | 1 | 1 | 1 | 0 | 0 | 2 | 5 |
| increased | (2.9%) | (2.9%) | (2.6%) | | | (5.3%) | (2.2%) |
| Arthralgia | 1 | 0 | 1 | 0 | 2 | 0 | 4 |
| | (2.9%) | | (2.6%) | | (5.0%) | | (1.8%) |
| Vomiting | 0 | 0 | 0 | 0 | 2 | 0 | 2 |
| | | | | | (5.0%) | | (0.9%) |
| Headache | 0 | 2 | 0 | 3 | 3 | 0 | 8 |
| | | (5.7%) | | (7.5%) | (7.5%) | | (3.5%) |
| Meniscus injury | 0 | 1 | 0 | 2 | 0 | 0 | 3 |
| | | (2.9%) | | (5.0%) | | | (1.3%) |
| Hypertension | 1 | 0 | 1 | 2 | 1 | 1 | 6 |
| | (2.9%) | | (2.6%) | (5.0%) | (2.5%) | (2.6%) | (2.6%) |
| Serious adverse events by | 1 | 0 | 2 | 3 | 2 | 1 | 9 |
| | (2.9%) | | (5.1%) | (7.5%) | (5.0%) | (2.6%) | (4.0%) |
| preferred term | | | | | | | |
| Coronary artery disease | 0 | 0 | 1 | 0 | 0 | 0 | 1 |
| | | | (2.6%) | | | | (0.4%) |
| Ventricular dysfunction | 0 | 0 | 0 | 1 | 0 | 0 | 1 |
| | | | | (2.5%) | | | (0.4%) |
| Foot deformity | 0 | 0 | 1 | 0 | 0 | 0 | 1 |
| | | | (2.6%) | | | | (0.4%) |
| Intervertebral disc protrusion | 0 | 0 | 0 | 0 | 1 | 0 | 1 |
| | | | | | (2.5%) | | (0.4%) |
| Non-cardiac chest pain | 0 | 0 | 1 | 0 | 0 | 0 | 1 |
| | | | (2.6%) | | | | (0.4%) |
| Diverticulitis | 0 | 0 | 0 | 0 | 1 | 0 | 1 |
| | | | | | (2.5%) | | (0.4%) |
| Ligament injury | 0 | 0 | 0 | 1 | 0 | 0 | 1 |
| | | | | (2.5%) | | | (0.4%) |
| Uterine leiomyoma | 0 | 0 | 0 | 0 | 0 | 1 | 1 |
| | | | | | | (2.6%) | (0.4%) |
| Cerebrovascular accident | 1 | 0 | 0 | 0 | 0 | 0 | 1 |
| | (2.9%) | | | | | | (0.4%) |
| Tonsillar hypertrophy | 0 | 0 | 0 | 1 | 0 | 0 | 1 |
| | | | | (2.5%) | | | (0.4%) |
| Infections and infestations | 16 | 12 | 16 | 17 | 20 | 14 | 95 |
| | (45.7%) | (34.3%) | (41.0%) | (42.5%) | (50.0%) | (36.8%) | (41.9%) |
| Serious Infections and infestations | 0 | 0 | 0 | 0 | 1 | 0 | 1 |
| | | | | | (2.5%) | | (0.4%) |

^{a}Includes all columns of treatment with the compound of Formula (I) (Placebo → 100 mg QD, 25 mg QD, 50 mg QD, 25 mg BID, 100 mg QD, and 100 mg BID).
Subjects are counted only once for any given event, regardless of the number of times they actually experienced the event. Adverse events are coded using MedDRA Version 25.1.

TABLE 49
Number of Subjects with 1 or More Treatment-emergent AEs from Week 16 through Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis set | 35 | 35 | 39 | 40 | 40 | 38 | 227 |
| Avg duration of follow-up (weeks) | 37.81 | 36.58 | 38.44 | 34.95 | 35.88 | 38.62 | 37.02 |
| Subjects with 1 or more treatment- | 23 | 18 | 19 | 27 | 27 | 19 | 133 |
| emergent adverse events | (65.7%) | (51.4%) | (48.7%) | (67.5%) | (67.5%) | (50.0%) | (58.6%) |
| System organ class | | | | | | | |
| Preferred term | | | | | | | |
| Infections and infestations | 16 | 12 | 16 | 17 | 20 | 14 | 95 |
| | (45.7%) | (34.3%) | (41.0%) | (42.5%) | (50.0%) | (36.8%) | (41.9%) |
| Nasopharyngitis | 9 | 3 | 7 | 6 | 11 | 5 | 41 |
| | (25.7%) | (8.6%) | (17.9%) | (15.0%) | (27.5%) | (13.2%) | (18.1%) |
| Upper respiratory tract infection | 4 | 6 | 3 | 3 | 2 | 4 | 22 |
| | (11.4%) | (17.1%) | (7.7%) | (7.5%) | (5.0%) | (10.5%) | (9.7%) |
| COVID-19 | 2 | 1 | 3 | 1 | 2 | 3 | 12 |
| | (5.7%) | (2.9%) | (7.7%) | (2.5%) | (5.0%) | (7.9%) | (5.3%) |
| Influenza | 1 | 0 | 1 | 3 | 1 | 1 | 7 |
| | (2.9%) | | (2.6%) | (7.5%) | (2.5%) | (2.6%) | (3.1%) |
| Urinary tract infection | 2 | 1 | 1 | 1 | 0 | 2 | 7 |
| | (5.7%) | (2.9%) | (2.6%) | (2.5%) | | (5.3%) | (3.1%) |
| Bronchitis | 1 | 1 | 3 | 1 | 0 | 0 | 6 |
| | (2.9%) | (2.9%) | (7.7%) | (2.5%) | | | (2.6%) |
| Gastroenteritis | 1 | 0 | 1 | 1 | 0 | 0 | 3 |
| | (2.9%) | | (2.6%) | (2.5%) | | | (1.3%) |
| Sinusitis | 0 | 0 | 1 | 2 | 0 | 0 | 3 |
| | | | (2.6%) | (5.0%) | | | (1.3%) |
| Oral herpes | 1 | 0 | 1 | 0 | 0 | 0 | 2 |
| | (2.9%) | | (2.6%) | | | | (0.9%) |
| Otitis media | 0 | 1 | 0 | 1 | 0 | 0 | 2 |
| | | (2.9%) | | (2.5%) | | | (0.9%) |
| Tooth infection | 0 | 0 | 0 | 1 | 1 | 0 | 2 |
| | | | | (2.5%) | (2.5%) | | (0.9%) |
| Acute sinusitis | 0 | 0 | 0 | 0 | 0 | 1 | 1 |
| | | | | | | (2.6%) | (0.4%) |
| Balanitis candida | 0 | 0 | 0 | 0 | 0 | 1 | 1 |
| | | | | | | (2.6%) | (0.4%) |
| Chlamydial infection | 0 | 0 | 0 | 0 | 1 | 0 | 1 |
| | | | | | (2.5%) | | (0.4%) |
| Cystitis | 0 | 0 | 0 | 0 | 0 | 1 | 1 |
| | | | | | | (2.6%) | (0.4%) |
| Cystitis bacterial | 0 | 1 | 0 | 0 | 0 | 0 | 1 |
| | | (2.9%) | | | | | (0.4%) |
| Diverticulitis | 0 | 0 | 0 | 0 | 1 | 0 | 1 |
| | | | | | (2.5%) | | (0.4%) |
| Folliculitis | 0 | 1 | 0 | 0 | 0 | 0 | 1 |
| | | (2.9%) | | | | | (0.4%) |
| Furuncle | 1 | 0 | 0 | 0 | 0 | 0 | 1 |
| | (2.9%) | | | | | | (0.4%) |
| Gastroenteritis viral | 0 | 0 | 1 | 0 | 0 | 0 | 1 |
| | | | (2.6%) | | | | (0.4%) |
| Gastrointestinal viral infection | 0 | 0 | 1 | 0 | 0 | 0 | 1 |
| | | | (2.6%) | | | | (0.4%) |
| Hand-foot-and-mouth disease | 0 | 0 | 1 | 0 | 0 | 0 | 1 |
| | | | (2.6%) | | | | (0.4%) |
| Helicobacter infection | 0 | 0 | 0 | 1 | 0 | 0 | 1 |
| | | | | (2.5%) | | | (0.4%) |
| Herpes zoster | 0 | 0 | 0 | 0 | 1 | 0 | 1 |
| | | | | | (2.5%) | | (0.4%) |
| Impetigo | 0 | 1 | 0 | 0 | 0 | 0 | 1 |
| | | (2.9%) | | | | | (0.4%) |
| Otitis media acute | 0 | 1 | 0 | 0 | 0 | 0 | 1 |
| | | (2.9%) | | | | | (0.4%) |
| Papilloma viral infection | 0 | 0 | 1 | 0 | 0 | 0 | 1 |
| | | | (2.6%) | | | | (0.4%) |
| Paronychia | 0 | 0 | 0 | 0 | 0 | 1 | 1 |
| | | | | | | (2.6%) | (0.4%) |
| Pharyngitis | 0 | 0 | 0 | 0 | 1 | 0 | 1 |
| | | | | | (2.5%) | | (0.4%) |
| Pneumonia mycoplasmal | 0 | 0 | 0 | 0 | 1 | 0 | 1 |
| | | | | | (2.5%) | | (0.4%) |
| Pulpitis dental | 0 | 0 | 0 | 0 | 1 | 0 | 1 |
| | | | | | (2.5%) | | (0.4%) |
| Respiratory tract infection | 0 | 0 | 0 | 0 | 0 | 1 | 1 |
| | | | | | | (2.6%) | (0.4%) |
| Skin candida | 0 | 0 | 1 | 0 | 0 | 0 | 1 |
| | | | (2.6%) | | | | (0.4%) |
| Tonsillitis | 0 | 0 | 1 | 0 | 0 | 0 | 1 |
| | | | (2.6%) | | | | (0.4%) |
| Tooth abscess | 0 | 0 | 0 | 0 | 0 | 1 | 1 |
| | | | | | | (2.6%) | (0.4%) |
| Investigations | 4 | 1 | 3 | 3 | 2 | 4 | 17 |
| | (11.4%) | (2.9%) | (7.7%) | (7.5%) | (5.0%) | (10.5%) | (7.5%) |
| Alanine aminotransferase increased | 2 | 1 | 1 | 0 | 0 | 2 | 6 |
| | (5.7%) | (2.9%) | (2.6%) | | | (5.3%) | (2.6%) |
| Aspartate aminotransferase increased | 1 | 1 | 1 | 0 | 0 | 2 | 5 |
| | (2.9%) | (2.9%) | (2.6%) | | | (5.3%) | (2.2%) |
| Blood creatine phosphokinase | 0 | 0 | 1 | 1 | 1 | 0 | 3 |
| increased | | | (2.6%) | (2.5%) | (2.5%) | | (1.3%) |
| Gamma-glutamyltransferase | 1 | 1 | 0 | 0 | 0 | 1 | 3 |
| increased | (2.9%) | (2.9%) | | | | (2.6%) | (1.3%) |
| Blood glucose increased | 0 | 0 | 0 | 1 | 1 | 0 | 2 |
| | | | | (2.5%) | (2.5%) | | (0.9%) |
| Blood pressure increased | 0 | 0 | 0 | 1 | 1 | 0 | 2 |
| | | | | (2.5%) | (2.5%) | | (0.9%) |
| SARS-CoV-2 test positive | 1 | 0 | 0 | 0 | 0 | 1 | 2 |
| | (2.9%) | | | | | (2.6%) | (0.9%) |
| Blood cholesterol increased | 0 | 0 | 0 | 0 | 0 | 1 | 1 |
| | | | | | | (2.6%) | (0.4%) |
| Blood pressure decreased | 0 | 0 | 1 | 0 | 0 | 0 | 1 |
| | | | (2.6%) | | | | (0.4%) |
| Blood triglycerides increased | 0 | 0 | 0 | 0 | 0 | 1 | 1 |
| | | | | | | (2.6%) | (0.4%) |
| Liver function test increased | 1 | 0 | 0 | 0 | 0 | 0 | 1 |
| | (2.9%) | | | | | | (0.4%) |
| Neutrophil count decreased | 0 | 0 | 0 | 0 | 0 | 1 | 1 |
| | | | | | | (2.6%) | (0.4%) |
| Red blood cells urine positive | 0 | 0 | 0 | 0 | 0 | 1 | 1 |
| | | | | | | (2.6%) | (0.4%) |
| Urine analysis | 0 | 0 | 1 | 0 | 0 | 0 | 1 |
| | | | (2.6%) | | | | (0.4%) |
| White blood cells urine positive | 1 | 0 | 0 | 0 | 0 | 0 | 1 |
| | (2.9%) | | | | | | (0.4%) |
| Musculoskeletal and connective | 2 | 0 | 4 | 1 | 8 | 1 | 16 |
| tissue disorders | (5.7%) | | (10.3%) | (2.5%) | (20.0%) | (2.6%) | (7.0%) |
| Arthralgia | 1 | 0 | 1 | 0 | 2 | 0 | 4 |
| | (2.9%) | | (2.6%) | | (5.0%) | | (1.8%) |
| Back pain | 1 | 0 | 0 | 0 | 0 | 1 | 2 |
| | (2.9%) | | | | | (2.6%) | (0.9%) |
| Chondromalacia | 0 | 0 | 0 | 1 | 0 | 0 | 1 |
| | | | | (2.5%) | | | (0.4%) |
| Foot deformity | 0 | 0 | 1 | 0 | 0 | 0 | 1 |
| | | | (2.6%) | | | | (0.4%) |
| Intervertebral disc degeneration | 0 | 0 | 0 | 0 | 1 | 0 | 1 |
| | | | | | (2.5%) | | (0.4%) |
| Intervertebral disc protrusion | 0 | 0 | 0 | 0 | 1 | 0 | 1 |
| | | | | | (2.5%) | | (0.4%) |
| Muscle contracture | 0 | 0 | 0 | 0 | 1 | 0 | 1 |
| | | | | | (2.5%) | | (0.4%) |
| Myalgia | 0 | 0 | 0 | 0 | 1 | 0 | 1 |
| | | | | | (2.5%) | | (0.4%) |
| Neck pain | 0 | 0 | 1 | 0 | 0 | 0 | 1 |
| | | | (2.6%) | | | | (0.4%) |
| Osteoarthritis | 0 | 0 | 0 | 0 | 1 | 0 | 1 |
| | | | | | (2.5%) | | (0.4%) |
| Periarthritis | 0 | 0 | 0 | 0 | 1 | 0 | 1 |
| | | | | | (2.5%) | | (0.4%) |
| Rotator cuff syndrome | 0 | 0 | 1 | 0 | 0 | 0 | 1 |
| | | | (2.6%) | | | | (0.4%) |
| Spinal pain | 0 | 0 | 0 | 0 | 1 | 0 | 1 |
| | | | | | (2.5%) | | (0.4%) |
| Tenosynovitis | 0 | 0 | 1 | 0 | 0 | 0 | 1 |
| | | | (2.6%) | | | | (0.4%) |
| Trigger finger | 0 | 0 | 0 | 0 | 1 | 0 | 1 |
| | | | | | (2.5%) | | (0.4%) |
| Gastrointestinal disorders | 1 | 1 | 3 | 3 | 5 | 0 | 13 |
| | (2.9%) | (2.9%) | (7.7%) | (7.5%) | (12.5%) | | (5.7%) |
| Diarrhea | 1 | 1 | 0 | 0 | 0 | 0 | 2 |
| | (2.9%) | (2.9%) | | | | | (0.9%) |
| Vomiting | 0 | 0 | 0 | 0 | 2 | 0 | 2 |
| | | | | | (5.0%) | | (0.9%) |
| Abdominal discomfort | 0 | 0 | 0 | 0 | 1 | 0 | 1 |
| | | | | | (2.5%) | | (0.4%) |
| Dyspepsia | 0 | 0 | 0 | 0 | 1 | 0 | 1 |
| | | | | | (2.5%) | | (0.4%) |
| Flatulence | 0 | 0 | 1 | 0 | 0 | 0 | 1 |
| | | | (2.6%) | | | | (0.4%) |
| Gastritis | 0 | 0 | 0 | 1 | 0 | 0 | 1 |
| | | | | (2.5%) | | | (0.4%) |
| Gastroesophageal reflux disease | 0 | 0 | 0 | 1 | 0 | 0 | 1 |
| | | | | (2.5%) | | | (0.4%) |
| Hemorrhoids | 0 | 0 | 0 | 0 | 1 | 0 | 1 |
| | | | | | (2.5%) | | (0.4%) |
| Large intestine polyp | 0 | 0 | 1 | 0 | 0 | 0 | 1 |
| | | | (2.6%) | | | | (0.4%) |
| Lip pain | 0 | 0 | 1 | 0 | 0 | 0 | 1 |
| | | | (2.6%) | | | | (0.4%) |
| Nausea | 0 | 0 | 0 | 0 | 1 | 0 | 1 |
| | | | | | (2.5%) | | (0.4%) |
| Proctalgia | 0 | 0 | 0 | 1 | 0 | 0 | 1 |
| | | | | (2.5%) | | | (0.4%) |
| Nervous system disorders | 1 | 3 | 0 | 4 | 4 | 1 | 13 |
| | (2.9%) | (8.6%) | | (10.0%) | (10.0%) | (2.6%) | (5.7%) |
| Headache | 0 | 2 | 0 | 3 | 3 | 0 | 8 |
| | | (5.7%) | | (7.5%) | (7.5%) | | (3.5%) |
| Sciatica | 0 | 0 | 0 | 1 | 1 | 0 | 2 |
| | | | | (2.5%) | (2.5%) | | (0.9%) |
| Carpal tunnel syndrome | 0 | 0 | 0 | 0 | 1 | 0 | 1 |
| | | | | | (2.5%) | | (0.4%) |
| Cerebrovascular accident | 1 | 0 | 0 | 0 | 0 | 0 | 1 |
| | (2.9%) | | | | | | (0.4%) |
| Dizziness postural | 0 | 0 | 0 | 0 | 0 | 1 | 1 |
| | | | | | | (2.6%) | (0.4%) |
| Dysgeusia | 0 | 1 | 0 | 0 | 0 | 0 | 1 |
| | | (2.9%) | | | | | (0.4%) |
| Injury, poisoning and procedural | 3 | 2 | 1 | 3 | 2 | 1 | 12 |
| complications | (8.6%) | (5.7%) | (2.6%) | (7.5%) | (5.0%) | (2.6%) | (5.3%) |
| Meniscus injury | 0 | 1 | 0 | 2 | 0 | 0 | 3 |
| | | (2.9%) | | (5.0%) | | | (1.3%) |
| Chest injury | 0 | 0 | 0 | 0 | 1 | 0 | 1 |
| | | | | | (2.5%) | | (0.4%) |
| Concussion | 1 | 0 | 0 | 0 | 0 | 0 | 1 |
| | (2.9%) | | | | | | (0.4%) |
| Contusion | 0 | 0 | 0 | 1 | 0 | 0 | 1 |
| | | | | (2.5%) | | | (0.4%) |
| Fall | 1 | 0 | 0 | 0 | 0 | 0 | 1 |
| | (2.9%) | | | | | | (0.4%) |
| Ligament injury | 0 | 0 | 0 | 1 | 0 | 0 | 1 |
| | | | | (2.5%) | | | (0.4%) |
| Ligament rupture | 0 | 0 | 1 | 0 | 0 | 0 | 1 |
| | | | (2.6%) | | | | (0.4%) |
| Ligament sprain | 0 | 1 | 0 | 0 | 0 | 0 | 1 |
| | | (2.9%) | | | | | (0.4%) |
| Limb injury | 1 | 0 | 0 | 0 | 0 | 0 | 1 |
| | (2.9%) | | | | | | (0.4%) |
| Skin laceration | 0 | 0 | 0 | 0 | 1 | 0 | 1 |
| | | | | | (2.5%) | | (0.4%) |
| Tooth fracture | 0 | 0 | 0 | 0 | 0 | 1 | 1 |
| | | | | | | (2.6%) | (0.4%) |
| Skin and subcutaneous tissue | 2 | 1 | 3 | 2 | 2 | 1 | 11 |
| disorders | (5.7%) | (2.9%) | (7.7%) | (5.0%) | (5.0%) | (2.6%) | (4.8%) |
| Dermatitis contact | 0 | 0 | 1 | 1 | 1 | 1 | 4 |
| | | | (2.6%) | (2.5%) | (2.5%) | (2.6%) | (1.8%) |
| Pruritus | 0 | 0 | 1 | 0 | 1 | 0 | 2 |
| | | | (2.6%) | | (2.5%) | | (0.9%) |
| Psoriasis | 0 | 0 | 0 | 1 | 0 | 1 | 2 |
| | | | | (2.5%) | | (2.6%) | (0.9%) |
| Actinic keratosis | 0 | 0 | 1 | 0 | 0 | 0 | 1 |
| | | | (2.6%) | | | | (0.4%) |
| Dermal cyst | 0 | 1 | 0 | 0 | 0 | 0 | 1 |
| | | (2.9%) | | | | | (0.4%) |
| Dermatitis acneiform | 1 | 0 | 0 | 0 | 0 | 0 | 1 |
| | (2.9%) | | | | | | (0.4%) |
| Hand dermatitis | 1 | 0 | 0 | 0 | 0 | 0 | 1 |
| | (2.9%) | | | | | | (0.4%) |
| General disorders and | 0 | 1 | 1 | 2 | 1 | 2 | 7 |
| administration site conditions | | (2.9%) | (2.6%) | (5.0%) | (2.5%) | (5.3%) | (3.1%) |
| Chills | 0 | 0 | 0 | 1 | 1 | 0 | 2 |
| | | | | (2.5%) | (2.5%) | | (0.9%) |
| Pyrexia | 0 | 1 | 0 | 0 | 0 | 1 | 2 |
| | | (2.9%) | | | | (2.6%) | (0.9%) |
| Asthenia | 0 | 0 | 0 | 1 | 0 | 0 | 1 |
| | | | | (2.5%) | | | (0.4%) |
| Influenza like illness | 0 | 0 | 0 | 1 | 0 | 0 | 1 |
| | | | | (2.5%) | | | (0.4%) |
| Non-cardiac chest pain | 0 | 0 | 1 | 0 | 0 | 0 | 1 |
| | | | (2.6%) | | | | (0.4%) |
| Oedema peripheral | 0 | 0 | 0 | 0 | 0 | 1 | 1 |
| | | | | | | (2.6%) | (0.4%) |
| Metabolism and nutrition | 1 | 0 | 0 | 3 | 2 | 1 | 7 |
| disorders | (2.9%) | | | (7.5%) | (5.0%) | (2.6%) | (3.1%) |
| Hyperglycemia | 0 | 0 | 0 | 0 | 1 | 1 | 2 |
| | | | | | (2.5%) | (2.6%) | (0.9%) |
| Diabetes mellitus | 0 | 0 | 0 | 1 | 0 | 0 | 1 |
| | | | | (2.5%) | | | (0.4%) |
| Haemochromatosis | 1 | 0 | 0 | 0 | 0 | 0 | 1 |
| | (2.9%) | | | | | | (0.4%) |
| Hypercholesterolemia | 0 | 0 | 0 | 0 | 1 | 0 | 1 |
| | | | | | (2.5%) | | (0.4%) |
| Hyperlipidemia | 0 | 0 | 0 | 1 | 0 | 0 | 1 |
| | | | | (2.5%) | | | (0.4%) |
| Vitamin D deficiency | 0 | 0 | 0 | 1 | 0 | 0 | 1 |
| | | | | (2.5%) | | | (0.4%) |
| Respiratory, thoracic and | 1 | 0 | 1 | 1 | 2 | 2 | 7 |
| mediastinal disorders | (2.9%) | | (2.6%) | (2.5%) | (5.0%) | (5.3%) | (3.1%) |
| Cough | 0 | 0 | 1 | 0 | 0 | 1 | 2 |
| | | | (2.6%) | | | (2.6%) | (0.9%) |
| Asthma | 0 | 0 | 0 | 0 | 1 | 0 | 1 |
| | | | | | (2.5%) | | (0.4%) |
| Bronchitis chronic | 0 | 0 | 0 | 0 | 1 | 0 | 1 |
| | | | | | (2.5%) | | (0.4%) |
| Chronic obstructive pulmonary | 0 | 0 | 0 | 0 | 1 | 0 | 1 |
| disease | | | | | (2.5%) | | (0.4%) |
| Oropharyngeal pain | 1 | 0 | 0 | 0 | 0 | 0 | 1 |
| | (2.9%) | | | | | | (0.4%) |
| Rhinorrhoea | 0 | 0 | 0 | 0 | 0 | 1 | 1 |
| | | | | | | (2.6%) | (0.4%) |
| Tonsillar hypertrophy | 0 | 0 | 0 | 1 | 0 | 0 | 1 |
| | | | | (2.5%) | | | (0.4%) |
| Renal and urinary disorders | 2 | 0 | 1 | 0 | 2 | 1 | 6 |
| | (5.7%) | | (2.6%) | | (5.0%) | (2.6%) | (2.6%) |
| Hematuria | 1 | 0 | 1 | 0 | 0 | 0 | 2 |
| | (2.9%) | | (2.6%) | | | | (0.9%) |
| Dysuria | 1 | 0 | 0 | 0 | 0 | 0 | 1 |
| | (2.9%) | | | | | | (0.4%) |
| Leukocyturia | 0 | 0 | 0 | 0 | 1 | 0 | 1 |
| | | | | | (2.5%) | | (0.4%) |
| Nephrolithiasis | 0 | 0 | 0 | 0 | 1 | 0 | 1 |
| | | | | | (2.5%) | | (0.4%) |
| Urinary tract disorder | 0 | 0 | 0 | 0 | 0 | 1 | 1 |
| | | | | | | (2.6%) | (0.4%) |
| Vascular disorders | 1 | 0 | 1 | 2 | 1 | 1 | 6 |
| | (2.9%) | | (2.6%) | (5.0%) | (2.5%) | (2.6%) | (2.6%) |
| Hypertension | 1 | 0 | 1 | 2 | 1 | 1 | 6 |
| | (2.9%) | | (2.6%) | (5.0%) | (2.5%) | (2.6%) | (2.6%) |
| Neoplasms benign, malignant and | 0 | 0 | 1 | 1 | 1 | 2 | 5 |
| unspecified (incl cysts and polyps) | | | (2.6%) | (2.5%) | (2.5%) | (5.3%) | (2.2%) |
| Acrochordon | 0 | 0 | 0 | 1 | 1 | 0 | 2 |
| | | | | (2.5%) | (2.5%) | | (0.9%) |
| Basal cell carcinoma | 0 | 0 | 1 | 0 | 0 | 0 | 1 |
| | | | (2.6%) | | | | (0.4%) |
| Skin papilloma | 0 | 0 | 0 | 0 | 0 | 1 | 1 |
| | | | | | | (2.6%) | (0.4%) |
| Uterine leiomyoma | 0 | 0 | 0 | 0 | 0 | 1 | 1 |
| | | | | | | (2.6%) | (0.4%) |
| Blood and lymphatic system | 0 | 1 | 1 | 0 | 0 | 1 | 3 |
| disorders | | (2.9%) | (2.6%) | | | (2.6%) | (1.3%) |
| Neutropenia | 0 | 0 | 1 | 0 | 0 | 0 | 1 |
| | | | (2.6%) | | | | (0.4%) |
| Thrombocytopenia | 0 | 1 | 0 | 0 | 0 | 0 | 1 |
| | | (2.9%) | | | | | (0.4%) |
| Thrombocytosis | 0 | 0 | 0 | 0 | 0 | 1 | 1 |
| | | | | | | (2.6%) | (0.4%) |
| Cardiac disorders | 0 | 0 | 1 | 1 | 1 | 0 | 3 |
| | | | (2.6%) | (2.5%) | (2.5%) | | (1.3%) |
| Atrioventricular block first degree | 0 | 0 | 0 | 0 | 1 | 0 | 1 |
| | | | | | (2.5%) | | (0.4%) |
| Coronary artery disease | 0 | 0 | 1 | 0 | 0 | 0 | 1 |
| | | | (2.6%) | | | | (0.4%) |
| Ventricular dysfunction | 0 | 0 | 0 | 1 | 0 | 0 | 1 |
| | | | | (2.5%) | | | (0.4%) |
| Eye disorders | 2 | 0 | 0 | 1 | 0 | 0 | 3 |
| | (5.7%) | | | (2.5%) | | | (1.3%) |
| Eyelid skin dryness | 0 | 0 | 0 | 1 | 0 | 0 | 1 |
| | | | | (2.5%) | | | (0.4%) |
| Ocular cyst | 1 | 0 | 0 | 0 | 0 | 0 | 1 |
| | (2.9%) | | | | | | (0.4%) |
| Retinal vein occlusion | 1 | 0 | 0 | 0 | 0 | 0 | 1 |
| | (2.9%) | | | | | | (0.4%) |
| Ear and labyrinth disorders | 0 | 1 | 0 | 0 | 1 | 0 | 2 |
| | | (2.9%) | | | (2.5%) | | (0.9%) |
| Vertigo | 0 | 1 | 0 | 0 | 1 | 0 | 2 |
| | | (2.9%) | | | (2.5%) | | (0.9%) |
| Hepatobiliary disorders | 1 | 0 | 0 | 0 | 1 | 0 | 2 |
| | (2.9%) | | | | (2.5%) | | (0.9%) |
| Cholelithiasis | 0 | 0 | 0 | 0 | 1 | 0 | 1 |
| | | | | | (2.5%) | | (0.4%) |
| Hypertransaminasemia | 1 | 0 | 0 | 0 | 0 | 0 | 1 |
| | (2.9%) | | | | | | (0.4%) |
| Immune system disorders | 0 | 0 | 1 | 0 | 0 | 1 | 2 |
| | | | (2.6%) | | | (2.6%) | (0.9%) |
| Seasonal allergy | 0 | 0 | 1 | 0 | 0 | 1 | 2 |
| | | | (2.6%) | | | (2.6%) | (0.9%) |
| Endocrine disorders | 0 | 0 | 0 | 1 | 0 | 0 | 1 |
| | | | | (2.5%) | | | (0.4%) |
| Goiter | 0 | 0 | 0 | 1 | 0 | 0 | 1 |
| | | | | (2.5%) | | | (0.4%) |
| Psychiatric disorders | 0 | 0 | 0 | 1 | 0 | 0 | 1 |
| | | | | (2.5%) | | | (0.4%) |
| Insomnia | 0 | 0 | 0 | 1 | 0 | 0 | 1 |
| | | | | (2.5%) | | | (0.4%) |

^{a}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 50
Number of Subjects with 1 or More Treatment-emergent
AEs with Frequency ≥5% from Week 16 through Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis | 35 | 35 | 39 | 40 | 40 | 38 | 227 |
| set | | | | | | | |
| Avg duration of follow-up | 37.81 | 36.58 | 38.44 | 34.95 | 35.88 | 38.62 | 37.02 |
| (weeks) | | | | | | | |
| Subjects with 1 or more | 23 | 18 | 19 | 27 | 27 | 19 | 133 |
| treatment-emergent adverse | (65.7%) | (51.4%) | (48.7%) | (67.5%) | (67.5%) | (50.0%) | (58.6%) |
| events | | | | | | | |
| System organ class | | | | | | | |
| Preferred term | | | | | | | |
| Infections and infestations | 16 | 12 | 16 | 17 | 20 | 14 | 95 |
| | (45.7%) | (34.3%) | (41.0%) | (42.5%) | (50.0%) | (36.8%) | (41.9%) |
| Nasopharyngitis | 9 | 3 | 7 | 6 | 11 | 5 | 41 |
| | (25.7%) | (8.6%) | (17.9%) | (15.0%) | (27.5%) | (13.2%) | (18.1%) |
| Upper respiratory tract infection | 4 | 6 | 3 | 3 | 2 | 4 | 22 |
| | (11.4%) | (17.1%) | (7.7%) | (7.5%) | (5.0%) | (10.5%) | (9.7%) |
| COVID-19 | 2 | 1 | 3 | 1 | 2 | 3 | 12 |
| | (5.7%) | (2.9%) | (7.7%) | (2.5%) | (5.0%) | (7.9%) | (5.3%) |
| Influenza | 1 | 0 | 1 | 3 | 1 | 1 | 7 |
| | (2.9%) | | (2.6%) | (7.5%) | (2.5%) | (2.6%) | (3.1%) |
| Urinary tract infection | 2 | 1 | 1 | 1 | 0 | 2 | 7 |
| | (5.7%) | (2.9%) | (2.6%) | (2.5%) | | (5.3%) | (3.1%) |
| Bronchitis | 1 | 1 | 3 | 1 | 0 | 0 | 6 |
| | (2.9%) | (2.9%) | (7.7%) | (2.5%) | | | (2.6%) |
| Sinusitis | 0 | 0 | 1 | 2 | 0 | 0 | 3 |
| | | | (2.6%) | (5.0%) | | | (1.3%) |
| Investigations | 4 | 1 | 3 | 3 | 2 | 4 | 17 |
| | (11.4%) | (2.9%) | (7.7%) | (7.5%) | (5.0%) | (10.5%) | (7.5%) |
| Alanine aminotransferase | 2 | 1 | 1 | 0 | 0 | 2 | 6 |
| increased | (5.7%) | (2.9%) | (2.6%) | | | (5.3%) | (2.6%) |
| Aspartate aminotransferase | 1 | 1 | 1 | 0 | 0 | 2 | 5 |
| increased | (2.9%) | (2.9%) | (2.6%) | | | (5.3%) | (2.2%) |
| Musculoskeletal and connective | 2 | 0 | 4 | 1 | 8 | 1 | 16 |
| tissue disorders | (5.7%) | | (10.3%) | (2.5%) | (20.0%) | (2.6%) | (7.0%) |
| Arthralgia | 1 | 0 | 1 | 0 | 2 | 0 | 4 |
| | (2.9%) | | (2.6%) | | (5.0%) | | (1.8%) |
| Gastrointestinal disorders | 1 | 1 | 3 | 3 | 5 | 0 | 13 |
| | (2.9%) | (2.9%) | (7.7%) | (7.5%) | (12.5%) | | (5.7%) |
| Vomiting | 0 | 0 | 0 | 0 | 2 | 0 | 2 |
| | | | | | (5.0%) | | (0.9%) |
| Nervous system disorders | 1 | 3 | 0 | 4 | 4 | 1 | 13 |
| | (2.9%) | (8.6%) | | (10.0%) | (10.0%) | (2.6%) | (5.7%) |
| Headache | 0 | 2 | 0 | 3 | 3 | 0 | 8 |
| | | (5.7%) | | (7.5%) | (7.5%) | | (3.5%) |
| Injury, poisoning and | 3 | 2 | 1 | 3 | 2 | 1 | 12 |
| procedural complications | (8.6%) | (5.7%) | (2.6%) | (7.5%) | (5.0%) | (2.6%) | (5.3%) |
| Meniscus injury | 0 | 1 | 0 | 2 | 0 | 0 | 3 |
| | | (2.9%) | | (5.0%) | | | (1.3%) |
| Vascular disorders | 1 | 0 | 1 | 2 | 1 | 1 | 6 |
| | (2.9%) | | (2.6%) | (5.0%) | (2.5%) | (2.6%) | (2.6%) |
| Hypertension | 1 | 0 | 1 | 2 | 1 | 1 | 6 |
| | (2.9%) | | (2.6%) | (5.0%) | (2.5%) | (2.6%) | (2.6%) |

^{a}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 51
Number of Subjects with 1 or More Treatment-emergent Serious AEs from Week 16 through Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis set | 35 | 35 | 39 | 40 | 40 | 38 | 227 |
| Avg duration of follow-up (weeks) | 37.81 | 36.58 | 38.44 | 34.95 | 35.88 | 38.62 | 37.02 |
| Subjects with 1 or more treatment- | 1 | 0 | 2 | 3 | 2 | 1 | 9 |
| emergent serious adverse events | (2.9%) | | (5.1%) | (7.5%) | (5.0%) | (2.6%) | (4.0%) |
| System organ class | | | | | | | |
| Preferred term | | | | | | | |
| Cardiac disorders | 0 | 0 | 1 | 1 | 0 | 0 | 2 |
| | | | (2.6%) | (2.5%) | | | (0.9%) |
| Coronary artery disease | 0 | 0 | 1 | 0 | 0 | 0 | 1 |
| | | | (2.6%) | | | | (0.4%) |
| Ventricular dysfunction | 0 | 0 | 0 | 1 | 0 | 0 | 1 |
| | | | | (2.5%) | | | (0.4%) |
| Musculoskeletal and connective | 0 | 0 | 1 | 0 | 1 | 0 | 2 |
| tissue disorders | | | (2.6%) | | (2.5%) | | (0.9%) |
| Foot deformity | 0 | 0 | 1 | 0 | 0 | 0 | 1 |
| | | | (2.6%) | | | | (0.4%) |
| Intervertebral disc protrusion | 0 | 0 | 0 | 0 | 1 | 0 | 1 |
| | | | | | (2.5%) | | (0.4%) |
| General disorders and | 0 | 0 | 1 | 0 | 0 | 0 | 1 |
| administration site conditions | | | (2.6%) | | | | (0.4%) |
| Non-cardiac chest pain | 0 | 0 | 1 | 0 | 0 | 0 | 1 |
| | | | (2.6%) | | | | (0.4%) |
| Infections and infestations | 0 | 0 | 0 | 0 | 1 | 0 | 1 |
| | | | | | (2.5%) | | (0.4%) |
| Diverticulitis | 0 | 0 | 0 | 0 | 1 | 0 | 1 |
| | | | | | (2.5%) | | (0.4%) |
| Injury, poisoning and procedural | 0 | 0 | 0 | 1 | 0 | 0 | 1 |
| complications | | | | (2.5%) | | | (0.4%) |
| Ligament injury | 0 | 0 | 0 | 1 | 0 | 0 | 1 |
| | | | | (2.5%) | | | (0.4%) |
| Neoplasms benign, malignant and | 0 | 0 | 0 | 0 | 0 | 1 | 1 |
| unspecified (incl cysts and polyps) | | | | | | (2.6%) | (0.4%) |
| Uterine leiomyoma | 0 | 0 | 0 | 0 | 0 | 1 | 1 |
| | | | | | | (2.6%) | (0.4%) |
| Nervous system disorders | 1 | 0 | 0 | 0 | 0 | 0 | 1 |
| | (2.9%) | | | | | | (0.4%) |
| Cerebrovascular accident | 1 | 0 | 0 | 0 | 0 | 0 | 1 |
| | (2.9%) | | | | | | (0.4%) |
| Respiratory, thoracic and | 0 | 0 | 0 | 1 | 0 | 0 | 1 |
| mediastinal disorders | | | | (2.5%) | | | (0.4%) |
| Tonsillar hypertrophy | 0 | 0 | 0 | 1 | 0 | 0 | 1 |
| | | | | (2.5%) | | | (0.4%) |

^{a}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 52
Number of Subjects with 1 or More Treatment-emergent AEs
Leading to Discontinuation from Week 16 through Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis set | 35 | 35 | 39 | 40 | 40 | 38 | 227 |
| Avg duration of follow-up (weeks) | 37.81 | 36.58 | 38.44 | 34.95 | 35.88 | 38.62 | 37.02 |
| Subjects with 1 or more treatment-emergent | 0 | 1 | 0 | 2 | 1 | 0 | 4 |
| adverse events leading to discontinuation of | | (2.9%) | | (5.0%) | (2.5%) | | (1.8%) |
| study agent | | | | | | | |
| System organ class | | | | | | | |
| Preferred term | | | | | | | |
| Gastrointestinal disorders | 0 | 0 | 0 | 1 | 1 | 0 | 2 |
| | | | | (2.5%) | (2.5%) | | (0.9%) |
| Proctalgia | 0 | 0 | 0 | 1 | 0 | 0 | 1 |
| | | | | (2.5%) | | | (0.4%) |
| Vomiting | 0 | 0 | 0 | 0 | 1 | 0 | 1 |
| | | | | | (2.5%) | | (0.4%) |
| General disorders and administration site | 0 | 0 | 0 | 1 | 1 | 0 | 2 |
| conditions | | | | (2.5%) | (2.5%) | | (0.9%) |
| Chills | 0 | 0 | 0 | 1 | 1 | 0 | 2 |
| | | | | (2.5%) | (2.5%) | | (0.9%) |
| Asthenia | 0 | 0 | 0 | 1 | 0 | 0 | 1 |
| | | | | (2.5%) | | | (0.4%) |
| Nervous system disorders | 0 | 0 | 0 | 1 | 1 | 0 | 2 |
| | | | | (2.5%) | (2.5%) | | (0.9%) |
| Headache | 0 | 0 | 0 | 1 | 1 | 0 | 2 |
| | | | | (2.5%) | (2.5%) | | (0.9%) |
| Ear and labyrinth disorders | 0 | 0 | 0 | 0 | 1 | 0 | 1 |
| | | | | | (2.5%) | | (0.4%) |
| Vertigo | 0 | 0 | 0 | 0 | 1 | 0 | 1 |
| | | | | | (2.5%) | | (0.4%) |
| Investigations | 0 | 1 | 0 | 0 | 0 | 0 | 1 |
| | | (2.9%) | | | | | (0.4%) |
| Alanine aminotransferase increased | 0 | 1 | 0 | 0 | 0 | 0 | 1 |
| | | (2.9%) | | | | | (0.4%) |
| Aspartate aminotransferase increased | 0 | 1 | 0 | 0 | 0 | 0 | 1 |
| | | (2.9%) | | | | | (0.4%) |
| Gamma-glutamyltransferase increased | 0 | 1 | 0 | 0 | 0 | 0 | 1 |
| | | (2.9%) | | | | | (0.4%) |
| Psychiatric disorders | 0 | 0 | 0 | 1 | 0 | 0 | 1 |
| | | | | (2.5%) | | | (0.4%) |
| Insomnia | 0 | 0 | 0 | 1 | 0 | 0 | 1 |
| | | | | (2.5%) | | | (0.4%) |
| Skin and subcutaneous tissue disorders | 0 | 0 | 0 | 1 | 0 | 0 | 1 |
| | | | | (2.5%) | | | (0.4%) |
| Psoriasis | 0 | 0 | 0 | 1 | 0 | 0 | 1 |
| | | | | (2.5%) | | | (0.4%) |

^{a}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 53
Number of Subjects with Selected Treatment-emergent AEs of Interest
Associated with COVID-19 Infection from Week 16 through Week 56

| | Placebo → | 25 mg | 50 mg | 25 mg | 100 mg | 100 mg | |
|---|---|---|---|---|---|---|---|
| Treatment Regimen | 100 mg QD | QD | QD | BID | QD | BID | Combined^{a} |
| Analysis set: LTE Safety analysis | 35 | 35 | 39 | 40 | 40 | 38 | 227 |
| set | | | | | | | |
| Avg duration of follow-up (weeks) | 37.81 | 36.58 | 38.44 | 34.95 | 35.88 | 38.62 | 37.02 |
| Subjects with 1 or more COVID-19 | 3 | 1 | 3 | 1 | 2 | 4 | 14 |
| associated adverse events | (8.6%) | (2.9%) | (7.7%) | (2.5%) | (5.0%) | (10.5%) | (6.2%) |
| Special interest category | | | | | | | |
| Preferred term | | | | | | | |
| COVID-19 associated | | | | | | | |
| COVID-19 | 2 | 1 | 3 | 1 | 2 | 3 | 12 |
| | (5.7%) | (2.9%) | (7.7%) | (2.5%) | (5.0%) | (7.9%) | (5.3%) |
| SARS-CoV-2 test positive | 1 | 0 | 0 | 0 | 0 | 1 | 2 |
| | (2.9%) | | | | | (2.6%) | (0.9%) |

^{a}Includes all columns of treatment with the compound of Formula (I) (Placebo → 100 mg QD, 25 mg QD, 50 mg QD, 25 mg BID, 100 mg QD, and 100 mg BID).
Note:
Subjects are counted only once for any given event, regardless of the number of times they actually experienced the event. Adverse events are coded using MedDRA Version 25.1.

TABLE 54
Number of Subjects with 1 or More Post-Baseline Most Severe Suicidal
Ideation or Suicidal Behavior from Week 16 through Week 56

| | Placebo → | 25 mg | 50 mg | 25 mg | 100 mg | 100 mg | |
|---|---|---|---|---|---|---|---|
| Treatment Regimen | 100 mg QD^{a} | QD | QD | BID | QD | BID | Combined^{b} |
| Analysis set: Safety analysis | 35 | 35 | 39 | 40 | 40 | 38 | 227 |
| set | | | | | | | |
| No suicidal ideation or | 35 | 35 | 39 | 40 | 40 | 38 | 227 |
| behavior | (100.0%) | (100.0%) | (100.0%) | (100.0%) | (100.0%) | (100.0%) | (100.0%) |
| Suicidal ideation or behavior | 0 | 0 | 0 | 0 | 1 | 0 | 1 |
| | | | | | (2.3%) | | (0.4%) |
| Suicidal ideation | 0 | 0 | 0 | 0 | 0 | 0 | 0 |
| 1 - Wish to be dead | 0 | 0 | 0 | 0 | 0 | 0 | 0 |
| 2 - Non-specific active | 0 | 0 | 0 | 0 | 0 | 0 | 0 |
| suicidal thoughts | | | | | | | |
| 3 - Active suicidal ideation | 0 | 0 | 0 | 0 | 0 | 0 | 0 |
| with any methods (not plan) | | | | | | | |
| without intent to act | | | | | | | |
| 4 - Active suicidal ideation | 0 | 0 | 0 | 0 | 0 | 0 | 0 |
| with some intent to act, | | | | | | | |
| without specific plan | | | | | | | |
| 5 - Active suicidal ideation | 0 | 0 | 0 | 0 | 0 | 0 | 0 |
| with specific plan and intent | | | | | | | |
| Suicidal behavior | 0 | 0 | 0 | 0 | 0 | 0 | 0 |
| 6 - Preparatory acts or | 0 | 0 | 0 | 0 | 0 | 0 | 0 |
| behavior | | | | | | | |
| 7 - Aborted attempt | 0 | 0 | 0 | 0 | 0 | 0 | 0 |
| 8 - Interrupted attempt | 0 | 0 | 0 | 0 | 0 | 0 | 0 |
| 9 - Non-fatal suicide attempt | 0 | 0 | 0 | 0 | 0 | 0 | 0 |
| 10 - Completed suicide | 0 | 0 | 0 | 0 | 0 | 0 | 0 |

The most frequent system organ-class involved in AEs in all groups was infections and infestations (41.9%). There was no clear evidence of a dose-dependent increase in the occurrence of AEs in this SOC across the treatment groups.

The most common AEs in this SOC were nasopharyngitis (18.1%), upper respiratory tract infection (9.7%), and COVID-19 (5.3%) in the combined group that received the compound of Formula (I) (Table 48 and Table 49). AEs reported by at least 5% of participants in any treatment group are summarized in Table 48 and Table 50. Again, the most common AEs in the combined group of participants receiving the compound of Formula (I) are nasopharyngitis, upper respiratory tract infection, and COVID-19.

The proportion of participants that received the compound of Formula (I) with one or more serious adverse events (SAEs) was low (4.0%), with 8 participants experiencing one SAE each and 1 participant experiencing 2 SAEs (Table 48 and Table 51). All SAE were singular and considered not related to study intervention by investigators.

The proportion of participants who discontinued study intervention due to one or more AEs was low (n=4). All events were singular in nature with no apparent dose-dependent increase (Table 48 and Table 52). The number of participants with abnormal hematology and chemistry laboratory values with CTCAE toxicity grade≥3 were low through the end of the study (Table 43 through Table 46). There was no apparent dose-dependent increase in laboratory abnormality occurrences.

There were no deaths reported during the LTE study period. A single case of basal cell skin cancer was reported in the 50 mg QD group. There was one SAE of worsening of coronary artery disease in the 50 mg QD arm (prior history of myocardium infarct), and one SAE of cerebrovascular accident in the placebo to 100 mg QD arm.

### Example 3. Single Dose Daily Administration for 16 Weeks in Adult Human Subjects with Moderate to Severe Plaque Psoriasis

IL-23 receptor antagonist peptide as used in this example refers to a compound of Formula (I) or a pharmaceutically acceptable salt or solvate thereof, specifically a hydrochloride salt of compound of Formula (I) in a crystalline form.

Described below is a multicenter, randomized, double-blind, placebo-controlled study to evaluate the efficacy and safety of the hydrochloride salt of the compound of Formula (I) for the treatment of moderate-to-severe plaque psoriasis.

### Objectives and Endpoints

| | |
|---|---|
| Objectives | Endpoints |

Primary

| | |
|---|---|
| To evaluate the efficacy of IL-23 | Proportion of subjects achieving an |
| receptor antagonist peptide in subjects | IGA score of 0 or 1 and a ≥2-grade |
| with moderate-to-severe plaque psoriasis | improvement from baseline at Week 16 |
| | Proportion of subjects achieving |
| | PASI 90 response (≥90% improvement |
| | from PASI in PASI) at Week 16 |

Secondary

| | |
|---|---|
| To evaluate the general and special | Proportion of subjects achieving an |
| area psoriasis efficacy of IL-23 receptor | IGA score of 0 at Week 16 |
| antagonist peptide in subjects with | Proportion of subjects achieving |
| moderate-to-severe plaque psoriasis | PASI 75 response (≥75% improvement |
| | from baseline in PASI) at Week 4 |
| | Proportion of subjects achieving |
| | PASI 90 response (≥90% improvement |
| | from baseline in PASI) at Week 8 |
| | Proportion of subjects achieving |
| | PASI 75 response at Week 16 |
| | Proportion of subjects achieving |
| | PASI 100 response (≥100% improvement |
| | from baseline in PASI) at Week 16 |
| | Proportion of subjects achieving |
| | ss-IGA score 0 or 1 and a ≥2-grade |
| | improvement from baseline at Week 16 |
| To evaluate the effect of IL-23 | Proportion of subjects achieving a |
| receptor antagonist peptide on PROs in | PSSD symptom score of 0 at Week 8 |
| participants with moderate-to-severe | Proportion of subjects achieving a |
| plaque psoriasis | PSSD symptom score of 0 at Week 16 |
| | Proportion of subjects achieving ≥4- |
| | point improvement from baseline in |
| | PSSD Itch score at Week 4 |
| | Proportion of subjects achieving ≥4- |
| | point improvement from baseline in |
| | PSSD Itch score at Week 16 |
| To evaluate the maintenance of | Proportion of subjects achieving a |
| efficacy of IL-23 receptor antagonist | DLQI of 0 or 1 at Week 16 among subjects |
| peptide compared with treatment | with baseline DLQI score >1 |
| withdrawal during the randomized | |
| withdrawal period | |
| To assess the safety and tolerability | Frequency and type of AEs and |
| of IL-23 receptor antagonist peptide in | SAEs |
| subjects with moderate-to-severe plaque | |
| psoriasis | |
| To further evaluate the general and | Change from baseline in BSA at Week 16 |
| special area psoriasis efficacy of IL-23 | Change from baseline in PASI at Week 16 |
| receptor antagonist peptide in subjects with | Percent improvement in PASI at Week 16 |
| moderate-to-severe plaque psoriasis | Proportion of subjects having achieved |
| | sPGA of Genitalia score of 0 or 1 |
| | and a ≥2-grade improvement from baseline |
| | at Week 16 |
| | Proportion of subjects having achieved |
| | hf-PGA score of 0 or 1 and a ≥2-grade |
| | improvement from baseline at Week 16 |
| | Percent change from baseline in |
| | mNAPSI score at Week 16 |
| | Proportion of subjects having achieved |
| | f-PGA score of 0 or 1 at Week 16 |
| To further evaluate the effect of IL- | Change from baseline in PSSD |
| 23 receptor antagonist peptide on PROs in | symptom score at Week 16 |
| participants with moderate-to-severe plaque | Change from baseline in PSSD |
| psoriasis | sign score at Week 16 |
| | Proportion of subjects having |
| | achieved PSSD sign score of 0 at Week 16 |
| | Proportion of subjects having |
| | achieved GenPs-SFQ Item 2 score of 0 or 1 |
| | at Week 16 |
| | Proportion of subjects having achieved |
| | DLQI score of 0 or 1 at Week 16 |
| | Change from baseline in DLQI |
| | total score at Week 16 |
| | Change from baseline in the domain scores |
| | of the PROMIS-29 score at Week 16 |
| | Proportion of subjects having achieved |
| | CDLQI score of 0 or 1 at Week 16 |
| | Change from baseline in CDLQI at Week 16 |
| | Change from baseline in the domain scores |
| | of the PROMIS-25 pediatric score at |
| | Week 16 |
| To further evaluate the maintenance | Proportion of subjects having achieved |
| of efficacy of IL-23 receptor antagonist | IGA score of 0 or 1 and a ≥2-grade |
| peptide compared with treatment | improvement from baseline at Week 52 |
| withdrawal during the randomized | Proportion of subjects having achieved |
| withdrawal period | IGA score of 0 at Week 52 |
| | Proportion of subjects having achieved |
| | PASI 100 response at Week 52 |
| To evaluate long-term psoriasis | Proportion of subjects having achieved |
| efficacy of IL-23 receptor antagonist | IGA score of 0 or 1 and ≥2-grade |
| peptide in adolescent participants with | improvement from baseline at Week 52 |
| moderate-to-severe plaque psoriasis | Proportion of subjects having achieved |
| | PASI 75 response at Week 52 |
| | Proportion of subjects having achieved |
| | PASI 90 response at Week 52 |

Exploratory

| | |
|---|---|
| To further assess the safety and | Frequency and type of related AEs and AEs |
| tolerability of IL-23 receptor antagonist | leading to discontinuation of study |
| peptide in participants with moderate-to- | intervention |
| severe plaque psoriasis | Laboratory parameters and change |
| | from baseline in laboratory parameters |
| | (hematology and chemistry) over time |
| To further evaluate psoriasis efficacy | Evaluation of IGA, PASI, BSA, ss-IGA, |
| of IL-23 receptor antagonist peptide | sPGA of Genitalia, hf-PGA, f-PGA, |
| | and mNAPSI scores of subjects |
| To further assess effect of IL-23 | Evaluation of PSSD, GenPs-SFQ, |
| receptor antagonist peptide on PROs in | TSQM-E efficacy domain, DLQI/CDLQI, |
| participants with moderate-to-severe plaque | EQ-5D-5L, PROMIS-29/PROMIS-25, PsA |
| psoriasis | Pain assessment, and PsA Disease |
| | assessment scores of subjects |
| To evaluate the PK and | IL-23 receptor antagonist peptide |
| immunogenicity of IL-23 receptor | PK parameters |
| antagonist peptide and explore the | Relationship between PK parameters |
| Exposure-Response (E-R) relationship in | and efficacy |
| participants with moderate-to-severe plaque | Incidence of anti-drug antibodies to |
| psoriasis | IL-23 receptor antagonist peptide |
| To explore biomarkers in participants | Change from baseline in cellular |
| with moderate-to-severe plaque psoriasis | and molecular biomarkers in skin and blood |
| To evaluate the efficacy of IL-23 | Evaluation of PASI, IGA, and |
| receptor antagonist peptide retreatment after | PSSD scores of subjects |
| relapse for adult participants who were | |
| withdrawn at Week 24 | |

### Overall Design

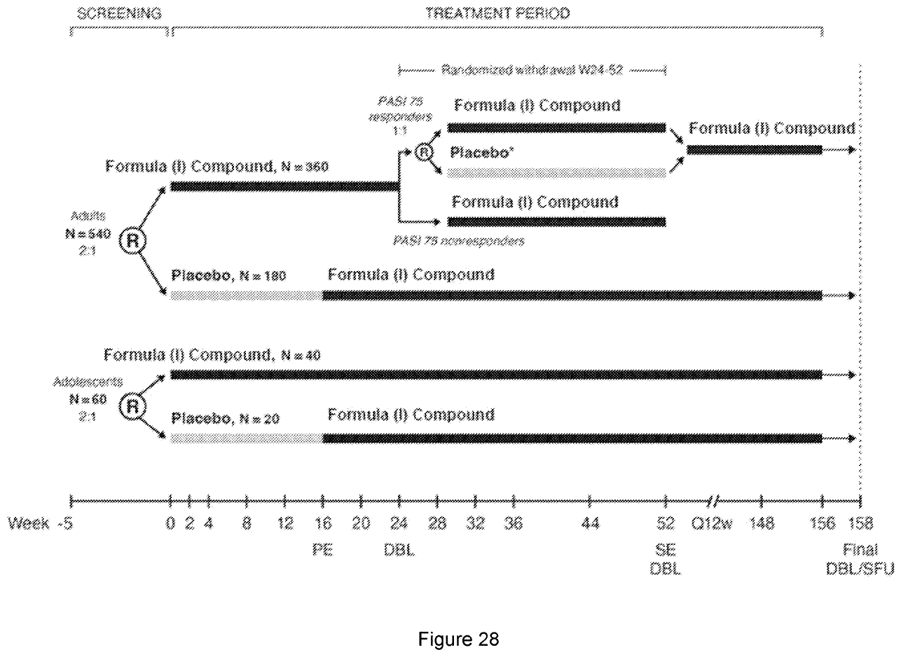

This is a randomized, double-blind, placebo-controlled, parallel group, multicenter, interventional study with randomized withdrawal and treatment in subjects with moderate-to-severe plaque psoriasis. A target of 600 subjects are enrolled in this study with ~10% adolescent participants and ~90% a adults, with each age group randomized with a 2:1 randomization to treatment or placebo. A schematic view of this study is shown in FIG. 28.

Subjects are instructed to take the study intervention at approximately the same time every day upon waking on an empty stomach with approximately 240 mL of noncarbonated water.

### Dosage

Example 1 and Example 2 demonstrated the clinical trial results among all 5 dose regimens (25 mg QD, 25 mg BID, 50 mg QD, 100 mg QD, 100 mg BID) evaluated in the study. Using the steady-state average (C_{avg}) and trough (C_{trough}) concentrations of IL-23 receptor antagonist peptide in the serum of subjects and the efficacy endpoints (proportion of participants with an IGA score of 0 or 1 or PASI 75/90/100), it was found that higher clinical efficacy is more tightly associated with C_{avg} than with C_{trough}.

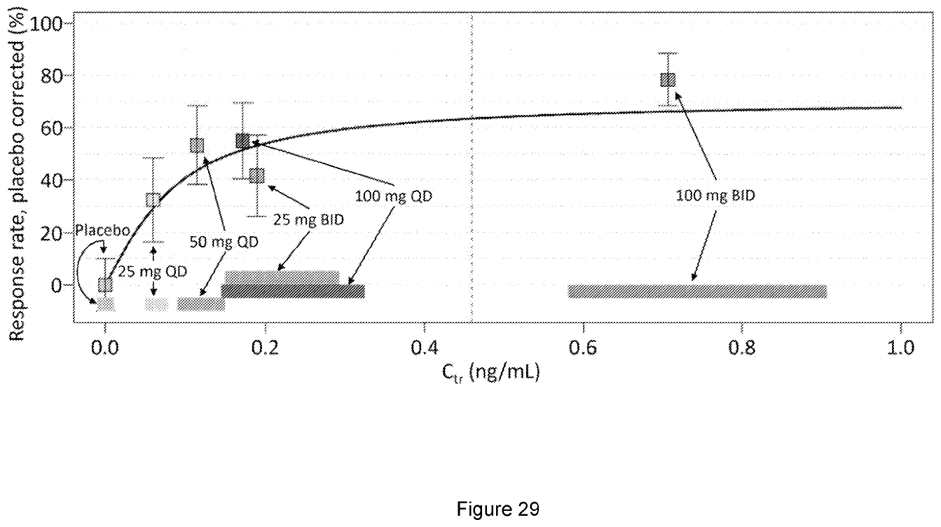

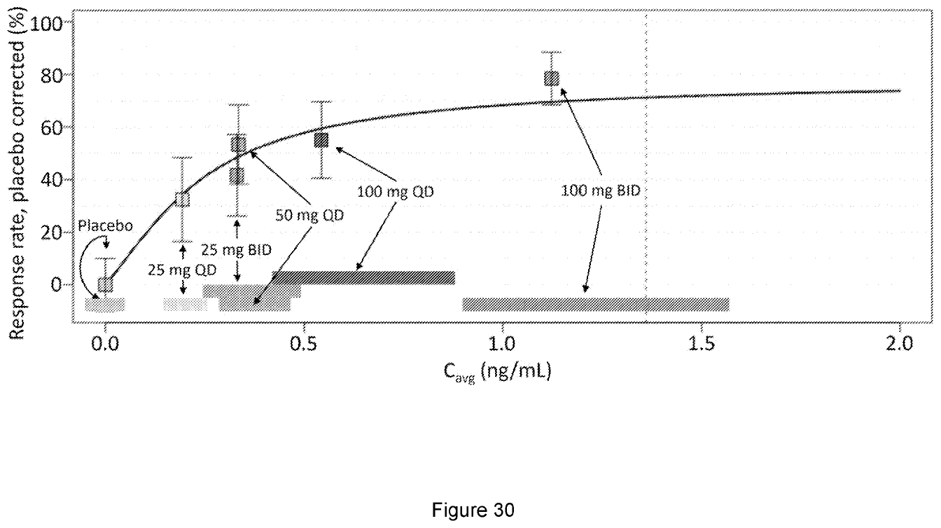

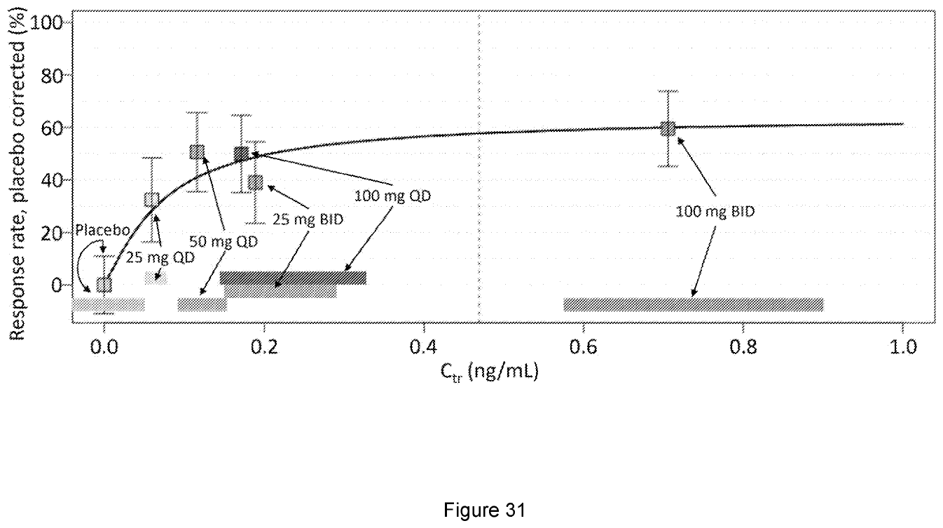

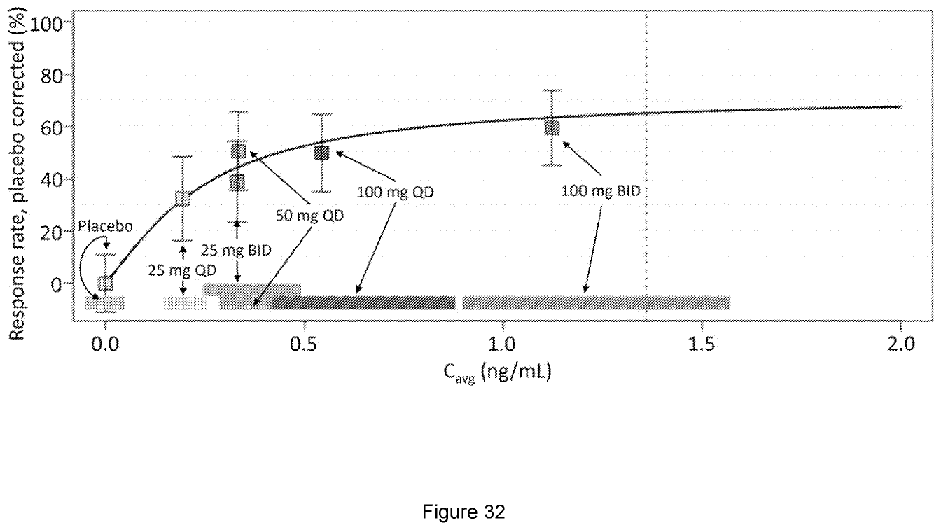

A population PK model was developed using the results of Example 1 and Example 2, incorporating body weight with standard allometric exponents and centering about 90 kg with the results of the population PK model shown in FIG. 29 through FIG. 32. Covariate analysis of renal and hepatic function variables was performed and none of these were found to be significant. Based on this model, post-hoc C_{trough} and C_{avg} values of IL-23 receptor antagonist peptide were obtained. The C_{trough} and C_{avg} values obtained were then used to fit ordinal logistic Emax regression models for PASI 75 (FIG. 29 and FIG. 30). and IGA 0/1 (FIG. 31 and FIG. 32) to yield exposure-response (E-R) models. In the E-R models for both PASI 75 and IGA 0/1, C_{avg} is found to be a better model parameter of clinical response than C_{trough}. Accordingly, a 200 mg QD regimen is modeled to achieve a clinical response rate similar to that of 100 mg BID with the ease of single-dose administration.

### Adult Participants:

IL-23 Receptor Antagonist Peptide 200 mg Daily

Week 0 to Week 24: Participants receive IL-23 receptor antagonist peptide 200 mg once daily (QD)

Week 24 to Week 52: Participants who are PASI 75 responders are re-randomized 1:1 to either continue IL-23 receptor antagonist peptide 200 mg daily or to be transitioned to placebo. Participants transitioned to placebo receive IL-23 receptor antagonist peptide 200 mg once daily upon loss of≥50% PASI improvement at Week 24 or at Week 52 if no loss of response is observed. All participants are treated with IL-23 receptor antagonist peptide 200 mg at Week 52. Participants who are PASI 75 non-responders at Week 24 continue to receive IL-23 receptor antagonist peptide 200 mg through week 52.

Week 52 to Week 156: Participants receive IL-23 receptor antagonist peptide 200 mg daily through Week 156.

Placebo to IL-23 Receptor Antagonist Peptide 200 mg Daily

Week 0 to Week 16: Participants receive placebo once daily through Week 16.

Week 16 to Week 156: Participants cross-over to receive IL-23 receptor antagonist peptide 200 mg once daily through Week 156.

### Adolescent Participants:

IL-23 Receptor Antagonist Peptide 200 mg Daily

Week 0 to Week 156: Participants are treated with IL-23 receptor antagonist peptide 200 mg once daily.

Adolescents do not participate in re-randomization regardless of PASI score at Week 24.

Placebo to IL-23 receptor antagonist peptide 200 mg Daily

Week 0 to Week 16: Participants receive placebo once daily through Week 16.

Week 16 to Week 156: Participants cross-over to receive IL-23 receptor antagonist peptide 200 mg once daily through Week 156.

The study includes a 5-week screening period, a placebo-controlled period through Week 16, and a randomized withdrawal ad re-treatment period from Week 24 through Week 52, at which point patients are transitioned to open-label IL-23 receptor antagonist peptide through Week 156. A 2-week safety follow-up period is included for participants who discontinue study intervention during the study or at the end of the treatment period.

Efficacy, safety, PK, immunogenicity, and biomarkers are assessed. In addition, there are three optional substudies for subjects who consented (where local regulations permit): a pharmacogenomic blood sample, skin biopsy, and photography collection.

### Inclusion Criteria

Each subject is ≥12 years old, and satisfied all of the following criteria:
(a) has a diagnosis of plaque psoriasis, with or without PsA, for at least 26 weeks prior to the first administration of study intervention;
(b) has a total BSA≥10% at screening and baseline;
(c) has a total PASI≥12 at screening and baseline;
(d) has a total IGA≥3 at screening and baseline; and
(e) is a candidate for phototherapy or systemic treatment for plaque psoriasis.

For subjects≥12 to <18 years of age, a weight requirement of ≥40 kg at baseline is required.

### Exclusion Criteria

A potential subject is excluded if the subject meets any of the following criteria:
(a) has a nonplaque form of psoriasis (e.g., erythrodermic, guttate, or pustular);
(b) has current drug-induced psoriasis (e.g., a new onset of psoriasis or an exacerbation of psoriasis from beta blockers, calcium channel blockers, or lithium);
(c) has known allergies, hypersensitivity, or intolerance to IL-23 receptor antagonist peptide or its excipients;
(d) had a major surgical procedure (e.g., requiring general anesthesia) within 8 weeks of screening or will not have fully recovered from a surgical procedure or has a surgical procedure planned during the time of the study; or
(e) has a transplanted organ (with the exception of corneal transplantation>12 weeks before the first administration;
(f) had a history of drug or alcohol abuse within 1 year before screening.

A subject is also excluded if the subject:
(a) had previously received IL-23 receptor antagonist peptide;
(b) experienced primary efficacy failure (no response within 16 weeks) to 1 or more agents directly targeting IL-23 or had a clinically significant AE to 1 or more agents directly targeting IL-23;
(c) had received agents that deplete B cells (including, but not limited to, rituximab, or alemtuzumab) within 26 weeks of the first administration of study intervention;
(d) had received biologic therapy or agents that modulate T cells (including, but not limited to abatacept or visilizumab) 12 weeks or 5 half-lives, whichever is longer, prior to the first administration of study intervention;
(e) had received any systemic immunosuppressants (including, but not limited to, methotrexate (MTX), azathioprine, cyclosporine A, corticosteroids, cyclophosphamide, tofacitinib, apremilast, and deucravacitinib) within 4 weeks of the first administration of study intervention;
(f) had received, or was expected to receive, any live virus or bacterial vaccination within 12 weeks before the first administration of study intervention, with the exception of the Bacille Calmette Guerin (BCG) vaccine below;
(g) had received the BCG vaccine within 12 months of the first administration of study intervention;
(j) had received phototherapy or any systemic medications that could affect psoriasis or the PASI or IGA evaluations (including, but not limited to, oral or injectable corticosteroids, acitretin, retinoids, 1,25-dihydroxy vitamin D3 and analogues, herbal treatments or traditional Taiwanese, Korean, or Chinese medicines) within 4 weeks of the first administration of study intervention; or
(k) had received topical therapies that could affect psoriasis or the PASI or IGA evaluation (including, but not limited to corticosteroids, calcineurin inhibitors, vitamin D analogs, vitamin A analogs, retinoids, tar, anthralin, calcipotriene, tazarotene, methoxsalen, trimethylpsoralens, fumarate, PDE4 inhibitors, aryl hydrocarbon receptor-modulating agents, and traditional Taiwanese, Korean, or Chinese medicines) within 2 weeks of the first administration of study intervention.

Subjects are also excluded if their screening laboratory test results reveal:
(a) hemoglobin<10 ng/dL;
(b) white blood cells<3.5×10³/μL;
(c) neutrophils<1.5×10³/μL;
(d) platelets<100×10³/μL;
(e) eGFR<60 mL/min/1.73 m²;
(f) aspartate aminotransferase>2×ULN; or
(g) alanine aminotransferase>2×ULN.

Subjects are additionally excluded if they present a positive test for Hepatitis B virus (HBV), Hepatitis C virus (HCV), or human immunodeficiency virus (HIV) at the time of screening.

Subjects are also excluded if they had a chest radiograph or CT scan within 12 weeks that showed evidence of ongoing infection or malignancy (chest radiograph is optional for participants<18 years of age at time of screening).

Subjects are additionally excluded if they meet one or more of the following criteria:
(a) have a history of chronic or recurrent infectious disease including but not limited to chronic renal infection, chronic chest infection, recurrent urinary tract infection, sever fungal infection, or open, draining, or infected skin wounds or ulcers;
(b) have a history of an infected joint prosthesis or have received antibiotics for a suspected infection of a joint prosthesis if the prosthesis has not been removed or replaced;
(c) have or suspect having immunodeficiency including but not limited to history of invasive opportunistic infections (e.g., active TB, nontuberculous mycobacterial infection, histoplasmosis, listeriosis, coccidioidomycosis, pneumocytosis, aspergillosis, HIV, etc.) or otherwise recurrent infections of abnormal frequency or prolonged duration;
(e) have a serious infection (e.g., disseminated herpes zoster, sepsis, pneumonia, or pyelonephritis) or have been hospitalized or received IV antibiotics during the 8 weeks before screening;
(f) tested positive for or have been exposed to COVID-19 within 4 weeks prior to the first dose (unless they lack symptoms and have a negative test at least 2 weeks after symptom onset or exposure;
(g) have a current malignancy or history of malignancy within 5 years before screening (except nonmelanoma skin cancer or cervical carcinoma in situ that has been adequately treated with no evidence of recurrence for at least 12 weeks prior to first administration);
(h) have: a history of lymphoproliferative disease including lymphoma, a monoclonal gammopathy of undetermined significance, or signs and symptoms of lymphoproliferative disease; or
(i) have a known active tuberculosis infection.

### Intervention Groups

Each subject in the groups is administered a tablet containing IL-23 receptor antagonist peptide or placebo. Subjects take the tablet with approximately 240 mL of noncarbonated water and on an empty stomach consistently throughout the study (including site visit days). An empty stomach is defined as abstaining from food intake for at least 2 hours before taking the study intervention and abstaining from food and liquid intake for at least 30 minutes after taking the study intervention.

Central randomization is implemented in conducting this study. Participants are assigned to 1 of 2 intervention groups based on an algorithm implemented before the study. Permuted block randomization with stratification by age group (adolescents<18 years and adults≥18 years), baseline weight category for adults (≤90 kg, >90 kg) and geographic region is used. Based on the algorithm, a unique intervention code is assigned, which dictates the intervention assignment and matching study intervention kit for the participant.

At Week 24, adult participants randomized to IL-23 receptor antagonist peptide 200 mg once daily and who are PASI 75 responders are re-randomized either to placebo or IL-23 receptor antagonist peptide 200 mg once daily in a 1:1 ratio. Participants are assigned to treatment groups using permuted block randomization and the randomization is stratified by geographic region and PASI 90 response status at Week 24.

### Study Evaluations

Efficacy, PK, immunogenicity, biomarker, pharmacogenomic, photography, and safety criteria are measured during the treatment. Subjects are provided with an electronic device to enter patient-reported outcome (PRO) data at each study visit. All visit-specific PRO assessments are conducted/completed before any tests, procedures or other clinical assessments, with the exception of the urine pregnancy test and urinalysis, to prevent influencing subject perceptions.

Electrocardiograms (ECGs) precede vital signs and both procedures are completed prior to any invasive procedures. Vital signs are recorded from the opposite arm from which blood samples are being taken.

All samples (including safety, efficacy, PK, photography, and biomarkers) are obtained after the PRO and ECG assessments.

Psoriasis Area and Severity Index (PASI), Body Surface Area (BSA), and Investigator's Global Assessment (IGA) are obtained at Weeks 0, 2, 3, 8, 12, 16, 20, 24, 28, 32, 36, 44, and 52. Nail Psoriasis Area and Severity Index (NAPSI), Fingernail Physician's Global Assessment (f-PGA), Static Physician's Global Assessment of Genitalia (s-PGA-G), Physician's Global Assessment of Hands and/or Feet (hf-PGA), and Scalp Specific Investigator Global Assessment (ss-IGA) are obtained at Weeks 0, 8, 16, 24, 36, and 52.

Adverse events are reported and followed by the investigator. Any clinically relevant changes occurring during the study are recorded. Any clinically significant abnormalities persisting at the end of the study/early withdrawal were followed by the investigator until resolution or until a clinically stable condition was reached if possible. The evaluations of safety and tolerability include: physical examinations, vital signs, electrocardiograms, clinical safety laboratory assessments, review of concomitant medication, pregnancy testing, suicidal ideation evaluation via the Columbia—Suicide Severity Rating Scale, and tuberculosis evaluation.

### Primary Endpoint Analysis

The primary efficacy endpoints are the proportion of participants achieving a PASI 90 response, defined as at least a 90% reduction from baseline in PASI total score, or achieve an IGA 0 or 1 and an at least 2-grade improvement in IGA score after Week 16.

For the primary analysis, composite strategy (non-responder imputation) is applied to address intercurrent event #1 (Discontinuation of study intervention due to lack of efficacy or due to an AE of worsening of psoriasis) and #2 (initiation of a protocol-prohibited medication or therapy that could improve psoriasis); and treatment policy strategy (observed data) is applied to intercurrent event #3 (Discontinuation of study intervention due to other reasons). Participants with missing data after application of ICEs are also considered as non-responders.

The MCP Mod is used to test if there is a positive overall treatment effect based on candidate response models.

### Safety:

Safety is assessed among all randomized and treated participants who received at least 1 dose of study agent (partial or complete) according to the assigned treatment received during the study. This is also referred to as the safety analysis set.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

Each aspect of the present invention defined in this or in any other section may incorporate definitions and limitations, such as those set forth throughout the originally filed disclosure, specification, and claims.

### Example 4. Preparation of a Crystalline Form of a Hydrochloride Salt of a Peptide of SEQ ID NO: 1

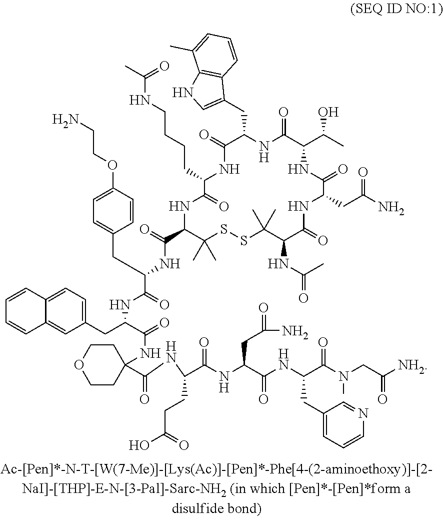 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]

18.9 kg Rink amide AM resin (substitution: 0.95 mmol/g) were loaded into a 1000 L SPPS reactor and the SPPS was performed. Each SPPS cycle consists of Fmoc cleavage, coupling with the respective building block and obligatory capping. For Fmoc cleavage the resin was treated with 20% piperidine in DM (10 ml/g resin each) for 5±2 min and 10±2 min at 25 20° C. Couplings were performed using the building blocks, coupling reagents and conditions depicted in Table 55. with DMF as solvent (10 ml/g resin). For capping, the resin was treated with acidic anhydride and pyridine in DMF (volumetric ratio DMF/Ac₂O/pyridine 50:1:1; DMF: 10 ml/g resin) for 20 min. Diisopropyl carbonate (DIC) was added in two portions, with the second portion was added after about 20 to 30 minutes after the first portion.

TABLE 55
Coupling Conditions

| | | Equivalents | Equivalents | Equivalents | Coupling | Coupling |
|---|---|---|---|---|---|---|
| Cycle | Building block | building block | Oxyma | DIC | duration | temp. |
| 1 | Fmoc-Sar-OH | 2.00 | 3.10 | 2.60 + 1.30 | 3 h | 25° C. |
| 2 | Fmoc-3-Pal-OH | 1.50 | 2.33 | 1.95 + 0.975 | 7 h | 25° C. |
| 3 | Fmoc-Asn(Trt)-OH | 2.00 | 3.10 | 2.60 + 1.30 | 2 h | 25° C. |
| 4 | Fmoc-Glu(OtBu)—OH•H₂O | 2.00 | 3.10 | 2.60 + 1.30 | 14 h | 25° C. |
| 5 | Fmoc-THP-OH | 1.50 | 2.33 | 1.95 + 0.975 | 17 h | 25° C. |
| 6 | Fmoc-2-Nal-OH | 1.50 | 2.33 | 1.95 + 0.975 | 28 h | 25° C. |
| 7 | Fmoc-Tyr(Boc-2- | 1.50 | 2.33 | 1.95 + 0.975 | 6 h | 25° C. |
| | aminoethyl)-OH | | | | | |
| 8 | Fmoc-Pen(Trt)-OH | 2.00 | 3.10 | 2.60 + 1.30 | 4 h | 25° C. |
| 9 | Fmoc-Lys(Ac)—OH | 2.00 | 3.10 | 2.60 + 1.30 | 2 h | 25° C. |
| 10 | Fmoc-7-Me-Trp-OH | 1.80 | 2.79 | 2.34 + 1.17 | 7 h | 25° C. |
| 11 | Fmoc-Thr(tBu)—OH | 2.00 | 3.10 | 2.60 + 1.30 | 2 h | 25° C. |
| 12 | Fmoc-Asn(Trt)-OH | 2.00 | 3.10 | 2.60 + 1.30 | 12 h | 25° C. |
| 13 | Fmoc-Pen(Trt)-OH | 2.00 | 3.10 | 2.60 + 1.30 | 4 h | 25° C. |

Final acetylation of the peptide resin was performed with acidic anhydride and pyridine in DMF (volumetric ratio DMF/Ac₂O/pyridine 10:1:1; DMF: 10 ml/g resin) for 20 min. After drying at 25-35° C. 72.8 kg linear peptide-Rink amide AM resin were obtained.

### TFA Cleavage

In a jacketed reactor 100 g of the above resin were added at 18° C. to 700 mL of the cleavage cocktail, consisting of 630 mL TFA, 35 mL TIS, 17.5 mL EDT, and 17.5 mL water. The temperature increased to 30° C. upon addition of the resin, and the mixture was stirred for further 35 min at 30° C. The mixture was cooled to 20° C., and the resin was filtered off and washed two times with 100 mL TFA each. The filtrates were combined and cooled to −15° C. For precipitation 4.0 L diisopropyl ether were added within 20 min maintaining the temperature of the solution/suspension at 7° C. After complete addition of diisopropyl ether, the temperature was increased to 22° C. and the suspension stirred for 2.5 h.

The suspension was then transferred to a filter dryer, and the precipitated crude product was filtered off at ambient temperature. The filter cake was subsequently washed three times with 300 mL of diisopropyl ether each and dried in vacuo at 30° C. over night to yield 52.87 g of linear peptide as the TFA salt.

### Oxidation and Purification by Preparative HPLC

28 g of linear peptide as the TFA salt was dissolved in 280 mL 30% AcOH in water at ambient temperature. 3.71 g iodine and 7.17 g potassium iodide were dissolved in 280 mL water. Both the peptide and the iodine/iodide-solution were added in parallel to a vigorously stirred mixture of 2.2 L 30% AcOH in water at ambient temperature within 60 min. After complete addition of both solutions, a brown oxidation mixture was obtained. After stirring for 30 min at ambient temperature, IPC indicated nearly full conversion of starting material. 3.5 g Vitamin C were added 1.5 h after complete addition of the peptide and iodine solutions. The then obtained yellow solution was stirred for 10 min. The oxidation mixture was filtrated over a fritted glass funnel before applying to the prep. RP-HPLC column. The chromatographic conditions were the same as used in Example 4. All fractions were adjusted with 18% HCl in water to pH 7. The fractions collected during prep. RP-HPLC were analyzed by UHPLC. Fractions containing>98% product were pooled for subsequent isolation.

### Isolation

420 mL of product pool had been obtained after oxidation and preparative HPLC purification. By a test lyophilization the product concentration of this solution was determined as approx. 29 g/L, which corresponds to a theoretical yield of approx. 12.2 g.

From the overall pool volume, 120 mL were transferred to a round-bottom flask, and the initial pH of 7.51 was adjusted with 4.5 mL of 1 M HCl aq. to pH 3.00. Acetonitrile was evaporated from the product solution at 40° C. in vacuo, until water started to evaporate. After evaporation, 80 mL of aqueous product solution were remaining (pH 2.54), of which 40 mL were transferred to a reactor connected to a heating/cooling system and applied for the subsequent isolation.

The pH of the solution was then adjusted to pH 3.75 by addition of 1.0 mL of 0.5 M NH₄HCO₃ within 65 min. To the clear yellow solution 1% (w w) the compound of Formula (I) was added as seeding material and the formed thin suspension was stirred for 60 min at 25° C. The pH of the suspension was then adjusted to pH 4.50 by addition of 2.9 mL of 0.5 M NH₄HCO₃ within 125 min. After stirring the suspension for 30 min at 25° C. the pH had dropped to pH 4.12. The pH was then re-adjusted to 4.50 by addition of 0.2 mL of 0.5 M NH₄HCO₃. After stirring for 16 h at 25° C. a thick suspension was present, which was filtered (1 min filtration time) over a glass nutsche filter (G4) and washed without stirring with 1.59 mL of water (1 vol. eq.; 1 min filtration time). The washed filter cake was dried in vacuo at 25° C. for 16 h in a vacuum oven. The dried product was finally unloaded from the filter.

In total, 1.77 g of the compound of Formula (I) (partial HCl salt) was isolated out of 60 mL of product pool, which calculates to 12.4 g of the compound of Formula (I) out of the entire 420 mL of product pool. For the isolated material a purity (HPLC) of 99.4%, a chloride content (titration) of 1.6%, a water content (KF) of 3.6%.

An X-ray powder diffraction (XRPD) pattern of the partial hydrochloride salt of the compound of Formula (I) confirmed its crystallinity. The following two theta peaks were observed: 4.2920, 6.9201, 7.6600, 8.5693, 9.2667, 9.9817, 10.7256, 11.5429, 11.9937, 13.0633, 13.3317, 13.9650, 14.7879, 15.8430, 17.1481, 17.6468, 18.1402, 18.6158, 19.3291, 20.4899, 20.7090, or 21.7813+/−0.2 degrees two theta.

### Example 5. Synthetic Procedure for a Crystalline Hydrochloride Salt of a Peptide of SEQ ID NO: 1

Crystalline hydrochloride salt of the compound of Formula (I) (30 g) prepared according to Example 4 was dissolved in 120 mL of methanol and 51.4 mL of water. The dissolution was carried out in an EasyMax 402, under stirring at 40° C. 57.1 mL of sodium chloride 1 M were dosed to the EasyMax reactor at over 1 h. The solution was then seeded with 300 mg of seeds of the hydrochloride salt of the compound of Formula (I) prepared according to Example 4. The slurry was then aged for 8 h under stirring at 40° C. The slurry was cooled to 5° C. at a cooling rate of 0.1K/min. An extra 114.31 mL of sodium chloride 1 M were dosed to the EasyMax reactor over 4 h and the slurry was aged for extra 5 h. The solid was isolated by vacuum filtration and washed twice with 30 mL of water and twice with 30 mL of isopropanol. The solid was dried atmospherically.

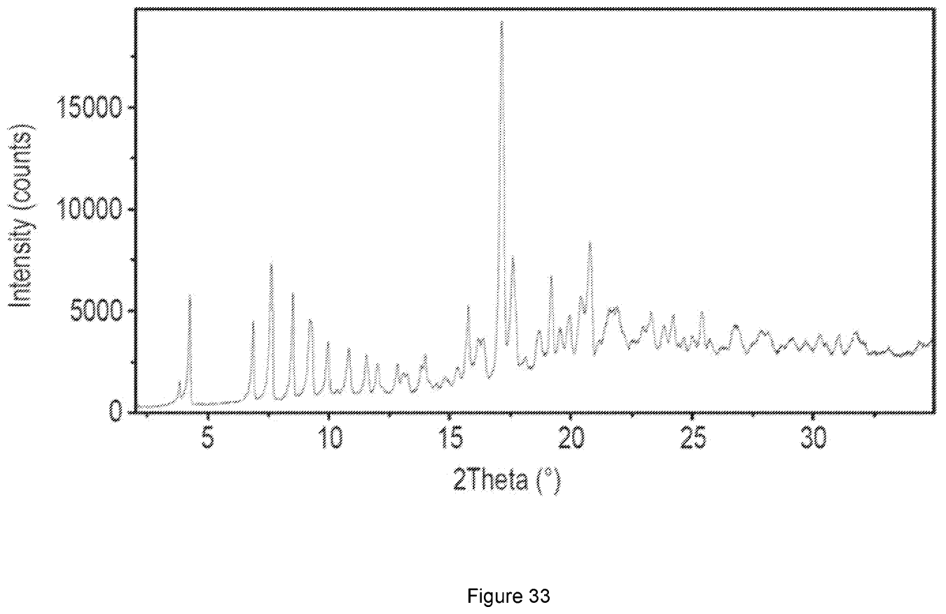

The isolated solid of the crystalline hydrochloride form of the compound of Formula (I) had a purity (UPLC) of 99.3 area % and a water content (KF) of 6.33 w/w %. The chloride content of the isolated crystalline hydrochloride form of the compound of Formula (I) was assayed by ion chromatography and found to be 0.64 molar equivalents to the compound of Formula (I). An XRPD pattern of the crystalline hydrochloride form of the compound of Formula (I) confirmed its crystallinity (FIG. 33). The following two theta peaks were observed: 3.8, 4.2, 6.9, 7.6, 8.5, 9.3, 9.4, 10.0, 10.8, 11.6, 12.0, 12.2, 12.8, 13.1, 13.3, 13.8, 13.9, 14.2, 14.4, 14.7, 15.3, 15.7, 16.2, 16.4, 17.2, 17.6, 18.2, 18.7, 19.2, 19.6, 19.9, 20.5, and 20.8 degrees two theta+/−0.2 degrees two theta.

Although the foregoing invention has been described in some detail by way of illustration and Example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference. Where a conflict exists between the instant application and a reference provided herein, the instant application shall dominate.

Each aspect of the present invention defined in this or in any other section may incorporate definitions and limitations, such as those set forth throughout the originally filed disclosure, specification and claims.

What is claimed is:

1. A method for treating psoriasis in a subject in need thereof, comprising administering a compound of Formula (I): 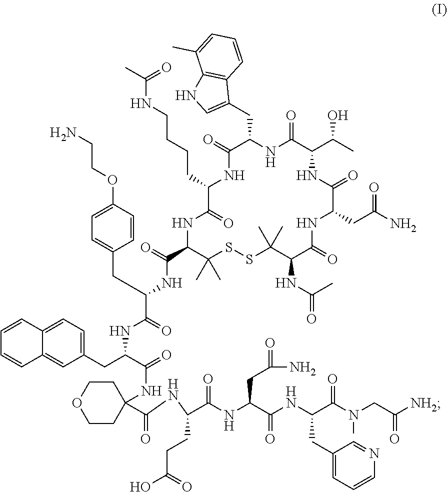 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or
a pharmaceutically acceptable salt or solvate thereof, to a patient in need thereof:
wherein after treating with the compound of Formula (I) or a pharmaceutically acceptable salt or solvate thereof, the subject is a responder to treatment by at least one psoriasis measure of response to treatment selected from the group consisting of:
(i) a 75% or higher reduction in Psoriasis Area and Severity Index (PASI) compared to a baseline PASI;
(ii) an Investigator's Global Assessment (IGA) Score of 2 or lower after treatment;
(iii) reduction from baseline in Psoriasis Symptoms and Signs Diary (PSSD) Symptoms Score by 1 or higher; and
(iv) a reduction of Dermatological Life Quality Index (DLQI) score.

2. A method of treating psoriasis in a subject in need thereof, comprising administering a compound of Formula (I): 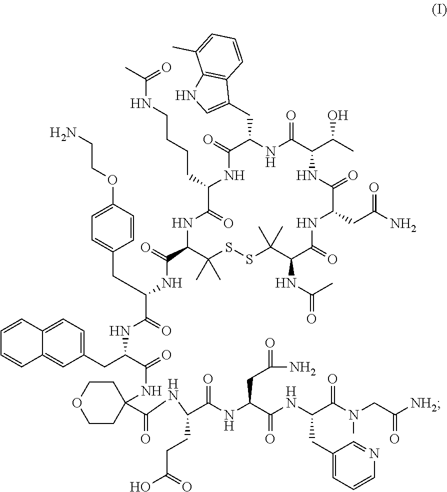 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or a pharmaceutically acceptable salt or solvate thereof,
wherein the subject achieves a 75% or higher reduction in Psoriasis Area and Severity Index (PASI) score compared to a baseline PASI score.

3. The method of claim 1 or 2, wherein the subject achieves a psoriasis measure of response of at least about a 90% reduction in PASI score compared to the baseline PASI score.

4. The method of claim 3, wherein the subject achieves a psoriasis measure of response of about 100% reduction in PASI score compared to the baseline PASI score.

5. The method of claim 1, wherein the subject achieves a psoriasis measure of response of an at least 2-point decrease in IGA score.

6. The method of claim 1 or 5, wherein the subject achieves an Investigator's Global Assessment (IGA) Score of 2 or lower.

7. The method of claim 6, wherein the subject achieves an IGA score of 1 or lower.

8. The method of claim 7, wherein the subject achieves an IGA score of 0.

9. The method of claim 1 or 5, wherein the patient achieves an Investigator's Global Assessment (IGA) Score of 2 or lower after treatment.

10. The method of claim 9, wherein the patient achieves an IGA score of 1 or lower after treatment.

11. The method of claim 10, wherein the patient achieves an IGA score of 0 after treatment.

12. The method of claim 1 or 2, wherein the psoriasis measure of response is measured at least 2 weeks after the beginning of treatment.

13. The method of claim 1 or 2, wherein the psoriasis measure of response is measured at least 4 weeks after the beginning of treatment.

14. The method of claim 1 or 2, wherein the psoriasis measure of response is measured at least 8 weeks after beginning of treatment.

15. The method of claim 1 or 2, wherein the psoriasis measure of response is measured at least 16 weeks after beginning of treatment.

16. The method of claim 1 or 2, wherein the psoriasis measure of response is measured at least 20 weeks after beginning of treatment.

17. The method of claim 1 or 2, wherein the psoriasis measure of response is measured at least 24 weeks after beginning of treatment.

18. The method of claim 1 or 2, wherein the psoriasis measure of response is measured at least 28 weeks after beginning of treatment.

19. The method of claim 1 or 2, wherein the psoriasis measure of response is measured at least 32 weeks after beginning of treatment.

20. The method of claim 1 or 2, wherein the psoriasis measure of response is measured at least 40 weeks after beginning of treatment.

21. The method of claim 1 or 2, wherein the psoriasis measure of response is measured at least 52 weeks after beginning of treatment.

22. The method of claim 1 or 2, wherein the psoriasis is plaque psoriasis.

23. The method of claim 22, wherein the psoriasis is moderate to severe plaque psoriasis.

24. The method of claim 1 or 2, wherein the subject is a candidate for phototherapy or systematic therapy.

25. The method of claim 1 or 2, wherein the compound of Formula (I) or pharmaceutically acceptable salt or solvate thereof is a hydrochloride salt of the compound of Formula (I) or solvate thereof.

26. The method of claim 1 or 2, comprising orally administering the compound of Formula (I) or pharmaceutically acceptable salt or solvate thereof.

27. The method of any claim 1 or 2, comprising administering the compound of Formula (I) or pharmaceutically acceptable salt or solvate thereof daily.

28. The method of claim 1 or 2, comprising administering the compound of Formula (I) or pharmaceutically acceptable salt or solvate thereof in an amount of about 5 mg to about 800 mg.

29. The method of claim 28, comprising administering the compound of Formula (I) or pharmaceutically acceptable salt or solvate thereof in an amount of about 25 mg to about 500 mg.

30. The method of claim 29, comprising administering the compound of Formula (I) or pharmaceutically acceptable salt or solvate thereof in an amount of about 50 mg to about 500 mg.

31. The method of claim 30, comprising administering the compound of Formula (I) or pharmaceutically acceptable salt or solvate thereof in an amount of about 25 mg.

32. The method of claim 30, comprising administering the compound of Formula (I) or pharmaceutically acceptable salt or solvate thereof in an amount of about 50 mg.

33. The method of claim 30, comprising administering the compound of Formula (I) or pharmaceutically acceptable salt or solvate thereof in an amount of about 100 mg.

34. The method of claim 30, comprising administering the compound of Formula (I) or pharmaceutically acceptable salt or solvate thereof in an amount of about 200 mg.

35. The method of claim 1 or 2, wherein the patient has a Body Surface Area (BSA) of at least 10% prior to treatment.

36. The method of claim 1 or 2, wherein the patient has a PASI score of about 12 to 72 prior to treatment.

37. The method of claim 1 or 2, wherein the subject has an Investigator's Global Assessment (IGA) of at least 3 prior to treatment.

38. The method of claim 1 or 2, wherein the subject has a Psoriasis Symptom and Sign Diary (PSSD) symptoms score of at least 1 prior to treatment.

39. The method of claim 1 or 2, wherein the subject has a Dermatological Life Quality Index (DLQI) score of greater than 1 prior to treatment.

40. The method of claim 1 or 2, wherein, before the start of treatment with the compound of Formula (I) or pharmaceutically acceptable salt or solvate thereof, the subject has not been treated with a biologic agent for psoriasis.

41. The method of claim 1 or 2, wherein, before the start of treatment with the compound of Formula (I) or pharmaceutically acceptable salt or solvate thereof, the subject has not been treated with any IL-23 receptor antagonist for psoriasis.

42. A pharmaceutical product for treating a subject diagnosed with psoriasis, comprising a compound of Formula (I): 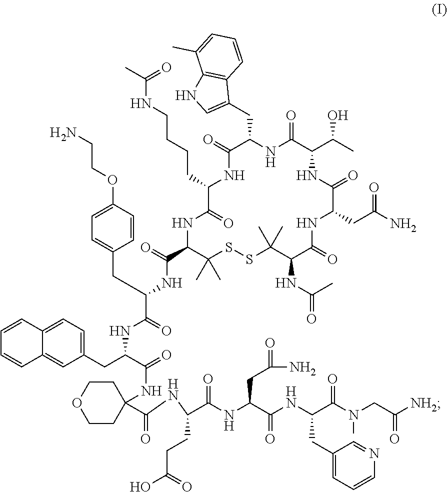 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or a pharmaceutically acceptable salt or solvate thereof, that when administered to said subject, in an amount of at least 25 mg, induces a mean reduction in the Psoriasis Area and Severity Index (PASI) by about 75% or greater, when measured from a baseline PASI score.

43. The pharmaceutical product of claim 42, wherein the subject achieves at least about a 90% reduction in Psoriasis Area and Severity Index score compared to the baseline.

44. The pharmaceutical product of claim 43, wherein the subject achieves about 100% reduction in Psoriasis Area and Severity Index score compared to the baseline.

45. The pharmaceutical product of claim 42, wherein the patient achieves an Investigator's Global Assessment (IGA) Score of 2 or lower after treatment.

46. The pharmaceutical product of any one of claims 42 to 45, wherein the compound of Formula (I) or pharmaceutically acceptable salt or solvate thereof is in an amount of about 25 mg to about 800 mg.

47. The pharmaceutical product of claim 46, wherein the compound of Formula (I) or pharmaceutically acceptable salt or solvate thereof is in an amount of about 50 mg to about 800 mg.

48. The pharmaceutical product of claim 47, wherein the compound of Formula (I) or pharmaceutically acceptable salt or solvate thereof is in an amount of about 50 mg to about 500 mg.

49. The pharmaceutical product of claim 46, wherein the compound of Formula (I) or pharmaceutically acceptable salt or solvate thereof is in an amount of about 25 mg.

50. The pharmaceutical product of claim 48, wherein the compound of Formula (I) or pharmaceutically acceptable salt or solvate thereof is in an amount of about 50 mg.

51. The pharmaceutical product of claim 48, wherein the compound of Formula (I) or pharmaceutically acceptable salt or solvate thereof is in an amount of about 100 mg.

52. The pharmaceutical product of claim 48, wherein the compound of Formula (I) or pharmaceutically acceptable salt or solvate thereof is in an amount of about 200 mg.

53. A method for treating psoriasis in a subject in need thereof, comprising administering a compound of Formula (I): 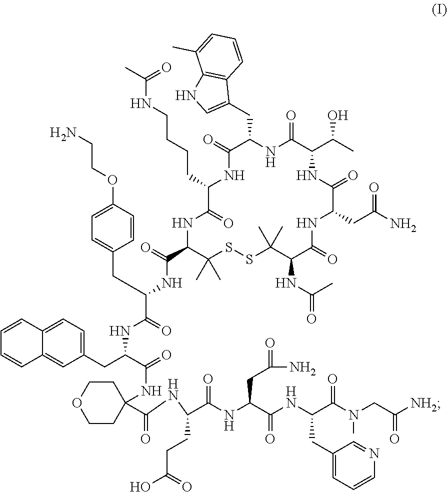 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or a pharmaceutically acceptable salt or solvate thereof, to a patient in need thereof in an amount of about 200 mg.

54. The method of claim 53, wherein the method comprises administering the compound of Formula (I) or a pharmaceutically acceptable salt or solvate thereof in an amount of about 200 mg once daily.

55. The method of claim 53, wherein the method further comprises a step of achieving one or more selected from the group consisting of:
(i) a 75% or higher reduction in Psoriasis Area and Severity Index (PASI) compared to a baseline PASI;
(ii) an Investigator's Global Assessment (IGA) Score of 2 or lower after treatment:
(iii) reduction from baseline in Psoriasis Symptoms and Signs Diary (PSSD) Symptoms Score by 1 or higher; and
(iv) a reduction of Dermatological Life Quality Index (DLQI) score.

56. The method of any one of claims 53 to 55, wherein the psoriasis is plaque psoriasis.

57. The method of claim 56, wherein the psoriasis is moderate to severe plaque psoriasis.

58. The method of any one of claims 53 to 55, wherein the subject is a candidate for phototherapy or systematic therapy.

59. The method of any one of claims 53 to 55, wherein the compound of Formula (I) or pharmaceutically acceptable salt or solvate thereof is a hydrochloride salt of the compound of Formula (I) or solvate thereof.

60. The method of any one of claims 53 to 55, comprising orally administering the compound of Formula (I) or pharmaceutically acceptable salt or solvate thereof.

61. The method of any one of claims 53 to 65, comprising administering the compound of Formula (I) or pharmaceutically acceptable salt or solvate thereof daily.

62. The method of any one of claims 53 to 55, wherein the patient has a Body Surface Area (BSA) of at least 10%/prior to treatment.

63. The method of any one of claims 53 to 55, wherein the patient has a PASI score of about 12 to 72 prior to treatment.

64. The method of any one of claims 53 to 55, wherein the subject has an Investigator's Global Assessment (IGA) of at least 3 prior to treatment.

65. The method of any one of claims 53 to 55, wherein the subject has a Psoriasis Symptom and Sign Diary (PSSD) symptoms score of at least 1 prior to treatment.

66. The method of any one of claims 53 to 55, wherein the subject has a PSSD signs score of at least 1 prior to treatment.

67. The method of any one of claims 53 to 55, wherein the subject has a Dermatological Life Quality Index (DLQI) score of greater than 1 prior to treatment.

68. The method of any one of claims 53 to 55, wherein, before the start of treatment with the compound of Formula (I) or pharmaceutically acceptable salt or solvate thereof, the subject has not been treated with a biologic agent for psoriasis.

69. The method of any one of claims 53 to 55, wherein, before the start of treatment with the compound of Formula (I) or pharmaceutically acceptable salt or solvate thereof, the subject has not been treated with any IL-23 receptor antagonist for psoriasis.

70. A method of treating a patient population diagnosed with psoriasis, comprising administering a compound of Formula (I): 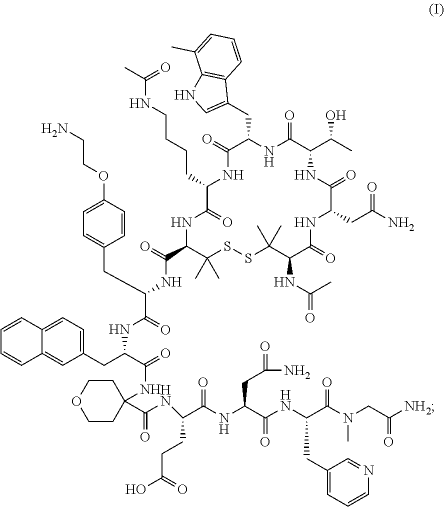 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or a pharmaceutically acceptable salt or solvate thereof, to said patient population in an amount of about 200 mg.

71. The method of claim 70, wherein the method comprises administering the compound of Formula (I) or a pharmaceutically acceptable salt or solvate thereof in an amount of about 200 mg daily.

72. The method of claim 70, wherein the method further comprises a step of achieving one or more selected from the group consisting of:
(i) an average of a 75% or higher reduction in Psoriasis Area and Severity Index (PASI) compared to a baseline PASI in the patient population;
(ii) an average Investigator's Global Assessment (IGA) Score of 2 or lower after treatment in the patient population;
(iii) an average reduction from baseline in Psoriasis Symptoms and Signs Diary (PSSD) Symptoms Score by 1 or higher in the patient population; and
(iv) a reduction of Dermatological Life Quality Index (DLQI) score in at least 50% of the patient population.

73. The method of any one of claims 70 to 72, wherein the psoriasis is plaque psoriasis.

74. The method of claim 73, wherein the psoriasis is moderate to severe plaque psoriasis.

75. The method of any one of claims 70 to 72, wherein the compound of Formula (I) or pharmaceutically acceptable salt or solvate thereof is a hydrochloride salt of the compound of Formula (I) or solvate thereof.

76. The method of any one of claims 70 to 72, comprising orally administering the compound of Formula (I) or pharmaceutically acceptable salt or solvate thereof.

77. The method of any one of claims 70 to 72, comprising administering the compound of Formula (I) or pharmaceutically acceptable salt or solvate thereof daily.

78. The method of any one of claims 70 to 72, wherein a patient in the patient population has a Body Surface Area (BSA) of at least 10% prior to treatment.

79. The method of any one of claims 70 to 72, wherein a patient in the patient population has a PASI score of about 12 to 72 prior to treatment.

80. The method of any one of claims 70 to 72, wherein a patient in the patient population has an Investigator's Global Assessment (IGA) of at least 3 prior to treatment.

81. The method of any one of claims 70 to 72, wherein a patient in the patient population has a Psoriasis Symptom and Sign Diary (PSSD) symptoms score of at least 1 prior to treatment.

82. The method of any one of claims 70 to 72, wherein, before the start of treatment with the compound of Formula (I) or pharmaceutically acceptable salt or solvate thereof, a patient in the patient population has not been treated with a biologic agent for psoriasis.

83. The method of any one of claims 70 to 72, wherein, before the start of treatment with the compound of Formula (I) or pharmaceutically acceptable salt or solvate thereof, a patient in the patient population has not been treated with any IL-23 receptor antagonist for psoriasis.

84. The method of any one of claims 70 to 72, wherein the subject is a patient.

85. The method of claim 84, wherein the subject is an adult or adolescent patient.

86. The method of any one of claims 70 to 85, wherein the compound of Formula (I) or pharmaceutically acceptable salt or solvate thereof is a hydrochloride salt.

87. The method of any one of claims 70 to 86, wherein the compound of Formula (I) or pharmaceutically acceptable salt or solvate thereof is a hydrochloride salt in a crystalline form.

88. A pharmaceutical product for treating a subject with psoriasis, comprising a compound of Formula (I): 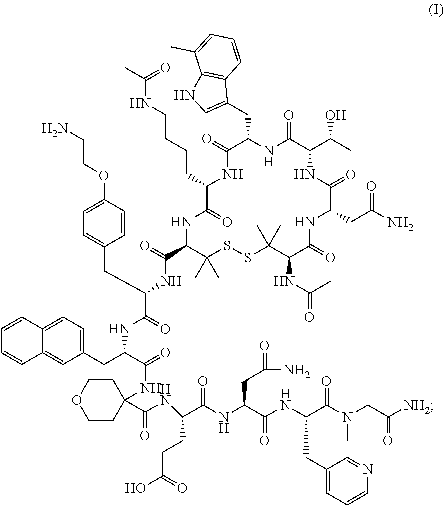 [SMILES: CC(=O)NCCCC[C@@H]1NC(=O)[C@H](Cc2c[nH]c3c(C)cccc23)NC(=O)[C@H]([C@@H](C)O)NC(=O)[C@H](CC(N)=O)NC(=O)[C@@H](NC(C)=O)C(C)(C)SSC(C)(C)[C@@H](C(=O)N[C@@H](Cc2ccc(OCCN)cc2)C(=O)N[C@@H](Cc2ccc3ccccc3c2)C(=O)NC2(C(=O)N[C@@H](CCC(=O)O)C(=O)N[C@@H](CC(N)=O)C(=O)N[C@@H](Cc3cccnc3)C(=O)N(C)CC(N)=O)CCOCC2)NC1=O]
or a pharmaceutically acceptable salt or solvate thereof, wherein the compound of Formula (I) or pharmaceutically acceptable salt or solvate thereof is in an amount of about 200 mg.
